(19) United States
(12) Patent Application Publication
(10) Pub. No.: US 2010/0093811 A1
(43) Pub. Date: Apr. 15, 2010

(54) HIV PROTEASE INHIBITORS

(76) Inventors: Craig A. Coburn, Royersford, PA (US); Joseph P. Vacca, Telford, PA (US); Hemaka A. Rajapakse, Wyncote, PA (US); Kristen L.G. Jones, Jeffersonville, PA (US); Philippe Nantermet, Lansdale, PA (US); James C. Barrow, Harleysville, PA (US); Keith P. Moore, Harleysville, PA (US); Steven S. Sharik, Ambler, PA (US); Cory Theberge, Elkins Park, PA (US); Abbas M. Walji, Lansdale, PA (US)

Correspondence Address: MERCK AND CO., INC, P O BOX 2000, RAHWAY, NJ, 07065-0907, US

(21) Appl. No.: 12/523,200

(22) PCT Filed: Sep. 22, 2008
(86) PCT No.: PCT/US08/10971

§ 371 Date: Jul. 15, 2009

Related U.S. Application Data
(60) Provisional application No. 60/995,202, filed on Sep. 25, 2007.
(60) Provisional application No. 61/188,684, filed on Aug. 12, 2008.

(51) Int. Cl. A61K 31/44; C07C 311/15; A61K 31/18; C07C 271/06; A61K 31/27; C07D 277/62; A61K 31/426; C07D 213/55; A61P 31/18

(52) U.S. Cl. 514/357; 564/86; 514/603; 560/115; 514/487; 548/178; 514/367; 546/335

(57) ABSTRACT

Compounds of Formula I are disclosed: (I), wherein X^{A}, k, R¹, R², R³, R⁴, R⁵, R^{5A}, R⁶, R^{6A}, R⁷ and R⁸ are defined herein. The compounds encompassed by Formula I include compounds which are HIV protease inhibitors and other compounds which can be metabolized in vivo to HIV protease inhibitors. The compounds and their pharmaceutically acceptable salts are useful for the prophylaxis or treatment of infection by HIV and the prophylaxis, treatment, or delay in the onset of AIDS. The compounds and their salts can be employed as ingredients in pharmaceutical compositions, optionally in combination with other antivirals, immunomodulators, antibiotics or vaccines.

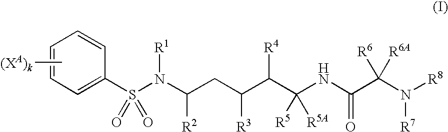

### FIELD OF THE INVENTION

The present invention is directed to certain lysine sulfonamide derivatives and their pharmaceutically acceptable salts. Some of these derivatives are compounds which are HIV protease inhibitors and the others can be metabolized in vivo to HIV protease inhibitors. The compounds are useful for the prophylaxis of HIV infection and HIV replication, the treatment of HIV infection and HIV replication, the prophylaxis of AIDS, the treatment of AIDS, and the delay in the onset and/or progression of AIDS.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of acquired immunodeficiency syndrome (AIDS), a disease characterized by the destruction of the immune system, particularly of CD4 T-cells, with attendant susceptibility to opportunistic infections, and its precursor AIDS-related complex (“ARC”), a syndrome characterized by symptoms such as persistent generalized lymphadenopathy, fever and weight loss. This virus was previously known as LAV, HTLV-III, or ARV. A common feature of retrovirus replication is the extensive post-translational processing of precursor polyproteins by a virally encoded protease to generate mature viral proteins required for virus assembly and function. Inhibition of this processing prevents the production of normally infectious virus. For example, Kohl et al., Proc. Nat'l Acad. Sci. 1988, 85: 4686, demonstrated that genetic inactivation of the HIV encoded protease resulted in the production of immature, non-infectious virus particles. These results indicated that inhibition of the HIV protease represents a viable method for the treatment of AIDS and the prevention or treatment of infection by HIV.

Nucleotide sequencing of HIV shows the presence of a pol gene in one open reading frame [Ratner et al., Nature 1985, 313: 277]. Amino acid sequence homology provides evidence that the pol sequence encodes reverse transcriptase, an endonuclease, HIV protease and gag, which encodes the core proteins of the virion (Toh et al., EMBO J 1985, 4: 1267; Power et al., Science 1986, 231: 1567; Pearl et al., Nature 1987, 329: 351].

Several HIV protease inhibitors are presently approved for clinical use in the treatment of AIDS and HIV infection, including indinavir (see U.S. Pat. No. 5,413,999), amprenavir (U.S. Pat. No. 5,585,397), saquinavir (U.S. Pat. No. 5,196,438), ritonavir (U.S. Pat. No. 5,484,801) and nelfinavir (U.S. Pat. No. 5,484,926). Each of these protease inhibitors is a peptide-derived peptidomimetic, competitive inhibitor of the viral protease which prevents cleavage of the HIV gag-pol polyprotein precursor. Tipranavir (U.S. Pat. No. 5,852,195) is a non-peptide peptidomimetic protease inhibitors also approved for use in treating HIV infection. The protease inhibitors are administered in combination with at least one and typically at least two other HIV antiviral agents, particularly nucleoside reverse transcriptase inhibitors such as zidovudine (AZT) and lamivudine (3TC) and/or non-nucleoside reverse transcriptase inhibitors such as efavirenz and nevirapine. Indinavir, for example, has been found to be highly effective in reducing HIV viral loads and increasing CD4 cell counts in HIV-infected patients, when used in combination with nucleoside reverse transcriptase inhibitors. See, for example, Hammer et al., New England J. Med. 1997, 337: 725-733 and Gulick et al., New England J. Med. 1997, 337: 734-739.

The established therapies employing a protease inhibitor are not suitable for use in all HIV-infected subjects. Some subjects, for example, cannot tolerate these therapies due to adverse effects. Many HIV-infected subjects often develop resistance to particular protease inhibitors. Accordingly, there is a continuing need for new compounds which are capable of inhibiting HIV protease and suitable for use in the treatment or prophylaxis of infection by HIV and/or for the treatment or prophylaxis or delay in the onset or progression of AIDS.

References disclosing amino acid derivatives with HIV aspartyl protease inhibiting properties, processes for preparing the derivatives, and/or therapeutic uses of the derivatives include: WO 01/68593, WO 02/064551 A1, WO 03/074467 A2, WO 2004/056764 A1, WO 2006/012725 A1, WO 2006/114001 A1, WO 2007/062526 A1, WO 2008/023273 A2, WO 2008/078200 A2, and U.S. Pat. No. 7,388,008 B2.

### SUMMARY OF THE INVENTION

The present invention is directed to certain lysine sulfonamide derivatives and their use in the inhibition of HIV protease, the prophylaxis of infection by HIV, the treatment of infection by HIV, and the prophylaxis, treatment, and delay in the onset or progression of AIDS. More particularly, the present invention includes compounds of Formula I:

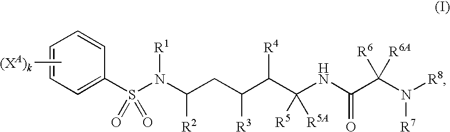

and pharmaceutically acceptable salts thereof, wherein:
R¹ is C₁₋₆ alkyl, C₁₋₆ fluoroalkyl, C₃₋₆ cycloalkyl, or C₁₋₆ alkyl substituted with C₃₋₆ cycloalkyl;

### R² is CH(R^{J})—Z, and Z is OH, NH₂, or OR^{P};

R^{J} is H, C₁₋₆ alkyl, C₁₋₆ fluoroalkyl, or C₁₋₆ alkyl substituted with C₃₋₅ cycloalkyl;

### R^{P} is P(O)(OH)₂, P(O)(OM)₂, or C(O)R^{Q};

M is an alkali metal or an alkaline earth metal;

### R^{Q} is:

(1) C₁₋₆ alkyl,

(2) C₃₋₆ cycloalkyl,

(3) C₁₋₆ alkyl substituted with C₃₋₆ cycloalkyl,

(4) O—C₁₋₆ alkyl,

(5) O—C₁₋₆ alkyl substituted with O—C₁₋₆ alkyl,

(6) O—C₁₋₆ fluoroalkyl,

(7) C(O)O—C₁₋₆ alkyl,

(8) C(O)—C₁₋₆ alkylene-N(H)—C₁₋₆ alkyl,

(9) C(O)—C₁₋₆ alkylene-N(—C₁₋₆ alkyl)₂,

(10) C₁₋₆ alkyl substituted with C(O)O—C₁₋₆ alkyl,

(11) C₁₋₆ alkyl substituted with C(O)OH,

(12) C₁₋₆ alkyl substituted with C(O)—C₁₋₆ alkyl,

(13) N(H)—C₁₋₆ alkyl,

(14) N(—C₁₋₆ alkyl)₂,

(15) C₁₋₆ alkyl substituted with NH₂, N(H)—C₁₋₆ alkyl, or N(—C₁₋₆ alkyl)₂,

(16) AryA,

(17) C₁₋₆ alkyl substituted with AryA,

(18) O—C₁₋₆ alkyl substituted with AryA,

(19) HetA,

(20) C₁₋₆ alkyl substituted with HetA,

(21) O—C₁₋₆ alkyl substituted with HetA,

(22) HetB, or

(23) O-HetB;

R³ is H, C₁₋₆ alkyl, C₁₋₆ fluoroalkyl, or C₁₋₆ alkyl substituted with C₃₋₆ cycloalkyl;
R⁴ is H, C₁₋₆ alkyl, C₁₋₆ fluoroalkyl, or C₁₋₆ alkyl substituted with C₃₋₆ cycloalkyl;
R⁵ is H, C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl;
R^{5A} is H or C₁₋₆ alkyl; alternatively, R⁵ and R^{5A} together with the carbon atom to which they are both attached form C₃₋₆ cycloalkyl;
and provided that:

(A) when R² is CH₂OH or CH₂OR^{P}, then at least one of R³, R⁴, R⁵ and R^{5A} is other than H;

(B) when either or both R⁵ and R^{5A} are other than H, then at least one of R³ and R⁴ is H; and

(C) when R³ and R⁴ are both, other than H, then R⁵ and R^{5A} are both H;

each X^{A} is independently:

(1) C₁₋₆ alkyl,

(2) C₃₋₆ cycloalkyl,

(3) C₁₋₆ haloalkyl,

(4) OH

(5) O—C₁₋₆ alkyl,

(6) O—C₁₋₆ haloalkyl,

(7) O—C₃₋₆ cycloalkyl,

(8) SH,

(9) S—C₁₋₆ alkyl,

(10) S—C₁₋₆ haloalkyl,

(11) S—C₃₋₆ cycloalkyl,

(12) halo,

(13) CN,

(14) NO₂,

(15) NH₂,

(16) N(H)—C₁₋₆ alkyl,

(17) N(—C₁₋₆ alkyl)₂,

(18) N(H)C(O)—C₁₋₆ alkyl,

(19) N(H)CH(O),

(20) CH(O),

(21) C(O)—C₁₋₆ alkyl,

(22) C(O)OH,

(23) C(O)O—C₁₋₆ alkyl,

(24) SO₂H,

(25) SO₂—C₁₋₆ alkyl, or

(26) C₁₋₆ alkyl substituted with:

(a) C₃₋₆ cycloalkyl,

(b)C₁₋₆ haloalkyl,

(c)OH

(d)O—C₁₋₆ alkyl,

(e)O—C₁₋₆ haloalkyl,

(f)O—C₃₋₆ cycloalkyl,

(g) SH,

(h) S—C₁₋₆ alkyl,

(i) S—C₁₋₆ haloalkyl,

(j) S—C₃₋₆ cycloalkyl,

(k) halo,

(l) CN,

(m) NO₂,

(n) NH₂,

(o) N(H)—C₁₋₆ alkyl,

(p) N(—C₁₋₆ alkyl)₂,

(q) N(H)C(O)—C₁₋₆ alkyl,

(r) N(H)CH(O),

(s)CH(O),

(t)C(O)—C₁₋₆ alkyl,

(u)C(O)OH,

(v) C(O)O—C₁₋₆ alkyl,

(w) SO₂H, or

(x) SO₂—C₁₋₆ alkyl;

or, alternatively, when two or more X^{A} substituents are present on the phenyl ring and two of the X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
k is an integer equal to 0, 1, 2, or 3;

### R⁶ is:

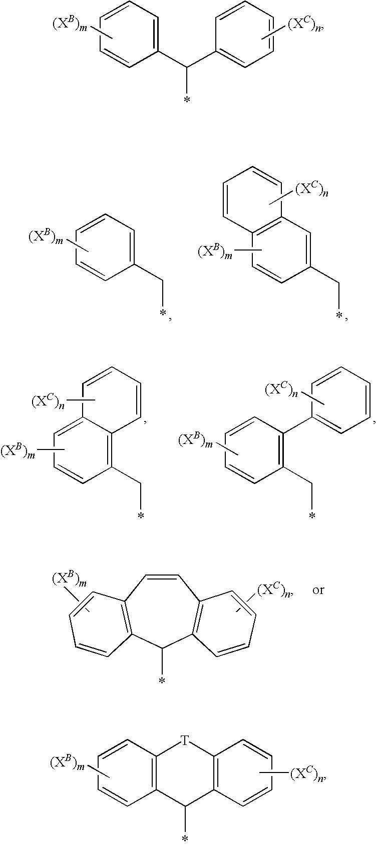

wherein the asterisk (*) denotes the point of attachment to the rest of the compound;
R^{6A} is H or C₁₋₆ alkyl;
alternatively, R⁶ and R^{6A} together with the carbon to which they are attached form a C₃₋₆ cycloalkyl which is optionally substituted with phenyl, wherein the phenyl is optionally substituted with from 1 to 3 X^{B}.
each X^{B} and each X^{C} are independently selected from the group consisting of:

(1) C₁₋₆ alkyl,

(2) C₃₋₆ cycloalkyl,

(3) C₁₋₆ haloalkyl,

(4) OH,

(5) O—C₁₋₆ alkyl,

(6) O—C₁₋₆ haloalkyl,

(7) O—C₃₋₆ cycloalkyl,

(8) SH,

(9) S—C₁₋₆ alkyl,

(10) S—C₁₋₆ haloalkyl,

(11) S—C₃₋₆ cycloalkyl,

(12) halo,

(13) CN,

(14) NO₂,

(15) NH₂,

(16) N(H)—C₁₋₆ alkyl,

(17) N(—C₁₋₆ alkyl)₂,

(18) N(H)C(O)—C₁₋₆ alkyl,

(19) N(H)CH(O),

(20) CH(O),

(21) C(O)—C₁₋₆ alkyl,

(22) C(O)OH,

(23) C(O)O—C₁₋₆ alkyl,

(24) SO₂H,

(25) SO₂—C₁₋₆ alkyl; and

(26) C₁₋₆ alkyl substituted with:

(a) C₁₋₆ haloalkyl,

(b) OH

(c) O—C₁₋₆ alkyl,

(d) O—C₁₋₆ haloalkyl,

(e) O—C₃₋₆ cycloalkyl,

(f) SH,

(g) S—C₁₋₆ alkyl,

(h) halo,

(i) CN,

(i) NO₂,

(k) NH₂,

(l) N(H)—C₁₋₆ alkyl,

(m) N(—C₁₋₆ alkyl)₂;

(n) C(O)—C₁₋₆ alkyl,

(o) C(O)OH,

(p) C(O)O—C₁₋₆ alkyl, or

(q) SO₂—C₁₋₆ alkyl;

### T is O, S, S(O), or SO₂;

m is an integer equal to 0, 1, 2, or 3;
n is an integer equal to 0, 1, 2, or 3;
R⁷ is H, C₁₋₆ alkyl, C₃₋₆ cycloalkyl, C₁₋₆ alkyl substituted with C₃₋₆ cycloalkyl, or C(O)—R^{K};
R⁸ is H or C₁₋₆ alkyl;

### R^{K} is:

(1) C₁₋₆ alkyl,

(2) C₃₋₆ cycloalkyl,

(3) C₁₋₆ alkyl substituted with C₃₋₆ cycloalkyl,

(4) O—C₁₋₆ alkyl,

(5) O—C₁₋₆ alkyl substituted with O—C₁₋₆ alkyl,

(6) O—C₁₋₆ fluoroalkyl,

(7) C(O)O—C₁₋₆ alkyl,

(8) C₁₋₆ alkyl substituted with C(O)O—C₁₋₆ alkyl,

(9) C₁₋₆ alkyl substituted with C(O)OH,

(10) C₁₋₆ alkyl substituted with C(O)—C₁₋₆ alkyl,

(11) N(H)—C₁₋₆ alkyl,

(12) N(—C₁₋₆ alkyl)₂,

(13) C₁₋₆ alkyl substituted with NH₂, N(H)—C₁₋₆ alkyl, or N(—C₁₋₆ alkyl)₂,

(14) AryA,

(15) C₁₋₆ alkyl substituted with AryA,

(16) O—C₁₋₆ alkyl substituted with AryA,

(17) HetA,

(18) C₁₋₆ alkyl substituted with HetA,

(19) O—C₁₋₆ alkyl substituted with HetA,

(20) HetB,

(21) O-HetB, or

(22) O—C₁₋₆ alkyl substituted with HetB;

each AryA is an aryl which is independently phenyl or naphthyl, wherein the phenyl or naphthyl is optionally substituted with from 1 to 4 Y^{B} wherein each Y^{B} independently has the same definition as X^{B};
each HetA is a heteroaryl which is independently (i) a 5- or 6-membered heteroaromatic ring containing from 1 to 3 heteroatoms independently selected from N, O and S, or (ii) is a heterobicyclic ring selected from quinolinyl, isoquinolinyl, and quinoxalinyl; wherein the heteroaromatic ring (i) or the bicyclic ring (ii) is optionally substituted with from 1 to 4 Y^{C} wherein each Y^{C} independently has the same definition as X^{B}; and
each HetB is independently a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, and wherein the saturated or unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, O—C₁₋₆ haloalkyl, C(O)NH₂, C(O)N(H)—C₁₋₆ alkyl, C(O)N(—C₁₋₆ alkyl)₂, C(O)H, C(O)—C₁₋₆ alkyl, CO₂H, CO₂—C₁₋₆ alkyl, SO₂H, or SO₂—C₁₋₆ alkyl.

Other embodiments, aspects and features of the present invention are either further described in or will be apparent from the ensuing description, examples and appended claims.

### DETAILED DESCRIPTION OF THE INVENTION

The present invention includes compounds of Formula I above and pharmaceutically acceptable salts thereof. The compounds encompassed by Formula I include compounds which are HIV protease inhibitors and other compounds which can be metabolized in vivo to HIV protease inhibitors. More particularly, the compounds of Formula I in which R² is CH(R^{J})—OR^{P} are believed to be prodrugs which are converted in vivo into the pharmaceutically active component. The in vivo conversion of the prodrug can be the result of an enzyme-catalyzed chemical reaction, a metabolic chemical reaction, and/or a spontaneous chemical reaction (e.g., solvolysis).

Unless expressly stated to the contrary or clear from the context, a reference to compounds of the present invention refers to all compounds encompassed by Formula I, whether or not they act as prodrugs.

A first embodiment of the present invention (alternatively referred to herein as “Embodiment E1”) is a compound of Formula I (alternatively and more simply referred to as “Compound I”), or a pharmaceutically acceptable salt thereof, wherein R¹ is C₁₋₆ alkyl or C₁₋₆ alkyl substituted with C₃₋₆ cycloalkyl; and all other variables are as originally defined (i.e., as defined for Compound I in the Summary of the Invention).

A second embodiment of the present invention (Embodiment E2) is a compound of Formula I, or a pharmaceutically acceptable salt thereof, wherein R¹ is C₁₋₆ alkyl, C₁₋₆ fluoroalkyl, C₃₋₅ cycloalkyl, or CH₂—C₃₋₅ cycloalkyl; and all other variables are as originally defined.

A third embodiment of the present invention (Embodiment E3) is a compound of Formula I, or a pharmaceutically acceptable salt thereof, wherein R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclopropyl, cyclobutyl, CH₂-cyclopropyl, or CH₂-cyclobutyl; and all other variables are as originally defined.

A fourth embodiment of the present invention (Embodiment E4) is a compound of Formula I, or a pharmaceutically acceptable salt thereof, wherein R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl; and all other variables are as originally defined.

A fifth embodiment of the present invention (Embodiment E5) is a compound of Formula I, or a pharmaceutically acceptable salt thereof, wherein R¹ is C₁₋₆ alkyl; and all other variables are as originally defined.

A sixth embodiment of the present invention (Embodiment E6) is a compound of Formula I, or a pharmaceutically acceptable salt thereof, wherein R¹ is CH(CH₃)₂, CH₂CH(CH₃)₂, or CH₂CH₂CH(CH₃)₂; and all other variables are as originally defined.

A seventh embodiment of this part of the present invention (Embodiment E7) is a compound of Formula I, or a pharmaceutically acceptable salt thereof, wherein R¹ is CH₂CH(CH₃)₂ or CH₂CH₂CH(CH₃)₂; and all other variables are as originally defined.

An eighth embodiment of the present invention (Embodiment E8) is a compound of Formula I, or a pharmaceutically acceptable salt thereof, wherein R¹ is CH(CH₃)₂; and all other variables are as originally defined.

A ninth embodiment of the present invention (Embodiment E9) is a compound of Formula I, or a pharmaceutically acceptable salt thereof, wherein R¹ is CH₂CH(CH₃)₂; and all other variables are as originally defined.

A tenth embodiment of the present invention (Embodiment E10) is a compound of Formula I, or a pharmaceutically acceptable salt thereof, wherein R¹ is CH₂CH₂CH(CH₃)₂; and all other variables are as originally defined.

An eleventh embodiment of the present invention (Embodiment E11) is a compound of Formula I, or a pharmaceutically acceptable salt thereof, wherein R² is CH₂—Z, CH(CH₃)—Z, or CH(CF₃)—Z (i.e., R^{J} is H, CH₃, or CF₃); wherein Z is OH, NH₂, or OR^{P}; and wherein R^{P} is P(O)(OH)₂, P(O)(ONa)₂, P(O)(OK)₂, C(O)—C₁₋₆ alkyl, C(O)O—C₁₋₆ alkyl, C(O)N(—C₁₋₆ alkyl)₂, C(O)-pyridyl, or C(O)—C₁₋₆ alkylene-NH₂; and provided that:

(A) when R² is CH₂OH or CH₂OR^{P}, then at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H; and all other variables are as originally defined or as defined in any one of the preceding embodiments.

Under the proviso as originally set forth for Compound I and as set forth in this embodiment, the present invention includes all compounds of Formula I in which R³, R⁴, R⁵, and R^{5A} are all H except for compounds in which R² is CH₂OH or CH₂OR^{P}; all compounds of Formula I (regardless of the value of R²) in which one of R³ and R⁴ is H, and the other of R³ and R⁴ is not H; all compounds of Formula I (regardless of the value of R²) in which both of R³ and R⁴ are H and one or both of R⁵ and R^{5A} are not H; and all compounds of Formula I (regardless of the value of R²) in which both R³ and R⁴ are not H, and R⁵ and R^{5A} are both H. Under the proviso, compounds in which R³ and R⁴ and either or both R⁵ and R^{5A} are other than H are excluded.

In an aspect of Embodiment E11, R³ is H, C₁₋₄ alkyl, C₁₋₄ fluoroalkyl, or CH₂—C₃₋₅ cycloalkyl; R⁴ is H, C₁₋₄ alkyl, C₁₋₄ fluoroalkyl, or CH₂—C₃₋₅ cycloalkyl; R⁵ is H,

C₁₋₄ alkyl, C₁₋₄ fluoroalkyl, C₁₋₄ alkyl substituted with OH, C₂₋₄ alkenyl, C₂₋₄ alkynyl, C₃₋₅ cycloalkyl, or CH₂—C₃₋₅ cycloalkyl; and R^{5A} is H or C₁₋₄ alkyl; and alternatively, R⁵ and R^{5A} together with the carbon atom to which they are both attached form C₃₋₅ cycloalkyl.

A twelfth embodiment of the present invention (Embodiment E12) is a compound of Formula I, or a pharmaceutically acceptable salt thereof, wherein R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃; and provided that:

(A) when R² is CH₂OH or CH₂OR^{P}, then at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H; and all other variables are as originally defined or as defined in any one of the preceding embodiments.

In an aspect of Embodiment E12, R³ is H or CH₃; R⁴ is H or CH₃; R⁵ is H, CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl; and R^{5A} is H or CH₃; and alternatively, R⁵ and R^{5A} together with the carbon atom to which they are both attached form C₃₋₅ cycloalkyl.

A thirteenth embodiment of the present invention (Embodiment E13) is a compound of Formula I, or a pharmaceutically acceptable salt thereof, wherein R² is CH₂OH, CH(CH₃)OH, or CH₂NH₂; and provided that:

(A) when R² is CH₂OH, then at least one of R³, R⁴, R⁵ and R^{5A} is other than

H;

(B) when either or both R⁵ and R^{5A} are other than H, then at least one of R³ and R⁴ is H; and

(C) when R³ and R⁴ are both other than H, then R⁵ and R^{5A} are both H; and all other variables are as originally defined or as defined in any one of the preceding embodiments.

In an aspect of Embodiment E13, R³ is H or CH₃; R⁴ is H or CH₃; R⁵ is H, CH₃, CH₂CH₃, CH(CH₃)₂, CH₂CH₂CH₃, C(CH₃)₃, CF₃, CF₂CF₃, CH₂OH, ethenyl, ethynyl, or cyclopropyl; and R^{5A} is H or CH₃, with the proviso that when R^{5A} is CH₃, then R⁵ is CH₃; and alternatively, R⁵ and R^{5A} together with the carbon atom to which they are both attached form cyclobutyl or cyclopentyl.

A fourteenth embodiment of the present invention (Embodiment E14) is a compound of Formula I, or a pharmaceutically acceptable salt thereof, wherein R² is CH₂OH; and provided that:

(A) at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H; and all other variables are as originally defined or as defined in any one of the preceding embodiments.

In an aspect of Embodiment E14, R³ is H or CH₃; R⁴ is H or CH₃; R⁵ is H, CH₃, CH₂CH₃, CH(CH₃)₂, CH₂CH₂CH₃, C(CH₃)₃, CF₃, CF₂CF₃, CH₂OH, ethenyl, ethynyl, or cyclopropyl; and R^{5A} is H or CH₃, with the proviso that when R^{5A} is CH₃, then R⁵ is CH₃; and alternatively, R⁵ and R^{5A} together with the carbon atom to which they are both attached form cyclobutyl or cyclopentyl.

A fifteenth embodiment of the present invention (Embodiment E15) is a compound of Formula I, or a pharmaceutically acceptable salt thereof, wherein R² is CH₂OH; R³ is H; R⁴ is H; R⁵ is H, CH₃, CH₂CH₃, CH(CH₃)₂, CH₂CH₂CH₃, C(CH₃)₃, CF₃, CF₂CF₃, CH₂OH, ethenyl, ethynyl, or cyclopropyl; and R^{5A} is H or CH₃, with the proviso that when R^{5A} is CH₃, then R⁵ is CH₃; alternatively, R⁵ and R^{5A} together with the carbon atom to which they are both attached form cyclobutyl or cyclopentyl; and provided that either or both R⁵ and R^{5A} are other than H; and all other variables are as originally defined or as defined in any one of the preceding embodiments.

A sixteenth embodiment of the present invention (Embodiment E16) is a compound of Formula I, or a pharmaceutically acceptable salt thereof, wherein R⁶ is:

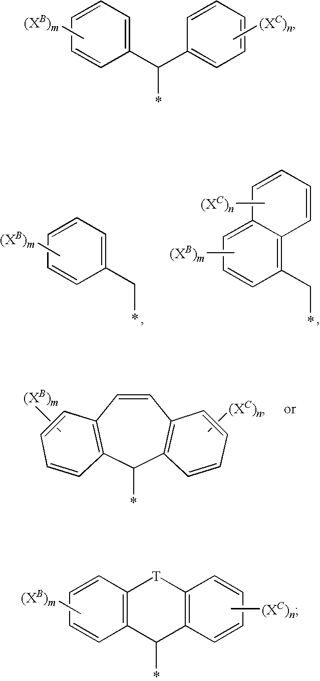

R^{6A} is H or C₁₋₄ alkyl; alternatively, R⁶ and R^{6A} together with the carbon to which they are attached form a C₃₋₅ cycloalkyl which is optionally substituted with phenyl, wherein the phenyl is optionally substituted with from 1 to 2 X^{B}; and all other variables are as originally defined or as defined in any one of the preceding embodiments.

A seventeenth embodiment of the present invention (Embodiment E17) is a compound of Formula I, or a pharmaceutically acceptable salt thereof, wherein R⁶ is:

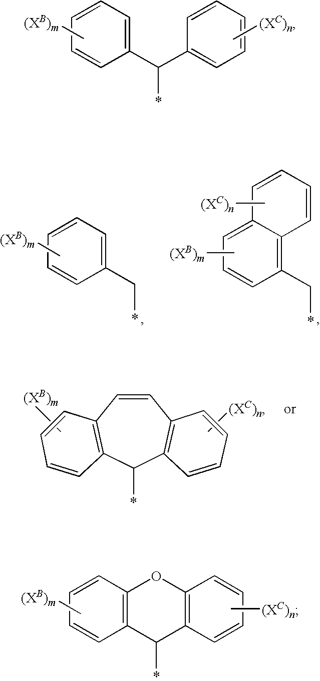

R^{6A} is H; alternatively, R⁶ and R^{6A} together with the carbon to which they are attached form cyclopropyl which is substituted with phenyl, wherein the phenyl is optionally substituted with from 1 to 2 X^{B}; and all other variables are as originally defined or as defined in any one of the preceding embodiments.

An eighteenth embodiment of the present invention (Embodiment E18) is a compound of Formula I, or a pharmaceutically acceptable salt thereof, wherein R⁶ is:

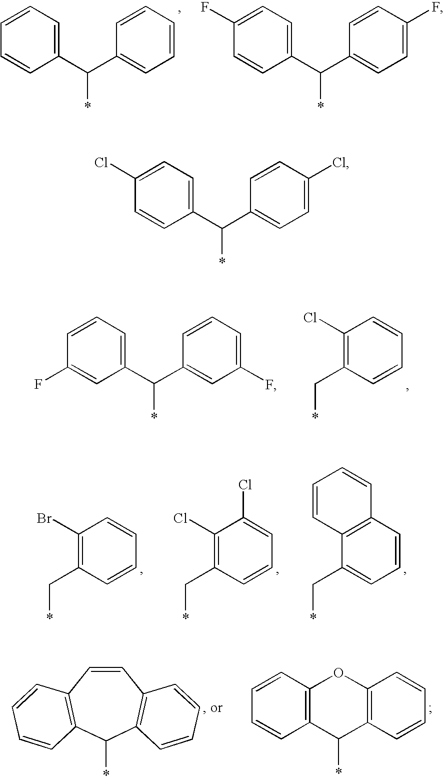

R^{6A} is H; alternatively, R⁶ and R^{6A} together with the carbon to which they are attached form cyclopropyl substituted with phenyl; and all other variables are as originally defined or as defined in any one of the preceding embodiments.

A nineteenth embodiment of the present invention (Embodiment E19) is a compound of Formula I, or a pharmaceutically acceptable salt thereof, wherein R⁶ is:

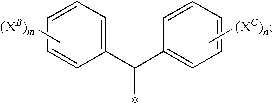

R^{6A} is H; and all other variables are as originally defined or as defined in any one of the preceding embodiments. In an aspect of this embodiment, m and n are either both 0 or both 1; and X^{B} and X^{C} are (i) both F and both para substituents, (ii) both F and both meta substituents, or (iii) both Cl and both para substituents.

A twentieth embodiment of the invention (Embodiment E20) is a compound of Formula I, or a pharmaceutically acceptable salt thereof, wherein each X^{B} and each X^{C} in the definition of R⁶ are independently selected from the group consisting of:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(O)C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl,

(18) CH₂OH, and

(19) CH₂O—C₁₋₃ alkyl;

m is an integer equal to 0, 1, or 2; n is an integer equal to 0, 1, or 2; and all other variables are as originally defined or as defined in any one of the preceding embodiments. In an aspect of this embodiment, R⁶ is

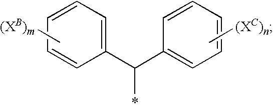

### and R^{6A} is H.

A twenty-first embodiment of the invention (Embodiment E21) is a compound of Formula I, or a pharmaceutically acceptable salt thereof, wherein each X^{B} and each X^{C} in the definition of R⁶ are independently selected from the group consisting of:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH, and

(17) CH₂OCH₃;

m is an integer equal to 0, 1, or 2; n is an integer equal to 0, 1, or 2; and all other variables are as originally defined or as defined in any one of the preceding embodiments. In an aspect of Embodiment E21, m is 0 or 1, and n is 0 or 1. In a further aspect of Embodiment E21, R⁶ is

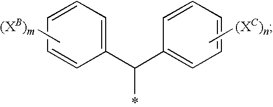

m is 0 or 1, and n is 0 or 1.

A twenty-second embodiment of the invention (Embodiment E22) is a compound of Formula I, or a pharmaceutically acceptable salt thereof, wherein each X^{A} is independently:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(H)—C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl, or

(18) C₁₋₃ alkyl substituted with

(a) cyclopropyl,

(b) CF₃,

(c) OH,

(d) O—C₁₋₃ alkyl,

(e) OCF₃,

(f) Cl,

(g) Br,

(h) F,

(i) CN,

(i) NO₂,

(k) NH₂,

(l) N(H)—C₁₋₃ alkyl,

(m) N(—C₁₋₃ alkyl)₂,

(n) C(O)—C₁₋₃ alkyl,

(o) CO₂H, or

(p) C(O)O—C₁₋₃ alkyl;

k is an integer equal to 0, 1, or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
and all other variables are as originally defined or as defined in any one of the preceding embodiments.

A twenty-third embodiment of the invention (Embodiment E23) is a compound of Formula I, or a pharmaceutically acceptable salt thereof, wherein each X^{A} is independently:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH,

(17) CH₂OCH₃,

(18) CH₂NH₂,

(19) CH₂N(H)CH₃,

(20) CH₂N(CH₃)₂,

(21) CH(CH₃)OH,

(22) CH(CH₃)OCH₃,

(23) CH(CH₃)NH₂,

(24) CH(CH₃)N(H)CH₃, or

(25) CH(CH₃)N(CH₃)₂;

k is an integer equal to 0, 1 or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
and all other variables are as originally defined or as defined in any one of the preceding embodiments.

A twenty-fourth embodiment of the invention (Embodiment E24) is a compound of Formula I, or a pharmaceutically acceptable salt thereof, wherein each X^{A} is independently selected from groups (1) to (25) as set forth in Embodiment E23; k is 0 or 1; and all other variables are as originally defined or as defined in any one of the preceding embodiments.

A twenty-fifth embodiment of the invention (Embodiment E25) is a compound of Formula I, or a pharmaceutically acceptable salt thereof, wherein there are 1 or 2×A groups on the phenylsulfonyl moiety wherein one X^{A} is in the para position on the phenyl ring and is CH₃, Cl, Br, F, NH₂, C(O)CH₃, CH₂OH, or CH(CH₃)OH; and the other, optional X^{A} is in the meta position on the phenyl ring and is Cl, Br, or F;

or, alternatively, when two X^{A} substituents are present on the phenyl ring and the two X^{A} are attached to adjacent carbon atoms, the two X^{A} are optionally taken together with the carbon atoms to which they are attached to form a thiazole that is fused to the phenyl ring to provide

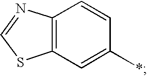

and all other variables are as originally defined or as defined in any one of the preceding embodiments.

A twenty-sixth embodiment of the invention (Embodiment E26) is a compound of Formula I, or a pharmaceutically acceptable salt thereof, wherein R⁷ is H, C₁₋₆ alkyl, C(O)—C₁₋₆ alkyl, C(O)O—C₁₋₆ alkyl, C(O)N(—C₁₋₆ alkyl)₂, C(O)—HetA, C(O)OCH₂-HetA, C(O)—HetB, or C(O)OCH₂-HetB; and all other variables are as originally defined or as defined in any one of the preceding embodiments.

A twenty-seventh embodiment of the invention (Embodiment E27) is a compound of Formula I, or a pharmaceutically acceptable salt thereof, wherein R⁷ is H, C(O)—C₁₋₆ alkyl, C(O)O—C₁₋₆ alkyl, C(O)N(—C₁₋₆ alkyl)₂, C(O)—HetA, or C(O)—HetB; and all other variables are as originally defined or as defined in any one of the preceding embodiments.

A twenty-eighth embodiment of the invention (Embodiment E28) is a compound of Formula I, or a pharmaceutically acceptable salt thereof, wherein R⁷ is H, CH₃, C(O)CH₃, C(O)OCH₃, C(O)OC(CH₃)₃, C(O)N(CH₃)₂, C(O)-morpholinyl, C(O)-pyridyl, or C(O)O—CH₂-pyridyl; and all other variables are as originally defined or as defined in any one of the preceding embodiments.

A twenty-ninth embodiment of the invention (Embodiment E29) is a compound of Formula I, or a pharmaceutically acceptable salt thereof, wherein R⁷ is H, C(O)CH₃, C(O)OCH₃, C(O)N(CH₃)₂, C(O)-pyridyl, or C(O)-morpholinyl; and all other variables are as originally defined or as defined in any one of the preceding embodiments.

A thirtieth embodiment of the invention (Embodiment E30) is a compound of Formula I, or a pharmaceutically acceptable salt thereof, wherein R⁷ is H, CH₃, C(O)OCH₃, C(O)OC(CH₃)₃, or C(O)O—CH₂-pyridyl; and all other variables are as originally defined or as defined in any one of the preceding embodiments.

A thirty-first embodiment of the invention (Embodiment E31) is a compound of Formula I, or a pharmaceutically acceptable salt thereof, wherein R⁷ is H or C(O)O—C₁₋₄ alkyl; and all other variables are as originally defined or as defined in any one of the preceding embodiments.

A thirty-second embodiment of the invention (Embodiment E32) is a compound of Formula I, or a pharmaceutically acceptable salt thereof, wherein R⁷ is H or C(O)OCH₃; and all other variables are as originally defined or as defined in any one of the preceding embodiments.

A thirty-third embodiment of the invention (Embodiment E33) is a compound of Formula I, or a pharmaceutically acceptable salt thereof, wherein R⁷ is C(O)OCH₃; and all other variables are as originally defined or as defined in any one of the preceding embodiments.

A thirty-fourth embodiment of the invention (Embodiment E34) is a compound of Formula I, or a pharmaceutically acceptable salt thereof, wherein R⁸ is H or C₁₋₄ alkyl; and all other variables are as originally defined or as defined in any one of the preceding embodiments.

A thirty-fifth embodiment of the invention (Embodiment E35) is a compound of Formula I, or a pharmaceutically acceptable salt thereof, wherein R⁸ is H or CH₃; and all other variables are as originally defined or as defined in any one of the preceding embodiments.

A thirty-sixth embodiment of the invention (Embodiment E36) is a compound of Formula I, or a pharmaceutically acceptable salt thereof, wherein R⁸ is H; and all other variables are as originally defined or as defined in any one of the preceding embodiments.

A thirty-seventh embodiment of the invention (Embodiment E37) is a compound of Formula I, or a pharmaceutically acceptable salt thereof, wherein:

each AryA is an aryl which is independently phenyl or naphthyl, wherein the phenyl or naphthyl is optionally substituted with from 1 to 3 substituents each of which is independently C₁₋₄ alkyl, CF₃, CH₂CF₃, OH, O—C₁₋₄ alkyl, OCF₃, OCH₂CF₃, Cl, Br, F, CN, NH₂, N(H)—C₁₋₄ alkyl, N(—C₁₋₄ alkyl)₂, CH(O), C(O)—C₁₋₄ alkyl, CO₂H, C(O)O—C₁₋₄ alkyl, SO₂H, or SO₂—C₁₋₄ alkyl;

each HetA is independently a heteroaryl selected from the group consisting of thienyl, furanyl, pyrrolyl, imidazolyl, pyrazolyl, triazolyl, tetrazolyl, oxazolyl, isooxazolyl, thiazolyl, isothiazolyl, oxadiazolyl, thiadiazolyl, pyridinyl, pyrazinyl, pyrimidinyl, pyridazinyl, quinolinyl, isoquinolinyl, and quinoxalinyl, wherein the heteroaryl is optionally substituted with from 1 to 3 substituents each of which is independently C₁₋₄ alkyl, CF₃, CH₂CF₃, OH, O—C₁₋₄ alkyl, OCF₃, OCH₂CF₃, Cl, Br, F, CN, NH₂, MID-C₁₋₄ alkyl, N(—C₁₋₄ alkyl)₂, CH(O), C(O)—C₁₋₄ alkyl, CO₂H, C(O)O—C₁₋₄ alkyl, SO₂H, or SO₂—C₁₋₄ alkyl; and

each HetB is independently a 5- or 6-membered, saturated heterocyclic ring containing from 1 to 2 heteroatoms independently selected from N, O and S, wherein each S atom is optionally oxidized to S(O) or S(O)₂, and wherein the saturated heterocyclic ring is optionally substituted with 1 to 3 substituents each of which is independently C₁₋₄ alkyl, oxo, C(O)NH₂, C(O)N(H)—C₁₋₄ alkyl, C(O)N(—C₁₋₄ alkyl)₂, CH(O), C(O)—C₁₋₄ alkyl, CO₂H, C(O)O—C₁₋₄ alkyl, SO₂H, or SO₂—C₁₋₄ alkyl;

and all other variables are as originally defined as defined in any one of the preceding embodiments.

A thirty-eighth embodiment of the invention (Embodiment E38) is a compound of Formula I, or a pharmaceutically acceptable salt thereof, wherein: each AryA is independently phenyl, which is optionally substituted with from 1 to 3 substituents each of which is independently CH₃, CF₃, OH, OCH₃, OCF₃, Cl, Br, F, CN, NH₂, N(H)CH₃, N(CH₃)₂, CH(O), C(O)CH₃, C(O)OCH₃, or SO₂CH₃;

each HetA is independently a heteroaryl selected from the group consisting of pyrrolyl, imidazolyl, pyridyl, pyrazinyl, quinolyl, isoquinolyl, and quinoxalinyl, wherein the heteroaryl is optionally substituted with from 1 to 3 substituents each of which is independently CH₃, CF₃, OH, OCH₃, OCF₃, Cl, Br, F, CN, NH₂, N(H)CH₃, N(CH₃)₂, C(O)CH₃, CO₂CH₃, or SO₂CH₃; and

each HetB is independently a saturated heterocyclic ring selected from the group consisting of tetrahydrofuranyl, pyrrolidinyl, piperidinyl, piperazinyl, morpholinyl, or thiomorpholinyl in which the S is optionally oxidized to S(O) or S(O)₂, and wherein the ring is optionally substituted with 1 or 2 substituents each of which is independently CH₃, CH₂CH₃, oxo, C(O)N(CH₃)₂, C(O)CH₃, CO₂CH₃, or S(O)₂CH₃;

and all other variables are as originally defined as defined in any one of the preceding embodiments.

A thirty-ninth embodiment of the invention (Embodiment E39) is a compound of Formula II:

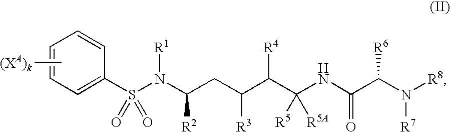

or a pharmaceutically acceptable salt thereof, wherein all of the variables are as originally defined or as defined in any one of the preceding embodiments.

A fortieth embodiment of the invention (Embodiment E40) is a compound of Formula III:

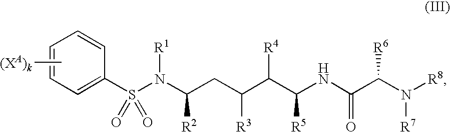

or a pharmaceutically acceptable salt thereof; wherein R⁵ is C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl; all other variables are as originally defined or as defined in any one of the preceding embodiments; and provided that at least one of R³ and R⁴ is H. In an aspect of this embodiment, R² is CH₂OH; R³ is H; R⁴ is H; R⁷ is C(O)OCH₃ and R⁸ is H.

A forty-first embodiment of the invention (Embodiment E41) is a compound of Formula IV:

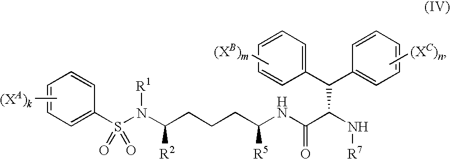

or a pharmaceutically acceptable salt thereof; wherein R⁵ is C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl; and all other variables are as originally defined or as defined in any one of the preceding embodiments. In an aspect of this embodiment, R⁵ is CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl. In another aspect, R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl. In another aspect of this embodiment, R² is CH₂OH, and R⁷ is C(O)OCH₃.

A forty-second embodiment of the invention (Embodiment E42) is a compound of Formula V:

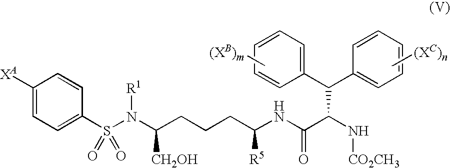

or a pharmaceutically acceptable salt thereof; wherein R⁵ is C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl; and all other variables are as originally defined or as defined in any one of the preceding embodiments. In an aspect of this embodiment, R⁵ is CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl. In another aspect, R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl.

A forty-third embodiment of the invention (Embodiment E43) is a compound of Formula VI:

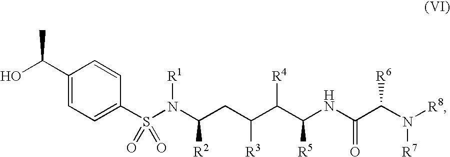

or a pharmaceutically acceptable salt thereof; wherein R⁵ is C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl; all other variables are as originally defined or as defined in any one of the preceding embodiments; and provided that at least one of R³ and R⁴ is H. In an aspect of this embodiment, R² is CH₂OH; R³ is H; R⁴ is H; R⁷ is C(O)OCH₃ and R⁸ is H.

A forty-fourth embodiment of the invention (Embodiment E44) is a compound of Formula I, or a pharmaceutically acceptable salt thereof, wherein all variables are as originally defined, with the proviso that:

(A) at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H.

The proviso in E44 defines a subset of the compounds of the invention in which at least one, but not all, of R³, R⁴, R⁵ and R^{5A} is other than H. More particularly it requires that (i) R³=H, or (ii) R⁴=H, or (iii) R⁵=R^{5A}=H, and it also requires that at least one of R³, R⁴,

R⁵ and R^{5A} be other than H. Aspects of Embodiment E44 include the compound of Formula I wherein all of the variables are as defined in any of the preceding embodiments except that this proviso is applied thereto, provided that such application defines a subset of the compounds that would otherwise be encompassed by the embodiment.

A first class of compounds of the present invention (alternatively referred to herein as Class C1) includes compounds of Formula I, and pharmaceutically acceptable salts thereof, wherein:

R¹ is C₁₋₆ alkyl, C₁₋₆ fluoroalkyl, C₃₋₅ cycloalkyl, or CH₂—C₃₋₅ cycloalkyl;
R² is CH₂—Z, CH(CH₃)—Z, CH(CF₃)—Z; wherein Z is OH, NH₂, or OR^{P}; and wherein R^{P} is P(O)(OH)₂, P(O)(ONa)₂, P(O)(OK)₂, C(O)—C₁₋₆ alkyl, C(O)O—C₁₋₆ alkyl, C(O)N(—C₁₋₆ alkyl)₂, C(O)-pyridyl, or C(O)—C₁₋₆ alkylene-NH₂;
R³ is H, C₁₋₄ alkyl, C₁₋₄ fluoroalkyl, or CH₂—C₃₋₅ cycloalkyl;
R⁴ is H, C₁₋₄ alkyl, C₁₋₄ fluoroalkyl, or CH₂—C₃₋₅ cycloalkyl;
R⁵ is H, C₁₋₄ alkyl, C₁₋₄ fluoroalkyl, C₁₋₄ alkyl substituted with OH, C₂₋₄ alkenyl, C₂₋₄ alkynyl, C₃₋₅ cycloalkyl, or CH₂—C₃₋₅ cycloalkyl;
R^{5A} is H or C₁₋₄ alkyl; alternatively, R⁵ and R^{5A} together with the carbon atom to which they are both attached form C₃₋₅ cycloalkyl;
and provided that:

(A) when R² is CH₂OH or CH₂OR^{P}, then at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H;

### R⁶ is:

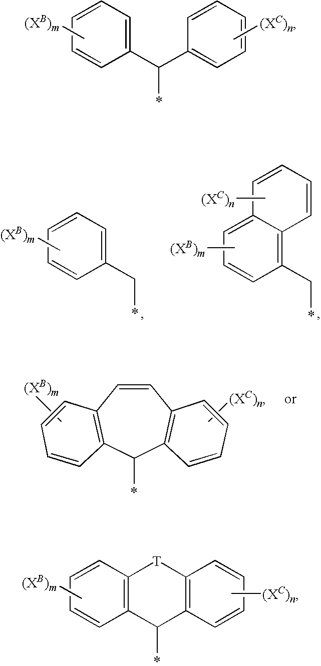

wherein the asterisk (*) denotes the point of attachment to the rest of the compound;
R^{6A} is H or C₁₋₄ alkyl;
alternatively, R⁶ and R^{6A} together with the carbon to which they are attached form a C₃₋₅ cycloalkyl which is optionally substituted with phenyl, wherein the phenyl is optionally substituted with from 1 to 2 X^{B};
each X^{B} and each X^{C} are independently selected from the group consisting of:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(H)—C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl,

(18) CH₂OH, and

(19) CH₂O—C₁₋₃ alkyl;

m is an integer equal to 0, 1, or 2;
n is an integer equal to 0, 1, or 2;
each X^{A} is independently:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(H)—C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl, or

(18) C₁₋₃ alkyl substituted with

(a) cyclopropyl,

(b) CF₃,

(c) OH,

(d) O—C₁₋₃ alkyl,

(e) OCF₃,

(f) Cl,

(g) Br,

(h) F,

(i) CN,

(j) NO₂,

(k) NH₂,

(l) N(H)—C₁₋₃ alkyl,

(m) N(—C₁₋₃ alkyl)₂,

(n) C(O)—C₁₋₃ alkyl,

(o) CO₂H, or

(p) C(O)O—C₁₋₃ alkyl;

k is an integer equal to 0, 1, or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
R⁷ is H, C₁₋₆ alkyl, C(O)—C₁₋₆ alkyl, C(O)O—C₁₋₆ alkyl, C(O)N(—C₁₋₆ alkyl)₂, C(O)—HetA, C(O)OCH₂-HetA, C(O)—HetB, or C(O)OCH₂-HetB;
R⁸ is H or C₁₋₄ alkyl;
HetA is a heteroaryl selected from the group consisting of pyrrolyl, imidazolyl, pyridyl, pyrazinyl, quinolyl, isoquinolyl, and quinoxalinyl, wherein the heteroaryl is optionally substituted with from 1 to 3 substituents each of which is independently CH₃, CF₃, OH, OCH₃, OCF₃, Cl, Br, F, CN, NH₂, N(H)CH₃, N(CH₃)₂, C(O)CH₃, CO₂CH₃, or SO₂CH₃; and
HetB is a saturated heterocyclic ring selected from the group consisting of tetrahydrofuranyl, pyrrolidinyl, piperidinyl, piperazinyl, morpholinyl, or thiomorpholinyl in which the S is optionally oxidized to S(O) or S(O)₂, and wherein the ring is optionally substituted with 1 or 2 substituents each of which is independently CH₃, CH₂CH₃, oxo, C(O)N(CH₃)₂, C(O)CH₃, CO₂CH₃, or S(O)₂CH₃.

A first subclass of Class C1 (Subclass SC1-1) includes compounds of Formula I and their pharmaceutically acceptable salts, wherein R² is CH₂OH; R³ is H; R⁴ is H; and provided that either or both R⁵ and R^{5A} are other than H; R^{6A} is H; R⁷ is C(O)OCH₃ and R⁸ is H; and all of the other variables are as originally defined in Class C1.

A second class of compounds of the present invention (Class C2) includes compounds of Formula I, and pharmaceutically acceptable salts thereof, wherein:

R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclopropyl, cyclobutyl, CH₂-cyclopropyl, or CH₂-cyclobutyl;
R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃;

### R³ is H or CH₃;

### R⁴ is H or CH₃;

R⁵ is H, CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl;

### R^{5A} is H or CH₃;

alternatively, R⁵ and R^{5A} together with the carbon atom to which they are both attached form C₃₋₅ cycloalkyl;
and provided that:

(A) when R² is CH₂OH or CH₂OR^{P}, then at least one of R³, R⁴, R⁵ and R^{5A} is other than H;

(B) when either or both R⁵ and R^{5A} are other than H, then at least one of R³ and R⁴ is H; and

(C) when R³ and R⁴ are both other than H, then R⁵ and R^{5A} are both H;

### R⁶ is:

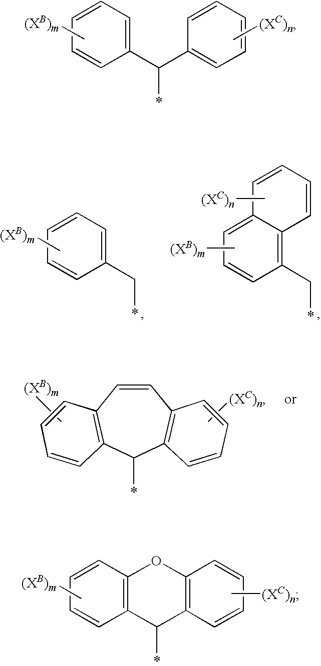

### R^{6A} is H;

alternatively, R⁶ and R^{6A} together with the carbon to which they are attached form cyclopropyl which is substituted with phenyl, wherein the phenyl is optionally substituted with from 1 to 2 X^{B};
each X^{B} and each X^{C} are independently selected from the group consisting of:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH, and

(17) CH₂OCH₃;

m is 0, 1 or 2;
n is 0, 1, or 2;
each X^{A} is independently:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH,

(17) CH₂OCH₃,

(18) CH₂NH₂,

(19) CH₂N(H)CH₃,

(20) CH₂N(CH₃)₂,

(21) CH(CH₃)OH,

(22) CH(CH₃)OCH₃,

(23) CH(CH₃)NH₂,

(24) CH(CH₃)N(H)CH₃, or

(25) CH(CH₃)N(CH₃)₂;

k is 0, 1, or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
R⁷ is H, CH₃, C(O)CH₃, C(O)OCH₃, C(O)OC(CH₃)₃, C(O)N(CH₃)₂, C(O)-morpholinyl, C(O)-pyridyl, or C(O)O—CH₂-pyridyl; and

### R⁸ is H or CH₃.

A first subclass of Class C2 (Subclass SC1-2) includes compounds of Formula I and their pharmaceutically acceptable salts, wherein R² is CH₂OH; R³ is H; R⁴ is H; and provided that either or both R⁵ and R^{5A} are other than H; R^{6A} is H; R⁷ is C(O)OCH₃ and R⁸ is H; and all of the other variables are as originally defined in Class C2.

A second subclass of Class C2 (Subclass SC2-2) includes compounds of Formula III and their pharmaceutically acceptable salts, wherein R³ is H or CH₃; R⁴ is H or CH₃; provided that at least one of R³ and R⁴ is H; R⁵ is CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl; R^{5A} is H; R^{6A} is H; and all other variables are as originally defined in Class C2.

A third subclass of Class C2 (Subclass SC3-2) includes compounds of Formula III and their pharmaceutically acceptable salts, wherein R² is CH₂OH; R³ is H; R⁴ is H; R⁷ is C(O)OCH₃ and R⁸ is H; and all of the other variables are as originally defined in Subclass SC2-2.

A third class of compounds of the present invention (Class C3) includes compounds of Formula I, and pharmaceutically acceptable salts thereof, wherein:

R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl;

### R² is CH₂OH, CH(CH₃)OH, or CH₂NH₂;

### R³ is H or CH₃;

### R⁴ is H or CH₃;

R⁵ is H, CH₃, CH₂CH₃, CH(CH₃)₂, CH₂CH₂CH₃, C(CH₃)₃, CF₃, CF₂CF₃, CH₂OH, ethenyl, ethynyl, or cyclopropyl;
R^{5A} is H or CH₃, with the proviso that when R^{5A} is CH₃, then R⁵ is CH₃;
alternatively, R⁵ and R^{5A} together with the carbon atom to which they are both attached form cyclobutyl or cyclopentyl;
and provided that:

(A) when R² is CH₂OH, then at least one of R³, R⁴, R⁵ and R^{5A} is other than

H;

(B) when either or both R⁵ and R^{5A} are other than H, then at least one of R³ and R⁴ is H; and

(C) when R³ and R⁴ are both other than H, then R⁵ and R^{5A} are both H;

### R⁶ is:

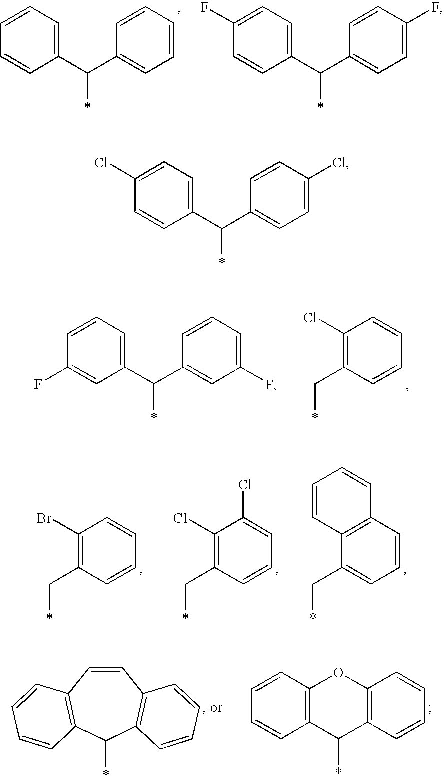

### R^{6A} is H;

alternatively, R⁶ and R^{6A} together with the carbon to which they are attached form cyclopropyl substituted with phenyl;
there are 1 or 2×A groups on the phenylsulfonyl moiety wherein one X^{A} is in the para position on the phenyl ring and is CH₃, Cl, Br, F, NH₂, C(O)CH₃, CH₂OH, or CH(CH₃)OH; and the other, optional X^{A} is in the meta position on the phenyl ring and is Cl, Br, or F;
or, alternatively, when two X^{A} substituents are present on the phenyl ring and the two X^{A} are attached to adjacent carbon atoms, the two X^{A} are optionally taken together with the carbon atoms to which they are attached to form a thiazole that is fused to the phenyl ring to provide

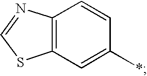

R⁷ is H, CH₃, C(O)OCH₃, C(O)OC(CH₃)₃, or C(O)O—CH₂-pyridyl; and

### R⁸ is H or CH₃.

A first subclass of Class C3 (Subclass SC1-3) includes compounds of Formula I and their pharmaceutically acceptable salts, wherein R² is CH₂OH; R³ is H; R⁴ is H; and provided that either or both R⁵ and R^{5A} are other than H; R^{6A} is H; R⁷ is C(O)OCH₃ and R⁸ is H; and all of the other variables are as originally defined in Class C3.

A second subclass of Class C3 (Subclass SC2-3) includes compounds of Formula III and their pharmaceutically acceptable salts, wherein R³ is H or CH₃; R⁴ is H or CH₃; provided that at least one of R³ and R⁴ is H; R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl; R^{5A} is H; R^{6A} is H; and all other variables are as originally defined in Class C3.

A third subclass of Class C3 (Subclass SC3-3) includes compounds of Formula III and their pharmaceutically acceptable salts, wherein R² is CH₂OH; R³ is H; R⁴ is H; R⁷ is C(O)OCH₃ and R⁸ is H; and all of the other variables are as originally defined in Subclass SC2-3.

A fourth class of compounds of the present invention (Class C4) includes compounds of Formula I, and pharmaceutically acceptable salts thereof, wherein R² is CH₂OH; R³ is H; R⁴ is H; and provided that either or both R⁵ and R^{5A} are other than H; R^{6A} is H; R⁷ is C(O)OCH₃R⁸ is H; and all other variables are as originally defined.

A fifth class of compounds of the present invention (Class C5) includes compounds of Formula V, and pharmaceutically acceptable salts thereof, wherein:

R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl;
R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl;

### X^{A} is NH₂, C(O)CH₃, CH₂OH, or CH(CH₃)OH;

each X^{B} and each X^{C} are independently selected from the group consisting of:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH, and

(17) CH₂OCH₃;

m is an integer equal to 0, 1, or 2; and
n is an integer equal to 0, 1, or 2.

A first subclass of Class C5 (Subclass SC1-5) includes compounds of Formula V and their pharmaceutically acceptable salts, wherein R¹ is CH(CH₃)₂, CH₂CH(CH₃)₂, or CH₂CH₂CH(CH₃)₂; and all of the other variables are as originally defined in Class C5.

A second subclass of Class C5 (Subclass SC2-5) includes compounds of Formula V and their pharmaceutically acceptable salts, wherein m and n are either both 0 or both 1; and X^{B} and X^{C} are (i) both F and both para substituents, (ii) both F and both meta substituents, or (iii) both C1 and both para substituents; and all of the other variables are as originally defined in Class C5.

A third subclass of Class C5 (Subclass SC3-5) includes compounds of Formula V and their pharmaceutically acceptable salts, wherein R¹ is CH(CH₃)₂, CH₂CH(CH₃)₂, or CH₂CH₂CH(CH₃)₂; and all of the other variables are as defined in Subclass SC2-5.

A forty-fifth embodiment of this part of the present invention (Embodiment E45) is a compound selected from the group consisting of the compounds set forth in Examples A1 to M1 (inclusive); and pharmaceutically acceptable salts thereof.

A forty-sixth embodiment of this part of the present invention (Embodiment E46) is a compound selected from the group consisting of the compounds set forth in Examples D2, E1, F1, F2, H1, H-3, J1, J27, K1, K4, L2, and pharmaceutically acceptable salts thereof.

The present invention also includes compounds of Formula I-A:

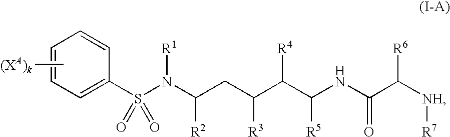

and pharmaceutically acceptable salts thereof, wherein:
R¹ is C₁₋₆ alkyl or C₁₋₆ alkyl substituted with C₃₋₆ cycloalkyl;
R³ is H, C₁₋₆ alkyl, C₁₋₆ fluoroalkyl, or C₁₋₆ alkyl substituted with C₃₋₅ cycloalkyl;
R⁴ is H, C₁₋₆ alkyl, C₁₋₆ fluoroalkyl, or C₁₋₆ alkyl substituted with C₃₋₅ cycloalkyl;
R⁵ is H, C₁₋₆ alkyl, C₁₋₆ fluoroalkyl, or C₁₋₆ alkyl substituted with C₃₋₅ cycloalkyl;
provided that:

(A) when R² is CH₂OH or CH₂OR^{P}, then at least one of R³, R⁴, and R⁵ is C₁₋₆ alkyl, C₁₋₆ fluoroalkyl, or C₁₋₆ alkyl substituted with C₃₋₅ cycloalkyl; and

(B) at least one of R³, R⁴, and R⁵ is H; and

each X^{A} is independently as originally defined for Compound I (see the Summary of the Invention)
or, alternatively, when two or more X^{A} substituents are present on the phenyl ring and two of the X^{A} are attached to adjacent carbon atoms of the phenyl ring, the two X^{A} are optionally taken together to form —OCH₂O— or —OCH₂CH₂O—;

### R⁶ is:

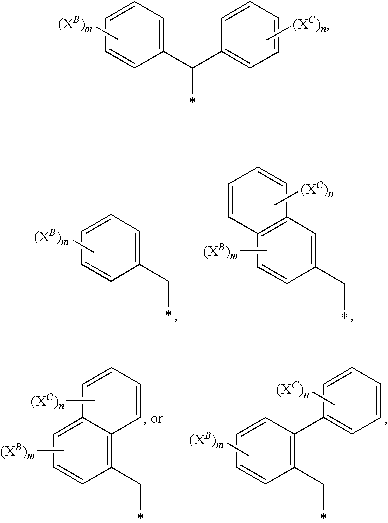

wherein the asterisk (*) denotes the point of attachment to the rest of the compound; and

### R^{K} is:

(1) C₁₋₆ alkyl,

(2) C₃₋₆ cycloalkyl,

(3) C₁₋₆ alkyl substituted with C₃₋₆ cycloalkyl,

(4) O—C₁₋₆ alkyl,

(5) O—C₁₋₆ alkyl substituted with O—C₁₋₆ alkyl,

(6) O—C₁₋₆ fluoroalkyl,

(7) C(O)O—C₁₋₆ alkyl,

(8) C₁₋₆ alkyl substituted with C(O)O—C₁₋₆ alkyl,

(9) C₁₋₆ alkyl substituted with C(O)OH,

(10) C₁₋₆ alkyl substituted with C(O)—C₁₋₆ alkyl,

(11) N(H)—C₁₋₆ alkyl,

(12) N(—C₁₋₆ alkyl)₂,

(13) C₁₋₆ alkyl substituted with NH₂, N(H)—C₁₋₆ alkyl, or N(—C₁₋₆ alkyl)₂,

(14) AryA,

(15) C₁₋₆ alkyl substituted with AryA,

(16) O—C₁₋₆ alkyl substituted with AryA,

(17) HetA,

(18) C₁₋₆ alkyl substituted with HetA,

(19) O—C₁₋₆ alkyl substituted with HetA,

(20) HetB, or

(21) O-HetB;

and all other variables are as originally defined (i.e., as defined with respect to Compound I in the Summary of the Invention).

A first embodiment of this part of the present invention (alternatively referred to herein as “Embodiment E1-A”) is a compound of Formula I-A (alternatively and more simply referred to as “Compound I-A”), or a pharmaceutically acceptable salt thereof, wherein R¹ is C₁₋₆ alkyl; and all other variables are as originally defined just above for a compound of Formula I-A.

A second embodiment of this part of the present invention (Embodiment E2-A) is a compound of Formula I-A, or a pharmaceutically acceptable salt thereof, wherein R¹ is CH₂CH(CH₃)₂ or CH₂CH₂CH(CH₃)₂; and all other variables are as originally defined for Compound I-A.

A third embodiment of this part of the present invention (Embodiment E3) is a compound of Formula I-A, or a pharmaceutically acceptable salt thereof, wherein R¹ is CH₂CH₂CH(CH₃)₂; and all other variables are as originally defined for Compound I-A.

A fourth embodiment of this part of the present invention (Embodiment E4-A) is a compound of Formula I-A, or a pharmaceutically acceptable salt thereof, wherein R² is CH₂—Z, CH(CH₃)—Z, CH(CF₃)—Z; wherein Z is OH, NH₂, or OR^{P}; and wherein R^{P} is P(O)(OH)₂, P(O)(ONa)₂, P(O)(OK)₂, C(O)—C₁₋₆ alkyl, C(O)O—C₁₋₆ alkyl, C(O)N(—C₁₋₆ alkyl)₂, C(O)-pyridyl, or C(O)—C₁₋₆ alkylene-NH₂; and provided that:

(A) when R² is CH₂OH or CH₂OR^{P}, then at least one of R³, R⁴, and R⁵ is C₁₋₆ alkyl, C₁₋₆ fluoroalkyl, or C₁₋₆ alkyl substituted with C₃₋₅ cycloalkyl; and

(B) at least one of R³, R⁴, and R⁵ is H;

and all other variables are as originally defined for Compound I-A or as defined in any one of the preceding embodiments of Compound I-A.

Under the proviso as originally set forth for Compound I-A and as set forth in this embodiment, the present invention includes all compounds of Formula I-A in which R³, R⁴, and R⁵ are all H except for compounds in which R² is CH₂OH or CH₂OR^{P}; all compounds of Formula I in which two of R³, R⁴, and R⁵ are H and the other is not H; and all compounds in which one of R³, R⁴, and R⁵ is H and the other two are not H.

A fifth embodiment of this part of the present invention (Embodiment E5-A) is a compound of Formula I-A, or a pharmaceutically acceptable salt thereof, wherein R³ is H or C₁₋₄ alkyl; R⁴ is H or C₁₋₄ alkyl; R⁵ is H or C₁₋₄ alkyl; and provided that:

(A) when R² is CH₂OH or CH₂OR^{P}, then at least one of R³, R⁴, and R⁵ is C₁₋₄ alkyl; and

(B) at least one of R³, R⁴, and R⁵ is H; and all other variables are as originally defined for Compound I-A or as defined in any one of the preceding embodiments of Compound I-A.

In an aspect of Embodiment E5-A, one of R³, R⁴ and R⁵ is C₁₋₄ alkyl; and the other two of R³, R⁴ and R⁵ are H.

A sixth embodiment of this part of the present invention (Embodiment E6-A) is a compound of Formula I-A, or a pharmaceutically acceptable salt thereof, wherein R³ is H, CH₃, CF₃, CH₂-cyclopropyl, or CH₂-cyclobutyl; R⁴ is H, CH₃, CF₃, CH₂-cyclopropyl, or CH₂-cyclobutyl; R⁵ is H, CH₃, CF₃, CH₂-cyclopropyl, or CH₂-cyclobutyl; and provided that:

(A) when R² is CH₂OH or CH₂OR^{P}, then at least one of R³, R⁴, and R⁵ is CH₃, CF₃, CH₂-cyclopropyl, or CH₂-cyclobutyl; and

(B) at least one of R³, R⁴, and R⁵ is H; and all other variables are as originally defined for Compound I-A or as defined in any one of the preceding embodiments of Compound I-A.

In an aspect of Embodiment E6-A, one of R³, R⁴ and R⁵ is CH₃, CF₃, CH₂-cyclopropyl, or CH₂-cyclobutyl; and the other two of R³, R⁴ and R⁵ are H.

A seventh embodiment of this part of the present invention (Embodiment E7-A) is a compound of Formula I-A, or a pharmaceutically acceptable salt thereof, wherein R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃; R³ is H or CH₃; R⁴ is H or CH₃; R⁵ is H or CH₃; and provided that:

(A) when R² is CH₂OH or CH₂OR^{P}, then at least one of R³, R⁴, and R⁵ is CH₃; and

(B) at least one of R³, R⁴, and R⁵ is H.

In an aspect of Embodiment E7-A, one of R³, R⁴ and R⁵ is CH₃, and the other two of R³, R⁴ and R⁵ are H; and all other variables are as originally defined for Compound I-A or as defined in any one of the preceding embodiments of Compound I-A.

An eighth embodiment of this part of the invention (Embodiment E8-A) is a compound of Formula I-A, or a pharmaceutically acceptable salt thereof, wherein R⁶ is:

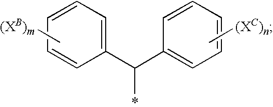

and all other variables are as originally defined for Compound I-A or as defined in any one of the preceding embodiments of Compound I-A.

A ninth embodiment of this part of the invention (Embodiment E9-A) is a compound of Formula I-A, or a pharmaceutically acceptable salt thereof, wherein each X^{B} and each X^{C} in the definition of R⁶ are independently selected from the group consisting of groups (1) to (19) as set forth in Embodiment E20 above; m is an integer equal to 0, 1, or 2; n is an integer equal to 0, 1, or 2; and all other variables are as originally defined for Compound I-A or as defined in any one of the preceding embodiments of Compound I-A. In an aspect of Embodiment E9-A, R⁶ is

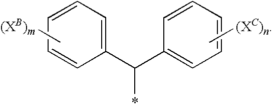

A tenth embodiment of this part of the invention (Embodiment E10-A) is a compound of Formula I-A, or a pharmaceutically acceptable salt thereof, wherein each X^{B} and each X^{C} in the definition of R⁶ are independently selected from the group consisting of the groups (1) to (17) as set forth in Embodiment E21 above; m is an integer equal to 0 or 1; n is an integer equal to 0 or 1; and all other variables are as originally defined for Compound I-A or as defined in any one of the preceding embodiments of Compound I-A. In an aspect of Embodiment E10-A, R⁶ is

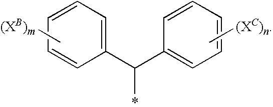

An eleventh embodiment of this part of the invention (Embodiment E11-A) is a compound of Formula I-A, or a pharmaceutically acceptable salt thereof, wherein in the definition of R⁶, X^{B} and X^{C} are both F; m is 0 or 1; n is 0 or 1; and all other variables are as originally defined for Compound I-A or as defined in any one of the preceding embodiments of Compound I-A. In an aspect of Embodiment E11-A, R⁶ is:

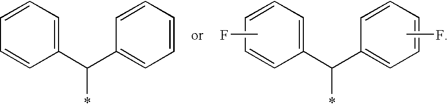

A twelfth embodiment of this part of the invention (Embodiment E12-A) is a compound of Formula I-A, or a pharmaceutically acceptable salt thereof, wherein each X^{A} is independently selected from groups (1) to (18) as set forth in Embodiment E22 above; k is an integer equal to 0, 1, or 2; and all other variables are as originally defined for Compound I-A or as defined in any one of the preceding embodiments of Compound I-A.

A thirteenth embodiment of this part of the invention (Embodiment E13-A) is a compound of Formula I-A, or a pharmaceutically acceptable salt thereof, wherein each X^{A} is independently selected from groups (1) to (25) as set forth in Embodiment E23 above; k is an integer equal to 0 or 1; and all other variables are as originally defined for Compound I-A or as defined in any one of the preceding embodiments of Compound I-A.

A fourteenth embodiment of this part of the invention (Embodiment E14-A) is a compound of Formula I-A, or a pharmaceutically acceptable salt thereof, wherein k is 0, or k is 1 and X^{A} is para to the sulfonyl; and all other variables are as originally defined for Compound I-A or as defined in any one of the preceding embodiments of Compound I-A.

A fifteenth embodiment of this part of the invention (Embodiment E15-A) is a compound of Formula I-A, or a pharmaceutically acceptable salt thereof, wherein k is 0, or k is 1 and X^{A} is 4-CH₃ or 4-NH₂; and all other variables are as originally defined for Compound I-A or as defined in any one of the preceding embodiments of Compound I-A.

A sixteenth embodiment of this part of the invention (Embodiment E16-A) is a compound of Formula I-A, or a pharmaceutically acceptable salt thereof, wherein R⁷ is H, C(O)—C₁₋₆ alkyl, C(O)O—C₁₋₆ alkyl, C(O)N(—C₁₋₆ alkyl)₂, C(O)—HetA, or C(O)—HetB; and all other variables are as originally defined for Compound I-A or as defined in any one of the preceding embodiments of Compound I-A.

A seventeenth embodiment of this part of the invention (Embodiment E17-A) is a compound of Formula I-A, or a pharmaceutically acceptable salt thereof, wherein R⁷ is H, C(O)CH₃, C(O)OCH₃, C(O)N(CH₃)₂, C(O)-pyridyl, or C(O)-morpholinyl; and all other variables are as originally defined for Compound I-A or as defined in any one of the preceding embodiments of Compound I-A.

An eighteenth embodiment of this part of the invention (Embodiment E18-A) is a compound of Formula I-A, or a pharmaceutically acceptable salt thereof, wherein R⁷ is H or C(O)O—C₁₋₄ alkyl; and all other variables are as originally defined for Compound I-A or as defined in any one of the preceding embodiments of Compound I-A.

A nineteenth embodiment of this part of the invention (Embodiment E19-A) is a compound of Formula I-A, or a pharmaceutically acceptable salt thereof, wherein R⁷ is H or C(O)OCH₃; and all other variables are as originally defined for Compound I-A or as defined in any one of the preceding embodiments of Compound I-A.

A twentieth embodiment of this part of the invention (Embodiment E20-A) is a compound of Formula I-A, or a pharmaceutically acceptable salt thereof, wherein AryA, HetA and HetB are as defined in Embodiment E37 above; and all other variables are as originally defined for Compound I-A or as defined in any one of the preceding embodiments of Compound I-A.

A twenty-first embodiment of this part of the invention (Embodiment 21-A) is a compound of Formula I-A, or a pharmaceutically acceptable salt thereof, wherein AryA, HetA, and HetB are as defined in Embodiment E38 above; and all other variables are as originally defined for Compound I-A or as defined in any one of the preceding embodiments of Compound I-A.

A twenty-second embodiment of this part of the invention (Embodiment E22-A) is a compound of Formula III-A:

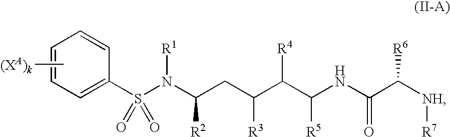

or a pharmaceutically acceptable salt thereof, wherein all of the variables are as originally defined for Compound I-A or as defined in any one of the preceding embodiments of Compound I-A.

A twenty-third embodiment of this part of the invention (Embodiment E23-A) is a compound of Formula III-A:

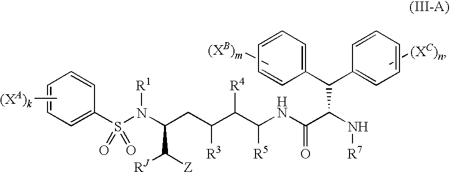

or a pharmaceutically acceptable salt thereof, wherein all of the variables are as originally defined for Compound I-A or as defined in any one of the preceding embodiments of Compound I-A.

A twenty-fourth embodiment of this part of the invention (Embodiment E24-A) is a compound of Formula IV-A:

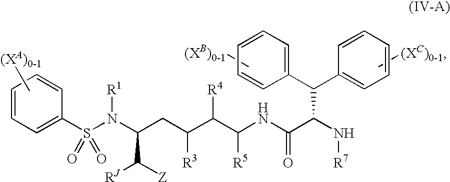

or a pharmaceutically acceptable salt thereof, wherein all of the variables are as originally defined for Compound I-A or as defined in any one of the preceding embodiments of Compound I-A.

A twenty-fifth embodiment of this part of the invention (Embodiment E25-A) is a compound of Formula V-A:

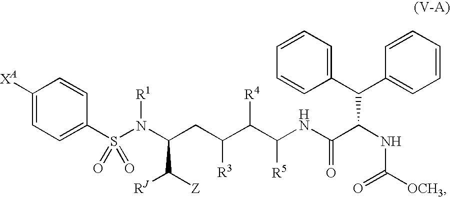

or a pharmaceutically acceptable salt thereof, wherein all of the variables are as originally defined for Compound I-A or as defined in any one of the preceding embodiments of Compound I-A.

A first class of compounds of this part of the present invention (alternatively referred to herein as Class C1-A) includes compounds of Formula I-A, and pharmaceutically acceptable salts thereof, wherein:

R¹ is C₁₋₆ alkyl;
R² is CH₂—Z, CH(CH₃)—Z, CH(CF₃)—Z; wherein Z is OH, NH₂, or OR^{P}; and wherein R^{P} is P(O)(OH)₂, P(O)(ONa)₂, P(O)(OK)₂, C(O)—C₁₋₆ alkyl, C(O)—C₁₋₆ alkyl, C(O)N(—C₁₋₆ alkyl)₂, C(O)-pyridyl, or C(O)—C₁₋₆ alkylene-NH₂;
R³ is H, CH₃, CF₃, CH₂-cyclopropyl, or CH₂-cyclobutyl;
R⁴ is H, CH₃, CF₃, CH₂-cyclopropyl, or CH₂-cyclobutyl;
R⁵ is H, CH₃, CF₃, CH₂-cyclopropyl, or CH₂-cyclobutyl;
provided that:

(A) when R² is CH₂OH or CH₂OR^{P}, then at least one of R³, R⁴, and R⁵ is CH₃, CF₃, CH₂-cyclopropyl, or CH₂-cyclobutyl; and

(B) at least one of R³, R⁴, and R⁵ is H.

### R⁶ is:

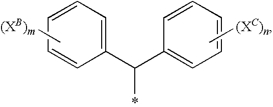

wherein the asterisk (*) denotes the point of attachment to the rest of the compound;
each X^{B} and each X^{C} are independently selected from the group consisting of:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(H)—C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl,

(18) CH₂OH, and

(19) CH₂O—C₁₋₃ alkyl;

m is an integer equal to 0, 1, or 2;
n is an integer equal to 0, 1, or 2;
each X^{A} is independently:

(1) C₁₋₃ alkyl,

(2) cyclopropyl,

(3) CF₃,

(4) OH,

(5) O—C₁₋₃ alkyl,

(6) OCF₃,

(7) Cl,

(8) Br,

(9) F,

(10) CN,

(11) NO₂,

(12) NH₂,

(13) N(H)—C₁₋₃ alkyl,

(14) N(—C₁₋₃ alkyl)₂,

(15) C(O)—C₁₋₃ alkyl,

(16) CO₂H,

(17) C(O)O—C₁₋₃ alkyl, or

(18) C₁₋₃ alkyl substituted with

(a) cyclopropyl,

(b) CF₃,

(c) OH,

(d) O—C₁₋₃ alkyl,

(e) OCF₃,

(f) Cl,

(g) Br,

(h) F,

(i) CN,

(j) NO₂,

(k) NH₂,

(l) N(H)—C₁₋₃ alkyl,

(m) N(—C₁₋₃ alkyl)₂,

(n) C(O)—C₁₋₃ alkyl,

(o) CO₂H, or

(p) C(O)O—C₁₋₃ alkyl; and

k is an integer equal to 0, 1, or 2;
R⁷ is H, C(O)—C₁₋₆ alkyl, C(O)O—C₁₋₆ alkyl, C(O)N(—C₁₋₆ alkyl)₂, C(O)—HetA, or C(O)—HetB;
HetA is a heteroaryl selected from the group consisting of pyrrolyl, imidazolyl, pyridyl, pyrazinyl, quinolyl, isoquinolyl, and quinoxalinyl, wherein the heteroaryl is optionally substituted with from 1 to 3 substituents each of which is independently CH₃, CF₃, OH, OCH₃, OCF₃, Cl, Br, F, CN, NH₂, N(H)CH₃, N(CH₃)₂, C(O)CH₃, CO₂CH₃, or SO₂CH₃; and

HetB is a saturated heterocyclic ring selected from the group consisting of tetrahydrofuranyl, pyrrolidinyl, piperidinyl, piperazinyl, morpholinyl, or thiomorpholinyl in which the S is optionally oxidized to S(O) or S(O)₂, and wherein the ring is optionally substituted with 1 or 2 substituents each of which is independently CH₃, CH₂CH₃, oxo, C(O)N(CH₃)₂, C(O)CH₃, CO₂CH₃, or S(O)₂CH₃.

A first subclass of Class C1-A (alternatively referred to herein as Subclass SC1-1-A) includes compounds of Formula VI-A:

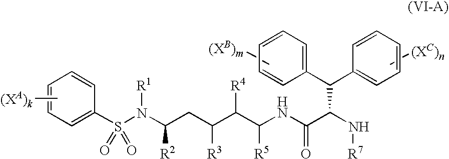

and pharmaceutically acceptable salts thereof, wherein all of the variables are as defined in Class C1-A.

A second subclass of Class C1-A (Subclass SC1-2-A) includes compounds of Formula VII-A:

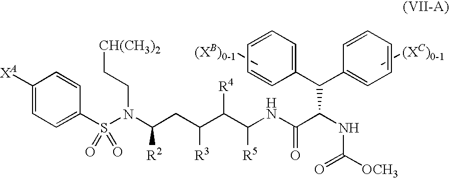

and pharmaceutically acceptable salts thereof, wherein all of the variables are as defined in Class C1.

A second class of compounds of this part of the present invention (Class C2-A) includes compounds of Formula I-A, and pharmaceutically acceptable salts thereof, wherein:

### R¹ is CH₂CH(CH₃)₂ or CH₂CH₂CH(CH₃)₂;

R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃;

### R³ is H or CH₃;

### R⁴ is H or CH₃;

### R⁵ is H or CH₃;

and provided that:

(A) when R² is CH₂OH or CH₂OR^{P}, then at least one of R³, R⁴, and R⁵ is CH₃; and

(B) at least one of R³, R⁴, and R⁵ is H;

### R⁶ is:

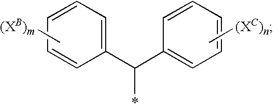

each X^{B} and each X^{C} are independently selected from the group consisting of:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH, and

(17) CH₂OCH₃;

m is an integer equal to 0 or 1;
n is an integer equal to 0 or 1;
each X^{A} is independently:

(1) CH₃,

(2) CH₂CH₃,

(3) CF₃,

(4) OH,

(5) OCH₃,

(6) OCF₃,

(7) Cl,

(8) Br,

(9) F,

(10) CN,

(11) NH₂,

(12) N(H)CH₃,

(13) N(CH₃)₂,

(14) C(O)CH₃,

(15) C(O)OCH₃,

(16) CH₂OH,

(17) CH₂OCH₃,

(18) CH₂NH₂,

(19) CH₂N(H)CH₃,

(20) CH₂N(CH₃)₂,

(21) CH(CH₃)OH,

(22) CH(CH₃)OCH₃,

(23) CH(CH₃)NH₂,

(24) CH(CH₃)N(H)CH₃, or

(25) CH(CH₃)N(CH₃)₂;

k is an integer equal to 0 or 1; and

### R⁷ is H, C(O)CH₃, C(O)OCH₃, C(O)N(CH₃)₂, C(O)-pyridyl, or C(O)-morpholinyl.

A first subclass of Class C2-A (alternatively referred to herein as Subclass SC2-1-A) includes compounds of Formula VI-A and pharmaceutically acceptable salts thereof, wherein all of the variables are as defined in Class C2-A.

A second subclass of Class C2-A (Subclass SC2-2-A) includes compounds of Formula VII-A and pharmaceutically acceptable salts thereof, wherein all of the variables are as defined in Class C2-A.

A twenty-sixth embodiment of this part of the present invention (Embodiment E26-A) is a compound selected from the group consisting of:
methyl [(1S)-2-({(5S)-5-[[4-aminophenyl)sulfonyl]-(3-methylbutyl)amino]-6-hydroxy-1-methylhexyl)-amino)-1-(diphenylmethyl)-2-oxoethyl]carbamate;
methyl {(1S)-1-(diphenylmethyl)-2-[((5S)-6-hydroxy-2-methyl-5-{(3-methylbutyl)[(4-methylphenyl)sulfonyl]amino}hexylamino]-2-oxoethyl}carbamate;
(2S)-2-amino-N-((5S)-6-hydroxy-3-methyl-5-{(3-methylbutyl)[(4-methylphenyl)sulfonyl]amino}hexyl)-3,3-diphenylpropanamide;
methyl {(1S)-1-(diphenylmethyl)-2-[((5S)-6-hydroxy-3-methyl-5-{(3-methylbutyl)[(4-methylphenyl)-sulfonyl]amino}hexylamino]-2-oxoethyl}carbamate;
(2S)-2-amino-N-{5-[[(4-aminophenyl)sulfonyl]-(3-methylbutyl)amino]-6-hydroxyheptyl}-3,3-diphenylpropanamide;
methyl [(1S)-2-({6-amino-5-[[(4-aminophenyl)-sulfonyl]-(3-methylbutyl)amino]-hexyl)-amino)-1-(diphenylmethyl)-2-oxoethyl]carbamate;

and pharmaceutically acceptable salts thereof.

Compounds of Formula I-A form a subset of the compounds included in Formula I. Any description which follows that refers to a compound of Formula I also applies to a compound of Formula I-A.

Another embodiment of the present invention is a compound of Formula I, or a pharmaceutically acceptable salt thereof, as originally defined or as defined in any of the foregoing embodiments, aspects, classes, or subclasses, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis. The compounds of the invention have two or more asymmetric centers and can occur as mixtures of stereoisomers. It is understood that a substantially pure compound can be either a substantially pure mixture of stereoisomers or a substantially pure individual diastereomer or enantiomer.

Other embodiments of the present invention include the following:

(a) A pharmaceutical composition comprising an effective amount of a compound of Formula I as defined above, or a pharmaceutically acceptable salt thereof, and a pharmaceutically acceptable carrier.

(b) A pharmaceutical composition which comprises the product prepared by combining (e.g., mixing) an effective amount of a compound of Formula I as defined above, or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(e) The pharmaceutical composition of (d), wherein the antiviral is selected from the group consisting of HIV reverse transcriptase inhibitors and HIV integrase inhibitors.

(f) A combination which is (i) a compound of Formula I as defined above, or a pharmaceutically acceptable salt thereof, and (ii) an anti-HIV agent selected from the group consisting of HIV antiviral agents, immunomodulators, and anti-infective agents; wherein Compound I and the anti-HIV agent are each employed in an amount that renders the combination effective for inhibition of HIV protease, for treatment or prophylaxis of infection by HIV, or for treatment, prophylaxis of, or delay in the onset or progression of AIDS.

(g) The combination of (f), wherein the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(h) The combination of (g), wherein the antiviral is selected from the group consisting of HIV reverse transcriptase inhibitors and HIV integrase inhibitors.

(i) A method for the inhibition of HIV protease in a subject in need thereof which comprises administering to the subject an effective amount of a compound of Formula I or a pharmaceutically acceptable salt thereof.

(j) A method for the prophylaxis or treatment of infection by HIV (e.g., HIV-1) in a subject in need thereof which comprises administering to the subject an effective amount of a compound of Formula I or a pharmaceutically acceptable salt thereof

(k) The method of (j), wherein the compound of Formula I is administered in combination with an effective amount of at least one other HIV antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(l) The method of (k), wherein the at least one other HIV antiviral is selected from the group consisting of HIV reverse transcriptase inhibitors and HIV integrase inhibitors.

(m) A method for the prophylaxis, treatment or delay in the onset or progression of AIDS in a subject in need thereof which comprises administering to the subject an effective amount of a compound of Formula I or a pharmaceutically acceptable salt thereof.

(n) The method of (m), wherein the compound is administered in combination with an effective amount of at least one other HIV antiviral, selected from the group consisting of HIV protease inhibitors, HIV reverse transcriptase inhibitors, HIV integrase inhibitors, HIV fusion inhibitors, HIV entry inhibitors, and HIV maturation inhibitors.

(o) The method of (n), wherein the at least one other HIV antiviral is selected from the group consisting of HIV reverse transcriptase inhibitors and HIV integrase inhibitors.

(p) A method for the inhibition of HIV protease in a subject in need thereof which comprises administering to the subject the pharmaceutical composition of (a), (b), (c) or (d) or the combination of (e) or (f).

(q) A method for the prophylaxis or treatment of infection by HIV (e.g., HIV-1) in a subject in need thereof which comprises administering to the subject the pharmaceutical composition of (a), (b), (c), (d) or (e).

(r) A method for the prophylaxis, treatment, or delay in the onset or progression of AIDS in a subject in need thereof which comprises administering to the subject the pharmaceutical composition of (a), (b), (c), (d) or (e).

The present invention also includes a compound of Formula I, or a pharmaceutically acceptable salt thereof, (i) for use in, (ii) for use as a medicament for, or (iii) for use in the manufacture/preparation of a medicament for: (a) therapy (e.g., of the human body), (b) medicine, (c) inhibition of HIV protease, (d) treatment or prophylaxis of infection by HIV, or (e) treatment, prophylaxis of, or delay in the onset or progression of AIDS. In these uses, the compounds of the present invention can optionally be employed in combination with one or more other anti-HIV agents selected from HIV antiviral agents, anti-infective agents, and immunomodulators.

Additional embodiments of the invention include the pharmaceutical compositions, combinations and methods set forth in (a)-(r) above and the uses (i)(a)-(e) through (iii)(a)-(e) set forth in the preceding paragraph, wherein the compound of the present invention employed therein is a compound of one of the embodiments, aspects, classes or subclasses described above. In all of these embodiments etc., the compound can optionally be used in the form of a pharmaceutically acceptable salt.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula I or a pharmaceutically acceptable carrier and optionally one or more excipients, it is understood that the term “substantially pure” is in reference to a compound of Formula I or its salt per se.

As used herein, the term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and iso-propyl, ethyl and methyl. As another example, “C₁₋₄ alkyl” refers to n-, iso-, sec- and t-butyl, n- and isopropyl, ethyl and methyl. As another example, “C₁₋₃ alkyl” refers to n-propyl, isopropyl, ethyl and methyl.

The term “alkylene” refers to any divalent linear or branched chain aliphatic hydrocarbon radical having a number of carbon atoms in the specified range. Thus, for example, “C₁₋₆ alkylene-” refers to any of the C₁ to C₆ linear or branched alkylenes, and “—C₁₋₄ alkylene-” refers to any of the C₁ to C₄ linear or branched alkylenes. A class of alkylenes of interest with respect to the invention is —(CH₂)₁₋₆—, and sub-classes of particular interest include —(CH₂)₁₋₄—, —(CH₂)₂₋₄—, —(CH₂)₁₋₃—, —(CH₂)₂₋₃—, —(CH₂)₁₋₂—, and —CH₂—. Another sub-class of interest is an alkylene selected from the group consisting of —CH₂—, —CH(CH₃)—, and —C(CH₃)₂—.

The term “cycloalkyl” refers to any monocyclic ring of an alkane having a number of carbon atoms in the specified range. Thus, for example, “C₃₋₆ cycloalkyl” (or “C₃-C₆ cycloalkyl”) refers to cyclopropyl, cyclobutyl, cyclopentyl, and cyclohexyl, and “C₃₋₅ cycloalkyl” refers to cyclopropyl, cyclobutyl, and cyclopentyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfinyl.

An asterisk (“*”) as the end of an open bond in a chemical group denotes the point of attachment of the group to the rest of the compound.

The term “aryl” refers to phenyl and naphthyl. The aryl of particular interest is phenyl.

The term “heteroaryl” refers to (i) a 5- or 6-membered heteroaromatic ring containing from 1 to 3 heteroatoms independently selected from N, O and S, or (ii) is a heterobicyclic ring selected from quinolinyl, isoquinolinyl, and quinoxalinyl. Suitable 5- and 6-membered heteroaromatic rings include, for example, pyridyl (also referred to as pyridinyl), pyrrolyl, pyrazinyl, pyrimidinyl, pyridazinyl, triazinyl, thienyl, furanyl, imidazolyl, pyrazolyl, triazolyl, oxazolyl, isooxazolyl, oxadiazolyl, oxatriazolyl, thiazolyl, isothiazolyl, and thiadiazolyl. Heteroaryls of particular interest are pyrrolyl, imidazolyl, pyridyl, pyrazinyl, quinolinyl (or quinolyl), isoquinolinyl (or isoquinolyl), and quinoxalinyl.

Examples of 4- to 7-membered, saturated heterocyclic rings within the scope of this invention include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated heterocyclic rings within the scope of this invention (see HetB) include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

It is understood that the specific rings listed above are not a limitation on the rings which can be used in the present invention. These rings are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, an aryl or heteroaryl described as optionally substituted with “from 1 to 4 substituents” is intended to include as aspects thereof, an aryl or heteroaryl substituted with 1 to 4 substituents, 2 to 4 substituents, 3 to 4 substituents, 4 substituents, 1 to 3 substituents, 2 to 3 substituents, 3 substituents, 1 to 2 substituents, 2 substituents, and 1 substituent.

When any variable (e.g., X^{A} or X^{B}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring (e.g., cycloalkyl, aryl, or heteroaryl) provided such ring substitution is chemically allowed and results in a stable compound.

The compounds of the invention contain chiral centers and, as a result of the selection of substituents and substituent patterns, can contain additional chiral centers, and thus can occur as mixtures of stereoisomers, or as individual diastereomers, or enantiomers. All isomeric forms of these compounds, whether individually or in mixtures, are within the scope of the present invention.

To the extent substituents and substituent patterns provide for the existence of tautomers (e.g., keto-enol tautomers) in the compounds of the invention, all tautomeric forms of these compounds, whether present individually or in mixtures, are within the scope of the present invention. Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

The methods of the present invention involve the use of compounds of the present invention in the inhibition of HIV protease (e.g., wild type HIV-1 and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, or benzoic acid. When compounds employed in the present invention carry an acidic moiety (e.g., —COOH or a phenolic group), suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts.

The term “administration” and variants thereof (e.g., “administering” a compound) in reference to a compound of Formula I mean providing the compound to the individual in need of treatment or prophylaxis. When a compound is provided in combination with one or more other active agents (e.g., antiviral agents useful for treating or prophylaxis of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV protease (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the methods of the present invention (i.e., inhibiting HIV protease, treating or prophylaxis of HIV infection or treating, prophylaxis of, or delaying the onset or progression of AIDS), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of a compound of Formula I with one or more anti-HIV agents. An “anti-HIV agent” is any agent which is directly or indirectly effective in the inhibition of HIV reverse transcriptase, protease, or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS, such as those disclosed in Table 1 of WO 01/38332 or in the Table in WO 02/30930. Suitable HIV antivirals for use in combination with the compounds of the present invention include, for example, those listed in Table A as follows:

TABLE A
Antiviral Agents for Treating HIV infection or AIDS

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., abacavir sulfate, indinavir sulfate, atazanavir sulfate, nelfinavir mesylate.

It is understood that the scope of combinations of the compounds of this invention with anti-HIV agents is not limited to the HIV antivirals listed in Table A and/or listed in the above-referenced Tables in WO 01/38332 and WO 02/30930, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson P D R, Thomson P D R, 57^{th} edition (2003), the 58^{th} edition (2004), or the 59^{th} edition (2005). The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV protease, e.g., by competitive inhibition. Thus the compounds of this invention are commercial products to be sold for these purposes.

Abbreviations employed herein include the following: Bn=benzyl; BOC (or Boc)=t-butyloxycarbonyl; Boc₂O=di-t-butyl carbonate; BOP=benzotriazol-1-yloxytris-(dimethylamino)phosphonium; BSA=bovine serum albumin; CBS=Corey, Bakshi, Shibata chiral oxazaborolidine mediated ketone reduction; Cbz=benzyloxycarbonyl; DBU=1,8-diazabicyclo[5.4.0]undec-7-one; DCAD=di-(4-chlorobenzyl) azodicarboxylate; DCE=1,2-dichloroethane; DCM=dichloromethane; DEAD=diethyl azodicarboxylate; DIAD=diisopropylazodicarboxylate; Dibal-H=diisobutylaluminum hydride; DMAP=4-dimethylaminopyridine; DMF=dimethylformamide; DMSO=dimethyl sulfoxide; EDC=1-ethyl-3-(3-dimethylaminopropyl)carbodiimide; Et=ethyl; EtOAc=ethyl acetate; EtOH=ethanol; G-2G=Grubbs catalyst, 2^{nd} generation; HOAt=1-hydroxy-7-azabenzotriazole; HPLC=high performance liquid chromatography; HSU=hydroxysuccinimide; i-PrOH=isopropanol; LAH=lithium aluminum hydride; LC-MS=liquid chromatography-mass spectroscopy; Me=methyl; MeOH=methanol; MOC=methoxycarbonyl; Ms=mesyl or methanesulfonyl; NMR=nuclear magnetic resonance; Ph=phenyl; RCM=ring closing metathesis; Piv=pivaloyl; PPTS=pyridinium p-toluene sulfonate; PyBrOP=bromo-tris-pyrrolidinophosphonium hexafluorophosphate; SCX=strong cation exchange resin; STP=standard temperature and pressure (i.e., 25° C. & 1 atmosphere); TBS=tert-butyldimethylsilyl; TBDPS=tert-butyl(diphenyl)silyl; TBDPSCl=tert-butyl(dimethyl)silyl chloride; TEA=triethylamine; TFA=trifluoroacetic acid; THF=tetrahydrofuran; TLC=thin layer chromatography; TMAF=tetramethyl ammonium fluoride; TMSCHN₂=trimethylsilyl diazomethane; TPAP=tetrapropylammonium perruthenate; TPP=triphenylphosphine.

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The term “Ar” appears in several of the schemes and refers to phenyl optionally substituted with one or more X^{A}.

Scheme A depicts the synthesis of alkylated lysine amine compounds of the invention, wherein carbamate protected amine A1 can be sulfonylated by reaction with an appropriate arylsulfonyl halide to provide A2 which can then be alkylated with an appropriate substituted alcohol using TPP and an azodicarboxylate to provide A3. Intermediate A3 can be deprotected by treatment with hydrogen in the presence of a palladium catalyst to afford amine A4, which can then be coupled to an appropriately substituted amino acid to provide amide A5 via a conventional amidation method such as treating with BOP. The ester group of A5 can be saponified with an hydroxyl base (e.g., NaOH or KOH) to give carboxylic acid A6 which, in turn can be converted to amide A7 using an amide bond forming reagent such as BOP. The amide functional group in A7 can then be reduced (e.g., treatment with a borane reducing agent) to provide desired compound A8.

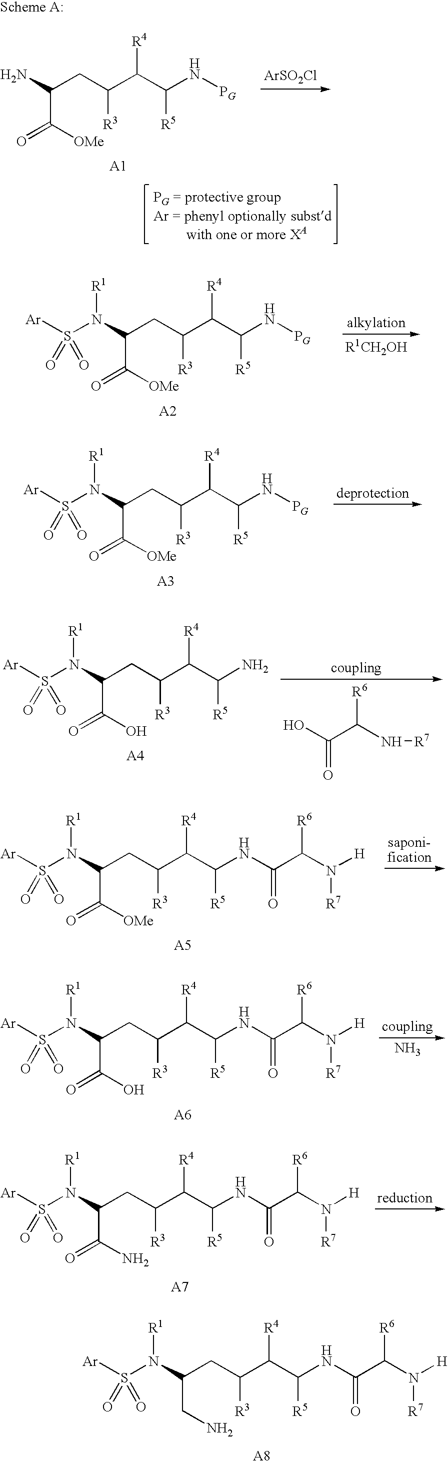

Scheme A′ depicts a method for synthesizing alkylated lysinol compounds of the invention, wherein the ester group in intermediate A5 can be reduced (e.g., by treatment with a metal hydride such as lithium borohydride) to provide desired alcohol A9.

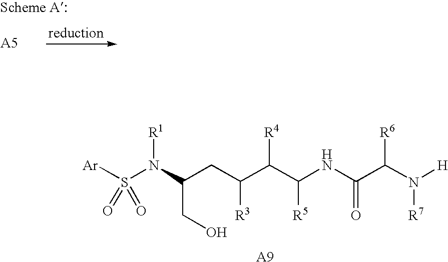

Scheme A″ depicts a method for synthesizing a secondary lysinol or a lysine carbinamine of the present invention, wherein compounds of type A9 can be oxidized to aldehydes A10 with the appropriate R⁷ group for the amine (R⁷=carbamate, carbonyl, sulfonyl etc.). A suitable oxidation method utilizes a sulfur trioxide-pyridine complex in the manner described in Parikh & Doering, J. Am. Chem. Soc 1967, 89: 5505. A10 can be treated with an organometal-derived nucleophile such as methyl magnesium bromide or methyl lithium to afford desired compound A11.

Also depicted in Scheme A″ is the reaction of aldehyde intermediate A10 with Ellman sulfinamide to obtain the corresponding sulfinamine derivative (Ellman et al, J. Am. Chem. Soc, 1967, 120, 8011-8012), which can then be treated with an organometallic nucleophile (identified as R^{J}-M in the scheme) and then with acid to remove the chiral auxiliary and afford desired compound A12.

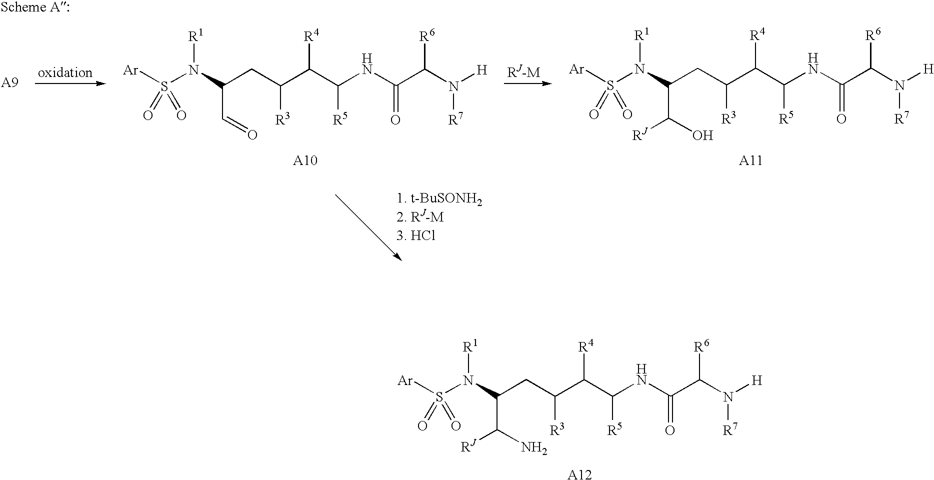

Scheme B depicts an alternative synthesis of alkyl-substituted lysinol compounds of the invention, wherein an appropriately substituted olefinic amino acid B1 can be protected with Boc anhydride and converted to amide B2 using an amide bond forming reagent such as EDC or BOP reagent and an appropriate amine such as an unsubstituted or substituted allyl amine. The Boc protecting group can be removed under acidic conditions and the resulting amine can be sulfonylated with an appropriate arylsulfonyl halide in the presence of a base scavenger such as a tertiary amine (e.g., TEA), a hydroxide (e.g., NaOH), or a carbonate (e.g., sodium bicarbonate) to give B3. The sulfonylamino nitrogen in B3 can be alkylated by reaction with an R^{Q} bearing alkyl alcohol under standard Mitsunobu conditions, and B3 can then be treated with Boc₂O/DMAP to afford B4 (see Brass et al., Tetrahedron 2006, 1777). Diene B4 can be converted to lactam B5 using standard reagents (e.g., a second generation Grubbs catalyst) that effect a ring closing metathesis reaction. Lactam B5 can be reduced (e.g., with a borohydride reagent in an alcoholic solvent) to give B6, which can subsequently be hydrogenated and deprotected under acidic conditions (e.g., HCl) to afford amino alcohol B7. The amino group in B7 can then be coupled with an appropriately substituted amino acid to afford the desired amide B8.

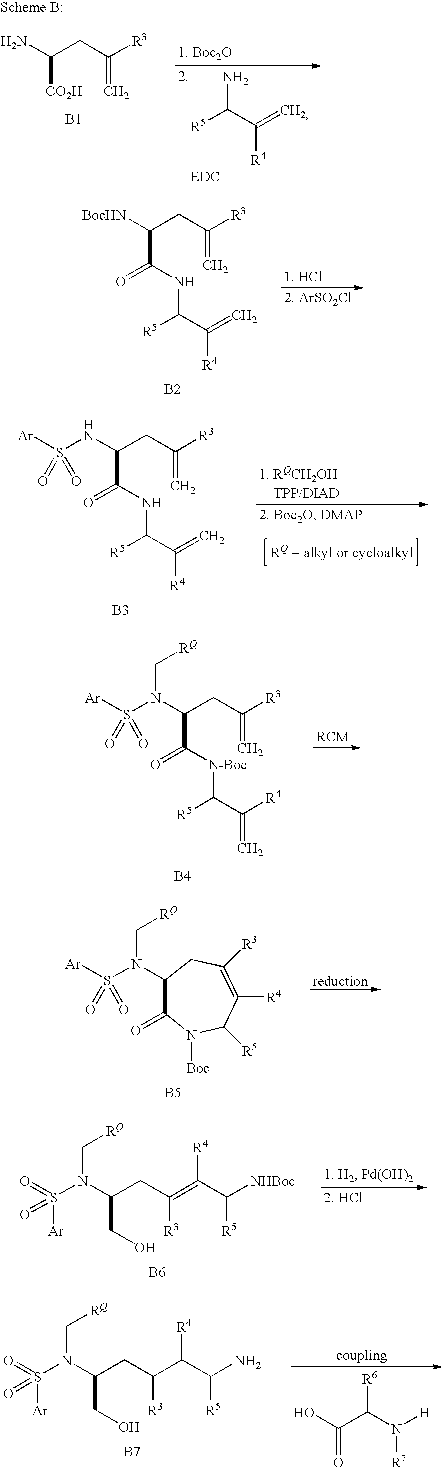

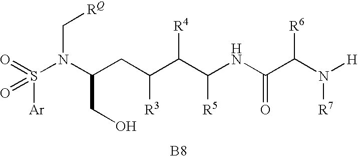

Scheme C depicts another synthesis of alkylated lysinol compounds of the invention, wherein an appropriately substituted olefinic amino acid C1 can be sulfonylated with an appropriate arylsulfonyl halide in the presence of a base scavenger such as a tertiary amine (e.g., TEA), a hydroxide (e.g., NaOH), or a carbonate (e.g., sodium bicarbonate) to give C2. Sulfonamide C2 can be alkylated with an appropriate alcohol in the presence of TPP and an azodicarboxylate using Mitsunobu conditions and then saponified with an hydroxyl base such as NaOH or KOH to give intermediate C4. Compound C4 can be coupled with an olefinic amine using an amide bond forming reagent such as BOP to afford amide C5. The diene in C5 can be converted to lactam C6 using standard reagents that effect a ring closing metathesis reaction such as a second generation Grubbs catalyst. The lactam protecting group can be removed by subjecting C6 to strongly acidic conditions, and then the double bond can be reduced using standard hydrogenation conditions (e.g, Pd on carbon or Pd(OH)₂ on carbon with hydrogen gas) to give C7. Lactam C7 can then be treated with Boc anhydride and the Boc-protected lactam subjected to reductive ring opening by reaction with a borohydride reagent in an alcoholic solvent such as methanol or ethanol to afford C8. Deprotection of C8 by treatment with an acid such as TFA, followed by coupling with an appropriately substituted amino acid derivative can provide the desired compound C9.

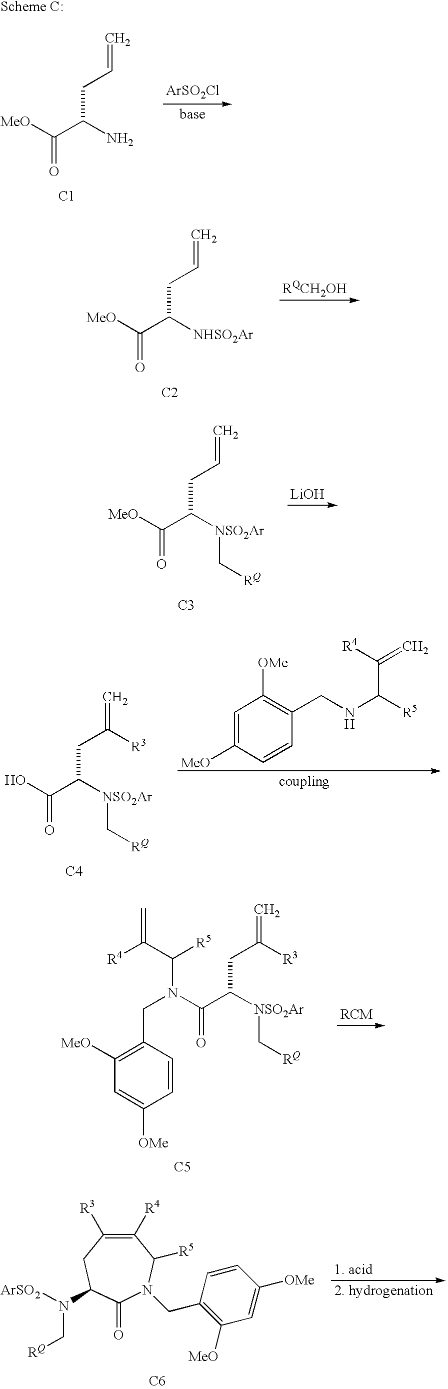

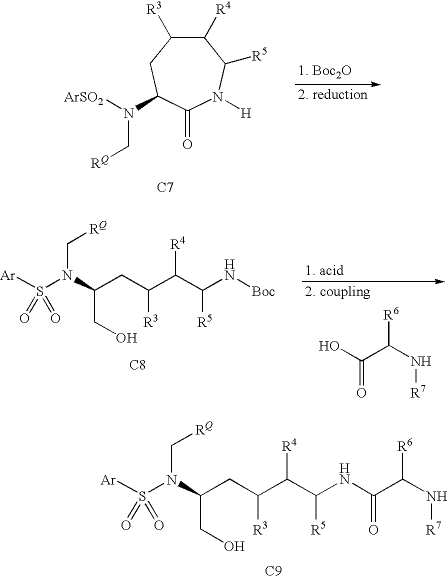

Scheme D depicts another synthesis of alkylated lysinol compounds of the invention, wherein an appropriately protected glutamic acid derivative such as D1 can be esterified and Boc protected to give fully protected glutamate derivative D2. Glutamate derivative D2 can be selectively reduced using an appropriate reducing agent such as diisobutylaluminum hydride to provide aldehyde D3 which can undergo a Henry reaction (see, e.g., Comp. Org. Syn. 1991, 2: 321) by treatment with an appropriately substituted nitroalkyl group and a catalytic base such as tetramethylguanidine. The resulting Henry adduct can be activated with a reagent such as mesyl chloride and then treated with an amine base such as TEA to provide D4. The double bond in D4 can be reduced by hydrogenation in the presence of a Pd source to afford amino acid D5, which can be sequentially protected and deprotected by treatment with an amino protecting agent such as Cbz chloride followed by treatment with alcoholic HCl to provide D6. D6 can be sulfonylated with a suitable arylsulfonyl halide in the presence of a base to provide D7, which can then be alkylated to afford D8 with an appropriately substituted alcohol under Mitsunobu alkylation conditions using TPP and an azodicarboxylate. Intermediate D8 can then be deprotected using hydrogen and a palladium catalyst to provide an amine which can be coupled to an appropriately substituted amino acid derivative to afford D9, which can then be reduced to provide the desired D10. Chiral separation can provide all stereoisomers which can be identified by enzymatic inhibition evaluation. Absolute assignment of stereochemistry at the R⁵ bearing epsilon center can be obtained by cocrystallization with HIV protease.

Alternatively, amine D5 can be coupled directly to an appropriately substituted amino acid derivative to provide intermediate D11, after concomitant Boc removal and esterification. Sulfonylation with a suitable arylsulfonyl halide in the presence of a base provides sulfonamide D12 at which point the diastereoisomers at the R⁵ bearing epsilon center can be separated by flash chromatography. The desired isomer (R⁵ being alpha, as shown on D12) can be identified by conversion of both diastereoisomers to the final compounds D13, using Mistunobu alkylation, nitro and ester reduction as described above, and enzymatic inhibition evaluation on both diastereoisomers. Absolute assignment of stereochemistry at the R⁵ bearing epsilon center can be obtained by cocrystallization with HIV protease.

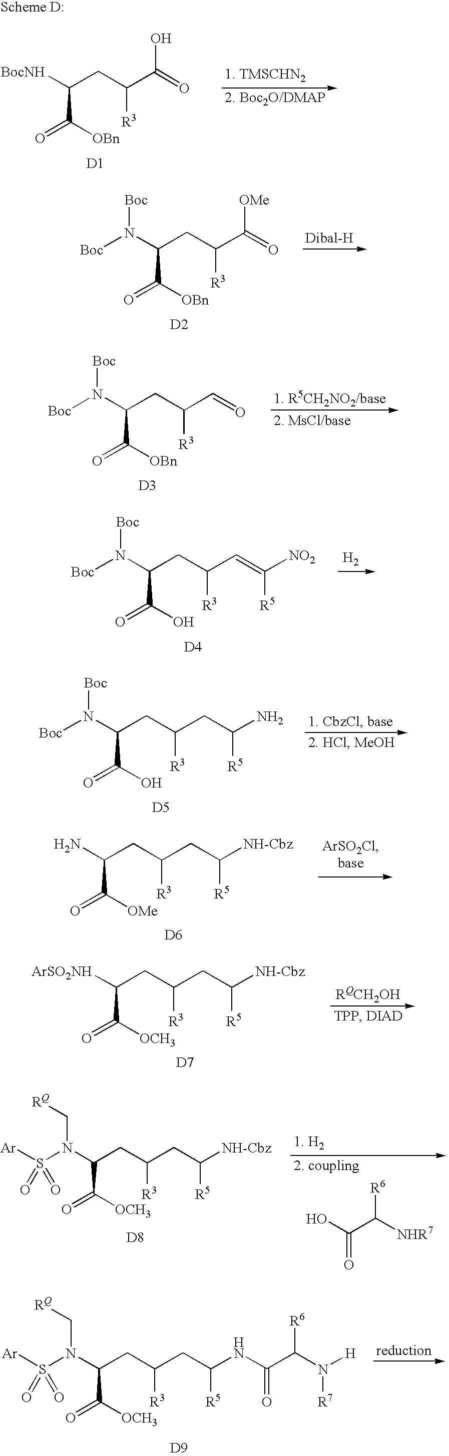

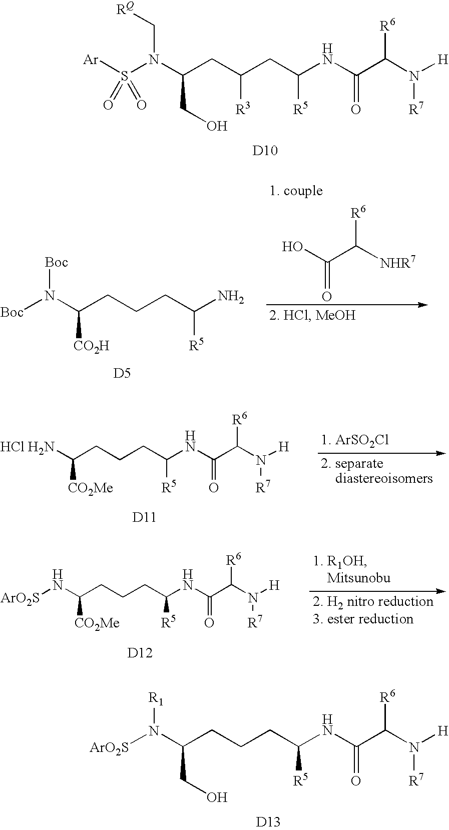

Scheme E depicts a first method used to introduce the R⁵ substituent with control of diastereoselectivity. Boc lysine E1 is converted to the corresponding bis-Boc intermediate on which the ester can be reduced and the resulting alcohol protected as a silyl ether to provide intermediate E2. Selective RuO₄ mediated oxidation, alpha to the terminal NHBoc, according to Tetrahedron Lett. 1998, 39, 5671, followed by reduction of the resulting imide provides alcohol E3. Protection of the terminal hydroxyl group as a pivalate or benzyl ether allows for subsequent alkylation of the NHBoc group with a R¹ containing halide, to provide intermediate E4. Pivalate or benzyl ether removal followed by oxidation of the resulting primary alcohol to the corresponding aldehyde, and its conversion to the corresponding diastereomerically pure Ellman sulfinimide of choice affords intermediate E5. Diastereoselective introduction of the R⁵ group can be achieved by addition of a R⁵ containing Grignard regent to the Ellman sulfinimide functionality. Treatment with a controlled amount of HCl in MeOH affords the amino-alcohol E6. Coupling of an appropriately substituted amino acid derivative, followed by Boc removal and sulfonylation provides the desired compounds of type E7. Reduction of nitro or ester functionalities on the Ar group can also be performed at this stage if necessary.

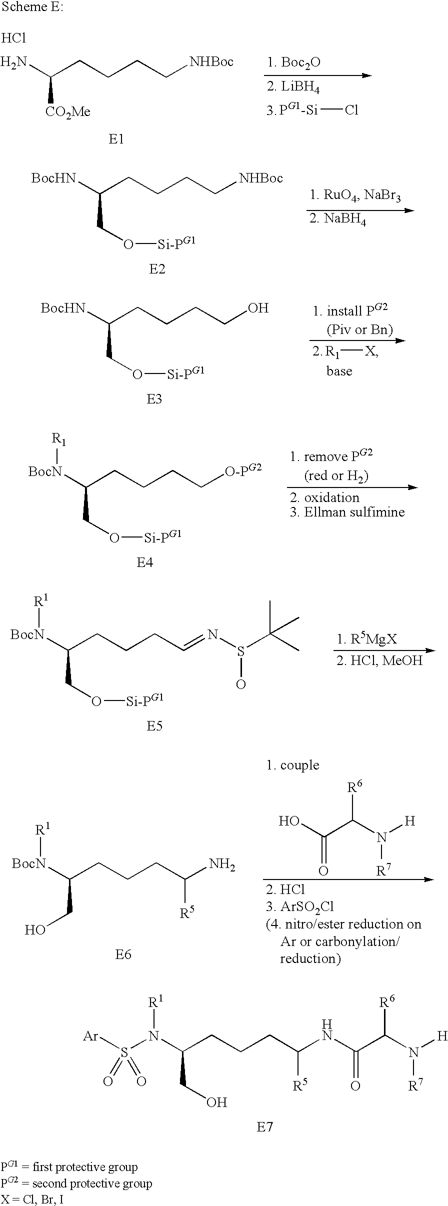

Scheme F depicts the utilization of cross metathesis methodology to introduce the substituted lysine side chain and the utilization of diastereoselective reduction of Ellman sulfinimide to control the stereochemistry at the R⁵ bearing center. Allyl glycine is converted to the corresponding methyl or ethyl ester and then sulfonylated and alkylated under Mistunobu conditions to provide intermediate F2. Cross metathesis (see Handbook of Metathesis; Grubbs, R. H., Ed.; Wiley-VCH: Weinheim, 2003) with a R⁵ bearing crotyl ketone and using Grubbs 2^{nd} generation catalyst affords, after hydrogenation of the double bond and nitro group, ketone F3. Conversion to the corresponding diastereomerically pure Ellman sulfinimide of choice followed by diastereoselective reduction and Ellman group removal under acidic conditions affords amine F4. Coupling of an appropriately substituted amino acid derivative and reduction of the ester group leads to the desired products of type F5.

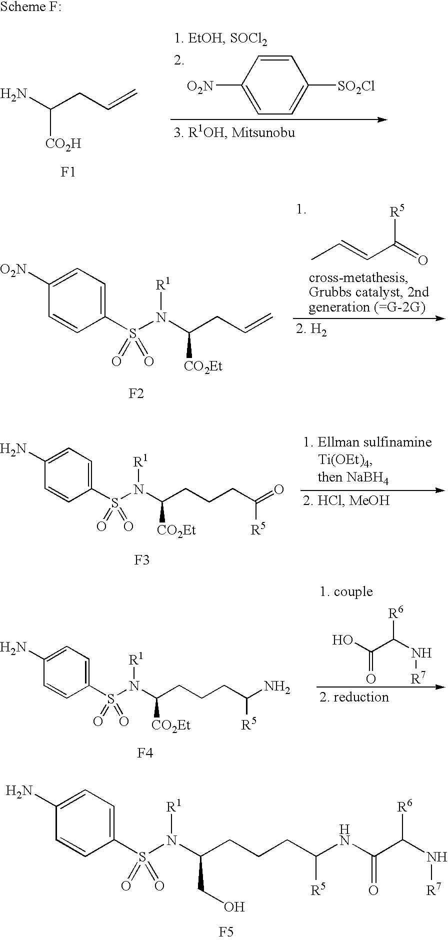

Scheme G depicts a variation around the methodology described in Scheme F that allows for the later introduction of the aryl sulfonamide and R¹ groups. Allyl glycine is converted to the Boc ester derivative G2 which is in turn converted to the ketone G3 via olefin cross metathesis and then the amine G4 in a similar manner as described earlier in Scheme F. Coupling of an appropriately substituted amino acid derivative and Boc removal provides intermediate G5 which is ready for sulfonylation and Mitsunobu alkylation to ultimately afford desired compounds of type G6 after ester reduction.

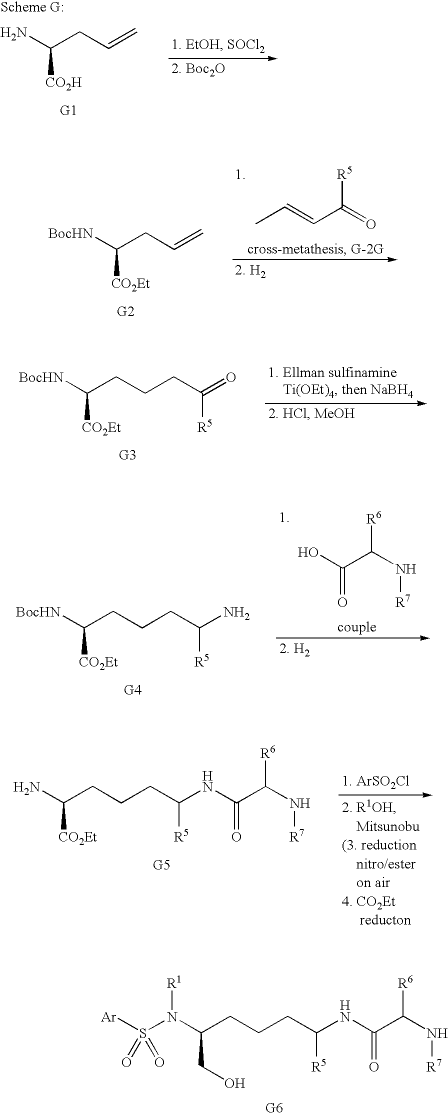

Scheme H depicts a variation around the methodology described in scheme G that allows for the introduction of CF₃ or CF₂-alkyl groups at the R⁵ position. Aldehyde H2 is prepared using methodology described in Schemes F and G, after which Ellman sulfinimide is prepared as described before, and can then be treated with CF₃-TMS and a fluoride source to afford a diastereoselective anti addition of a CF₃ group, which, after HCl/MeOH treatment affords amine H3. Coupling of an appropriately substituted amino acid derivative followed by Mitsunobu alkylation, nitro and ester reduction provides the desired compounds of type H4.

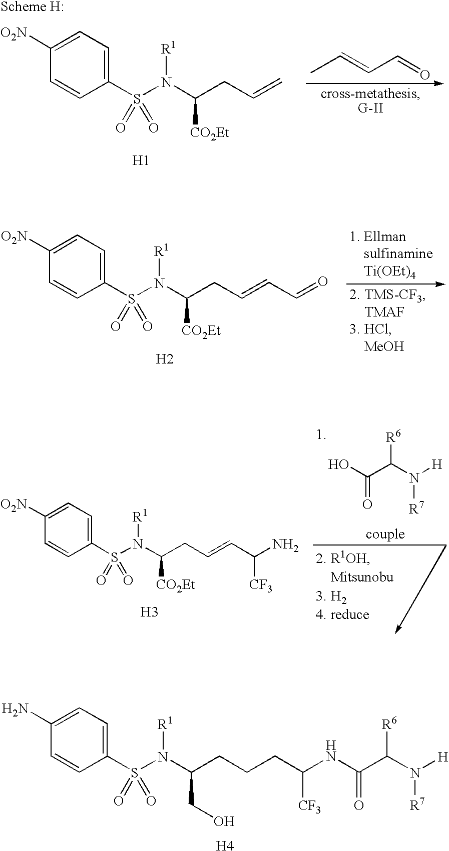

Scheme I depicts yet another approach to the preparation of ketones of type I2. Cyclic imide I1 can be converted to its corresponding ester-Boc-imide which can in turn be regioselectively opened by the addition of a R⁵ containing Grignard to afford ketone I2. The conversion of ketone I2 to the desired product of type I5 proceeds as described earlier in scheme G.

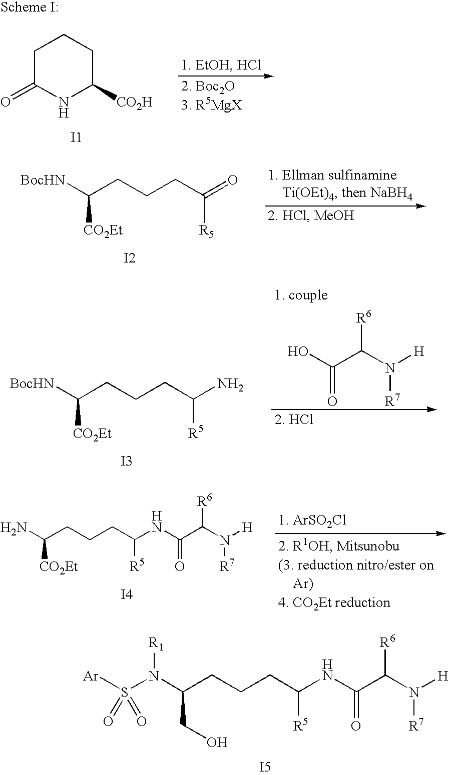

In Part 1 of Scheme J an alternative strategy is depicted that provides access aldehyde intermediates as precursors of Ellman sulfinimides. Amino-acid J1 (commercially available) is converted to benzyl ether J2 via esterification, sulfonylation and Mitsunobu alkylation. Concomitant reduction of both methyl esters and protection of the resulting alcohols as silyl ethers allows for the selective hydrogenolysis of the terminal benzyl ether which can then be oxidized to the corresponding aldehyde J3. At this point the Ellman sulfinimide can be prepared and treated with either R⁵ containing Grignard or CF₃-TMS and a fluoride source to allow for the diastereoselective introduction of the R⁵ group. Acidic deprotection of the sulfimine group and the silyl ethers, and coupling of an appropriately substituted amino acid derivative affords desired products of type J4. Part 2 of Scheme J, a modified version of Part 1, depicts the preparation of branched benzyl alcohol derivatives of type J7. Preparation of acetophenones of type J5 is conducted utilizing similar methodology to that just described for the conversion of J1 to J2. The acetophenone group can be diastereoselectively reduced using Corey's CBS methodology (J. Am. Chem. Soc. 1987, 109, 5551-5553 and 7925-7926) and protected as the corresponding silyl ether. At this point the ester is reduced and protected as the corresponding silyl ether, and then the terminal alcohol is deprotected and oxidized to the aldehyde intermediate J6. Conversion to desired product of type J7 follows the same methodology as just described for the conversion of J3 to J4.

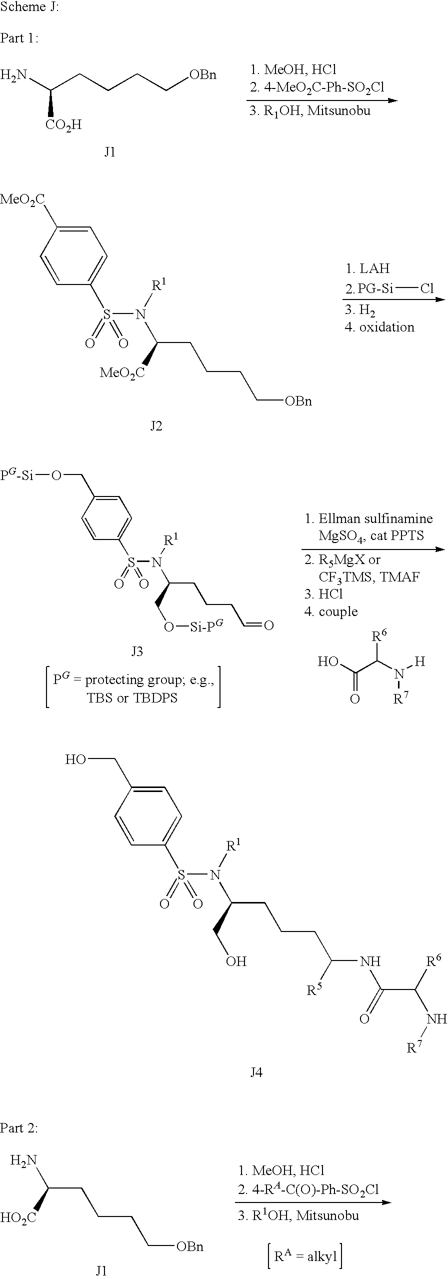

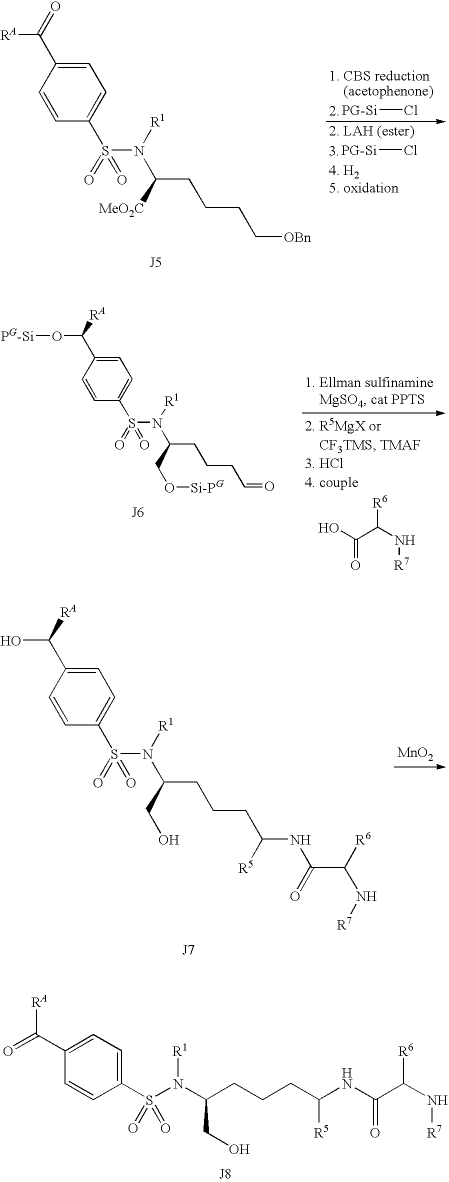

Scheme K depicts a combination of methodologies utilized in schemes F and J. Allyl glycine is converted to the bis ester K2 which can be reduced and protected as the bis silyl ether K3. Olefin cross metathesis (Handbook of Metathesis; Grubbs, R. H., Ed.; Wiley-VCH: Weinheim, 2003) with crotonaldehyde followed by hydrogenation of the double bond affords aldehydes of type K4 which in turn can be converted to desired products of type K5 by following a similar procedure as described in Scheme J. As described in Scheme J, a minor variation allows for the conversion of K1 to branched benzyl alcohols of type K9. Selective benzylic oxidation provides acetophenones of type K10.

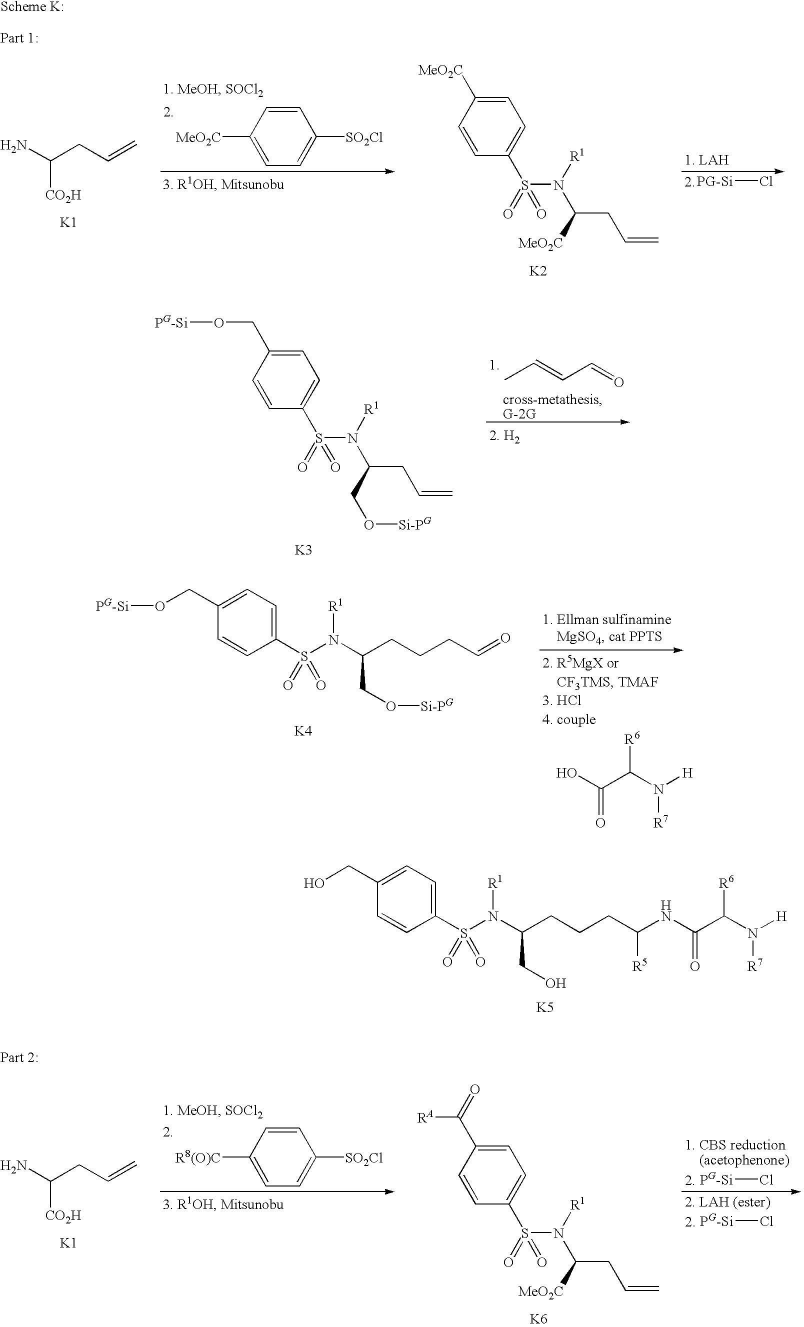

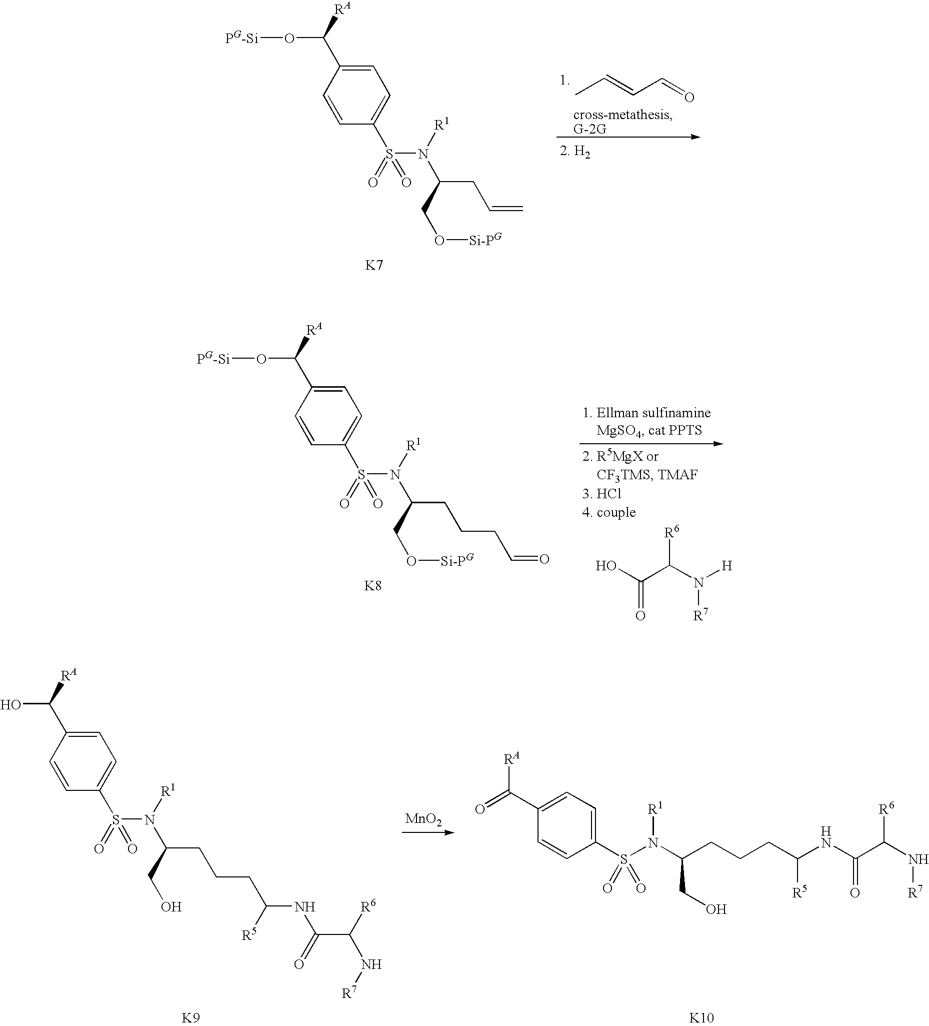

Scheme L depicts the preparation of spiro epsilon-substituted compounds of type L4 and gem-disubstituted compounds of type L9. Part 1 depicts the spiro compounds, wherein Michael addition of nitro derivatives of type L1 to acrolein (Org. Lett. 2003, 5(17), 3155-3158) followed by Horner-Emmons addition to the aldehyde functionality affords intermediates of type L2. Concomitant nitro reduction and Cbz removal followed by sulfonylation gives access to intermediates of type L3. Coupling of an appropriately substituted amino acid derivative, R¹ group installation, nitro and ester reductions provide desired products of type L4. Part 2 depicts a methodology similar to that of Part 1 for the preparation of gem-disubstituted intermediates of type L7 from which desired products of type L9 can be obtained.

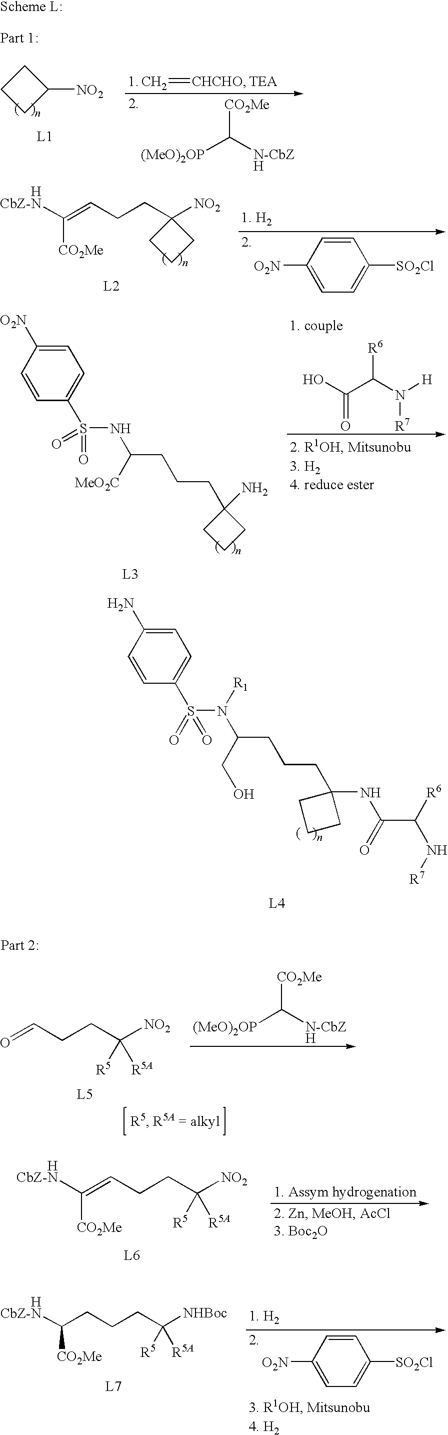

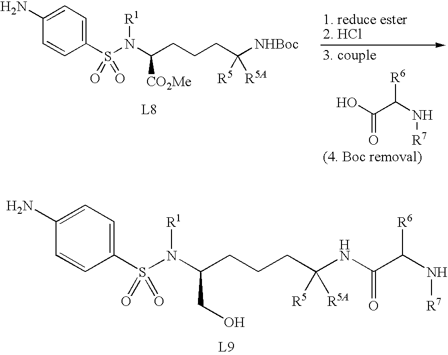

Scheme M depicts the preparation of hydroxymethyl derivatives of type M3. 2,6-diaminoheptanedioic acid can be converted to the bis ester and then monosulfonylated followed by Cbz installation to provide intermediate M2. Installation of R¹, followed by coupling of an appropriately substituted amino acid derivative and reduction of the ester groups provides derivatives of type M3.

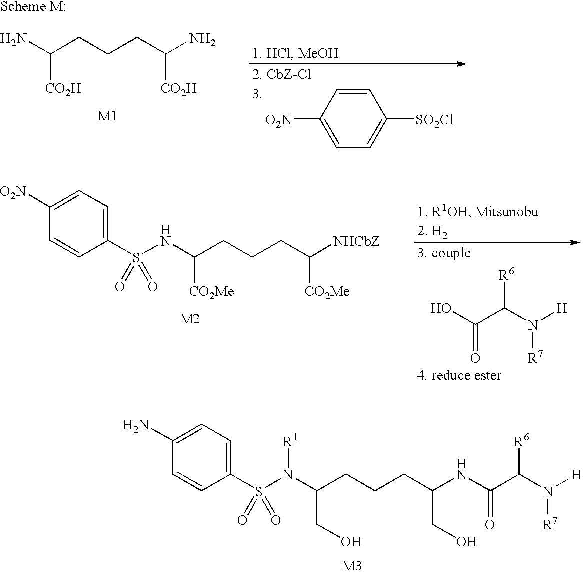

In compounds of Formula I in which R² is CH(R^{J})—OR^{P}, the R^{P} group can be introduced using procedures similar or identical to those described in WO 2006/012725 (see, e.g., Schemes 1, 1A, 2, 3, 4 and 5 in WO'725).

The following examples serve only to illustrate the invention and its practice. The examples are not to be construed as limitations on the scope or spirit of the invention.

The term “room temperature” in the examples refers to the ambient temperature which was typically in the range of about 19° C. to 26° C.

### Preparative Example S

### Intermediate 1: N-(2,4-dimethoxybenzy)-2-methylprop-2-en-1-amine

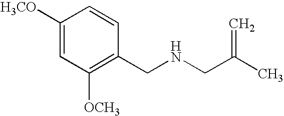

To a solution containing 5.55 g (27.3 mmol) of 2,4-dimethoxybenzylamine hydrochloride and 3.68 g (27.3 mmol) of propenyl bromide in 100 mL of DCM was added 8.0 mL (57.2 mmol) of TEA. The reaction mixture was stirred for 16 hours, diluted with 50 mL of NaHCO₃ solution and washed with DCM (3×). The organic extracts were dried, concentrated and the residue was chromatographed (95/5/0.5) DCM/MeOH/NH₄OH to give the desired amine. LCMS [M+H]⁺=222.5.

### Example A1

### (2S)-2-amino-N-{5-[[(4-aminophenyl)sulfonyl]-(3-methylbutyl)amino]-6-hydroxyheptyl}-3,3-diphenylpropanamide

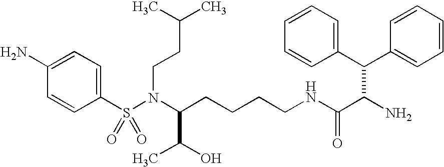

### Step A1-1: tert-Butyl[(1S)-2-({(5S)-5-[[(4-aminophenyl)sulfonyl](3-methylbutyl)amino]-6-oxohexyl}amino)-1-(diphenylmethyl)-2-oxoethyl]carbamate

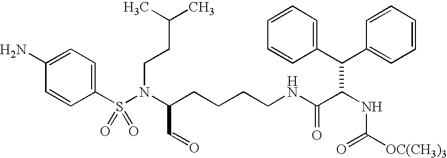

To a solution of tert-butyl[(1S)-2-({(5S)-5-[[4-aminophenyl)sulfonyl](3-methylbutyl)amino-6-hydroxyhexyl)amino)-1-(diphenylmethyl)-2-oxoethyl]carbamate (500 mg, 0.734 mmol); prepared as described in Stranix et al. Bioorg. Med. Chem. Lett. 2006, 16(13): 3459) and Hunig's base (0.641 mL, 3.67 mmol) in 5 mL of DMSO and 2.6 mL of CH₂Cl₂ at −10° C. was added SO₃-Py (584 mg, 3.67 mmol) in 2.8 mL DMSO via cannula. The bath was removed, and the reaction was allowed to proceed at room temperature for 3 hours. The reaction mixture was quenched by the addition of 2M Na₂S₂O₃ and stirred vigorously at room temperature for 30 minutes. The reaction mixture was diluted with EtOAc, the layers were separated, and the organics were washed with 2M Na₂S₂O₃ (1×), 3M LiCl (3×) and brine. The organics were dried over Na₂SO₄, filtered and concentrated. The residue was purified by silica gel chromatography (0->15% MeOH/CH₂Cl₂, linear gradient) to yield the desired product as a white solid.

LCMS [M+H]⁺=679.

### Step A1-2: tert-Butyl[(1S)-2-({5-[[(4-aminophenyl)sulfonyl](3-methylbutyl)amino]-6-hydroxyheptyl}amino)-1-(diphenylmethyl)-2-oxoethyl]carbamate

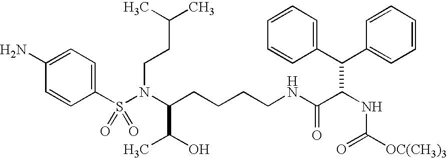

To a solution of the aldehyde from step A1-1 (100 mg, 0.147 mmol) in 2.9 mL THF at −78° C. was added MeMgBr (0.49 mL as a 3M solution in Et₂O, 1.47 mL). The reaction mixture was allowed to warm to −15° C. over 2 hours, and the reaction mixture was quenched by the addition of saturated NH₄Cl, followed by EtOAc. The layers were separated, and the organics were washed with brine, dried over Na₂SO₄, filtered and concentrated. The residue was purified by silica gel chromatography (0->15% MeOH/CH₂Cl₂, linear gradient) to yield the desired product as a white solid which was a 1:1 mixture of diastereomers by ¹H NMR. LCMS [M+H]⁺=695.

### Step A1-3: (2S)-2-amino-N-(5-[[(4-aminophenyl)sulfonyl]-(3-methylbutyl)amino]-6-hydroxyheptyl}-3,3-diphenylpropanamide

To a solution of product from step A1-2 (25 mg, 0.036 mmol) in 0.72 mL CH₂Cl₂ was added 0.67 mL 4M HCl in dioxane. After 2 hours, the reaction mixture was concentrated, redissolved in 1 mL DMF and purified by preparative HPLC (Sunfire column, 15 mL/min) to yield the title compound as an inseparable 1:1 mixture of diastereomers. The ¹H NMR data tabulated below is for this diastereomeric mixture. ¹H NMR (400 MHz, d₄-MeOH) δ 7.82 (m, 1H), 7.50-7.43 (m, 4H), 7.39 (t, J=7.5 Hz, 2H), 7.32-7.21 (m, 6H), 6.74 (dd, J=8.6, 1.5 Hz, 1H), 4.52 (d, J=11.6 Hz, 1H), 4.31 (d, J=11.7 Hz, 1H), 3.69 (m, 1H from one diastereomer), 3.55 (m, 1H from one diastereomer), 3.44 (m, 1H from 1 diastereomer), 3.33 (m, 1H from 1 diastereomer), 3.17-3.00 (m, 4H), 2.71 (m, 1H), 1.58-1.28 (m, 6H), 1.11 (d, J=6.2 Hz, 3H from one diastereomer), 1.04 (d, J=6.4 Hz, 3H from one diastereomer), 0.92 (m, 2H), 0.85 (m, 6H); LCMS [M+H]⁺=595.

### Example A2

### Methyl [(1S)-2-({6-amino-5-[[(4-aminophenyl)sulfonyl](3-methylbutyl)amino]-hexyl)amino)-1-(diphenylmethyl)-2-oxoethyl]carbamate

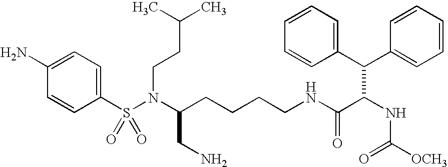

### Step A2-1: Methyl (2S)-6-{[(benzyloxy)carbonyl]amino}-2-([(4-nitrophenyl)sulfonyl]amino}hexanoate

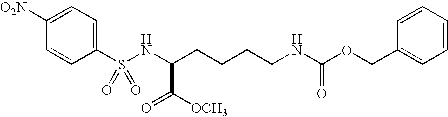

To a solution containing 5.0 g (17 mmol) of ester in 100 mL of DCM was added 4.7 mL (34 mmol) of triethylamine followed by 3.7 g (17 mmol) of p-nitrobenzenesulfonyl chloride and the resulting mixture was allowed to stir at room temperature for 16 hours. The solution was washed with 1 N HCl (2×20 mL), saturated NaHCO₃ (2×10 mL), water (10 mL), and brine (10 mL). The organic phase was dried over MgSO₄, concentrated and chromatographed (33% to 50% to 100% EtOAc/hexanes) to afford the desired product. LCMS (M+1)=480.1.

### Step A2-2: Methyl (2S)-6-{[(benzyloxy)carbonyl]amino}-2-{(3-methylbutyl)[(4-nitrophenyl)sulfonyl]amino}hexanoate

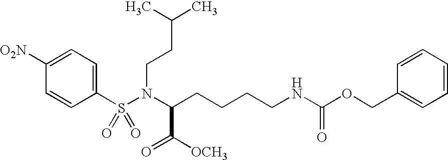

Sulfonamide A2-1 (1.0 g, 2.09 mmol) was dissolved in 10 mL of THF and treated sequentially with triphenylphosphine (656 mg, 2.5 mmol), isoamyl alcohol (221 mg, 2.5 mmol), and DIAD (506 mg, 2.5 mmol) and the resulting solution was allowed to stir for 72 hours at room temperature. The reaction mixture was concentrated and chromatographed (50% EtOAc/hexanes) to afford the desired product. LCMS (M+1)=550.2.

### Step A2-3: Methyl (2S)-6-amino-2-[[(4-aminophenyl)sulfonyl](3-methylbutyl)amino]hexanoate

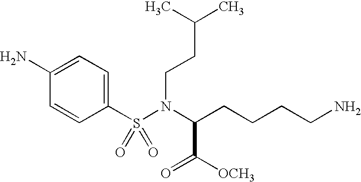

A degassed solution containing 2.0 g (3.64 mmol) of compound A2-2 dissolved in 50 mL of MeOH was treated with 500 mg of 20% Pd(OH)₂ and hydrogenated at STP for 2 hours. The reaction mixture was filtered through Celite and evaporated to leave the desired compound. LCMS (M+1)=386.0.

### Step A2-4: Methyl 2-[[(4-aminophenyl)sulfonyl](3-methylbutyl)amino]-6-({(2S)-2-[(methoxycarbonyl)amino]-3,3-diphenylpropanoyl}amino)hexanoate

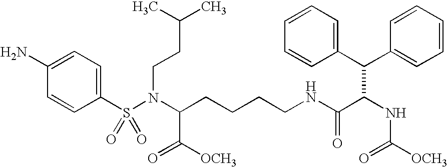

To a solution of the amine from step A2-3 (1.0 g, 2.59 mmol) and N-Moc-(S)-diphenylalanine (854 mg, 2.85 mmol) in 20 mL DCM was added diisopropylethylamine (805 mg, 6.23 mmol) and BOP reagent (1.38 g, 3.11 mmol). After 60 minutes, the reaction mixture was diluted with DCM and washed with saturated NaHCO₃ The organic phase was separated, dried and evaporated. Column chromatography (80% EtOAc/hexanes) afforded the desired adduct as a white solid. LCMS (M+1)=667.8.

### Step A2-5: 2-[[(4-Aminophenyl)sulfonyl](3-methylbutyl)amino]-6-({(2S)-2-[(methoxycarbonyl)amino]-3,3-diphenylpropanoyl}amino)hexanoic acid

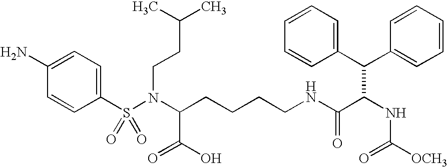

A solution containing 667 mg (1.00 mmol) of ester dissolved in 3 mL of THF and 3 mL of water was treated with 3 mL (6.0 mmol) of 2N LiOH and the resulting mixture was stirred at room temperature for 16 hours. The mixture was acidified to pH=5 with 1N HCl and washed with EtOAc (3×10 mL). The combined organics were dried over MgSO₄ and concentrated to give the desired acid. LCMS [M+H]⁺=653.

### Step A2-6: Methyl [(1S)-2-({6-amino-5-[[(4-aminophenyl)sulfonyl](3-methylbutyl)amino]-6-oxohexyl}amino)-1-(diphenylmethyl)-2-oxoethyl]carbamate

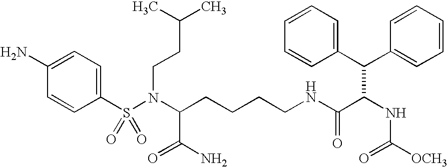

To a solution of the carboxylic acid from step A2-5 above (65 mg, 0.10 mmol) and ammonium chloride 10.4 mg, 0.2 mmol) in 1 mL DMF was added triethylamine (0.040 mL, 0.285 mmol) and BOP reagent (88 mg, 0.200 mmol). After 30 minutes, the reaction mixture was diluted with EtOAc, and the organics were washed with H₂O and brine, dried over Na₂SO₄, filtered and concentrated. The residue was purified by reverse phase chromatography to afford the desired adduct was a viscous oil. LCMS [M+H]⁺=652.8.

### Step A2-7: Methyl [(1S)-2-({6-amino-5-[[(4-aminophenyl)sulfonyl](3-methylbutyl)amino]-hexyl)amino)-1-(diphenylmethyl)-2-oxoethyl]carbamate

To a solution containing 50 mg (0.07 mmol) of the amide from step A2-6 above in 1 mL of THF was added 0.04 mL (0.08 mmol) of 2M borane in THF. The resulting mixture was stirred at room temperature for 16 hours, quenched with 1 mL of MeOH and evaporated to dryness. The residue was subjected to reverse phase chromatography to afford the desired amine as a white foam.

¹H NMR (CDCl₃): δ 7.60-7.58 (d, J=9 Hz, 2H), 7.34-7.19 (m, 10H), 6.72-6.70 (d, J=9 Hz, 2H), 5.57 (br s, 1H), 5.23-5.21 (d, J=8 Hz, 1H), 4.81-4.77 (m, 1H), 4.46-4.39 (m, 3H), 3.74-3.32 (m, 6H), 3.19-3.11 (m, 2H), 2.90-2.85 (m, 1H), 2.68-2.56 (m, 3H), 1.57-0.86 (m, 13H), 0.57 (br s, 2H). LCMS [M+H]⁺=638.8.

### Example B1

### (2S)-2-amino-N-((5S)-6-hydroxy-3-methyl-5-{(3-methylbutyl)[(4-methylphenyl)-sulfonyl]amino}hexyl)-3,3-diphenylpropanamide

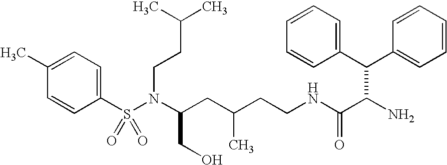

### Step B1-1: (2S)-2-[(tert-Butoxycarbonyl)amino]-4-methylpent-4-enoic acid

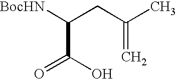

To a solution of (2S)-2-amino-4-methyl-4-pentenoic acid (500 mg, 3.87 mmol) in 13 mL dioxane and 3.9 mL 3M NaOH was added Boc₂O (887 mg, 4.06 mmol) in one portion. The reaction was allowed to proceed at room temperature for 16 hours, then acidified to pH˜2 by the addition of 1N HCl. The aqueous was extracted with CHCl₃ (4×), the combined organics were dried over Na₂SO₄, filtered and concentrated to yield the desired protected amino acid as a white solid. LCMS [M+H]⁺=230.

### Step B1-2: tert-Butyl{(1S)-1-[(allylamino)carbonyl]-3-methylbut-3-en-1-yl}carbamate

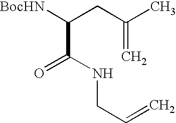

To a solution of N-Boc protected amino acid from Step B1-1 above (893 mg, 3.89 mmol) in 13 mL CHCl₃ was added allylamine (0.35 mL, 4.67 mmol), followed by EDC-HCl (896 mg, 4.67 mmol) and HOAt (53 mg, 0.389 mmol). The reaction was allowed to proceed at room temperature for 16 hours, then diluted with EtOAc. The organics were washed with 1N H HCl, saturated aqueous NaHCO₃ and brine, dried over Na₂SO₄, filtered and concentrated to obtain the desired coupled adduct as a white solid, which was used without further purification. LCMS [M+H]⁺=269.

### Step B1-3: (2S)—N-Allyl-4-methyl-2-amino-4-methylpent-4-enamide

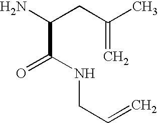

Adduct from Step B1-2 was dissolved in 17 mL EtOAc and cooled to 0° C. HCl gas was bubbled through the reaction for 5 minutes, and the reaction mixture was warmed to room temperature for 1 hour. The reaction mixture was cooled back to 0° C., and HCl gas was bubbled through the reaction again for 2 minutes. The reaction mixture was warmed to room temperature for 1 hour and concentrated to afford the desired product as a white solid. LCMS [M+H]⁺=169.

### Step B1-4: (2S)—N-Allyl-4-methyl-2-[(4-methylphenyl)sulfonyl]amino}pent-4-enamide

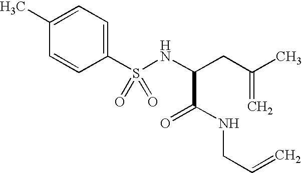

To a solution of product from Step B1-3 (610 mg, 2.98 mmol) in 15 mL CH₂Cl₂ was added triethylamine (0.831 mL, 5.96 mmol). Tosyl chloride (568 mg, 2.98 mmol) was added in one portion, and the reaction was allowed to proceed at room temperature for 36 hours. The reaction mixture was diluted with EtOAc and the organics were washed with brine, dried over Na₂SO₄, filtered and concentrated. The residue was purified using silica gel chromatography (10->65% EtOAc/hexanes, linear gradient) to obtain the desired product as a viscous oil. LCMS [M+H]⁺=323.

### Step B1-5: (2S)—N-Allyl-4-methyl-2-{(3-methylbutyl)[(4-methylphenyl)sulfonyl]amino}pent-4-enamide

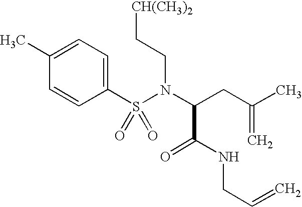

To a solution of product from Step B1-5 (546 mg, 1.69 mmol) in 5.6 mL THF was added 3-methylbutanol (0.26 mL, 2.37 mmol), Ph₃P (622 mg, 2.37 mmol) and DIAD (0.46 mL, 2.37 mmol) in that order. The reaction was allowed to proceed at room temperature for 3 hours, then concentrated, and the residue was purified by silica gel chromatography (0->20% EtOAc/hexanes, linear gradient) to yield the desired product as a white solid. LCMS [M+H]⁺=393.

### Step B1-6: tert-Butyl allyl((2S)-4-methyl-2-{(3-methylbutyl)[(4-methylphenyl)sulfonyl]amino}pent-4-enoyl)carbamate

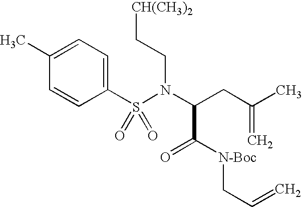

To a solution of amide from Step B1-5 (173 mg, 0.441 mmol) in 2.2 mL CH₃CN was added Boc₂O (289 mg, 1.32 mmol) and DMAP (162 mg, 1.32 mmol). After 45 minutes, the reaction mixture was concentrated and purified by silica gel chromatography (0->15% EtOAc/hexanes, linear gradient) to obtain the desired product as a viscous oil. LCMS [M+H]⁺=493.

### Step B1-7: tert-Butyl (3S)-5-methyl-3-{(3-methylbutyl)[(4-methylphenyl)sulfonyl]amino}-2-oxo-2,3,4,7-tetrahydro-1H-azepine-1-carboxylate

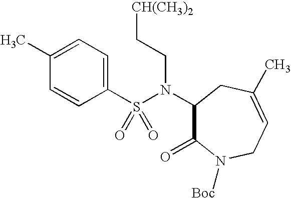

To a solution of diene from Step B1-6 (122 mg, 0.248 mmol) in 4 mL degassed CH₂Cl₂ was added Grubbs 2^{nd} generation metathesis catalyst (14.7 mg, 0.017 mmol) (Handbook of Metathesis; Grubbs, R. H., Ed.; Wiley-VCH: Weinheim, 2003; Diedrich, Tetrahedron Lett. 2006, 62, 1777-1786) in 1 mL degassed CH₂Cl₂. The reaction mixture was heated to 40° C. for 2 hours, then cooled to room temperature and purified directly via silica gel chromatography (0->20% EtOAc/hexanes, linear gradient) to afford the desired lactam B-7 as a white solid. LCMS [M+H]⁺=465.

### Step B1-8: tert-Butyl ((5S)-6-hydroxy-3-methyl-5-{(3-methylbutyl)[(4-methylphenyl)sulfonyl]amino}hex-2-en-1-yl)carbamate

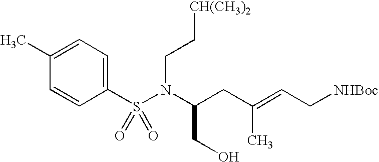

To a solution of lactam from Step B1-7 above (49 mg, 0.105 mmol) in 2.1 mL EtOH was added NaBH₄ (16 mg, 0.422 mmol) in one portion. The reaction was allowed to proceed at room temperature for 16 hours then diluted with EtOAc. The organics were washed with brine, dried over Na₂SO₄, filtered and concentrated. The residue was purified by silica gel chromatography (5->30% EtOAc/hexanes, linear gradient) to afford the desired product as a viscous oil. LCMS [M+H]⁺=469.

### Step B1-9: tert-Butyl ((5S)-6-hydroxy-3-methyl-5-{(3-methylbutyl)[(4-methylphenyl)sulfonyl]amino}hexyl)carbamate

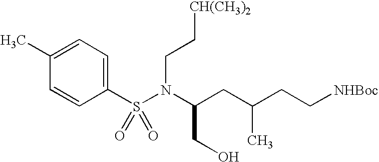

To a solution of product from Step B1-8 (35 mg, 0.075 mmol) in 1.5 mL EtOH was added 20% Pd(OH)₂ on carbon (5.2 mg, 7.47 μmol). A hydrogen balloon was attached, and the reaction mixture was evacuated/opened to hydrogen (3×). After 3 hours, the vessel was evacuated/refilled with argon (3×), then filtered through a pad of Celite, rinsing with EtOAc. The combined filtrates were concentrated to afford the desired 4-methyl lysine derivative as a viscous oil and as a 1:1 mixture of diastereomers at the newly created methyl bearing stereocenter. LCMS [M+H]⁺=471.

### Step B1-10: N-[(1S)-5-Amino-1-(hydroxymethyl)-3-methylpentyl]-4-methyl-N-(3-methylbutyl)benzenesulfonamide

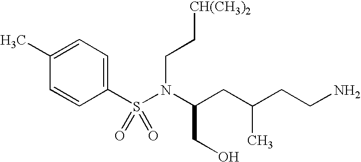

To a solution of product from Step B1-9 (30 mg, 0.064 mmol) in 1.2 mL CH₂Cl₂ was added 0.4 mL of 4M HCl in dioxane. After 2 hours, the reaction mixture was concentrated to afford the title compound as a white solid. LCMS [M+H]⁺=371.

### Step B1-11: tert-Butyl (1S)-1-(diphenylmethyl)-2-[((5S)-6-hydroxy-3-methyl-5-(3-methylbutyl)[(4-methylphenyl)sulfonyl]amino}hexyl)amino]-2-oxoethyl}carbamate

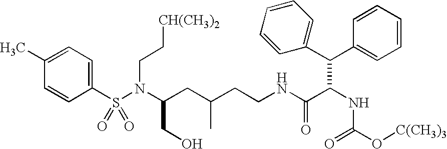

To a solution of the amine from step B1-10 (29 mg, 0.071 mmol) and N-Boc-(S)-diphenylalanine (29 mg, 0.086 mmol) in 1.4 mL DMF was added triethylamine (0.040 mL, 0.285 mmol) and BOP-reagent (44.1 mg, 0.100 mmol). After 50 minutes, the reaction mixture was diluted with EtOAc, and the organics were washed with H₂O, 3M LiCl (3×) and brine, dried over Na₂SO₄, filtered and concentrated. The residue was purified by silica gel chromatography (30->70% EtOAc/hexanes, linear gradient) to afford the desired adduct was a viscous oil. LCMS [M+H]⁺=694.

### Step B1-12: (2S)-2-amino-N-((5S)-6-hydroxy-3-methyl-5-{(3-methylbutyl)[(4-methylphenyl)-sulfonyl]amino}hexyl)-3,3-diphenylpropanamide

To a solution of product from Step B1-11 (34 mg, 0.049 mmol) in 0.7 mL CH₂Cl₂ was added 0.6 mL 4M HCl in dioxane. After 1.5 hours at room temperature, the reaction mixture was concentrated to obtain the desired product as a white solid. The ¹H NMR data tabulated below is for the 1:1 ratio of diastereomers carried forth from the synthesis of the lysine derivative used in the above step. ¹H NMR (400 MHz, d₄-MeOH) δ 8.17 (m, 4H), 7.72 (m, 2H), 7.52-7.43 (m, 4H), 7.26-7.21 (m, 2H), 3.82 (m, 1H), 3.76 (d, J=5.5 Hz, 1H), 3.61 (d, J=5.7 Hz), 3.39 (m, 2H), 3.11-2.80 Hz (m, 4H), 2.38 (d, J=8.5 Hz, 3H), 2.15-1.99 (m, 2H), 1.47 (m, 3H), 0.85 (m, 6H), 0.70 (m, 2H), LCMS [M+H]⁺=593.

### Example B2

### Methyl {(1S)-1-(diphenylmethyl)-2-[((5S)-6-hydroxy-3-methyl-5-{(3-methyl-butyl) [(4-methylphenyl)sulfonyl]amino}hexylamino]-2-oxoethyl}carbamate

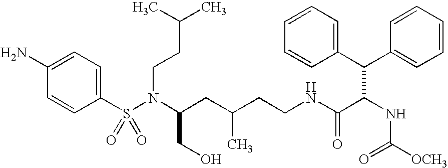

To a solution of the amine from Example B1, Step 10 (26 mg, 0.041 mmol) in 0.83 mL CHCl₃ was added 0.21 mL saturated NaHCO₃ solution. Methyl chloroformate (0.007 mL, 0.083 mmol) was added, and the reaction was allowed to proceed at room temperature for 3 hours. The mixture was diluted with CHCl₃ and brine and the layers were separated. The aqueous phase was washed with CHCl₃ (3×), and the combined organics were dried over Na₂SO₄, filtered and concentrated. Separation of the diastereomers via preparative HPLC (Sunfire column, 15 mL/min) afforded the desired products as white solids after lyophilization. Characterization data for faster eluting diastereomer by reverse-phase: ¹H NMR (400 MHz, d₄-MeOH) δ 7.70 (d, J=8.3 Hz, 2H), 7.34-7.31 (m, 4H), 7.22-7.10 (m, 8H), 4.87 (d, J=10.3 Hz, 1H), 4.26 (d, J=11.4 Hz), 3.73 (m, 1H), 3.50 (s, 3H), 3.36-3.28 (m, 2H), 3.17-3.00 (m, 4H), 2.80 (m, 2H), 1.48 (m, 2H), 1.39-1.29 (m, 3H), 1.21-1.18 (m, 2H), 1.00 (m, 1H), 0.87 (d, J=6.1 Hz, 6H), 0.65 (d, J=5.6 Hz, 3H); LCMS [M+H]⁺=652. Characterization data for slower eluting diastereomer by reverse-phase: ¹H NMR (400 MHz, d₄-MeOH) δ 7.70 (d, J=8.0 Hz, 2H), 7.30-7.19 (m, 10H), 7.16-7.12 (m, 2H), 4.87 (d, J=10.1 Hz, 1H), 4.28 (d, J=11.2 Hz), 3.72 (m, 1H), 3.48 (s, 3H), 3.63-3.53 (m, 2H), 3.19-3.10 (m, 2H), 2.94 (m, 1H), 2.64 (m, 1H), 1.53 (m, 3H), 1.43-1.37 (m, 2H), 1.10 (m, 3H), 0.87 (d, J=5.6 Hz, 6H), 0.69 (d, J=6.1 Hz, 3H); LCMS [M+H]⁺=652.

### Example C1

### Methyl {(1S)-1-(diphenylmethyl)-2-[((5S)-6-hydroxy-2-methyl-5-{(3-methylbutyl) [(4-methylphenyl)sulfonyl]amino}hexylamino]-2-oxoethyl}carbamate

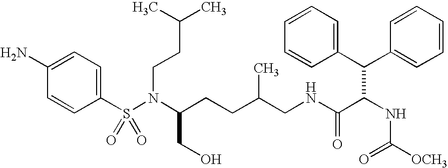

### Step C1-1: Methyl (2S)-2-{[(4-methylphenyl)sulfonyl]amino}pent-4-enoate

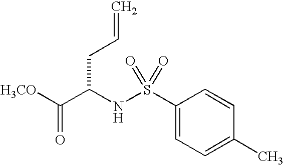

To a solution containing 7.35 g (44.4 mmol) of allyl glycine methyl ester hydrochloride in 400 mL of DCM was added 12.3 mL (89 mmol) of triethylamine followed by 8.5 g (44.4 mmol) of tosyl chloride and the resulting mixture was allowed to stir at room temperature for 16 hours. The solution was washed with 1 N HCl (2×50 mL), saturated NaHCO₃ (2×50 mL), water (50 mL), and brine (50 mL). The organic phase was dried over MgSO₄, concentrated and chromatographed (0% to 100% EtOAc/hexanes) to afford the desired product. LCMS (M+1)=284.3.

### Step C1-2: Methyl (2S)-2-{(3-methylbutyl)[(4-methylphenyl)sulfonyl]amino}pent-4-enoate

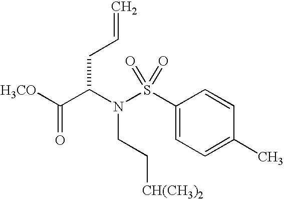

The sulfonamide from step C1-1 (6.0 g, 21.2 mmol) was dissolved in 85 mL of DCM and treated sequentially with triphenylphosphine (6.66 g, 25.4 mmol), isoamyl alcohol (2.8 mL, 25.4 mmol), and DCAD (9.33 g, 25.4 mmol) and the resulting solution was allowed to stir for 72 hours at room temperature. The resulting solids were filtered and discarded and the filtrate was concentrated and chromatographed (0% to 100% EtOAc/hexanes) to afford the desired product. LCMS (M+1)=354.5.

### Step C1-3: (2S)-2-{(3-Methylbutyl)[(4-methylphenyl)sulfonyl]amino}pent-4-enoic acid

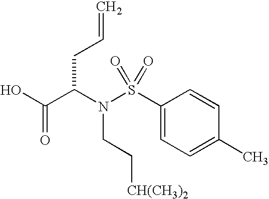

A solution containing 2.89 g (8.18 mmol) of ester from step C1-2 dissolved in 10 mL of THF and 10 mL of water was treated with 8.18 mL (16.3 mmol) of 2N LiOH and the resulting mixture was stirred at room temperature for 16 hours. The mixture was acidified with 3N HCl and washed with ether (3×3 mL). The combined organics were dried over MgSO₄ and concentrated to afford the desired acid. LCMS (M+1)=340.4.

### Step C1-4: (2S)—N-(2,4-Dimethoxybenzyl)-2-(3-methylbutyl)[4-methylphenyl)sulfonyl]amino}-N-(2-methylprop-2-en-1-yl)pent-4-enamide

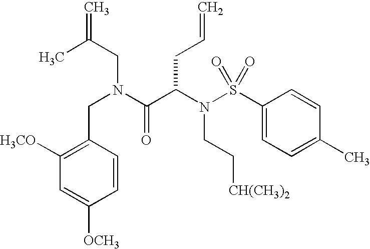

To a solution of the carboxylic acid from step C1-3 (1.57 g (4.62 mmol) in 80 mL of DCM was added 0.93 g (4.2 mmol) of N-(2,4-dimethoxybenzy)-2-methylprop-2-en-1-amine (Intermediate 1), and 0.89 g (4.62 mmol) of EDC. The resulting mixture was stirred at room temperature for 16 hours, concentrated and chromatographed directly (0% to 100% EtOAc/hexanes) to afford the desired amide as a white solid. LCMS (M+1)=543.7.

### Step C1-5: (3S)-1-(2,4-Dimethoxybenzyl)-6-methyl-3-[(3-methylbutyl) (4-methylphenyl)sulfonyl)amino]-1,3,4,7-tetrahydro-2H-azepin-2-one

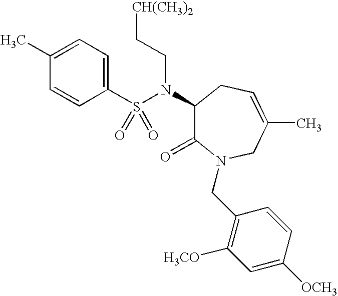

A solution containing 0.988 g (1.82 mmol) of the diene obtained from step C1-4 above was dissolved in 270 mL of DCM and treated with 0.386 g (0.455 mmol) of 2^{nd} generation Grubb's catalyst. The reaction mixture was heated at 40° C. for 16 hours before being cooled, concentrated and chromatographed (gradient: 0% to 100% EtOAc/hexanes) to afford the desired lactam. LCMS (M+1)=515.7.

### Step C1-6: 4-Methyl-N-(3-methylbutyl)-N-[(3S)-6-methyl-2-oxo-2,3,4,7-tetrahydro-1H-azepin-3-yl]benzenesulfonamide

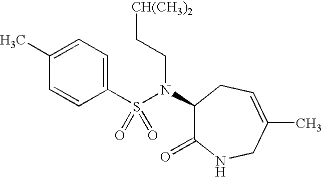

A solution containing 0.78 g (1.51 mmol) of lactam C1-5 dissolved in 9 mL of DCM was treated with 13 mL of TFA and stirred for 16 hours. The resulting purple solution was concentrated and treated with 30 mL of methanol then filtered. The filtrate was concentrated, diluted with 20 mL of DCM and washed with water (×2), saturated bicarbonate solution (×2) and brine. The organic extract was dried, concentrated and chromatographed (gradient: 0% to 100% EtOAc/hexanes) to afford the desired lactam. LCMS (M+1)=365.5.

### Step C1-7: 4-methyl-N-(3-methylbutyl)-N-[(3S)-6-methyl-2-oxoazepan-3-yl]benzenesulfonamide

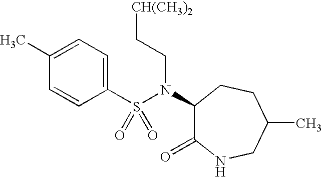

A degassed solution containing 0.51 g (1.4 mmol) of lactam from step C1-6 dissolved in 10 mL of EtOAc was treated with 17 mg of 10% Pd on carbon and hydrogenated at STP for 16 hours. The reaction mixture was filtered through Celite and evaporated to leave the desired compound. LCMS (M+1)=367.5.

### Step C1-8: tert-Butyl (3S)-6-methyl-3-{(3-methylbutyl)[(4-methylphenyl)sulfonyl]amino}-2-oxoazepane-1-carboxylate

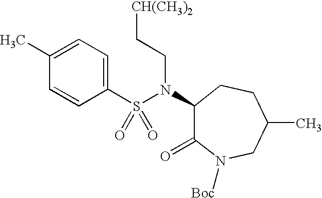

The lactam obtained form step C1-7 above (0.51 g, 1.4 mmol) was dissolved in 8 mL of MeCN and treated with 0.911 g (4.17 mmol) of Boc₂O then 17 mg (0.14 mmol) of DMAP. The resulting mixture was stirred for 16 hours then concentrated. Column chromatography (gradient: 0% to 100% EtOAc/hexanes) afforded the desired lactam. LCMS (M+1)=467.7.

### Step C1-9: tert-Butyl ((5S)-6-hydroxy-2-methyl-5-{(3-methylbutyl)[4-methylphenyl)sulfonyl]amino}hexyl)carbamate

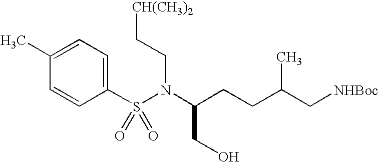

To a solution of lactam from step C1-8 (0.345 g, 0.739) in 4 mL of EtOH was added 0.078 g (2.07 mmol) of NaBH₄. The resulting mixture was stirred for 5 hours and concentrated. The residue was treated with 2 mL of 1N NaOH and extracted with EtOAc×3, dried, concentrated and chromatographed (gradient: 0% to 100% EtOAc/hexanes) to afford the desired alcohol. LCMS (M+1)=471.7.

### Step C1-10: N-[(1S)-5-Amino-1-(hydroxymethyl)-4-methylpentyl]-4-methyl-N-(3-methylbutyl)benzenesulfonamide

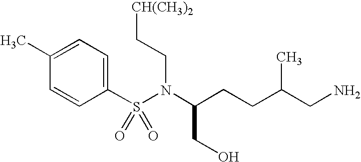

To a solution containing 0.23 g (0.49 mmol) of the protected amine from step C1-9 dissolved in 4 mL of DCM was added 2 mL of TFA. The reaction mixture was stirred for 30 minutes then made basic by the addition of solid K₂CO₃. Extraction with DCM (3×5 mL) afforded the desired amino alcohol as an oil LCMS (M+1)=371.5.

### Step C1-11: Methyl {(1S)-1-(diphenylmethyl)-2-[((5S)-6-hydroxy-2-methyl-5-{(3-methyl butyl) [(4-methylphenyl)sulfonyl]amino}hexylamino]-2-oxoethyl}carbamate

To a solution of the amine from step C1-10 (181 mg, 0.488 mmol) and N-Moc-(S)-diphenylalanine (146 mg, 0.488 mmol) in 3 mL DMF was added diisopropylethylamine (164 mg, 1.27 mmol) and BOP-reagent (281 mg, 0.635 mmol). After 60 minutes, the reaction mixture was filtered and the residue was purified by reverse phase chromatography. Pure fractions were diluted with EtOAc and rendered basic by the addition of saturated NaHCO₃. The organic phase was separated, dried and evaporated to afford the desired adduct as a white solid. ¹H NMR (CDCl₃): δ 7.73-7.71 (d, J=7 Hz, 2H), 7.50-7.05 (m, 12 H), 5.71 (br s, 1H), 5.11-5.10 (m, 1H), 4.82-4.76 (m, 1H), 4.49-4.47 (d, J=10 Hz, 1H), 3.61-3.59 (m, 4H), 3.53-3.29 (m, 2H), 3.22-3.20 (m, 1H), 3.08-3.05 (m, 1H), 2.93-2.82 (m, 1H), 2.76-2.66 (m, 1H), 2.42 (s, 3H), 2.34-2.24 (m, 1H), 1.56-1.21 (m, 6H), 0.91-0.90 (m, 6H), 0.77-0.65 (m, 2H), 0.53-0.48 (m, 3H). LCMS [M+H]⁺=652.9.

### Example D1

### Methyl [(1S)-2-({(5S)-5-[[4-aminophenyl)sulfonyl]-((3S)-3-methylbutyl)amino]-6-hydroxy-1-methylhexyl)amino)-1-(diphenylmethyl)-2-oxoethyl]carbamate and Methyl [(1S)-2-({(5S)-5-[[4-aminophenyl)sulfonyl]-((3R)-3-methylbutyl)amino]-6-hydroxy-1-methylhexyl)amino)-1-(diphenylmethyl)-2-oxoethyl]carbamate

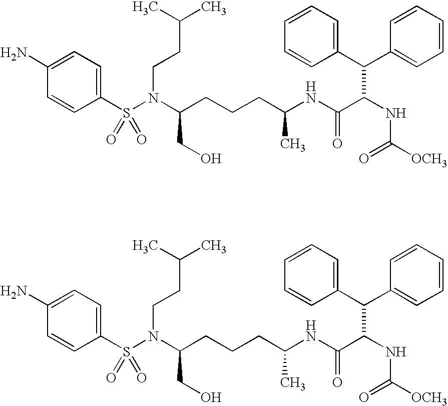

### Step D1-1: 1-Benzyl 5-methyl (2S)-2-[bis(tert-butoxycarbonyl)amino]pentanedioate

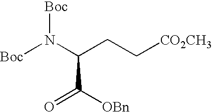

To a solution containing 10.0 g (28.5 mmol) of benzyl 5-methyl (2S)-2-[(tert-butoxy carbonyl)amino]pentanedioate (Schoenfelder et al., Syn Comm. 1990, 20(17), 2585) in 100 mL of acetonitrile was added 9.3 g (42.7 mmol) of Boc₂O then 1.73 g (14.2 mmol) of DMAP. The resulting mixture was stirred for 16 hours then concentrated. Column chromatography (30% EtOAc/hexanes) afforded the bis Boc amine. LCMS (M+Na)=474.0.

### Step D1-2: Benzyl (2S)-2-[bis(tert-butoxycarbonyl)amino]-5-oxopentanoate

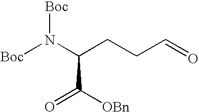

To a −70° C. solution containing 11.0 g (24.4 mmol) of diester from step D1-1 in 250 mL of ether was added 31.7 mL of DIBAL-H (1 M in toluene). The reaction mixture was stirred for 5 minutes, treated 10 mL of water and warmed to room temperature. The reaction mixture was filtered through Celite and evaporated. Column chromatography (30% EtOAc/hexanes) afforded the desired aldehyde. LCMS (M+Na)=444.0.

### Step D1-3: Benzyl (2S)-2-[bis(tert-butoxycarbonyl)amino]-6-nitrohept-5-enoate

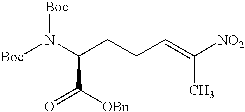

To a solution of the aldehyde from step D1-2 (8.3 g, 19.7 mmol) in 50 mL of toluene at 0° C. was added 14.8 g (197 mmol) of nitroethane then 0.42 mL (3.35 mmol) of tetramethylguanidine. The reaction mixture was stirred for 30 minutes then treated with 4.1 mL (29.5 mmol) of TEA and 2.3 mL (29.5 mmol) of methanesulfonyl chloride. After an additional 2 hours of stirring, 122 mg (1.00 mmol) of DMAP was added and the reaction mixture was heated to 60° C. for 16 hours. The reaction mixture was cooled and diluted with 100 mL of ether. The solution was washed with water (2×25 mL), saturated NaHCO₃ (2×25 mL) and brine. Column chromatography (30% EtOAc/hexanes) afforded the desired nitro olefin.

LCMS (M+Na)=500.9.

### Step D1-4: (2S)-6-Amino-2-[bis(tert-butoxycarbonyl)amino]heptanoic acid

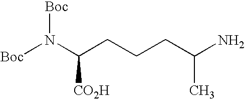

The nitro olefin from step 3 above (9.0 g, 18.8 mmol) was dissolved in 225 mL of MeOH and treated with 3 g of 10% Pd(OH)₂. The resulting mixture was hydrogenated at STP for 72 hours, filtered through a pad of Celite and evaporated to afford the desired amino acid as a white foam. LCMS (M+1)=361.1.

### Step D1-5: (2S)-6-[(Benzyloxy)carbonyl]amino}-2-[bis(tert-butoxycarbonyl)amino]heptanoic acid

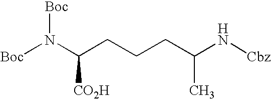

Cbz chloride (1.28 mL, 9.0 mmol) was dissolved in 7 mL of dioxane and was added to 2.7 g (7.49 mmol) of the amine from step D1-4 dissolved in 171 mL of water/dioxane/acetonitrile (72/54/45) and 794 mg (7.49 mmol) of sodium carbonate. The reaction mixture was stirred for 16 hours and concentrated. The residue was redissolved in 50 mL of DCM and washed with 1% citric acid solution then brine. The organic extract was dried and concentrated to leave the desired N-Cbz protected amine D1-5. LCMS (M+1)=495.6

### Step D1-6: Methyl (2S)-2-amino-6-{[(benzyloxy)carbonyl]amino}heptanoate

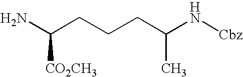

Compound D1-5 (2.8 g, 5.66 mmol) was dissolved in 50 mL of MeOH at 0° C. and a stream of HCl gas was passed through the solution for 2 minutes. After stirring the reaction mixture an additional 30 minutes, the solvent was removed to afford the desired amino ester HCl salt which was used in the next reaction without further purification. LCMS (M+1)=310.4

### Step D1-7: Methyl (2S)-6-[(benzyloxy)carbonyl]amino}-2-{[(4-nitrophenyl)sulfonyl]amino}heptanoate

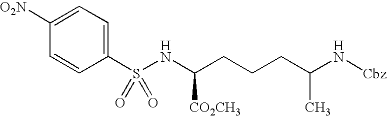

To a solution containing 2.43 g (7.05 mmol) of ester D1-6 in 35 mL of DCM was added 2 mL (14 mmol) of triethylamine followed by 1.5 g (7.05 mmol) of p-nitrobenzenesulfonyl chloride and the resulting mixture was allowed to stir at room temperature for 16 hours. The solution was washed with 1 N HCl (2×10 mL), saturated NaHCO₃ (2×10 mL), water (10 mL), and brine (10 mL). The organic phase was dried over MgSO₄, concentrated and chromatographed (0% to 100% EtOAc/hexanes) to afford the desired product D1-7. LCMS (M+1)=494.5.

### Step D1-8: Methyl (2S)-6-{[(benzyloxy)carbonyl]amino}-2-{(3-methylbutyl)[(4-nitrophenyl)sulfonyl]amino}heptanoate

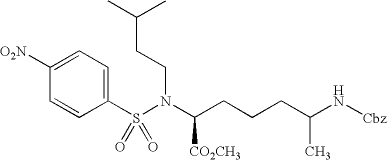

Sulfonamide D1-7 (0.88 g, 1.78 mmol) was dissolved in 7 mL of DCM and treated sequentially with triphenylphosphine (561 mg, 2.1 mmol), isoamyl alcohol (0.233 mL, 2.14 mmol), and DCAD (0.786 g, 2.14 mmol) and the resulting solution was allowed to stir for 72 hours at room temperature. The resulting solids were filtered and discarded and the filtrate was concentrated and chromatographed (0% to 100% EtOAc/hexanes) to afford the desired product. LCMS (M+1)=564.6

### Step D1-9: Methyl (2S)-6-amino}-2-[[(4-aminophenyl)sulfonyl]3-methylbutyl)amino}heptanoate

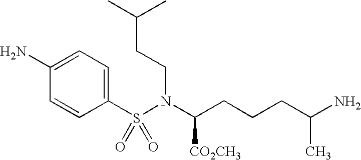

A degassed solution containing 0.748 g (1.36 mmol) of compound D1-8 dissolved in 20 mL of MeOH was treated with 956 mg of 10% Pd(OH)₂ and hydrogenated at STP for 2 hours. The reaction mixture was filtered through Celite and evaporated to leave the desired compound D1-9. LCMS (M+1)=400.5.

### Step D1-10: Methyl (2S)-2-[[(4-aminophenyl)sulfonyl]3-methylbutyl)amino]-6-({(2S)-2-[(methoxycarbonyl)amino]-3,3-diphenylpropanoyl}aminoheptanoate

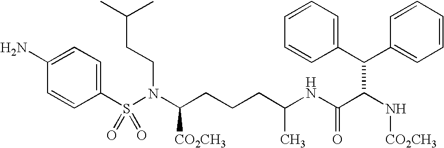

To a solution of the amine from step D1-9 (300 mg, 0.751 mmol) and N-Moc-(S)-diphenylalanine (225 mg, 0.751 mmol) in 3 mL DCM was added diisopropylethylamine (252 mg, 1.295 mmol) and BOP-reagent (432 mg, 0.976 mmol). After 60 minutes, the reaction mixture was diluted with DCM and washed with saturated NaHCO₃. The organic phase was separated, dried and evaporated. Column chromatography (gradient: 0% to 100% EtOAc/hexanes) afforded the desired adduct as a white solid. LCMS (M+1)=681.8.

### Step D1-11: Methyl [(1S)-2-({(5S)-5-[[4-aminophenyl)sulfonyl]-((3S)-3-methylbutyl)amino]-6-hydroxy-1-methylhexyl)amino)-1-(diphenylmethyl)-2-oxoethyl]carbamate and Methyl [(1S)-2-({(5S)-5-[[4-aminophenyl)sulfonyl]-((3R)-3-methylbutyl)amino]-6-hydroxy-1-methylhexyl)amino)-1-(diphenylmethyl)-2-oxoethyl]carbamate

To a solution containing 485 mg (0.712 mmol) of the ester obtained from step D1-10 in 5 mL of THF was added 0.71 mL of 2M LiBH₄. The reaction mixture was allowed to stir for 5 minutes before 0.5 mL of MeOH was added. After an additional 1 hour of stirring, 2 mL of NaHCO₃ was added and the reaction mixture was diluted with EtOAc. The organic phase was separated and dried then subjected to reverse phase chromatography. Pure fractions were diluted with EtOAc and rendered basic by the addition of saturated NaHCO₃. The organic phase was separated, dried and evaporated to afford 313 mg (67%) of the desired adduct was a white solid.

¹H NMR (CDCl₃): δ 7.61-7.59 (m, 2H), 7.33-7.17 (m, 10H), 6.72-6.66 (m, 2H), 5.33-5.15 (m, 1H), 4.78-4.74 (m, 1H), 4.47-4.40 (m, 2H), 4.22 (s, 1H), 3.70-3.50 (m, 8H), 3.21-3.16 (m, 1H), 3.02-3.00 (m, 1H), 2.50-2.42 (m, 1H), 1.57-1.50 (m, 4H), 1.30-0.82 (m, 12H), 0.56-0.55 (m, 2H). LCMS [M+H]⁺=653.8.

The mixture of diastereomers was separated by chiral chromatography (Kromasil Chiral TBB, 25% IPA in CO2, first eluting compound collection time: 21.0-24.30 minutes, second eluting compound collection time: 25.0-28.0 minutes).

N-{(1R,5S)-5-[[(4-aminophenyl)sulfonyl](3-methylbutyl)amino]-6-hydroxy-1-methylhexyl}-Nα-(methoxycarbonyl)-β-phenyl-L-phenylalaninamide (first eluting compound, D1-(S)). ¹H NMR (CDCl₃): δ 7.61-7.59 (m, 2H), 7.31-7.17 (m, 10H), 6.68-6.66 (m, 2H), 5.33-5.27 (m, 2H), 4.78-4.74 (t, J=10 Hz, 1H), 4.40-4.38 (d, J=10 Hz, 2H), 3.61-3.50 (m, 8H), 3.20-3.15 (m, 1H), 3.04-2.77 (m, 3H), 1.55-1.49 (m, 3H), 1.32-0.86 (m, 12H), 0.56-0.55 (d, J=6 Hz, 2H), LCMS [M+H]⁺=653.1.

N-{(1S,5S)-5-[[(4-aminohenyl)sulfonyl](3-methylbutyl)amino]-6-hydroxy-1-methylhexyl}-Nα-(methoxycarbonyl)-β-phenyl-L-phenylalaninamide (second eluting compound, D1-(R)). ¹H-NMR (CDCl₃): δ 7.61-7.58 (m, 2H), 7.34-7.20 (m, 10H), 6.72-6.66 (m, 2H), 5.28-5.26 (d, J=8 Hz, 1H), 5.16-5.15 (d, J=8 Hz, 1H), 4.78-4.74 (t, J=10 Hz, 1H), 4.47-4.45 (d, J=10 Hz, 1H), 3.72-3.49 (m, 8H), 3.24-3.18 (m, 1H), 3.02-2.60 (m, 3H), 1.56-1.50 (m, 3H), 1.26-0.83 (m, 13H), 0.55-0.51 (m, 2H); LCMS [M+H]⁺=653.0.

### Example D2

### Methyl [(1S)-2-({(5S)-5-[[4-aminophenyl)sulfonyl]-((3S)-3-ethylbutyl)amino]-6-hydroxy-1-methylhexyl)amino)-1-(diphenylmethyl)-2-oxoethyl]carbamate

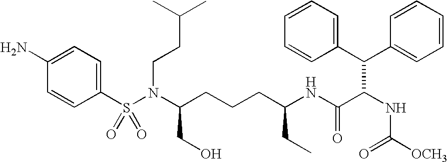

### Step D2-1: Methyl (2S)-2-[bis(tert-butoxycarbonyl)amino]-6-nitrooctan-5-enoate

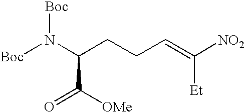

To a solution containing 3.4 g (9.8 mmol) of (2S)-2-(bis(tert-butoxycarbonyl)amino)-5-oxopentanoate (Tetrahedron: Asymmetry 1998, 9(19), 3381-3394) in 35 mL of toluene at 0° C. was added 8.8 g (97 mmol) of nitropropane, followed by 0.113 g (0.984 mmol) of tetramethylguanidine. The reaction mixture was stirred for 30 minutes and then treated with 1.5 g (14.8 mmol) of TEA and 1.7 mL (14.8 mmol) of methanesulfonyl chloride. After an additional 2 hours of stirring, 122 mg (1.00 mmol) of DMAP was added and the reaction mixture was heated to 60° C. for 16 hours. The reaction mixture was then cooled to room temperature and diluted with 100 mL of Et₂O. The solution was washed with water (2×25 mL), saturated NaHCO₃ (2×25 mL) and brine. Column chromatography (20% EtOAc/hexanes) of the washed solution afforded the desired nitro olefin. LCMS (M+Na)=440.

### Step D2-2: Methyl (2S)-7-Amino-2-[bis(tert-butoxycarbonyl)amino]octanoate

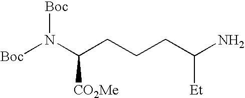

The nitro olefin from D1-1 (1.8 g, 4.32 mmol) was dissolved in 35 mL of MeOH and treated with 1.5 g of 10% Pd(OH)₂. The resulting mixture was hydrogenated at STP for 72 hours, filtered through a pad of Celite and evaporated to afford the desired amino ester as a white foam. LCMS (M+1)=389.0.

### Step D2-3: Methyl (2S)-2-[bis(tert-butoxycarbonyl)amino]-6-({(2S)-2-[(methoxycarbonyl)amino]-3,3-diphenylpropanoyl}amino)octanoate

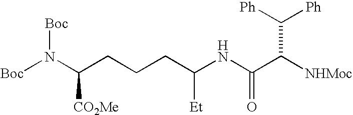

To a solution containing 2.2 g (5.66 mmol) of the amine above in a 1/1/1 mixture of saturated NaHCO₃, acetone, and THF (24 mL) was added Moc-di-Phe-HSU ester (2.2 g, 5.66 mmol) and the mixture stirred for 5 hours. The product was extracted into EtOAc and the organic phase was dried and concentrated and used directly without further purification. LCMS (M+Na)=692

### Step D2-4: Methyl (2S)-2-amino-6-({(2S)-2-[(methoxycarbonyl)amino]-3,3-diphenylpropanoyl}amino)octanoate

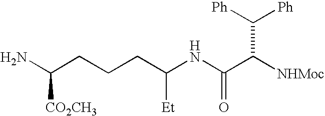

Compound D2-3 (3.7 g, 5.52 mmol) was dissolved in 10 mL of ether at 24° C. and treated with 13.8 mL (55.2 mmol) of 4N HCl in dioxane. After the dissolution was complete, the reaction mixture for 30 minutes, and then the solvent was removed to afford the desired amino ester HCl salt which was used in the next reaction without further purification. LCMS (M+1)=470.0

### Step D2-5: Methyl (2S,6S)-6-({(2S)-2-[(methoxycarbonyl)amino]-3,3-diphenylpropanoyl}amino)-2-{[(4-nitrophenyl)sulfonyl]amino}octanoate

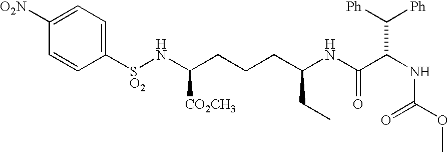

To a solution containing 2.8 g (5.53 mmol) of ester D2-4 in 50 mL of chloroform was added 2 mL (14 mmol) of triethylamine followed by 1.54 g (6.1 mmol) of p-nitrobenzenesulfonyl chloride, and the resulting mixture was allowed to stir at room temperature for 16 hours. The solution was then washed with 1 N HCl (2×10 mL), saturated NaHCO₃ (2×10 mL), water (10 mL), and brine (10 mL). The organic phase was dried over MgSO₄, concentrated and chromatographed (0% to 100% EtOAc/hexanes) to afford each diastereomeric product as a white foam. The less polar product (80% EtOAc/hexanes) is the S,R-isomer and the more polar product (80% EtOAc/hexanes) is the S,S-isomer. LCMS (M+1)=655.

### Step D2-6: Methyl (2S,6S)-6-({(2S)-2-[(methoxycarbonyl)amino]-3,3-diphenyl-propanoyl}amino)-2-{(3-methylbutyl) [(4-nitrophenyl)sulfonyl]amino}octanoate

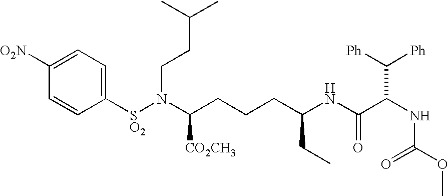

The more polar diastereomeric sulfonamide D2-5 (1.2 g, 1.8 mmol) was dissolved in 10 mL of THF and treated sequentially with triphenylphosphine (577 mg, 2.2 mmol), isoamyl alcohol (0.233 mL, 2.14 mmol), and DIAD (0.445 g, 2.2 mmol) and the resulting solution was allowed to stir for 72 hours at room temperature. The mixture was concentrated and chromatographed (0% to 100% EtOAc/hexanes) to afford the desired product. LCMS (M+1)=725.0

### Step D2-7: Methyl (2S,6S)-2-[(4-aminophenyl)sulfonyl](3-methylbutyl)amino-6-({(2S)-2-[(methoxycarbonyl)amino]-3,3-diphenylpropanoyl}amino)octanoate

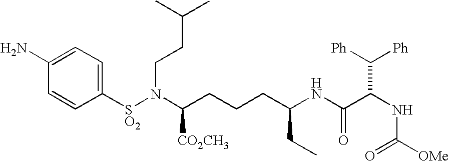

A degassed solution containing 1.0 g (1.38 mmol) of compound D2-6 dissolved in 30 mL of MeOH was treated with 956 mg of 10% Pd(OH)₂ and hydrogenated at STP for 1 hours at room temperature. The reaction mixture was filtered through Celite and evaporated to leave the desired compound. LCMS (M+1)=695.0

### Step D2-8: Methyl [(1S)-2-({(5S)-5-[[4-aminophenyl)sulfonyl]-((3S)-3-ethylbutyl)amino]-6-hydroxy-1-methylhexyl)amino)-1-(diphenylmethyl)-2-oxoethyl]carbamate

To a solution containing 700 mg (1.0 mmol) of the ester obtained from step D2-7 in 10 mL of THF was added 1.2 mL of 2M LiBH₄. The reaction mixture was allowed to stir for 5 minutes before 0.5 mL of MeOH was added. After an additional 1 hour of stirring, 2 mL of NaHCO₃ was added and the reaction mixture was diluted with EtOAc. The organic phase was separated and dried then subjected to reverse phase chromatography. Pure fractions were diluted with EtOAc and rendered basic by the addition of saturated NaHCO₃. The organic phase was separated, dried and evaporated to afford the desired adduct was a white solid.

¹H NMR (CDCl₃): δ 7.61 (d, J=7.8 Hz, 2H), 7.4-7.1 (m, 10H), 6.72 (d, J=7.8 Hz, 2H), 5.25 (bt, 1H), 4.78 (t, 1H), 4.40 (d, J=8 Hz, 2H), 4.21 (bs, 2H), 3.70-3.50 (m, 8H), 3.21-3.16 (m, 1H), 3.02-3.00 (m, 1H), 1.60-1.48 (m, 4H), 1.40-0.82 (m, 4H), 0.75 (d, 6H), 0.56-0.55 (t, 3H). LCMS [M+H]⁺=667.8.

### Example D3

### N-{(1S,5S)-5-[[(4-amino-3-fluorophenyl)sulfonyl](3-methylbutyl)amino]-1-ethyl-6-hydroxyhexyl}-Nα-(methoxycarbonyl)-b-phenyl-L-phenylalaninamide

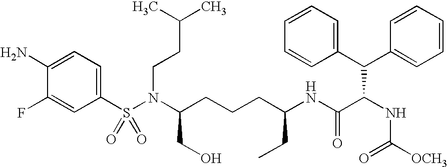

To a solution of methyl [(1S)-2-({(5S)-5-[[4-aminophenyl)sulfonyl]-((3S)-3-ethylbutyl)amino]-6-hydroxy-1-methylhexyl)amino)-1-(diphenylmethyl)-2-oxoethyl]carbamate (220 mg, 0.33 mmol, Example D2) in 8 mL of MeCN was added 117 mg (0.330 mmol) of Selectfluor® (i.e., 1-chloromethyl-4-fluoro-1,4-diazoniabicyclo[2.2.2]octane bis-(tetrafluoroborate); available from Air Products & Chemicals). The reaction mixture was stirred for 1 h then subjected directly to RPLC to afford the desired monofluorinated product as colorless foam. LCMS (M+Na)=685.3.

¹H-NMR (CDCl₃): δ 7.44 (m, 2H), 7.4-7.15 (m, 10H), 6.82 (bt, J=8.2 Hz, 1H), 5.36 (bd, J=8.6 Hz, 1H), 5.23 (bd, J=8.3 Hz, 1H), 4.82 (t, J=9.5 Hz, 1H), 4.44 (d, J=10.6 Hz, 2H), 3.61 (s, 3H), 3.40 (m, 5H), 3.21 (m, 1H), 3.02 (m, 1H), 1.60-1.48 (m, 2H), 1.20-0.92 (m, 4H), 0.75 (d, 6H), 0.75 (t, J=7.3 Hz, 3H), 0.55 (m, 1H), 0.21 (m, 1H).

### Example D4

### N-{(1S,5S)-6-amino-5-[[(4-aminophenyl)sulfonyl](3-methylbutyl)amino]-1-methylhexyl}-2-chloro-Nα-(methoxycarbonyl)-L-phenylalaninamide

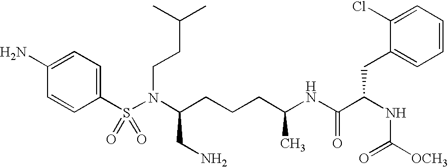

(2S)-6-Amino-2-[bis(tert-butoxycarbonyl)amino]heptanoic acid from Step D1-4 was elaborated to N-{(1S,5S)-6-amino-5-[[(4-aminophenyl)sulfonyl](3-methylbutyl)amino]-1-methylhexyl}-2-chloro-Nα-(methoxycarbonyl)-L-phenylalaninamide using steps D2-3 (Moc-2-Cl-Phe HSU ester was used in the place of Moc-di-Phe-HSU ester), F1-1 (MeOH was used in place of EtOH), D2-5, D2-6, A2-5, A2-6 and A2-7. MS: M+H=610, 611 (Cl pattern). ¹H NMR (400 MHz, MeOD) δ 7.54 (d, J=7.2 Hz, 2H), 7.39-37 (m, 1H), 7.28-7.17 (m, 3H), 6.65 (d, J=7.2 Hz, 2H), 4.34 (t, J=8.2 Hz, 1H), 3.72-3.45 (m, 5H), 3.55 (s, 3H), 3.21 (d, J=6.3 Hz, 1H), 3.18 (d, J=6.3 Hz, 1H), 3.10-3.03 (m, 1H), 3.97-3.92 (m, 3H), 1.58-1.48 (m, 3H), 1.39-1.32 (m, 1H), 1.18-1.03 (m, 3H), 0.97 (d, J=6.7 Hz, 3H), 0.92 (d, J=6.0 Hz, 6H), 0.86-0.80 (m, 2H).

The following examples (Table D) were prepared using procedures similar to those described in the preparation of Examples D1 to D4, using the appropriate building blocks (R⁵CH₂NO₂, ArSO₂Cl, R¹OH, HO₂C—CHR⁶—NHR⁷ or corresponding activated amino acid such as hydroxysuccinate ester). In some cases NHR⁷ is originally protected as Boc which necessitates an acidic Boc removal in the last step.

TABLE D

| Example | | |
|---|---|---|
| No. | Structure | M + 1 |
| D5 | N-{(1S,5S)-5-[(1,3-benzothiazol-6-ylsulfonyl)(3- | 695 |
| | methylbutyl)amino]-6-hydroxy-1-methylhexyl}-Nα- | |
| | (methoxycarbonyl)-β-phenyl-L-phenylalaninamide | |
| | 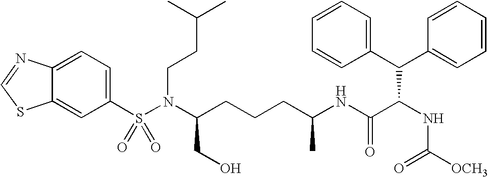 | |
| D6 | N-{(1S,5S)-5-[[(4-aminophenyl)sulfonyl](3- | 681 |
| | methylbutyl)amino]-6-hydroxy-1-propylhexyl}-Nα- | |
| | (methoxycarbonyl)-β-phenyl-L-phenylalaninamide | |
| | 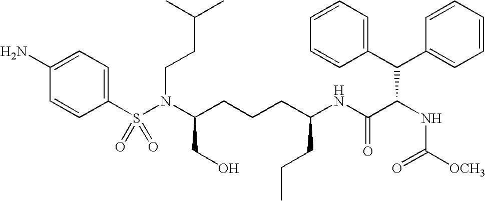 | |
| D7 | N-{(1S,5S)-5-[[(4-aminophenyl)sulfonyl](3- | 611 |
| | methylbutyl)amino]-6-hydroxy-1-methylhexyl}-2-chloro-Nα- | |
| | (methoxycarbonyl)-L-phenylalaninamide | |
| | 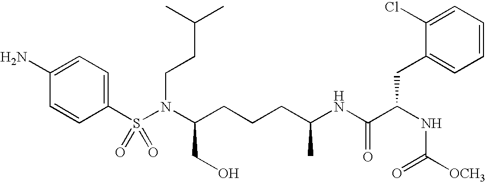 | |
| D8 | N-{(1S,5S)-5-[[(4-aminophenyl)sulfonyl](isobutyl)amino]-6- | 597 |
| | hydroxy-1-methylhexyl}-2-chloro-Nα-(methoxycarbonyl)-L- | |
| | phenylalaninamide | |
| | 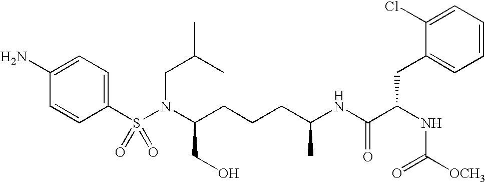 | |
| D9 | N-{(1S,5S)-5-[[(4-aminophenyl)sulfonyl](isobutyl)amino]-6- | 639 |
| | hydroxy-1-methylhexyl}-Nα-(methoxycarbonyl)-β-phenyl-L- | |
| | phenylalaninamide | |
| | 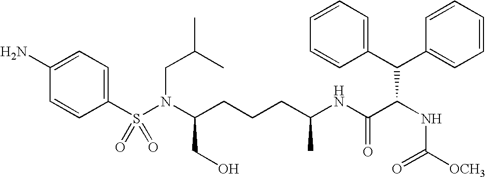 | |
| D10 | N-{(1R,5S)-5-[[(4-aminophenyl)sulfonyl](3- | 681 |
| | methylbutyl)amino]-6-hydroxy-1-isopropylhexyl}-Nα- | |
| | (methoxycarbonyl)-β-phenyl-L-phenylalaninamide | |
| | 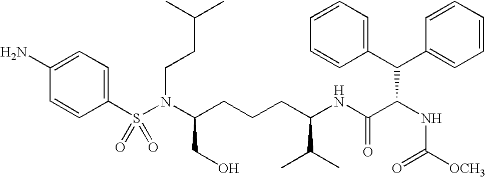 | |
| D11 | N-{(1S,5S)-5-[[(4-aminophenyl)sulfonyl](isobutyl)amino]-1- | 653 |
| | ethyl-6-hydroxyhexyl}-Nα-(methoxycarbonyl)-β-phenyl-L- | |
| | phenylalaninamide | |
| | 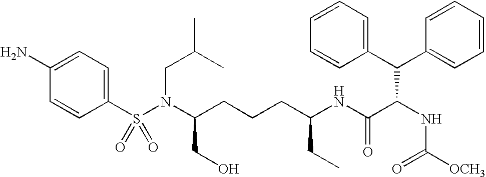 | |
| D12¹ | N-{(1S,5S)-5-[[(4-amino-3-fluorophenyl)sulfonyl](3- | 699 |
| | methylbutyl)amino]-6-hydroxy-1-methylhexyl}-Nα- | |
| | (methoxycarbonyl)-β-phenyl-L-phenylalaninamide | |
| | 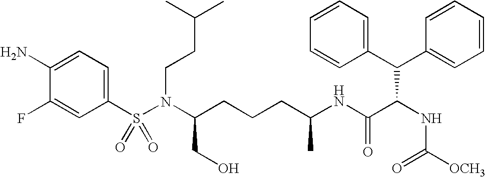 | |
| D13 | N-{(1S,5S)-5-[[(4-aminophenyl)sulfonyl](3- | 671 |
| | fluoropropyl)amino]-6-hydroxy-1-propylhexyl}-Nα- | |
| | (methoxycarbonyl)-β-phenyl-L-phenylalaninamide | |
| | 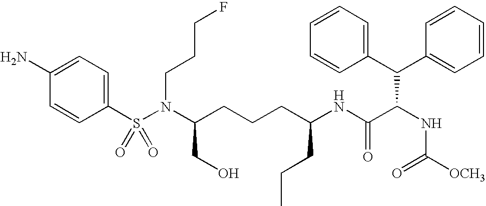 | |
| D14 | N-{(1R,5S)-5-[[(4-aminophenyl)sulfonyl](isobutyl)amino]-6- | 667 |
| | hydroxy-1-isopropylhexyl}-Nα-(methoxycarbonyl)-β-phenyl- | |
| | L-phenylalaninamide | |
| | 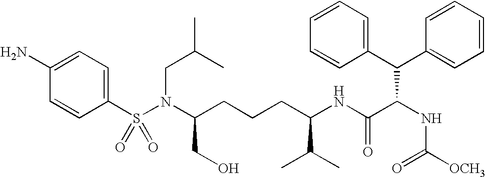 | |
| D15 | N-{(1S,5S)-5-[[(4-aminophenyl)sulfonyl](3- | 657 |
| | fluoropropyl)amino]-1-ethyl-6-hydroxyhexyl}-Nα- | |
| | (methoxycarbonyl)-β-phenyl-L-phenylalaninamide | |
| | 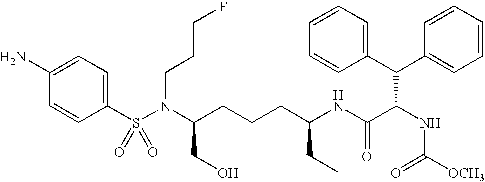 | |
| D16² | N-{(1R,5S)-5-[[(4-amino-3-fluorophenyl)sulfonyl](3- | 699 |
| | methylbutyl)amino]-6-hydroxy-1-isopropylhexyl}-Nα- | |
| | (methoxycarbonyl)-β-phenyl-L-phenylalaninamide | |
| | 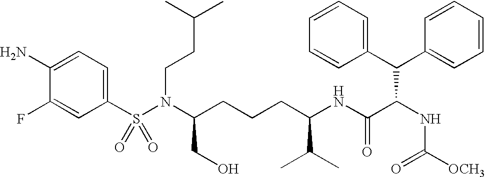 | |
| D17³ | N-{(1S,5S)-5-[[(4-amino-3-bromophenyl)sulfonyl](3- | 745 |
| | methylbutyl)amino]-1-ethyl-6-hydroxyhexyl}-Nα- | |
| | (methoxycarbonyl)-β-phenyl-L-phenylalaninamide | |
| | 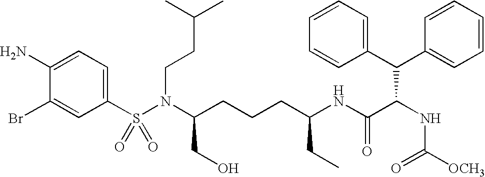 | |

¹The compound was obtained by the fluorination of the compound of Example D1, using the fluorination process described in Example D3.
²The compound was obtained by the fluorination of the compound of Example D10, using the fluorination procedure described in Example D3.
³The compound was obtained by the bromination of the compound of Example D2 using NBS.

### Example E1

### Methyl [(1S)-2-({(5S)-5-[[3-fluoro-4-aminophenyl)sulfonyl]-((3S)-3-cyclopropylbutyl)amino]-6-hydroxy-1-methylhexyl)amino)-1-(diphenylmethyl)-2-oxoethyl]carbamate

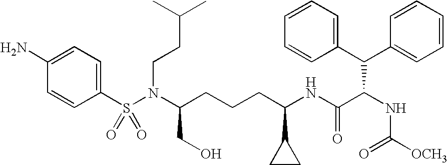

### Step E1-1: Methyl N²,N⁶-bis(tert-butoxycarbonyl)-L-lysinate

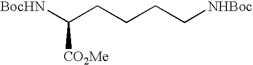

To a solution of epsilon-Boc Lysine methyl ester 1 (35.9 g, 121 mmol) suspended in the CH₂Cl₂ (250 mL) and stirred at room temperature, was added Boc₂O, 99% (28.1 mL, 121 mmol) followed by careful addition of triethylamine, 99.5% (20.23 mL, 145 mmol). The solids dissolved and gentle gas evolotuion was noted. After 1 hr the reaction mixture was clear pale yellow with no noticeable gas evoluiton. An aliquot was concentrated under N₂. The reaction mixture was allowed to sit overnight at room temperature. Transfer to a sep funnel and wash with water (2×250 mL), NaHCO₃ (50 mL 50% saturated), and brine. Dry over MgSO₄, filter and concentrate to an off white solid, wt 42 g. MS: M+Na=383.

### Step E1-2: Bis-Boc-Lysinol

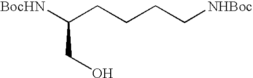

To a solution of methyl N²,N⁶-bis(tert-butoxycarbonyl)-L-lysinate (235 g, 652 mmol) in the THF (2000 mL) cooled with an ice/water bath to 10° C. was added lithium borohydride, >90% (22 g, 1010 mmol) in portions over 45 minutes. After the addition was complete the reaction mixture was aged for 20 minutes then warmed to 50° C. for 1 hour. The reaction mixture was cooled to 0° C. and quenched by dropwise addition of MeOH (50 mL). After 15 minutes at 0° C. the bath was removed and 50 mL of 5N NaOH was added with 250 mL of brine. After 30 minutes of stirring at room temperature, the reaction mixture was diluted with 500 mL of water and partitioned. The aqueous layer was diluted with more water until the salts dissolved and was then extracted once with ether (500 mL). The combined organic layer was dried over Na₂SO₄. Ethyl acetate was added and the mixture was stirred at room temperature for 15 minutes, filtered and concentrated to afford a colorless viscous oil. MS: M+Na=355.

### Step E1-3: Bis-Boc-Lysinol TBS-ether

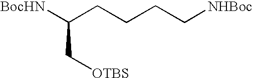

To a solution of bis-Boc-Lysinol (215 g, 647 mmol) in the CH₂Cl₂ (2500 mL) was added imidazole, >99% (86 g, 1263 mmol) followed by TBS-Cl, 97% (107 g, 711 mmol) in portions over a few minutes, while keeping the internal temperature below 30° C. The reaction mixture was stirred overnight for 2 days. 20 g of imdazole and 20 g of TBSCl were then added and the reaction mixture was aged for 3 hours, and then 20 g of imdazole, 20 g of TBSCl and 1 g DMAP were added and the reaction mixture was stirred at 35° C. for 3 hours. The reaction mixture was transferred to a separatory funnel and washed with 2N HCl (2×500 mL) and brine, dried over MgSO₄, filtered and concentrated to afford a colorless viscous oil. MS: M+Na=469.

### Step E1-4: N-Boc tert-butyl ((5S)-5-amino-6-{[tert-butyl(dimethyl)silyl]oxy}hexanoyl)carbamate

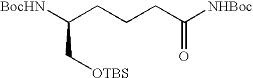

To a solution of bis-Boc-Lysinol TBS-ether 1 (289 g, 647 mmol) in EtOAc (1000 mL) was added Water (1400 mL) and ruthenium(IV) oxide hydrate (4.3 g, 28.5 mmol). 100 g of the sodium bromate was then added and the reaction mixture was stirred at 40-45° C. for 5 hours, filtered on celite, partitioned and extracted with ethyl acetate. The combined organic layer was washed with aqueous sodium bisulfite, then brine, dried over MgSO₄, treated with charcoal (Darco G-60), filtered through circa 2-inches of silica in a funnel and concentrated to a thick slurry. A small amount of hexane was added, the mixture was cooled to 0° C. and filtered. The cake was washed with 1:1 EtOAc/hexanes then hexanes, and then dried to a white solid which was not the desired product. The filtrate was concentrated then diluted with hexanes and filtered again. The filtrate was pumped onto a 1500 g column equilibrated with heptane then eluded with one column volume heptane, then a gradient to 50%. The first column volume was not collected; 450 mL fractions were then collected. Appropriate fractions were concentrated to a colorless oil:N-Boc tert-butyl ((5S)-5-amino-6-{[tert-butyl(dimethyl)silyl]oxy}hexanoyl)carbamate. MS: M+Na=483

### Step E1-5: tert-butyl [(15)-1-({[tert-butyl(dimethyl)silyl]oxy}methyl)-5-hydroxypentyl]carbamate

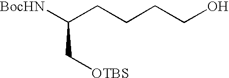

To a solution of N-Boc tert-butyl ((5S)-5-amino-6-{[tert-butyl(dimethyl)silyl]oxy}hexanoyl)carbamate (47 g, 102 mmol) in 2-propanol (900 mL) and water (90 mL) was added sodium borohydride 98+% (4.7 g, 124 mmol) and the reaction mixture was stirred at room temperature over a weekend, concentrated, diluted with ethyl acetate and 100 mL of 1 N NaOH, partitioned, washed with brine, dried over MgSO₄, filtered and concentrated to an oily solid. The residue diluted with hexanes containing a little EtOAc and filtered, washing with hexanes. The filtrate was pumped onto a 750 g column equilibrated with heptane and eluded with 1 column volume heptane, then with gradient to 50% EtOAc/heptane. The appropriate fractions were concentrated to an oily solid, dried over high vacuum to a solid that sublimes but was not the desired product. The solid was diluted with a hexanes and the mixture cooled to 0° C., inducing crystallization of the desired product. MS: M+Na=370, M-Boc+1=248.

### Step E1-6 (5S)-5-[(tert-butoxycarbonyl)amino]-6-{[tert-butyl(dimethyl)silyl]oxy}hexyl pivalate

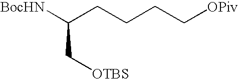

To a solution of tert-butyl [(1S)-1-({[tert-butyl(dimethyl)silyl]oxy}methyl)-5-hydroxypentyl]carbamate (2.5 g, 7.19 mmol) in 36 mL CH₂Cl₂ was added pyridine (1.10 mL, 13.67 mmol) and pivaloyl chloride (1.60 mL, 12.95 mmol). After 2 hours stirring at room temperature, further aliquots of pyridine (0.55 mL, 6.83 mmol) and pivaloyl chloride (0.80 mL, 6.47 mmol) were added. The reaction mixture was quenched after another 1 hour by diluting with EtOAc. The organics were washed in succession with 0.5M KHSO₄, saturated. aqueous NaHCO₃ and brine, then dried over Na₂SO₄, filtered and concentrated. The residue was purified by silica gel chromatography (0->15% EtOAc/hexanes) to give the desired compound as a viscous oil.

### Step E1-7 (5S)-5-[(-butoxycarbonyl)(3-methylbutyl)amino]-6-{[tert-butyl(dimethyl)silyl]oxy}hexyl pivalate

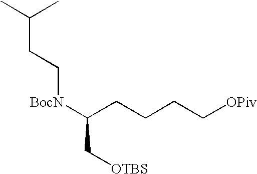

To a solution of (5S)-5-[(tert-butoxycarbonyl)amino]-6-{[tert butyl(dimethyl)silyl]oxy}hexyl pivalate (2.85 g, 6.60 mmol) in 33 mL of DMF was added NaH (95%, 0.334 g, 12.2 mmol). After 30 minutes at room temperature, 1-iodo-3-methyl butane (2.63 mL, 19.81 mmol) was added, and the reaction mixture was heated at 50° C. for 3 hours. The reaction mixture was then cooled to room temperature, quenched by adding saturated. aqueous NH₄Cl and diluted with EtOAc and H₂O. The layers were separated, and the organics were washed with 3M LiCl (3×) and brine, dried over Na₂SO₄, filtered and concentrated. The residue was purified by silica gel chromatography (0->15% EtOAc/hexanes) to give 2.39 g of the desired compound as a viscous oil.

### Step E1-8 [(1S)-1-({[tert-butyl(dimethyl)silyl]oxy 1 methyl)-5-hydroxypentyl](3-methylbutyl)carbamate

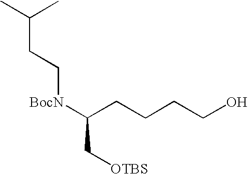

To a solution of (5S)-5-[(-butoxycarbonyl)(3-methylbutyl)amino]-6-{[tert-butyl(dimethyl)silyl]oxy}hexyl pivalate (2.29 g, 4.56 mmol) in 23 mL THF was added LiBH₄ (2M in THF, 9.13 mL, 18.25 mmol). The mixture was heated at 50° C. for 3 hours, after which a further aliquot of LiBH₄ (2M in THF, 4.6 mL, 9.1 mmol) was added, and the reaction mixture heated at 50° C. for a further 1 hour. The mixture was then cooled to room temperature, quenched by adding EtOAc, and then saturated aqueous NH₄Cl. The quenched mixture was then diluted with EtOAc, and the organics were washed organics with H₂O and brine, dried over Na₂SO₄, filtered and concentrated. The residue was purified by silica gel chromatography (10->45% EtOAc/hexanes) to give the desired compound as a viscous oil.

### Step E1-9 [(1S)-1-({[tert-butyl(dimethyl)silyl]oxy}methyl)-5-oxopentyl](3-methylbutyl)carbamate

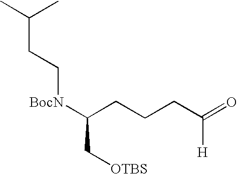

To a solution of [(1S)-1-({[tert-butyl(dimethyl)silyl]oxy}methyl)-5-hydroxypentyl](3-methylbutyl)carbamate (1.05 g, 2.51 mmol) in 17 mL CH₂Cl₂ was added N-methylmorpholine N-oxide (0.383 g, 3.27 mmol) and activated 4 Å molecular sieves (1.05 g). After 10 minutes, TPAP (0.044 g, 0.126 mmol) was added in one portion. After 45 minutes, the reaction mixture was purified by silica gel chromatography (0->25% EtOAc/hexanes) to afford 0.89 g of the desired product.

### Step E1-10 tert-butyl((1S,5E)-1-({[tert-butyl(dimethyl)silyl]oxy}methyl)-5-{[(R-tert-butylsulfinyl]imino}pentyl)(3-methylbutyl)carbamate

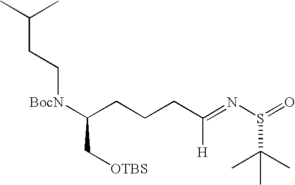

To a solution of [(1S)-1-({[tert-butyl(dimethyl)silyl]oxy}methyl)-5-oxopentyl](3-methylbutyl)carbamate (0.890 g, 2.14 mmol) in 14 mL CH₂Cl₂ was added MgSO₄ (1.29 g, 10.71 mmol), PPTS (0.054 g, 0.214 mmol) and (R)-tertbutane sulfinamide (0.337 g, 2.78 mmol) in that order. After 16 hours, the reaction mixture was filtered through a pad of celite, rinsing with fresh CH₂Cl₂. The filtrate was concentrated and purified by silica gel chromatography (10->45% EtOAc/hexanes) to afford the desired product as a viscous oil.

### Step E1-11 tert-butyl((1S,5R)-1-({[tert-butyl(dimethyl)silyl]oxy}methyl)-5-{[(R-tert-butylsulfinyl]amino}-5-cyclopropylpentyl)(3-methylbutyl)carbamate

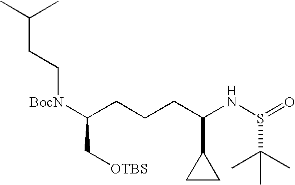

To a solution of tert-butyl((1S,5E)-1-({[tert-butyl(dimethyl)silyl]oxy}methyl)-5-{[(R-tert-butylsulfinyl]imino}pentyl)(3-methylbutyl)carbamate (0.268 g, 0.517 mmol) in 5.1 mL hexanes at −10° C. was added cyclopropylmagnesium bromide (0.5M in THF, 1.55 mL, 0.775 mmol). After 3 hours, the batch had warmed to ˜3° C., at which point the reaction mixture was quenched by adding saturated aqueous NH₄Cl and EtOAc. The layers were separated, and the organic layer was washed with brine, dried over Na₂SO₄, filtered and concentrated. The concentrate was purified by silica gel chromatography (25->75% EtOAc/hexanes) to afford desired product as a viscous oil.

### Step E1-12 tert-butyl((1S,5R)-5-amino-1-(hydroxymethyl)-5-cyclopropylpentyl)(3-methylbutyl)carbamate hydrochloride

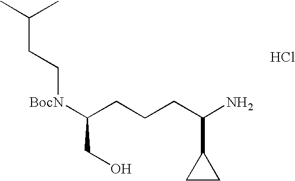

To a solution of tert-butyl((1S,5R)-1-({[tert-butyl(dimethyl)silyl]oxy}methyl)-5-{[(R-tert-butylsulfinyl]amino}-5-cyclopropylpentyl)(3-methylbutyl)carbamate (440 mg, 0.784 mmol) in 7.8 mL MeOH maintained at 0° C. was added HCl in Et₂O (1M, 1.57 mL, 1.57 mmol). After 1 hour, the bath was removed, and the reaction mixture was allowed to proceed at room temperature for 2 hours. The reaction mixture was then concentrated to obtain the desired product as a white solid. MS: M+H=343. M-Boc+1=243.

### Step E1-13 Methyl[1S 2-[((1R,5S)-5-[(tert-butoxycarbonyl)(3-methylbutyl)amino]-1-cyclopropyl-6-hydroxyhexyl}amino)-1-(diphenylmethyl)-2-oxoethyl]carbamate

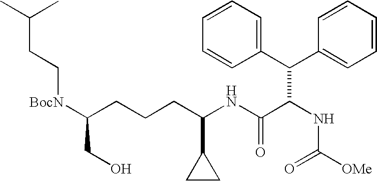

To a solution of tert-butyl((1S,5R)-5-amino-1-(hydroxymethyl)-5-cyclopropylpentyl)(3-methylbutyl)carbamate hydrochloride (371 mg, 0.979 mmol) and 2S-2-[(methoxycarbonyl)amino]-3,3-diphenylpropanoic acid (322 mg, 1.077 mmol) in 9.8 mL DMF was added diisopropylethylamine (0.170 mL, 0.979 mmol), EDC (263 mg, 1.37 mmol) and HOAt (13.3 mg, 0.098 mmol). After 16 hours of stirring at room temperature, the reaction mixture was diluted with EtOAc, and the organics were washed with 0.5M KSHO₄, 1H NaOH, 3M LiCl (3×) and brine, dried over Na₂SO₄, filtered and concentrated. The concentrate was purified by silica gel chromatography (30->80% EtOAc/hexanes) to afford the desired product as a white solid. MS: M+H=624, M-Boc+H=524.

### Step E1-14 Methyl[1S-2-[((1R,5S)-)-1-cyclopropyl-6-hydroxy-5-[(3-methylbutyl)amino]hexyl}amino)-1-(diphenylmethyl)-2-oxoethyl]carbamate hydrochloride

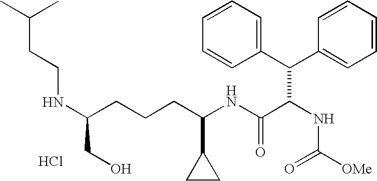

To a solution of methyl[1S 2-[((1R,5S)-5-[(tert-butoxycarbonyl)(3-methylbutyl)amino]-1-cyclopropyl-6-hydroxyhexyl}amino)-1-(diphenylmethyl)-2-oxoethyl]carbamate was added 7.1 mL of 4M HCl in dioxane. After 4 hours of stirring at room temperature, the reaction mixture was concentrated to obtain the desired product (406 mg) as a white solid. MS: M+1=524.

### Step E1-15 Methyl[1S-2-[((1R,5S)-1-cyclopropyl-6-hydroxy-5-{(3-methylbutyl)[(4-nitrophenyl)sulfonyl]amino}hexyl)amino]-1-(diphenylmethyl)-2-oxoethyl]carbamate

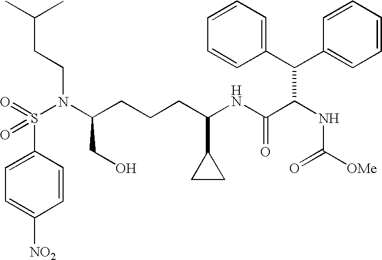

To a slurry of methyl[1S-2-[((1R,5S)-)-1-cyclopropyl-6-hydroxy-5-[(3-methylbutyl)amino]hexyl}amino)-1-(diphenylmethyl)-2-oxoethyl]carbamate hydrochloride (203 mg, 0.362 mmol) and diisopropylethylamine (0.133 mL, 0.761 mmol) in 2.4 mL CH₂Cl₂ was added 4-nitrophenylsulfonyl chloride (84 mg, 0.381 mmol). After 16 hours of stirring at room temperature, a further aliquot of 4-nitrophenylsulfonyl chloride (35 mg, 0.158 mmol) was added and after an additional 3.5 hours the reaction mixture was diluted with EtOAc, and the organics were washed with saturated aqueous NaHCO₃ and brine, dried over Na₂SO₄, filtered and concentrated. The concentrate was purified by silica gel chromatography (30->80% EtOAc/hexanes) to afford the desired product as a white solid. MS: M+H=709.

### Step E-16 Methyl[1S-2-[((1R,5S)-1-cyclopropyl-6-hydroxy-5-{(3-methylbutyl)[(4-aminophenyl)sulfonyl]amino}hexyl)amino]-1-(diphenylmethyl)-2-oxoethyl]carbamate

To a solution of methyl[1S-2-[((1R,5S)-1-cyclopropyl-6-hydroxy-5-{(3-methylbutyl)[(4-nitrophenyl)sulfonyl]amino}hexyl)amino]-1-(diphenylmethyl)-2-oxoethyl]carbamate (102 mg, 0.144 mmol) in 0.72 mL EtOH and 0.72 mL THF was added SnCl₂ (136 mg, 0.719 mmol). The reaction mixture was placed in a pre heated oil bath at 85° C. for 2.5 hours. The reaction mixture was then cooled to room temperature, and quenched by the addition of saturated aqueous NaHCO₃. The quenched reaction mixture was diluted with EtOAc and H₂O, the layers were separated. After washing the aqueous layer with EtOAc (4×), the combined organics were dried over Na₂SO₄, filtered through a pad of celite and concentrated. The concentrate was purified by silica gel chromatography (40->100% EtOAc/hexanes) to afford the desired product as a white foam. MS: M+H=679. ¹H NMR (400 MHz, CDCl₃) δ 7.58 (d, J=8.6 Hz, 2H), 7.30-7.18 (m, 5H), 6.69 (d, J=8.6 Hz, 1H), 5.47 (d, J=8.2 Hz, 1H), 5.10 (d, J=8.2 Hz, 1H), 4.78 (t, J=9.7 Hz, 1H), 4.49 (d, J=10.1 Hz, 1H), 4.40 (s, 2H), 3.61 (s, 3H), 3.48-3.47 (m, 3H), 3.17 (ddd, J=15.0 Hz, 9.6 Hz, 5.3 Hz, 1H), 3.05-2.95 (m, 2H), 2.47 (s, 1H), 1.56-1.48 (m, 5H), 1.14 (m, 1H), 0.99 (m, 3H), 0.90 (d, J=1.7 Hz, 3H), 0.88 (d, J=2.1 Hz, 3H), 0.61 (m, 1H), 0.48-0.29 (m, 3H), 0.161 (ddd, J=9.3, 4.6, 4.6 Hz, 1H), 0.045 (m, 1H).

### Example E2

### N-{(1R,5S)-1-cyclopropyl-5-[{[3-fluoro-4-(hydroxymethyl)phenyl]sulfonyl}(3-methylbutyl)amino]-6-hydroxyhexyl}-4-fluoro-β-(4-fluorophenyl)-Nα-(methoxycarbonyl)-L-phenylalaninamide

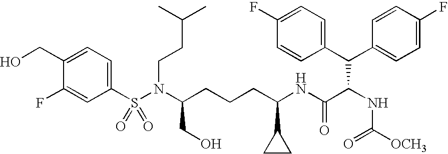

### Step E2-1: N-{(1R,5S)-5-[[(4-bromo-3-fluorophenyl)sulfonyl](3-methylbutyl)amino]-1-cyclopropyl-6-hydroxyhexyl}-4-fluoro-β-(4-fluorophenyl)-Nα-(methoxycarbonyl)-L-phenylalaninamide

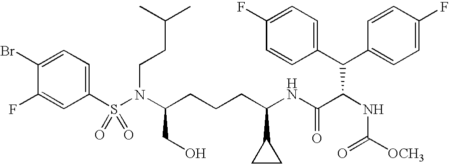

N-{(1R,5S)-5-[[(4-bromo-3-fluorophenyl)sulfonyl](3-methylbutyl)amino]-1-cyclopropyl-6-hydroxyhexyl}-4-fluoro-β-(4-fluorophenyl)-Nα-(methoxycarbonyl)-L-phenylalaninamide was prepared from 3-fluoro-4-bromo-benzene sulfonyl chloride using a procedure similar to that described in the preparation of Example E1.

### Step E2-2: Methyl 4-{[[(1S,5R)-5-cyclopropyl-5-[4-fluoro-β-(4-fluorophenyl)-N-(methoxycarbonyl)-L-phenylalanyl]amino}-1-(hydroxymethyl)pentyl](3-methylbutyl)amino]sulfonyl}-2-fluorobenzoate

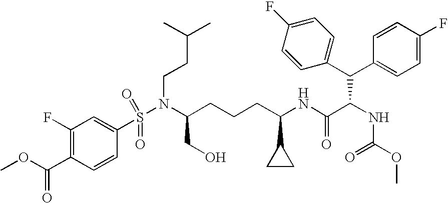

N-{(1R,5S)-5-[[(4-bromo-3-fluorophenyl)sulfonyl](3-methylbutyl)amino]-1-cyclopropyl-6-hydroxyhexyl}-4-fluoro-β-(4-fluorophenyl)-Nα-(methoxycarbonyl)-L-phenylalaninamide (40 mg, 0.05 mmol) was dissolved in a 1:2 solution of DMSO:MeOH (1.5 mL) and the solution was purged with argon. Triethylamine (20 mg, 0.20 mmol), palladium acetate (2.25 mg, 10 μmol), 1,3-bis(diphenylphosphino)propane (4.1 mg, 10 μmol) were added to the stirring solution. Carbon monoxide was introduced via balloon and the resulting apparatus was fitted with an air condenser and heated to 80° C. for 16 hours. The crude reaction mixture was then filtered over celite, and partitioned between brine and ethyl acetate. The combined organics were dried over sodium sulfate and concentrated in vacuo. The crude mixture was purified using reverse phase chromatography. The appropriate fractions were extracted into ethyl acetate and washed with saturated sodium bicarbonate and brine to yield the title product as a clear oil. LCMS (M+1)=776.

### Step E2-3: N-{(1R,5S)-1-cyclopropyl-5-[{[3-fluoro-4-(hydroxymethyl)phenyl]sulfonyl}(3-methylbutyl)amino]-6-hydroxyhexyl}-4-fluoro-β-(4-fluorophenyl)-Nα-(methoxycarbonyl)-L-phenylalaninamide

Methyl 4-{[[(1S,5R)-5-cyclopropyl-5-{[4-fluoro-β-(4-fluorophenyl)-N-(methoxycarbonyl)-L-phenylalanyl]amino}-1-(hydroxymethyl)pentyl](3-methylbutyl)amino]sulfonyl}-2-fluorobenzoate (4 mg, 5.16 μmol) was dissolved in THF (100 μL) and lithium borohydride (0.11 mg, 5.2 pimp added dropwise to the stirring solution. The reaction was stirred for 8 hours and then quenched with saturated ammonium chloride solution, extracted into ethyl acetate, dried over sodium sulfate and concentrated in vacuo. The crude mixture was purified using reverse phase chromatography. The appropriate fractions were extracted into ethyl acetate and washed with saturated sodium bicarbonate and brine to the desired product. ¹H NMR (CDCl₃): δ 7.6 (m, 3H), 7.25 (m, 5H), 7.1-6.9 (m, 5H), 5.15 (m, 1H), 5.05 (m, 1H), 4.8 (m, 2H), 4.4 (m, 1H), 3.7 (s, 3H), 3.6-3.4 (m, 2H), 3.25-3.05 (m, 2H), 2.8 (m, 1H), 1.7-1.5 (m, 4H), 1.35-1.25 (m, 4H), 1.0-0.8 (m, 8H), 0.55-0.35 (m, 2H), 0.15-0.25 (m, 2H). LCMS (M+1)=748.

### Example E3

### N-{(1R,5S)-5-[[(4-amino-3-fluorophenyl)sulfonyl](3-methylbutyl)amino]-1-cyclopropyl-6-hydroxyhexyl}-Nα-(methoxycarbonyl)-β-phenyl-L-phenylalaninamide

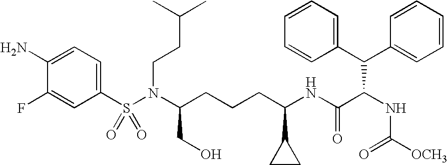

The title product was prepared from methyl [(1S)-2-({(5S)-5-[[3-fluoro-4-aminophenyl)sulfonyl]-((3S)-3-cyclopropylbutyl)amino]-6-hydroxy-1-methylhexyl)amino)-1-(diphenylmethyl)-2-oxoethyl]carbamate (Example E1) by fluorination in the manner described in Example D3. MS M+1=697.

The following examples (Table E) were prepared using procedures similar to those described in the preparation of Examples E1 to E3, using the appropriate building blocks (R⁵MgX, ArSO₂Cl, R¹X, HO₂C—CHR⁶—NHR⁷ or corresponding activated amino acid such as hydroxysuccinate ester). In some cases NHR⁷ is originally protected as Boc which necessitates an acidic Boc removal in the last step.

TABLE E

| Example | | |
|---|---|---|
| No. | Structure | M + 1 |
| E4 | N-{(1R,5S)-5-[[(4-chlorophenyl)sulfonyl](3- | 698 |
| | methylbutyl)amino]-1-cyclopropyl-6-hydroxyhexyl}- | |
| | Nα-(methoxycarbonyl)-β-phenyl-L-phenylalaninamide | |
| | 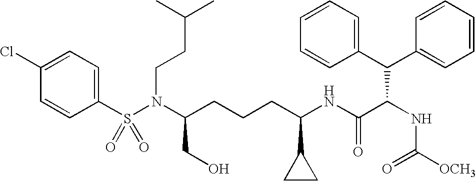 | |
| E5¹ | N-{(1R,5S)-5-[[(4-acetylphenyl)sulfonyl](3- | 742 |
| | methylbutyl)amino]-1-cyclopropyl-6-hydroxyhexyl}-4-fluoro-β- | |
| | (4-fluorophenyl)-Nα-(methoxycarbonyl)-L-phenylalaninamide | |
| | 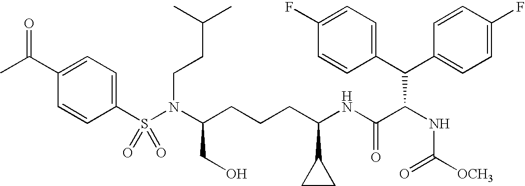 | |
| E6¹ | N-{(1R,5S)-5-[(1,3-benzothiazol-6-ylsulfonyl)(isobutyl) | 707 |
| | amino]-1-cyclopropyl-6-hydroxyhexyl}-Nα-(methoxycarbonyl)- | |
| | β-phenyl-L-phenylalaninamide | |
| | 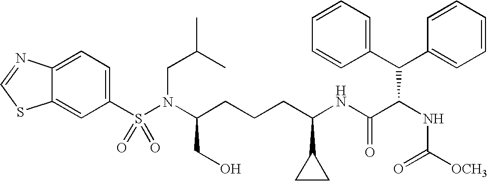 | |
| E7 | N-{(1R,5S)-5-[[(4-aminophenyl)sulfonyl](3-methylbutyl) | 715 |
| | amino]-1-cyclopropyl-6-hydroxyhexyl}-4-fluoro-β- | |
| | (4-fluorophenyl)-Nα-(methoxycarbonyl)-L-phenylalaninamide | |
| | 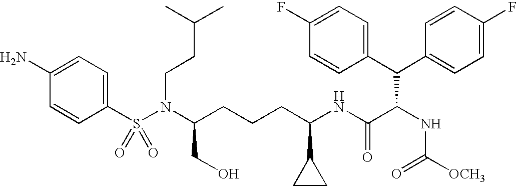 | |
| E8¹ | N-{(1R,5S)-5-[(1,3-benzothiazol-6-ylsulfonyl)(3-methylbutyl) | 679 |
| | amino]-1-cyclopropyl-6-hydroxyhexyl}-2-chloro-Nα- | |
| | (methoxycarbonyl)-L-phenylalaninamide | |
| | 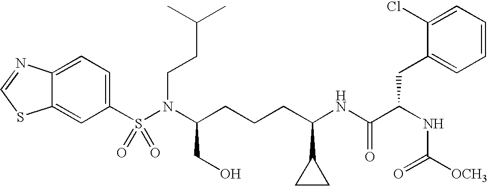 | |
| E9 | methyl [(1S)-2-({(1R,5S)-5-[[(4-aminophenyl)sulfonyl] | 653 |
| | (3-methylbutyl)amino]-1-cyclopropyl-6-hydroxyhexyl}amino)-1- | |
| | (1-naphthylmethyl)-2-oxoethyl]carbamate | |
| | 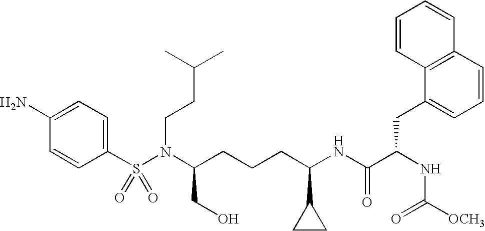 | |

¹No reduction after sulfonylation.

### Example F1

### N-{(1S,5S)-5-[[(4-aminophenyl)sulfonyl](isopropyl)amino]-6-hydroxy-1-methylhexyl}-Nα-(methoxycarbonyl)-β-phenyl-L-phenylalaninamide

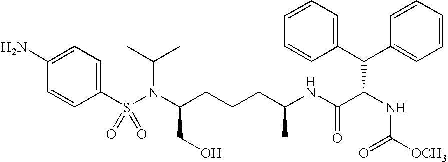

### Step F1-1 Ethyl (2S)-2-amino-4-pentenoate hydrochloride

To a solution of (2S)-2-aminopenenoic acid (10.5 g, 91 mmol) in 100 mL EtOH at 0° C. was added thionyl chloride (20.0 mL, 274 mmol) dropwise over 30 minutes. The bath was removed, and the reaction was allowed to proceed at room temperature over 16 hours. The reaction mixture was concentrated, and the brownish residue was titurated with Et₂O (2×) to obtain the desired product as a white solid. MS: M+H=144.

### Step F1-2: Ethyl (2S)-2-{[(4-nitrophenyl)sulfonyl]amino-4-pentenoate

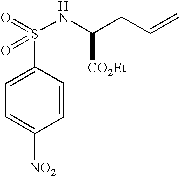

To a slurry of ethyl (2S)-2-amino-4-pentenoate hydrochloride (12.01 g, 66.9 mmol) in 223 mL CH₂Cl₂ was added triethylamine (20.0 mL, 140 mmol) and 4-nitrophenylsulfonyl chloride (14.67 g, 66.2 mmol). The reaction was allowed to proceed at room temperature for 16 hours, then diluted with EtOAc and washed with 0.5M HCl (2×), saturated aqueous NaHCO₃ and brine, dried over Na₂SO₄, filtered and concentrated to obtain the desired product which was subsequently used without further purification.

### Step F1-3 Ethyl (2S)-)-2-{isopropyl[(4-nitrophenyl)sulfonyl]amino}-4-pentenoate

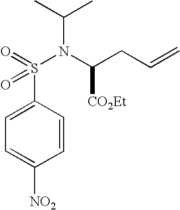

To a solution of ethyl (2S)-2-{[(4-nitrophenyl)sulfonyl]amino}-4-pentenoate (2.45 g, 7.46 mmol) in 75 mL THF was added Ph₃P (5.87 g, 22.4 mmol), i-PrOH (11.5 mL, 149 mmol) and DIAD (4.35 mL, 22.4 mmol). After 1 hour of stirring at room temperature, the reaction mixture was concentrated and the residue was purified by silica gel chromatography (20->100% EtOAc/hexanes) to obtain the desired product. MS: M+H=371.

### Step F1-4 Ethyl (2S,4E)-2-{isopropyl[(4-nitrophenyl)sulfonyl]amino}-6-oxo-4-heptenoate

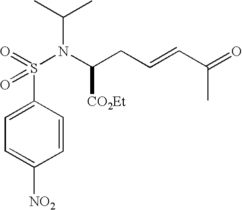

To a solution of ethyl (2S)-)-2-{isopropyl[(4-nitrophenyl)sulfonyl]amino}-4-pentenoate (2.95 g, 7.96 mmol) in 80 mL CH₂Cl₂ was added methyl crotyl ketone (60% purity, 13.0 mL, 80.0 mmol) and Grubbs 2″ generation catalyst (0.676 g, 0.796 mmol). The reaction mixture was heated to 65° C. for 16 hours. Concentrated and purified residue by silica gel chromatography (20->100% EtOAc/hexanes) to obtain the desired product (1.70 g). MS: M+H=413.

### Step F1-5 Ethyl (2S)-)-2-{isopropyl[(4-aminophenyl)sulfonyl]amino}-6-oxoheptanoate

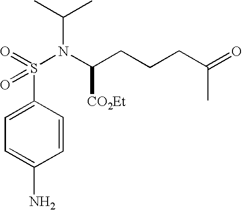

To a solution of ethyl (2S,4E)-2-{isopropyl[(4-nitrophenyl)sulfonyl]amino}-6-oxo-4-heptenoate (2.24 g, 5.42 mmol) in 54 mL EtOH was added 20% Pd(OH)₂ on carbon (0.762 g, 1.08 mmol). A H₂ balloon was attached, and the flask was evacuated/backfilled with H₂ (3×). After 2.5 hours of stirring at room temperature, the flask was evacuated/backfilled with N₂, and the reaction mixture was filtered through a pad of celite under N₂, rinsing with CH₂Cl₂. The organics were concentrated to provide the desired product. MS: M+H=385.

### Step F1-6 Ethyl(2S,6S)-2-[[(4-aminophenyl)sulfonyl](isopropyl)amino]-6-{[(S-tert-butylsulfinyl]amino}heptanoate

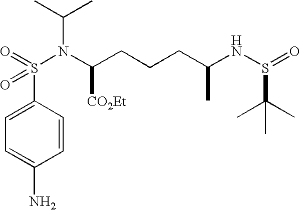

To a solution of ethyl (2S)-)-2-{isopropyl[(4-aminophenyl)sulfonyl]amino}-6-oxoheptanoate (2.07 g, 5.38 mmol) in 41 mL THF was added S-tert-butane sulfinamide (1.96 g, 16.1 mmol), followed by Ti(OEt)₄ (5.60 mL, 26.9 mmol). The reaction mixture was heated at 65° C. for 16 hours, then cooled to −50° C. Sodium borohydride (1.63 g, 43.0 mmol) was added in one portion, and the reaction mixture was allowed to warm to −10° C. over 3 hours. The reaction mixture was then quenched by adding MeOH at −10° C., and then diluted with EA. Brine (10 mL) was then added and the mixture warmed to room temperature, stirred at room temperature for 20 minutes, then filtered through a pad of celite, rinsing with fresh EA. The filtrate was then concentrated and used in Step F1-7 without further purification. MS: M+H=490, 4-5:1 ratio of diastereomers by HPLC.

### Step F1-7 Ethyl(2S,6S)-2-[[(4-aminophenyl)sulfonyl](isopropyl)amino]heptanoate hydrochloride

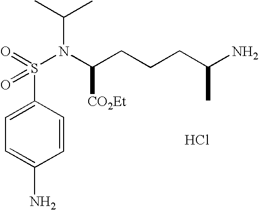

Unpurified ethyl(2S,6S)-2-[[(4-aminophenyl)sulfonyl](isopropyl)amino]-6-{[(S-tert-butylsulfinyl]amino}heptanoate from Step F1-6 was dissolved in 41 mL MeOH, after which 1M HCl in Et₂O (32.0 mL, 32.0 mmol) was added. After 30 minutes of stirring at room temperature, the reaction mixture was concentrated, and the resulting amine was subsequently used without further purification. MS: M+H=386.

### Step F1-8 Ethyl (2S,6S)-2-[[(4-aminophenyl)sulfonyl](isopropyl)amino]-6-({(2S)-2-[(methoxycarbonyl)amino]-3,3-diphenylpropanoyl}amino)heptanoate

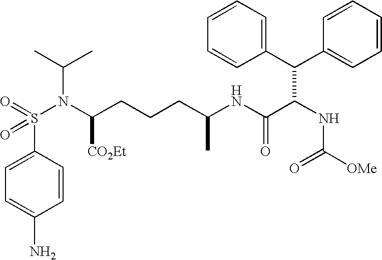

To a solution of ethyl(2S,6S)-2-[[(4-aminophenyl)sulfonyl](isopropyl)-amino]heptanoate hydrochloride in 27 mL THF and 27 mL saturated aqueous NaHCO₃ was added methyl[(1S)-2-[(2,5-dioxo-1-pyrrolidinyl)oxy]-1-(diphenylmethyl)-2-oxoethyl]carbamate (2.13 g, 5.38 mmol). After 16 hours of stirring at room temperature, the reaction mixture was diluted with EtOAc and H₂O. The resulting layers were separated, and the organic layer was washed with brine, dried over Na₂SO₄, filtered and concentrated. The concentrate was purified by silica gel chromatography (30->90% EtOAc/hexanes) to cleanly obtain the desired 6-methyl diastereomer generated in Step F1-6 via reduction of the in situ formed Ellman sulfinyl imine MS: M+H=667.

### Step F1-9 Methyl[(1S)-2-({(1S,5S)-5-[[(4-aminophenyl)sulfonyl](isopropyl)amino]-6-hydroxy-1-methylhexyl}amino)-1-(diphenylmethyl)-2-oxoethyl]carbamate

To a solution of ethyl (2S,6S)-2-[[(4-aminophenyl)sulfonyl](isopropyl)amino]-6-({(2S)-2-[(methoxycarbonyl)amino]-3,3-diphenylpropanoyl}amino)heptanoate (2.07 g, 3.10 mmol) in 21 mL THF was added LiBH₄ in THF (2M, 7.76 mL, 15.52 mmol). After 16 hours of stirring at room temperature, the reaction mixture was cooled to 0° C. and quenched by the addition of EtOAc, MeOH and saturated aqueous NH₄Cl in that order. The quenched mixture was then diluted with more EtOAc, and the resulting organic layer was washed with H₂O and brine, dried over Na₂SO₄, filtered and concentrated. The concentrate was purified by silica gel chromatography (30->90% EtOAc/hexanes) to obtain desired the desired product as a white foam. MS: M+H=625. ¹H NMR (400 MHz, CDCl₃) δ 7.61 (d, J=8.6 Hz, 2H), 7.32-7.18 (m, 5H), 6.67 (d, J=8.6 Hz, 2H), 5.31 (d, J=8.4 Hz, 1H), 5.12 (d, J=8.4 Hz, 1H), 4.75 (t, J=9.8 Hz, 1H), 4.45 (d, J=9.8 Hz, 1H), 4.35 (s, 1H), 3.68-3.55 (m, 5H), 3.59 (s, 3H), 3.25-3.19 (m, 1H), 2.81 (s, 1H), 1.38 (d, J=6.6 Hz, 3H), 1.26 (d, J=6.8 Hz, 3H), 1.31-1.23 (m, 3H), 87 (d, J=6.5 Hz), 0.84 (m, 3H), 0.615 (s, 1H), 0.38 (s, 1H).

The following examples (Table F) were prepared using procedures similar to those described in the preparation of Example F1, using the appropriate building blocks (RSCOCH═CHMe, ArSO₂Cl, R¹OH, HO₂C—CHR⁶—NHR⁷ or corresponding activated amino acid such as hydroxysuccinate ester). In some cases NHR⁷ is originally protected as Boc which necessitates an acidic Boc removal in the last step.

TABLE F

| Example | | |
|---|---|---|
| No. | Structure | M + 1 |
| F2 | N-{(1S,5S)-5-[[(4- aminophenyl)sulfonyl](propyl)amino]-6-hydroxy-1- methylhexyl}-Nα-(methoxycarbonyl)-β-phenyl-L- phenylalaninamide 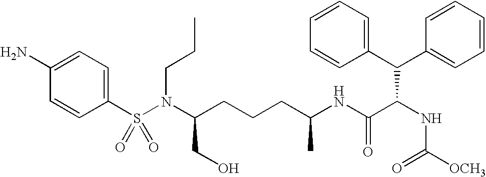 | 625 |
| F3 | N-{(1S,5S)-5-[[(4-aminophenyl)sulfonyl](3- methylbutyl)amino]-1-ethyl-6-hydroxyhexyl}-2- bromo-Nα-(methoxycarbonyl)-L-phenylalaninamide 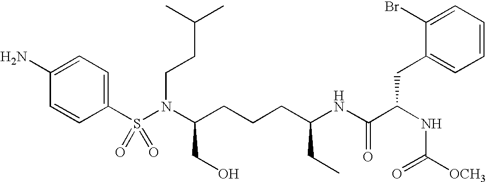 | 669 |
| F4 | N-{(1S,5S)-5-[[(4-aminophenyl)sulfonyl](3- fluoropropyl)amino]-6-hydroxy-1-methylhexyl}-Nα- (methoxycarbonyl)-β-phenyl-L-phenylalaninamide 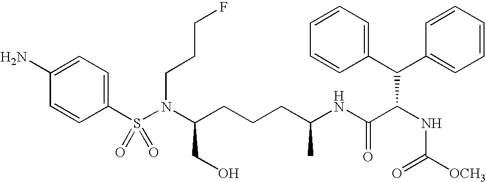 | 643 |
| F5 | N-{(1S,5S)-5-[[(4- aminophenyl)sulfonyl](propyl)amino]-1-ethyl-6- hydroxyhexyl}-Nα-(methoxycarbonyl)-β-phenyl-L- phenylalaninamide 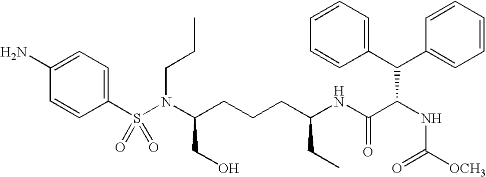 | 639 |
| F6 | methyl [2-({(1S,5S)-5-[[(4-aminophenyl)sulfonyl](3- methylbutyl)amino]-1-ethyl-6- hydroxyhexyl}amino)-1-(5H- dibenzo[a,d][7]annulen-5-yl)-2-oxoethyl]carbamate 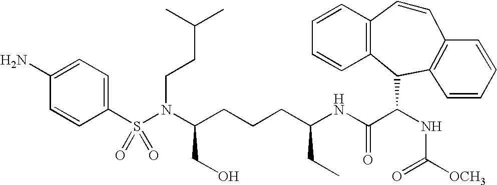 | 691 |
| F7 | N-{(1S,5S)-5-[[(4- aminophenyl)sulfonyl](propyl)amino]-1-ethyl-6- hydroxyhexyl}-2-bromo-Nα-(methoxycarbonyl)-L- phenylalaninamide 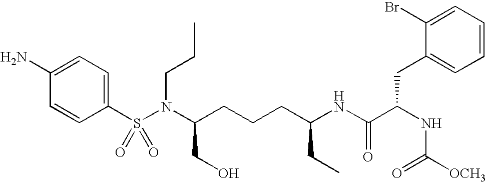 | 641 |
| F8 | N-{(1S,5S)-5-[[(4- aminophenyl)sulfonyl](propyl)amino]-1-ethyl-6- hydroxyhexyl}-2-chloro-Nα-(methoxycarbonyl)-L- phenylalaninamide 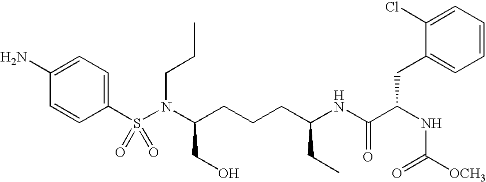 | 597 |
| F9 | tert-butyl {(1R,2R)-1-[({(1S,5S)-5-[[(4- aminophenyl)sulfonyl](3-methylbutyl)amino]-1- ethyl-6-hydroxyhexyl}amino)carbonyl]-2- phenylcyclopropyl}carbamate 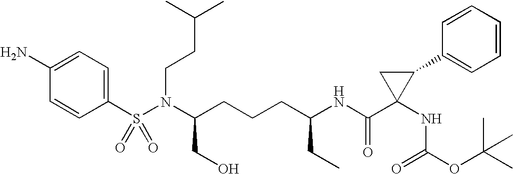 | 645 |
| F10 | N-{(1S,55)-5-[[(4-aminophenyl)sulfonyl](3- methylbutyl)amino]-1-ethyl-6-hydroxyhexyl}-β- phenyl-Nα-[(pyridin-4-ylmethoxy)carbonyl]-L- phenylalaninamide 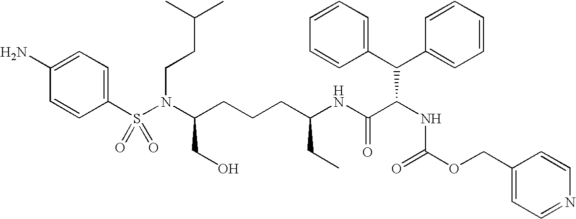 | 744 |
| F11 | methyl [2-({(1S,5S)-5-[[(4-aminophenyl)sulfonyl](3- methylbutyl)amino]-1-ethyl-6- hydroxyhexyl}amino)-2-oxo-1-(9H-xanthen-9- yl)ethyl]carbamate 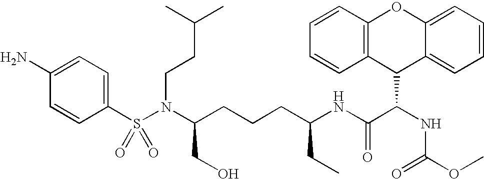 | 681 |
| F12¹ | N{(5S)-5-[[(4-aminophenyl)sulfonyl](3- methylbutyl)amino]-1-ethyl-6-hydroxyhexyl}-Nα- (methoxycarbonyl)-Nα-methyl-β-phenyl-L- phenylalaninamide 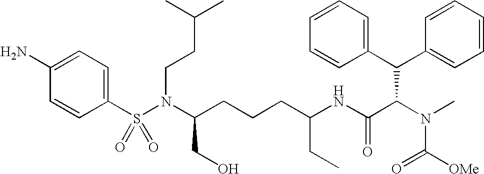 | 681 |
| F13² | N-{(1S,5S)-5-[[(4-aminophenyl)sulfonyl](3- methylbutyl)amino]-1-ethyl-6-hydroxyhexyl}-β- phenyl-L-phenylalaninamide 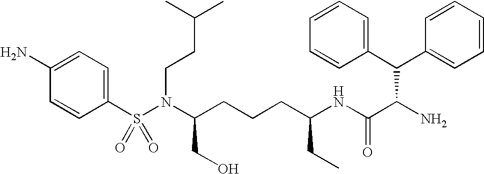 | 609 |
| F14 | N-{(1R,5S)-5-[[(4- aminophenyl)sulfonyl](isopropyl)amino]-6-hydroxy- 1-isopropylhexyl}-Nα-(methoxycarbonyl)-β-phenyl- L-phenylalaninamide 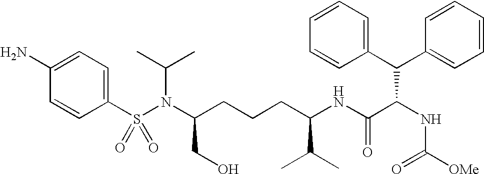 | 653 |
| F15 | N-{(1S,5S)-5-[[(4- aminophenyl)sulfonyl](isopropyl)amino]-6-hydroxy- 1-methylhexyl}-3-fluoro-β-(3-fluorophenyl)-Nα- (methoxycarbonyl)-L-phenylalaninamide 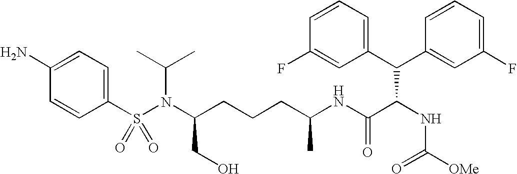 | 661 |
| F16 | N-{(1S,5S)-5-[[(4- aminophenyl)sulfonyl](isopropyl)amino]-6-hydroxy- 1-methylhexyl}-2,3-dichloro-Nα-(methoxycarbonyl)- L-phenylalaninamide 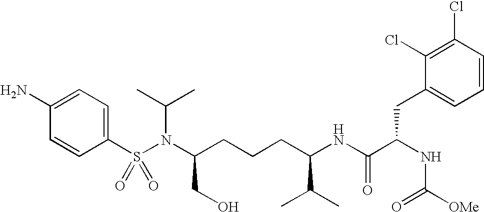 | 617 |
| F17 | N-{(1S,5S)-5-[[(4- aminophenyl)sulfonyl](propyl)amino]-6-hydroxy-1- methylhexyl}-3-fluoro-β-(3-fluorophenyl)-Nα- (methoxycarbonyl)-L-phenylalaninamide 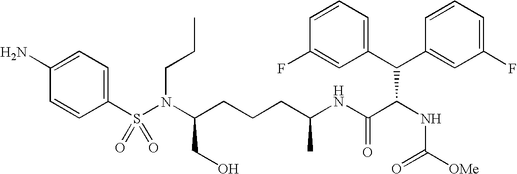 | 661 |

¹The product was a diastereomeric mixture, RS at C5 lysine side-chain.
²Boc removal was the last step in the preparation of the compound.

### Example G1

### N-[(1S,5S)-5-(ethyl{[4-(hydroxymethyl)phenyl]sulfonyl}amino)-6-hydroxy-1-methylhexyl]-Nα-(methoxycarbonyl)-β-phenyl-L-phenylalaninamide

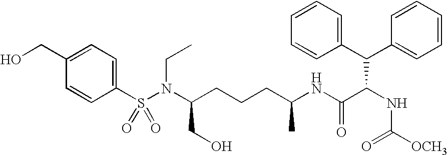

### Step G1-1 Methyl (2S)-2-[(tert-butoxycarbonyl)amino]-4-pentenoate

To a stirring solution of N-Boc-L-allyl glycine (2 g, 9.29 mmol) in acetone was added K₂CO₃ (2.57 g, 18.58 mmol) and methyl iodide (2.64 g, 18.58 mmol). The reaction mixture was heated to reflux overnight, concentrated under vacuum, dissolved in ethyl acetate and washed with saturated NaHCO₃ then brine. The organic extracts where dried with Na₂SO₄, filtered and concentrated under vacuum. All spectroscopic analysis are consistent with the literature and the crude material was used without further purification.

### Step G1-2 Methyl (2S,4E)-2-[(tert-butoxycarbonyl)amino]-6-oxo-4-heptenoate

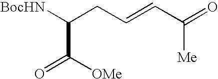

To a solution of methyl (2S)-2-[(tert-butoxycarbonyl)amino]-4-pentenoate (6.54 g, 28.5 mmol) in 60 mL CH₂Cl₂ was added methyl crotyl ketone (70% purity, 20.0 mL, 143.0 mmol) and Grubbs 2^{nd} generation catalyst (1.21 g, 1.43 mmol). The reaction mixture was heated to 65° C. for 16 hours. The mixture was then concentrated and the concentrate was purified by silica gel chromatography (gradient: 40 to 80% EA/hexanes) to obtain the desired product.

### Step G1-3 Methyl (2S,4E)-2-[(tert-butoxycarbonyl)amino]-6-oxoheptanoate

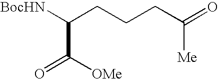

To a stirring solution of methyl (2S,4E)-2-[(tert-butoxycarbonyl)amino]-6-oxoheptanoate (3.32 g, 12.24 mmol) in 30 mL EtOH was added Pd(OH)₂ (20% on carbon, 1.72 g, 1.22 mmol). A H2 balloon was attached, and the flask was evacuated/backfilled with H2 (3×). After 3 hours, the flask was evacuated/backfilled with N₂, and the reaction mixture was filtered through a pad of celite under N₂, rinsing with CH₂Cl₂. The organics were concentrated to provide the desired product.

### Step G1-4 Ethyl-(2S,6S)-2-[(tert-butoxycarbonyl)amino]-6-{[(S)-tert-butylsulfinyl]amino}heptanoate

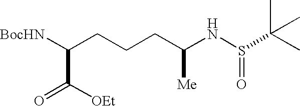

To a stirring solution of methyl (2S,4E)-2-[(tert-butoxycarbonyl)amino]-6-oxoheptanoate (1.5 g, 5.49 mmol) in 20 mL THF was added S-tert-butane sulfinamide (1.0 g, 8.23 mmol), followed by Ti(OEt)₄ (3.46 mL, 16.5 mmol). The reaction mixture was heated at 65° C. for 16 hours, then cooled to −50° C. Sodium borohydride (1.04 g, 27.0 mmol) was added in one portion, and the reaction mixture was allowed to warm to −10° C. over 3 hours. The mixture was then quenched by adding MeOH at −10° C., then diluted with EA, after which brine (10 mL) was added. The mixture was then warmed to room temperature, stirred at room temperature for 20 minutes, then filtered through a pad of celite, rinsing with fresh ethyl acetate. The reaction mixture was concentrated and used in Step G1-5 without further purification. MS (M+H=392), 4-5:1 ratio of diastereomers by HPLC.

### Step G1-5 Ethyl-(2S,6S)-6-amino-2-[(tert-butoxycarbonyl)amino]heptanoate

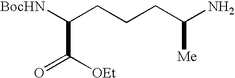

To a stirring solution of ethyl-(2S,6S)-2-[(tert-butoxycarbonyl)amino]-6-{[(S)-tert-butylsulfinyl]amino}heptanoate from Step G1-4 in 41 mL MeOH at room temperature was added 1M HCl in Et₂O (7.64 mL, 7.64.0 mmol). After 30 minutes, the reaction mixture was quenched with saturated NaHCO₃ (neutralized to pH 7) and extracted with DCM. The organic extracts where combined, dried with Na₂SO₄, filtered and concentrated under vacuum. The resulting amine was carried on without further purification. MS (M+H=288).

### Step G1-6 Ethyl (2S,6S)-2-[(tert-butoxycarbonyl)amino]-6-({(2S)-2-[(methoxycarbonyl)amino]-3,3-diphenylpropanoyl)amino)heptanoate

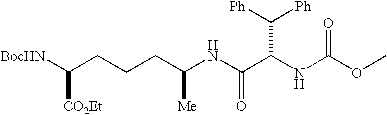

To a stirring solution of ethyl-(2S,6S)-6-amino-2-[(tert-butoxycarbonyl)amino]heptanoate (0.300 g, 1.04 mmol) in 2 mL THF and 2 mL saturated aqueous NaHCO₃ at room temperature was added methyl[(1S)-2-[(2,5-dioxo-1-pyrrolidinyl)oxy]-1-(diphenylmethyl)-2-oxoethyl]carbamate (2.13 g, 5.38 mmol). After 16 hours, the reaction mixture was diluted with EA and H₂O, Separated layers, washed organics with brine, dried over Na₂SO₄, filtered and concentrated (550 mg). The resulting compound was used without further purification. MS (M+H=569).

### Step G1-7 Ethyl(2S,6S)-2amino-6-({(2S)-2-[(methoxycarbonyl)amino]-3,3-diphenylpropanoyl)amino)heptanoate

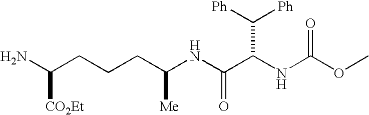

To a stirring solution of ethyl(2S,6S)-2-[(tert-butoxycarbonyl)amino]-6-({(2S)-2-[(methoxycarbonyl)amino]-3,3-diphenylpropanoyl)amino)heptanoate (90 mg, 0.158 mmol) in 10 mL of DCM at room temperature was added 4M HCl in dioxane (118 μL, 0.474 mmol). After 30 minutes, the reaction mixture was concentrated, and the resulting amine was carried on without further purification. MS (M+H=470).

### Step G1-8: Methyl 4-{[[1S,5S-1-(ethoxycarbonyl)-5-({(2S)-2-[(methoxycarbonyl)amino]-3,3-diphenylpropanoyl)amino)hexyl](ethyl)amino]sulfonyl}benzoate

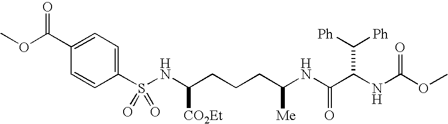

To a solution of ethyl (2S,6S)-2amino-6-({(2S)-2-[(methoxycarbonyl)amino]-3,3-diphenylpropanoyl)amino)heptanoate (0.120 g, 0.237 mmol) in 10 mL CH₂Cl₂ was added triethylamine (132 μL, 0.950 mmol) and 4-estersulfonyl chloride (52.9 mg, 0.225 mmol). The reaction mixture was allowed to proceed at room temperature for 16 hours, then diluted with EA and washed with 0.5M HCl (2×), saturated aqueous NaHCO₃ and brine, dried over Na₂SO₄, filtered and concentrated to obtain the desired product that was used without further purification.

### Step G1-9 (5S,8S,12S)-ethyl-5-benzhydryl-13-(4-(methoxycarbonyl)phenylsulfonyl)-8-methyl-3,6-dioxo-2-oxa-4,17,13-triazapentadecane-12-carboxylate

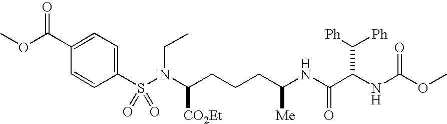

To a stirring solution of methyl 4-{[[1S,5S-1-(ethoxycarbonyl)-5-({(2S)-2-[(methoxycarbonyl)amino]-3,3-diphenylpropanoyl)amino)hexyl](ethyl)amino]sulfonyl}benzoate (0.075 g, 0.112 mmol) in 2 mL THF at room temperature was added Ph₃P (0.088 g, 0.337 mmol), EtOH (25.9 mg, 0.562 mmol) and DIAD (65.5 μL, 0.337 mmol). After 1 hour, the reaction mixture was concentrated and the concentrate was purified by silica gel chromatography (20->100% EA/hexanes) to obtain the desired product. MS (M+H=695).

### Step G1-10 N-[(1S,5S)-5-(ethyl{[4-(hydroxymethyl)phenyl]sulfonyl}amino)-6-hydroxy-1-methylhexyl]-Nα-(methoxycarbonyl)-β-phenyl-L-phenylalaninamide

To a stirring solution of ethyl (5S,8S,12S)-ethyl-5-benzhydryl-13-(4-(methoxycarbonyl)phenylsulfonyl)-8-methyl-3,6-dioxo-2-oxa-4,17,13-triazapentadecane-12-carboxylate (0.050 g, 0.072 mmol) in 1.5 mL THF at room temperature was added LiBH₄ in THF (2M, 719 μL, 0.719 mmol). After 16 hours, the reaction mixture was cooled to 0° C. and quenched by the addition of EA, MeOH and saturated aqueous NH₄Cl in that order. The mixture was then diluted with more EA, and the organics were washed with H₂O and brine, dried over Na₂SO₄, filtered and concentrated. The concentrate was purified by silica gel chromatography (30->90% EA/hexanes) to obtain the desired product as a white solid. MS (M+H=626). ¹H NMR (400 MHz, CDCl₃) δ 7.78 (d, J=8.4 Hz, 2H), 7.52 (d, J=8.4 Hz, 2H), 7.32-7.18 (m, 10H), 6.23 (d, J=7.2 Hz, 1H), 5.30 (d, J=9.2 Hz, 1H), 4.91-4.74 (m, 3H), 3.57 (s, 3H), 3.53-3.49 (m, 5H), 3.35 (m, 2H), 3.13-3.07 (m, 1H), 2.89-2.60 (br s, 5H), 1.31-1.23 (m, 3H), 0.89 (m, 2H), 0.70 (d, J=6.8 Hz, 214).

The following examples (Table G) were prepared using procedures similar to those described in the preparation of Example G1, using the appropriate building blocks (R⁵COCH═CHMe, ArSO₂Cl, R¹OH, HO₂C—CHR⁶—NHR⁷ or corresponding activated amino acid such as hydroxysuccinate ester). In some cases NHR⁷ is originally protected as Boc which necessitates an acidic Boc removal in the last step. See also the footnote to Table G describing the modified procedure to prepare the compound of Example G15.

TABLE G

| Example | | |
|---|---|---|
| No. | Structure | M + 1 |
| G2 | 2-chloro-N-{(1S,5S)-6-hydroxy-5-[{[4- | 612 |
| | (hydroxymethyl)phenyl]sulfonyl}(isobutyl)amino]- | |
| | 1-methylhexyl}-Nα-(methoxycarbonyl)-L- | |
| | phenylalaninamide | |
| | 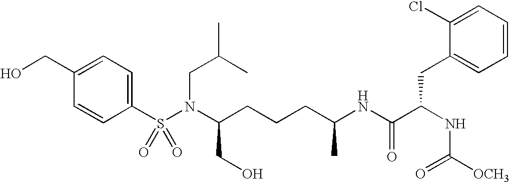 | |
| G3 | N-{(1R,5S)-5-[(1,3-benzothiazol-6-ylsulfonyl)(3- | 721 |
| | metbylbutyl)amino]-1-cyclopropyl-6- | |
| | hydroxyhexyl}-Nα-(methoxycarbonyl)-β-phenyl-L- | |
| | phenylalaninamide | |
| | 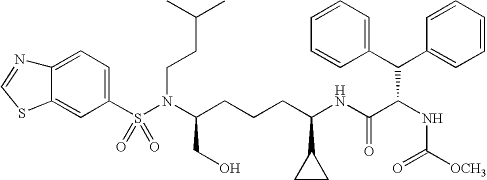 | |
| G4 | N-{(1S,5S)-5-[[(4- | 597 |
| | aminophenyl)sulfonyl](methyl)amino]-6-hydroxy-1- | |
| | methylhexyl}-Nα-(metboxycarbonyl)-β-phenyl-L- | |
| | phenylalaninamide | |
| | 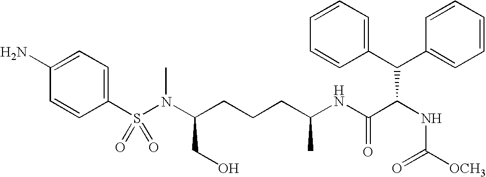 | |
| G5 | N-{(1S,5S)-5-[[(4- | 611 |
| | aminophenyl)sulfonyl](ethyl)amino]-6-hydroxy-1- | |
| | methylhexyl}-Nα-(methoxycarbonyl)-β-phenyl-L- | |
| | phenylalaninamide | |
| | 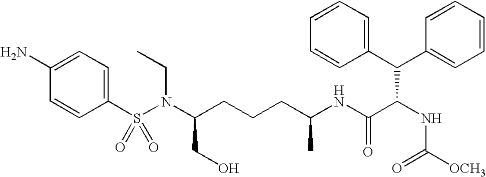 | |
| G6 | N-{(1S,5S)-6-hydroxy-5-[{[4- | 612 |
| | (hydroxymethyl)phenyl]sulfonyl}(methyl)amino]-1- | |
| | methylhexyl}-Nα-(methoxycarbonyl)-β-phenyl-L- | |
| | phenylalaninamide | |
| | 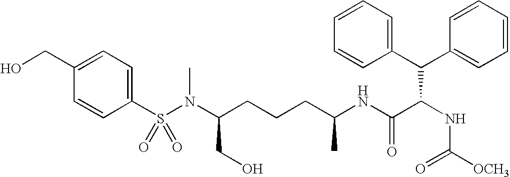 | |
| G7 | N-{(1S,5S)-5-[[(4- | 637 |
| | aminophenyl)sulfonyl](cyclopropylmethyl)amino]- | |
| | 6-hydroxy-1-methylhexyl}-Nα-(methoxycarbonyl)- | |
| | β-phenyl-L-phenylalaninamide | |
| | 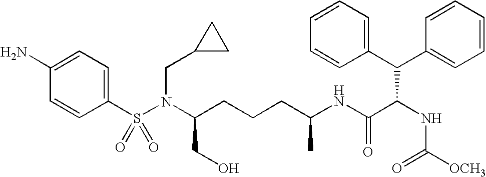 | |
| G8 | 2-chloro-N-{(1S,5S)-6-hydroxy-5-[{[4- | 570 |
| | (hydroxymethyl)phenyl]sulfonyl}(methyl)amino]-1- | |
| | methylhexyl}-Nα-(methoxycarbonyl)-L- | |
| | phenylalaninamide | |
| | 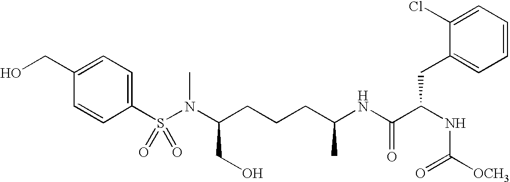 | |
| G9 | 2-chloro-N-[(1S,5S)-5-(ethyl{[4- | 584 |
| | (hydroxymethyl)phenyl]sulfonyl}amino)-6-hydroxy- | |
| | 1-methylhexyl]-Nα-(methoxycarbonyl)-L- | |
| | phenylalaninamide | |
| | 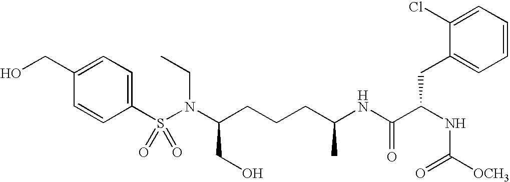 | |
| G10 | 2-chloro-N-{(1S,5S)-6-hydroxy-5-[{[4- | 598 |
| | (hydroxymethyl)phenyl]sulfonyl}(isopropyl)amino]- | |
| | 1-methylhexyl}-Nα-(methoxycarbonyl)-L- | |
| | phenylalaninamide | |
| | 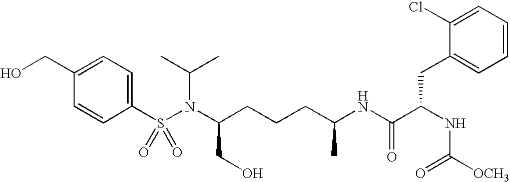 | |
| G11 | N-{(1S,5S)-5-[[(4- | 583 |
| | aminophenyl)sulfonyl](isopropyl)amino]-6-hydroxy- | |
| | 1-methylhexyl}-2-chloro-Nα-(methoxycarbonyl)-L- | |
| | phenylalaninamide | |
| | 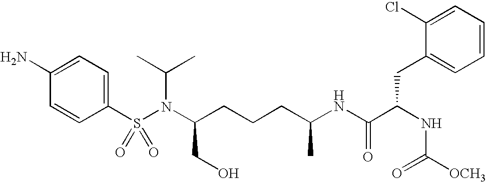 | |
| G12 | 2-bromo-N-{(1S,5S)-6-hydroxy-5-[{[4- | 656 |
| | (hydroxymethyl)phenyl]sulfonyl}(isobutyl)amino]- | |
| | 1-methylhexyl}-Nα-(methoxycarbonyl)-L- | |
| | phenylalaninamide | |
| | 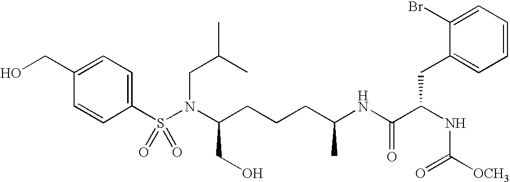 | |
| G13 | 2-bromo-N-{(1S,5S)-6-hydroxy-5-[{[4- | 642 |
| | (hydroxymethyl)phenyl]sulfonyl}(isopropyl)amino]- | |
| | 1-methylhexyl}-Nα-(methoxycarbonyl)-L- | |
| | phenylalaninamide | |
| | 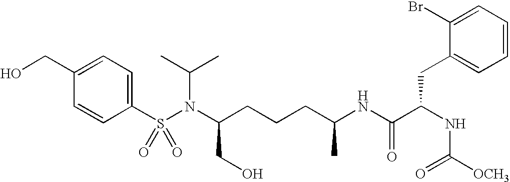 | |
| G14 | N-[(1S,5S)-5-((3-fluoropropyl){[4- | 658 |
| | (hydroxymethyl)phenyl]sulfonyl}amino)-6-hydroxy- | |
| | 1-methythexyl]-Nα-(methoxycarbonyl)-β-phenyl-L- | |
| | phenylalaninamide | |
| | 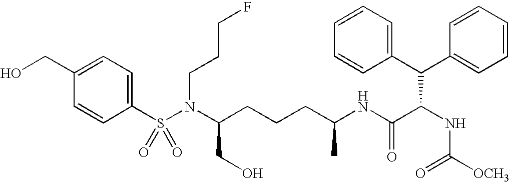 | |
| G15¹ | N-{(1S,SS)-5-[[(4- | 627 |
| | aminophenyl)sulfonyl](isopropyl)amino]-6-hydroxy- | |
| | 1-methylhexyl}-2-bromo-Nα-(methoxycarbonyl)-L- | |
| | phenylalaninamide | |
| | 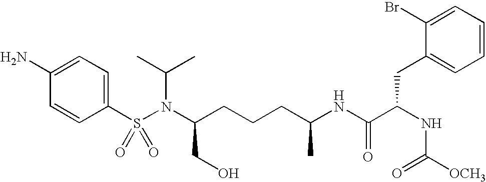 | |
| G16 | N-{(1S,5S)-6-hydroxy-5-[{[4- | 654 |
| | (hydroxymethyl)phenyl]sulfonyl}(isobutyl)amino]- | |
| | 1-methylhexyl}-Nα-(methoxycarbonyl)-β-phenyl-L- | |
| | phenylalaninamide | |
| | 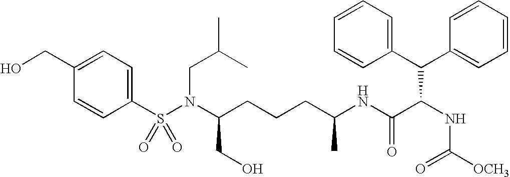 | |
| G17 | N-[(1S,5S)-5-(cyclobutyl{[4- | 652 |
| | (hydroxymethyl)phenyl]sulfonyl}amino)-6-hydroxy- | |
| | 1-methylhexyl]-Nα-(methoxycarbonyl)-β-phenyl-L- | |
| | phenylalaninamide | |
| | 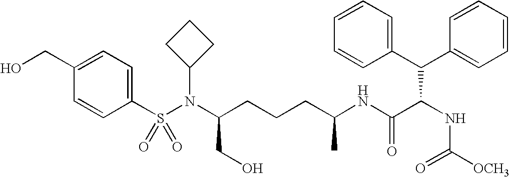 | |
| G18 | N-{(1S,5S)-5-[[(4- | 639 |
| | aminophenyl)sulfonyl](isopropyl)amino]-1- | |
| | ethyl-6-hydroxyhexyl}-Nα-(methoxycarbonyl)-β- | |
| | phenyl-L-phenylalaninamide | |
| | 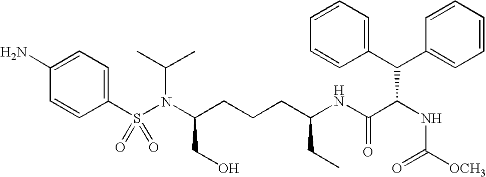 | |
| G19 | N-{(1S,5S)-1-ethyl-6-hydroxy-5-[{[4- | 654 |
| | (hydroxymethyl)phenyl]sulfonyl}(isopropyl)amino] | |
| | hexyl}-Nα-(methoxycarbonyl)-β-phenyl-L- | |
| | phenylalaninamide | |
| | 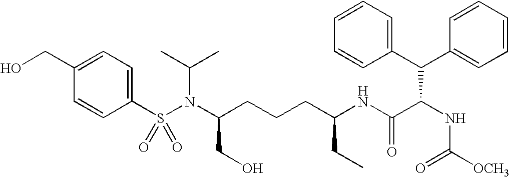 | |
| G20 | N-{(1S,5S)-6-hydroxy-5-[{[4- | 640 |
| | (hydroxymethyl)phenyl]sulfonyl}(propyl)amino]-1- | |
| | methylhexyl}-Nα-(metboxycarbonyl)-β-phenyl-L- | |
| | phenylalaninamide | |
| | 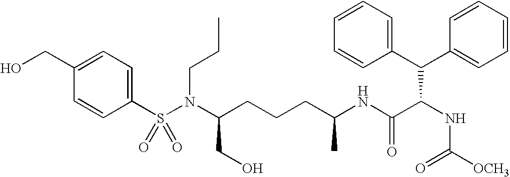 | |

¹G15 was synthesized using a variation of Scheme G: Ester reduction of ethyl(2S, 6S)-6-({(2-3-(2-bromophenyl)-2-[(methoxycarbonyl)amino]propanoyl}amino)-2-[(tert-butoxycarbonyl)amino]heptanoate (synthesized using Steps G1-1 through G1-6) with LiBH₄ (similar to Step G1-10), Boc deprotection with HCl (Step G1-7), amine sulfonylation with 4-nitrophenylsulfonyl chloride (Step G1-8), primary alcohol protection with TBS-Cl (Step K1-5), Mistunobu with i-PrOH (Step G1-9) and nitro reduction/TBS deprotection with SnCl₂ in EtOH

### Example H1

### N-[(1R,5S)-5-[[(4-aminophenyl)sulfonyl](propyl)amino]-6-hydroxy-1-(trifluoromethyl)hexyl]-Nα-(methoxycarbonyl)-β-phenyl-L-phenylalaninamide

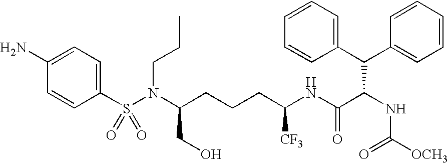

### Step H1-1 Methyl(2S,4E)-2-{[(4-nitrophenyl)sulfonyl]amino}-6-oxo-4-hexenoate

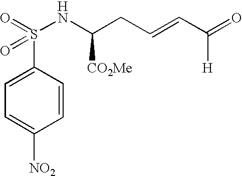

To a solution of methyl (2S)-)-2-{[(4-nitrophenyl)sulfonyl]amino}-4-pentenoate (5.00 g, 15.91 mmol) (synthesized as described in Step F1-1 and Step F1-2, with EtOH being substituted with MeOH in the first step) in 200 mL CH₂Cl₂ was added crotonaldehyde (5.27 mL, 63.6 mmol) and Grubbs 2^{nd} generation catalyst (1.35 g, 1.59 mmol). The reaction mixture was heated at 60° C. for 30 minutes, concentrated and purified by silica gel chromatography (10->70% EtOAc/hexanes) to afford the title compound.

### Step H1-2 Methyl(2S,4E,6E)-6-{[(S-tert-butylsulfinyl]amino}-2-{[(4-nitrophenyl)sulfonyl]amino}-4-hexenoate

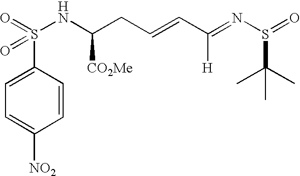

To a solution of methyl(2S,4E)-2-{[(4-nitrophenyl)sulfonyl]amino}-6-oxo-4-hexenoate in 58 mL THF at 0° C. was added S-tert-butane sulfinamide, followed by Ti(OEt)₄. The reaction mixture was allowed to warm slowly to room temperature over several hours. After 18 hours, the reaction mixture was cooled to 0° C. and diluted with EtOAc. Brine (˜10 mL) was then added and the mixture stirred vigorously at room temperature for 20 minutes. The reaction mixture was then filtered through a pad of celite, rinsing with fresh EtOAc. The filtrate was concentrated and the concentrate was purified by silica gel chromatography to obtain (30->80% EtOAc/hexanes) to obtain desired product. MS: M+H=460.

### Step H1-3 Methyl(2S,4E,6R)-6-{[(tert-butylsulfinyl]amino}-7,7,7-trifluoro-2-{[(4-nitrophenyl)sulfonyl]amino}-4-heptenoate

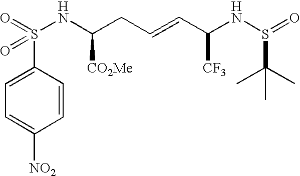

To a solution of methyl(2S,4E,6E)-6-{[(5-tert-butylsulfinyl]imino}-2-{[(4-nitrophenyl)sulfonyl]amino}-4-hexenoate (1.62 g, 3.59 mmol) in 36 mL THF at 0° C. was added TMS-CF₃ (1.28 g, 8.98 mmol), followed by TMAF (0.87 mL, 8.98 mmol). After 1.5 hours of stirring at 0° C., the reaction mixture was quenched by the addition of saturated aqueous NH₄Cl and diluted with EtOAc and H₂O. The layers were then separated and the organics were washed with brine, dried over Na₂SO₄, filtered and concentrated to obtain the title product which was subsequently used in Step H1-4 without further purification. MS: M+H=516.

### Step H1-4 Methyl(2S,4E,6R)-6-amino-7,7,7-trifluoro-2-{[(4-nitrophenyl)sulfonyl]amino}-4-heptenoate hydrochloride

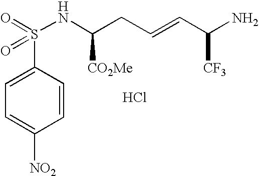

To a solution of unpurified methyl(2S,4E,6R)-6-{[(tert-butylsulfinyl]amino}-7,7,7-trifluoro-2-([(4-nitrophenyl)sulfonyl]amino}-4-heptenoate (1.80 g, 3.49 mmol) from Step H1-3 in 29 mL MeOH was added 4M HCl in dioxane (7.0 mL, 27.9 mmol). After 2 hours of stirring at room temperature, the reaction mixture was concentrated and used without further purification in Step H1-5. MS: M+H=412.

### Step H1-5 Methyl(2S,4E,6R)-)-7,7,7-trifluoro-6-({(2S)-2-[(methoxycarbonyl)amino]-3,3-diphenylpropanoyl}amino)-2-[(4-nitrophenyl)sulfonyl]amino}heptenoate

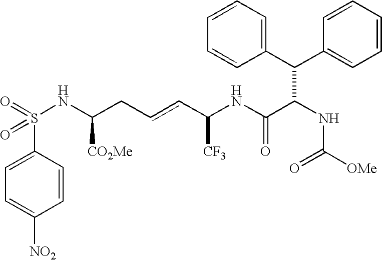

To a solution of unpurified methyl (2S,4E,6R)-6-amino-7,7,7-trifluoro-2-{[(4-nitrophenyl)sulfonyl]amino}-4-heptenoate hydrochloride (1.40 g, 3.40 mmol) from Step H1-4 and 2S-2-[(methoxycarbonyl)amino]-3,3-diphenylpropanoic acid (1.12 g, 3.74 mmol) in 38 mL DMF was added diisopropylethylamine (1.50 mL, 8.51 mmol) and PyBrOP (2.06 g, 4.42 mmol). The reaction mixture was allowed to proceed at room temperature with stirring for 16 hours, and was then quenched by the addition of saturated. aqueous NaHCO₃. The quenched reaction mixture was then diluted with EtOAc, the resulting layers were separated, and the organics were washed with 3M LiCl (3×) and brine, dried over Na₂SO₄, filtered and concentrated. The concentrate was purified by silica gel chromatography (20->80% EtOAc/hexanes) to obtain desired product. MS: M+H=693.

### Step H1-6 Methyl(2S,4E,6R)-7,7,7-trifluoro-6-({(2S)-2-[(methoxycarbonyl)amino]-3,3-diphenylpropanoyl}amino)-2-[[(4-nitrophenyl)sulfonyl](propyl)amino]heptenoate

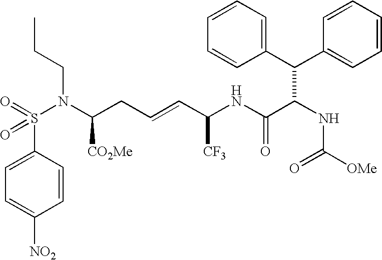

To a solution of methyl(2S,4E,6R)-7,7,7-trifluoro-6-({(2S)-2-[(methoxycarbonyl)amino]-3,3-diphenylpropanoyl}amino)-2-{[(4-nitrophenyl)sulfonyl]amino}heptenoate (0.240 g, 0.340 mmol) in 4 mL THF was added n-propanol (0.130 mL, 1.70 mmol), Ph₃P (267 mg, 1.02 mmol) and DIAD (0.200 mL, 1.02 mmol). After 16 hours, the reaction was concentrated and purified by silica gel chromatography (20->55% EtOAc/hexanes) to afford the desired product (0.250 g) as a white foam. MS: M+H=735.

### Step H1-7 Methyl(2S,6R)-)-7,7,7-trifluoro-6-({(2S)-2-[(methoxycarbonyl)amino]-3,3-diphenylpropanoyl}amino)-2-[[(4-aminophenyl) sulfonyl](propyl)amino]heptanoate

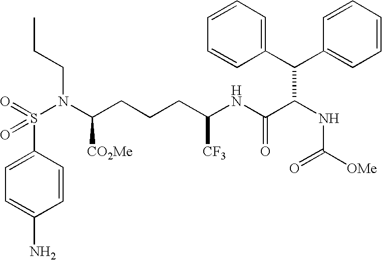

To a solution of methyl(2S,4E,6R)-)-7,7,7-trifluoro-6-({(2S)-2-[(methoxycarbonyl)amino]-3,3-diphenylpropanoyl}amino)-2-[[(4-nitrophenyl)sulfonyl](propyl)amino]heptenoate (0.250 g, 0.340 mmol) in 3 mL EtOH was added Pd(OH)₂ (20% on carbon, 71.7 mg, 0.102 mmol). A H2 balloon was attached, and the flask was evacuated/backfilled with H2 (3×). After 3 hours of stirring at room temperature, the flask was evacuated/backfilled with N₂, and the reaction mixture was filtered through a pad of celite under N₂, rinsing with CH₂Cl₂. The organics were concentrated to provide the desired product. MS: M+H=707.

### Step H1-8 Methyl[(1S)-2-{[(1R,5S)-5-[[(4-aminophenyl)sulfonyl](propyl)amino]-6-hydroxy-1-(trifluoromethyl)hexyl]amino]-1-(diphenylmethyl)-2-oxoethyl]carbamate

To a solution of methyl(2S,6R)-)-7,7,7-trifluoro-6-({(2S)-2-[(methoxycarbonyl)amino]-3,3-diphenylpropanoyl}amino)-2-[[(4-aminophenyl)sulfonyl](propyl)amino]heptanoate (0.240 g, 0.340 mmol) in 3.5 mL THF was added 2M LiBH₄ (0.680 mL, 1.36 mmol). After 16 hours of stirring at room temperature, the reaction mixture was cooled to 0° C. and quenched by the addition of EtOAc, MeOH and saturated aqueous NH₄Cl in that order. The quenched mixture was then diluted with more EtOAc, and the organics were washed with H₂O and brine, dried over Na₂SO₄, filtered and concentrated. The concentrate was purified by preparative HPLC (5->95% CH3CN/H2O). MS: M+H=679. ¹H NMR (400 MHz, MeOD) δ 8.48 (d, J=9.4 Hz, 1H), 7.54 (d, J=9.8 Hz, 2H), 7.37-7.26 (m, 4H), 7.24-7.14 (m, 6H), 6.76 (d, J=9.8 Hz, 2H), 5.08 (d, J=11.7 Hz, 1H), 4.34 (d, J=11.7 Hz, 1H), 4.12-4.11 (m, 1H), 3.52 (s, 3H), 3.49-3.31 (m, 4H), 3.09-2.96 (m, 2H), 1.63-1.43 (m, 3H), 1.33-1.27 (m, 1H), 1.18-1.09 (m, 2H), 0.97 (t, J=7.5 Hz, 3H), 0.58 (m, 2H).

The following examples (Table H) were prepared using similar procedures as described in the preparation of Example H1, using the appropriate building blocks (R¹OH, HO₂C—CHR⁶—NHR⁷ or corresponding activated amino acid such as hydroxysuccinate ester). In some cases NHR⁷ is originally protected as Boc which necessitates an acidic Boc removal in the last step.

TABLE H

| Example | | |
|---|---|---|
| No. | Structure | M + 1 |
| H2 | N-[(1R,5S)-5-[[(4-aminophenyl)sulfonyl](3- | 707 |
| | methylbutyl)amino]-6-hydroxy-1- | |
| | (trifluoromethyl)hexyl]-Nα-(methoxycarbonyl)-β- | |
| | phenyl-L-phenylalaninamide | |
| | 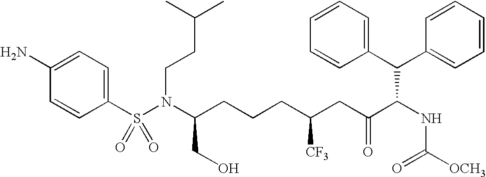 | |
| H3 | N-[(1R,55)-5-[[(4- | 679 |
| | aminophenyl)sulfonyl](isopropyl)amino]-6-hydroxy-1- | |
| | (trifluoromethyl)hexyl]-Nα-(methoxycarbonyl)-β- | |
| | phenyl-L-phenylalaninamide | |
| | 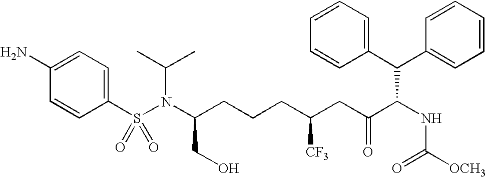 | |
| H4 | N-[(1R,5S)-5-[[(4- | 665 |
| | aminophenyl)sulfonyl](ethyl)amino]-6-hydroxy-1- | |
| | (trifluoromethyl)hexyl]-Nα-(methoxycarbonyl)-β- | |
| | phenyl-L-phenylalaninamide | |
| | 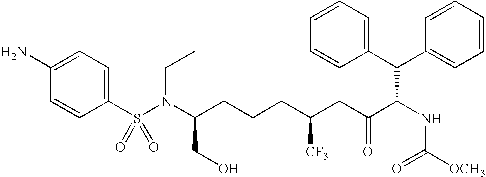 | |
| H5 | N-[(1R,5S)-5-[[(4- | 693 |
| | aminophenyl)sulfonyl](isobutyl)amino]-6-hydroxy-1- | |
| | (trifluoromethyl)hexyl]-Nα-(methoxycarbonyl)-β- | |
| | phenyl-L-phenylalaninamide | |
| | 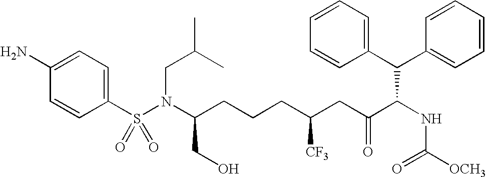 | |
| H6 | N-[(1R,5S)-5-[[(4- | 651 |
| | aminophenyl)sulfonyl](methyl)amino]-6-hydroxy-1- | |
| | (trifluoromethyl)hexyl]-Nα-(methoxycarbonyl)-β- | |
| | phenyl-L-phenylalaninamide | |
| | 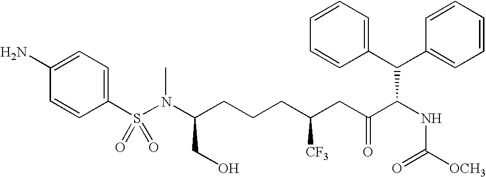 | |
| H7¹ | N-{(1S,5S)-5-[[(4- | 581 |
| | aminophenyl)sulfonyl](isopropyl)amino]-6-hydroxy-1- | |
| | methylhexyl}-Nα-methyl-β-phenyl-L- | |
| | phenylalaninamide hydrochloride | |
| | 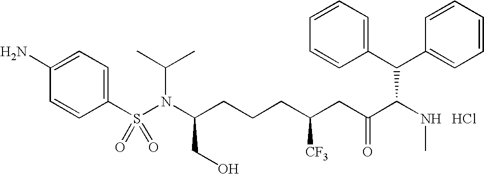 | |

¹Boc removal as last step in the preparation of the compound.

### Example I1

### 2-chloro-N-{(1R,5S)-1-cyclopropyl-6-hydroxy-5-[{[4-(hydroxymethyl)phenyl]sulfonyl}-(isobutyl)amino]hexyl}-Nα-(methoxycarbonyl)-L-phenylalaninamide

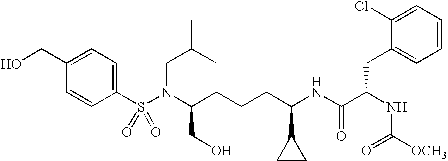

### Step I-1 Ethyl (2S)-6-oxo-2-piperidinecarboxylate

To a solution of 6-oxo-L-picolinic acid (5.00 g, 34.9 mmol) in 120 mL EtOH added thionyl chloride (25.5 mL, 349 mmol) in 1 mL aliquots over 30 minutes. After 16 hours of stirring at room temperature, the reaction mixture was concentrated to obtain the desired product containing unidentified impurities as a yellow oil. MS: M+H=172. The product was used in Step I-2 without further purification.

### Step I-2 1-tert-butyl 2-ethyl-(2S)-6-oxo-2-piperidinecarboxylate

To a solution of ethyl (2S)-6-oxo-2-piperidinecarboxylate (3.79 g, 22.1 mmol) in 15.8 mL CH₃CN was added Boc₂O (9.66 g, 44.3 mmol) and DMAP (5.41 g, 44.3 mmol). After 6 hours of stirring at room temperature, the reaction mixture was concentrated and purified by silica gel chromatography (0->70% EtOAc/hexanes) to afford the desired product. MS: M+H=272.

### Step I-3 Ethyl (2S)-2-[(tert-butoxycarbonyl)amino]-6-cyclopropyl-6-oxohexanoate

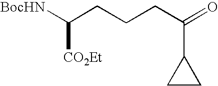

To a solution of 1-tert-butyl 2-ethyl-(2S)-6-oxo-2-piperidinecarboxylate (0.460 g, 1.69 mmol) in 2.83 mL THF at −78° C. was added cyclopropylmagnesium bromide in THF (0.5M, 4.07 mL, 2.03 mmol). The reaction mixture was allowed to warm to −30° C. over 3 hours, and was then quenched by the addition of saturated aqueous NH₄Cl and EtOAc. The layers were separated, and the organics were washed with brine, dried over Na₂SO₄, filtered and concentrated. The concentrate was purified by silica gel chromatography (40->100% EtOAc/hexanes) to afford the desired product. MS: M+H=314.

The ketone obtained from Step I-3 was elaborated to the final compound 2-chloro-N-{(1R,5S)-1-cyclopropyl-6-hydroxy-5-[{[4-(hydroxymethyl)phenyl]sulfonyl](isobutyl)amino]hexyl}-Nα-(methoxycarbonyl)-L-phenylalaninamide via (R)-tert-butane sulfinyl imine formation (as in Step F1-6), diastereoselective imine reduction (as in Step F1-7), auxiliary deprotection (as in Step F1-8), coupling with (2S)-3-(2-chlorophenyl)-2-[(methoxycarbonyl)amino]propanoic acid (as in Step B1-11 with EDC and HOAt being used in place of BOP reagent), Boc removal (as in Step B1-10), sulfonylation with 4-carbomethoxysulfonyl chloride (as in Step F1-2), Mitsunobu reaction as in (Step F1-3 with isobutanol being used in place if isopronanol) and diester reduction (as in Step F1-9 with 7 equiv. LiBH₄). MS: M+H=638, 639 (Cl pattern). ¹H NMR (400 MHz, CDCl₃) δ 7.72 (d, J=7.8 Hz, 2H), 7.49-7.44 (m, 3H), 7.28-7.16 (m, 3H), 7.23 (s, 1H), 5.34 (d, J=8.2 Hz, 1H), 4.80 (dd, J=8.7, 4.0 Hz, 1H), 4.69 (d, J=12.6 Hz, 1H), 4.40 (d, J=7.4 Hz, 1H), 4.28 (s, 1H), 3.65 (s, 3H), 3.61-3.49 (m, 2H), 3.38 (s, 1H), 3.16-3.10 (m, 3H), 2.77-2.73 (m, 2H), 2.60 (m, 1H), 1.884 (m, 1H), 1.16 (m, 2H), 0.99 (d, J=6.5 Hz, 3H), 0.92 (d, J=6.6 Hz, 3H), 0.99-0.91 (m, 2H), 0.39-0.30 (m, 4H), 0.14 (m, 1H), 0.03 (m, 2H).

The following examples (Table I) were prepared using similar procedures as described in the preparation of Example 11, using the appropriate building blocks (R⁵MgX, ArSO₂Cl, R¹OH, HO₂C—CHR⁶—NHR⁷ or corresponding activated amino acid such as hydroxysuccinate ester). In some cases NHR⁷ wax originally protected as Boc which necessitated an acidic Boc removal in the last step.

TABLE I

| Example | | |
|---|---|---|
| No. | Structure | M + 1 |
| I2 | 2-chloro-N-{(1R,5S)-1-cyclopropyl-6-hydroxy-5- | 624 |
| | [{[4- | |
| | (hydroxymethyl)phenyl]sulfonyl}(isopropyl)amino] | |
| | hexyl}-Nα-(methoxycarbonyl)-L-phenylalaninamide | |
| | 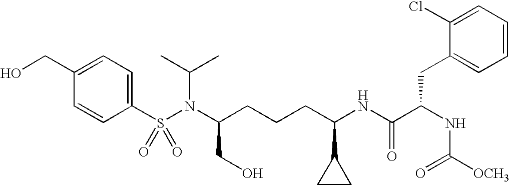 | |
| I3 | N-{(1R,5S)-1-cyclopropyl-6-hydroxy-5-[{[4- | 666 |
| | (hydroxymethyl)phenyl]sulfonyl}(isopropyl)amino] | |
| | hexyl }-Nα-(methoxycarbonyl)-β-phenyl-L- | |
| | phenylalaninamide | |
| | 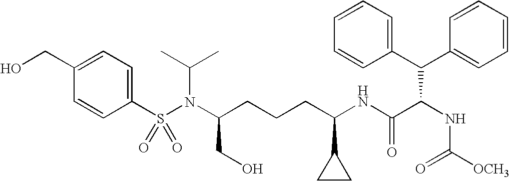 | |
| I4 | N-{(1R,5S)-1-cyclopropyl-6-hydroxy-5-[{[4- | 666 |
| | (hydroxymethyl)phenyl]sulfonyl}(propyl)amino]hexyl}- | |
| | Nα-(methoxycarbonyl)-β-phenyl-L- | |
| | phenylalaninamide | |
| | 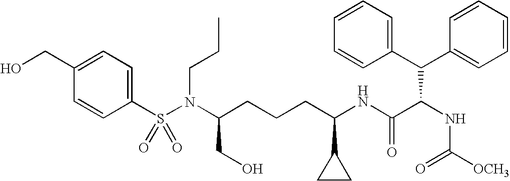 | |
| I5 | N-[(1R,5S)-1-cyclopropyl-5-(ethyl{[4- | 652 |
| | (hydroxymethyl)phenyl]sulfonyl}amino)-6- | |
| | hydroxyhexyl]-Nα-(methoxycarbonyl)-β-phenyl-L- | |
| | phenylalaninamide | |
| | 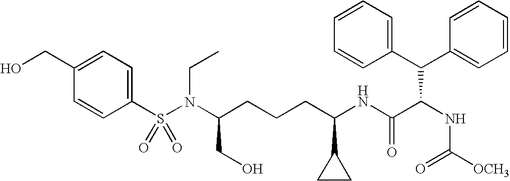 | |
| I6 | N-{(1R,5S)-1-cyclopropyl-6-hydroxy-5-[{[4- | 638 |
| | (hydroxymethyl)phenyl]sulfonyl}(methyl)amino]hexyl}- | |
| | Nα-(methoxycarbonyl)-β-phenyl-L- | |
| | phenylalaninamide | |
| | 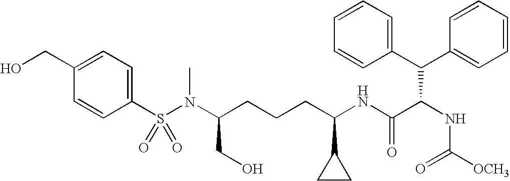 | |
| I7¹ | N-{(1R,5S)-5-[[(4- | 651 |
| | aminophenyl)sulfonyl](propyl)amino]-1- | |
| | cyclopropyl-6-hydroxyhexyl}-Nα- | |
| | (methoxycarbonyl)-β-phenyl-L-phenylalaninamide | |
| | 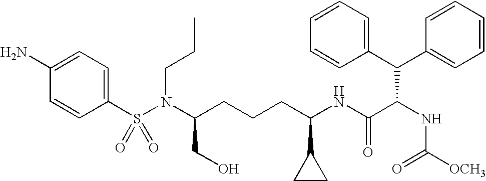 | |
| I8¹ | N-{(1R,5S)-5-[[(4- | 651 |
| | aminophenyl)sulfonyl](isopropyl)amino]-1- | |
| | cyclopropyl-6-hydroxyhexyl}-Nα- | |
| | (methoxycarbonyl)-β-phenyl-L-phenylalaninamide | |
| | 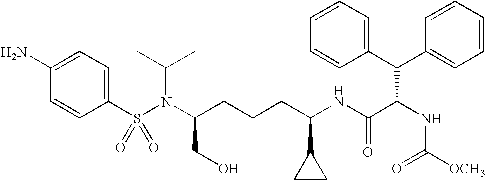 | |
| I9¹ | N-{(1R,5S)-5-[[(4- | 623 |
| | aminophenyl)sulfonyl](methyl)amino]-1- | |
| | cyclopropyl-6-hydroxyhexyl}-Nα- | |
| | (methoxycarbonyl)-β-phenyl-L- | |
| | phenylalaninamide | |
| | 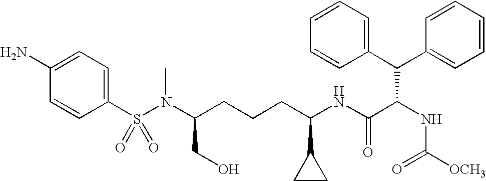 | |
| I10¹ | N-{(1R,5S)-5-[[(4- | 637 |
| | aminophenyl)sulfonyl](ethyl)amino]-1-cyclopropyl- | |
| | 6-hydroxyhexyl}-Nα-(methoxycarbonyl)-β-phenyl- | |
| | L-phenylalaninamide | |
| | 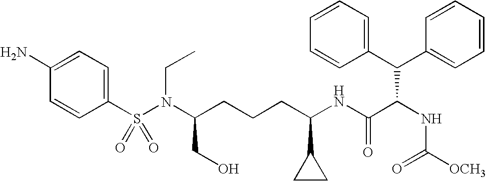 | |

¹Nitro reduction was conducted before ester reduction.

### Example J1

### N-{(1S,5S)-1-ethyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide

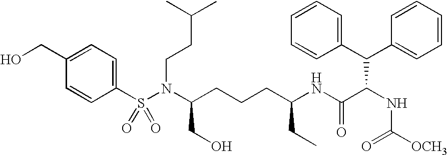

### Step J1-1: methyl 6-(benzyloxy)-L-norleucinate hydrochloride

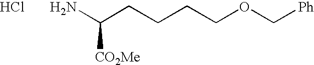

Through a suspension of methyl 6-(benzyloxy)-L-norleucine (AdvancedChemTech YL2375, 10.92 g, 46 mmol) in MeOH (460 mL) was bubbled HCl (g) for 10 minutes. The reaction mixture was then heated at 50° C. for 4 hours, then allowed to cool to room temperature. Nitrogen gas was then bubbled through the mixture for 10 minutes, after which the mixture was concentrate in vacuo, reconcentrated from DCM three times to give the desired product as a white solid. MS M+1=252.

### Step J1-2: methyl 6-(benzyloxy)-N-{[4-(methoxycarbonyl)phenyl]sulfonyl}-L-norleucinate

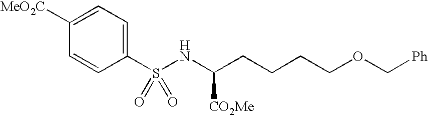

To a solution of methyl 6-(benzyloxy)-L-norleucinate hydrochloride (15 g, 52.1 mmol) in DCM (261 mL) was added triethylamine (15.98 mL, 115 mmol) followed by 4-carbomethoxy-phenylsulfonyl chloride (12.48 g, 53.2 mmol) by portions. After 15 minutes of stirring at room temperature, the reaction mixture was concentrated in vacuo to ⅓ volume, diluted with EtOAc, washed with 10% KHSO₄, saturated aqueous NaHCO₃, and then brine, and then dried over sodium sulfate and concentrated in vacuo to give the desired product as a crude solid. MS M+1=450.

### Step J1-3: methyl 6-(benzyloxy)-N-{[4-(methoxycarbonyl)phenyl]sulfonyl}-N-(3-methylbutyl)-L-norleucinate

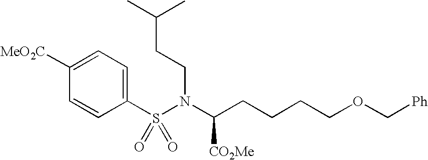

To a solution of methyl 6-(benzyloxy)-N-{[4-(methoxycarbonyl)phenyl]-sulfonyl}-L-norleucinate (3 g, 6.67 mmol), triphenylphosphine (2.63 g, 10.01 mmol) and 3-methyl-1-butanol (3.64 mL, 33.4 mmol) in THF (66.7 mL) was added DEAD (1.585 mL, 10.01 mmol) dropwise. The reaction mixture was stirred at room temperature overnight, concentrated in vacuo and purified by flash chromatography (300 g silica, 10 to 40% EtOAc in hexane) to give of the desired product as a clear oil. MS M+Na=542.

### Step J1-4: N-[(1S)-5-(benzyloxy)-1-(hydroxymethyl)pentyl]-4-(hydroxymethyl)-N-(3-methylbutyl)benzenesulfonamide

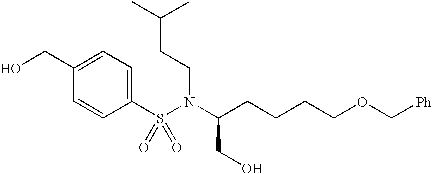

To a solution of methyl 6-(benzyloxy)-N-{[4-(methoxycarbonyl)phenyl]-sulfonyl}-N-(3-methylbutyl)-L-norleucinate (3 g, 5.77 mmol) in THF (38.5 mL), cooled to 0° C. was added LAH (1M in Et₂O, 11.55 mL, 11.55 mmol) and the reaction mixture was stirred at 0° C. for 20 minutes. Water (456 μL) was then added dropwise, followed by the dropwise addition of 456 μL 15% NaOH and then 1368 μL water. After 5 minutes of vigorous stirring at room temperature, the reaction mixture was filtered on cellite and concentrated in vacuo to give the desired product. MS M+1=464.

### Step J1-5: N-[(1S)-5-(benzyloxy)-1-({[tert-butyl(diphenyl)silyl]oxy}methyl)pentyl]-4-({[tert-butyl(diphenyl)silyl]oxy}methyl)-N-(3-methylbutyl)benzenesulfonamide

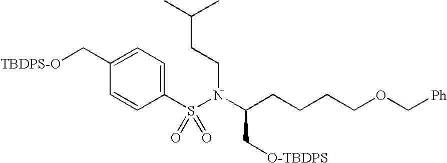

To a solution of N-[(1S)-5-(benzyloxy)-1-(hydroxymethyl)pentyl]-4-(hydroxymethyl)-N-(3-methylbutyl)benzenesulfonamide (2.68 g, 5.78 mmol), imidazole (866 mg, 12.72 mmol) and DMAP (71 mg, 0.578 mmol) in DCM (58 mL) was added TBDPSCl (3.04 mL, 11.85 mmol). The reaction mixture was stirred at room temperature overnight, concentrated in vacuo to ⅓ volume, diluted with Et₂O, washed with 10% KHSO₄, saturated aqueous NaHCO₃, and brine, then dried over sodium sulfate, concentrated in vacuo and purified by flash chromatography (300 g silica, o to 30% EtOAc in hexane) to give the desired product.

### Step J1-6: 4-({[tert-butyl(diphenyl)silyl]oxy}methyl)-N-[(1S)-1-({[tert-butyl(diphenyl)silyl]oxy}methyl)-5-hydroxypentyl]-N-(3-methylbutyl)benzenesulfonamide

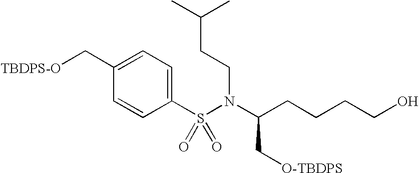

A solution of N-[(1S)-5-(benzyloxy)-1-({[tert-butyl(diphenyl)silyl]oxy}methyl)-pentyl]-4-({[tert-butyl(diphenyl)silyl]oxy}methyl)-N-(3-methylbutyl)benzenesulfonamide (5.44 g, 5.78 mmol) in EtOH (116 mL) was vacuum purged with argon, 10% Pd/C was added (3.08 g) very carefully under an argon flow. The reaction mixture was hydrogenated under 1 atm H2, at room temperature for 16 hours. The reaction mixture was vacuum purged with argon, 10% Pd/C was added (5 g) very carefully under an argon flow and the reaction mixture resubmitted to 1 atm H2, at room temperature for 4 days. The reaction mixture was then filtered carefully under N2 flow, rinsed with EtOH, and concentrated in vacuo to give the desired product.

### Step J1-7: 4-({[tert-butyl(diphenyl)silyl]oxy}methyl)-N-[(1S)-1-({[tert-butyl(diphenyl)silyl]oxy}methyl)-5-oxopentyl]-N-(3-methylbutyl)benzenesulfonamide

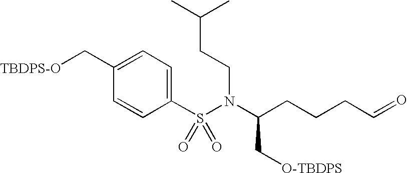

To a solution of 4-({[tert-butyl(diphenyl)silyl]oxy}methyl)-N-[(1S)-1-({[tert-butyl(diphenyl)silyl]oxy}methyl)-5-hydroxypentyl]-N-(3-methylbutyl)benzenesulfonamide (4 g, 4.7 mmol) and NMO (661 mg, 5.65 mmol) in DCM (47 mL) was added 3 g 4A sieves, activated, and the reaction mixture was stirred at room temperature for 5 minutes. TPAP (165 mg, 0.47 mmol) was then added by portions, and the reaction mixture was stirred at room temperature for 45 minutes, filtered on a plug of silica gel, eluting with 25% EtOAc in hexane, to give after concentration the desired product.

### Step J1-8: (6S,10E)-6-[{[4-({[tert-butyl(diphenyl)silyl]oxy}methyl)phenyl]sulfonyl}(3-methylbutyl)amino]-2,2,13,13-tetramethyl-3,3-diphenyl-4-oxa-12-thionia-11-aza-3-silatetradec-10-en-12-olate

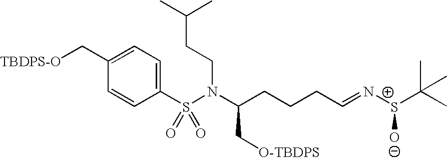

4-({[tert-butyl(diphenyl)silyl]oxy}methyl)-N-[(1S)-1-({[tert-butyl(diphenyl)silyl]oxy}methyl)-5-oxopentyl]-N-(3-methylbutyl)benzenesulfonamide (2.0 g, 2.7 mmol) was dissolved in methylene chloride (25 mL) under a nitrogen atmosphere. Magnesium sulfate (2.8 g, 23.6 mmol), (R)-(+)-tert-butanesulfinamide (430 mg, 3.5 mmol), pyridinium p-toluene-sulfonate (30 mg, 0.11 mmol) were all added portionwise as solids to the stirring solution. The reaction was stirred for 36 hours at room temperature, and was then filtered over a celite pad and concentrated in vacuo. The resulting crude oil was purified using silica gel chromatography (300 g, using 0-40% ethyl acetate in hexane gradient) to yield the desired product.

### Step J1-9: (6S,10S)-6-[{[4-({[tert-butyl(diphenyl)silyl]oxy}methyl)phenyl]sulfonyl}(3-methylbutyl)amino]-10-ethyl-2,2,13,13-tetramethyl-3,3-diphenyl-4-oxa-12-thionia-11-aza-3-silatetradecan-12-olate

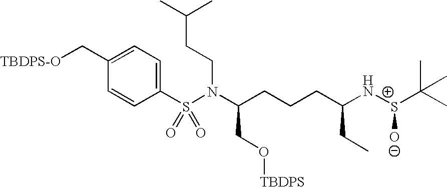

6S,10E)-6-[{[4-({[tert-butyl(diphenyl)silyl]oxy}methyl)phenyl]sulfonyl}(3-methylbutyl)amino]-2,2,13,13-tetramethyl-3,3-diphenyl-4-oxa-12-thionia-11-aza-3-silatetradec-10-en-12-olate (1.6 g, 1.6 mmol) was dissolved in anhydrous methylene chloride (16 mL) and cooled to 0° C. under nitrogen atmosphere. Ethyl magnesium bromide (0.82 mL, 2.4 mmol, 3 M solution) was added dropwise to the stirring solution. The reaction mixture was stirred at 0° C. for 3 hours, and then quenched with saturated ammonium chloride solution. The desired product was extracted from the biphasic system with methylene chloride, and the organics were combined, dried over sodium sulfate and concentrated in vacuo. The crude oil was purified using silica gel chromatography (300 g, using a 15-70% ethyl acetate in hexane gradient) to afford the desired isomer as a clear oil. The desired isomer was the second isomer to elute via normal phase chromatography.

### Step J1-10: N-[(1S,5S)-5-amino-1-(hydroxymethyl)heptyl]-4-(hydroxymethyl)-N-(3-methylbutyl)benzenesulfonamide

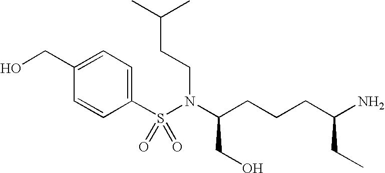

(6S,108)-6-[{[4-([tert-butyl(diphenyl)silyl]oxy}methyl)phenyl]sulfonyl}(3-methylbutyl)amino]-10-ethyl-2,2,13,13-tetramethyl-3,3-diphenyl-4-oxa-12-thionia-11-aza-3-silatetradecan-12-olate (1.5 g, 1.5 mmol) was dissolved in methanol (15 mL) and hydrochloric acid (3.7 mL, 15 mmol, 4 M solution) was added dropwise to the solution. The reaction was stirred for 6 hours at room temperature. The solution was concentrated in vacuo and the resulting crude oil was purified using SCX using methanol followed by 2 M ammonia in methanol solution to elute the desired compound. LCMS (M+1)=401.

### Step J 1-11: N-{(1S,5S)-1-ethyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide

N-[(1S,5S)-5-amino-1-(hydroxymethyl)heptyl]-4-(hydroxymethyl)-N-(3-methylbutyl)benzenesulfonamide (450 mg, 1.1 mmol), N-(methoxycarbonyl)-β-phenyl-L-phenylalanine (335 mg, 1.1 mmol), EDC (237 mg, 1.2 mmol), and HOAt (43 mg, 0.3 mmol) were dissolved in DMF (11 mL) under nitrogen atmosphere and allowed to stir at room temperature for 16 hours. The solution was diluted with ethyl acetate, washed with 10% potassium monohydrogen sulfate, saturated sodium bicarbonate, lithium chloride, dried over sodium sulfate and concentrated in vacuo. The crude oil was purified using silica gel chromatography (100 g, using a 70-100% ethyl acetate in hexane gradient) to afford the desired product as a clear oil. ¹H NMR (CDCl₃): δ 7.8 (d, J=8.1 Hz, 2H), 7.55 (d, J=8.1 Hz, 2H), 7.4-7.2 (m, 10H), 6.1 (br s, 1H), 5.2 (m, 1H), 4.95 (d, J=12 Hz, 1H), 4.85 (m, 1H), 4.75 (d, J=12 Hz, 1H), 4.65 (br s, 1H), 4.4 (m, 1H), 3.6 (s, 3H), 3.75 (s, 2H), 3.4 (s, 2H), 3.3 (m, 1H), 3.0 (m, 1H), 2.6 (br s, 1H), 1.8-1.6 (m, 2H), 1.55 (q, J=7.3 Hz, 2H), 1.1-0.8 (m, 13H), 0.7 (t, J=7.3 Hz, 31-1). LCMS (M+1)=682.

### Example J2

### N-[(1R,55S)-6-hydroxy-5-[{[4-(hydroxymethyl)phenyl]sulfonyl}(3-methylbutyl)amino]-1-(trifluoromethyl)hexyl]-Nα-(methoxycarbonyl)-β-phenyl-L-phenylalaninamide

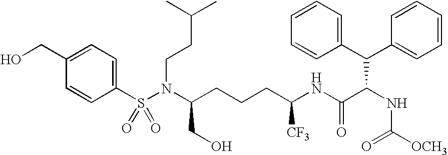

### Step J2-1: 4-({[tert-butyl(diphenyl)silyl]oxy}methyl)-N-((1S,5E)-1-({[tert-butyl(diphenyl)silyl]oxy}methyl)-5-[(S)-tert-butyl sulfinyl]imino}pentyl)-N-(3-methylbutyl)benzenesulfonamide

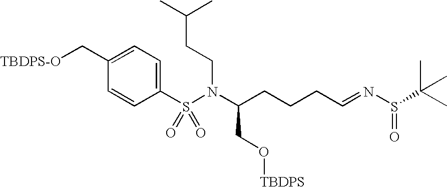

To a solution of 4-({[tert-butyl(diphenyl)silyl]oxy}methyl)-N-[(1S)-1-({[tert-butyl(diphenyl)silyl]oxy}methyl)-5-oxopentyl]-N-(3-methylbutyl)benzenesulfonamide (Step J1-7, 240 mg, 0.283 mmol), S-2-methylpropane-2-sulfinamide (S-Ellman sulfonamide, 38 mg, 0.311 mmol) and PPTS (7 mg, 0.028 mmol) was added magnesium sulfate (340 mg, 2.83 mmol). The reaction mixture was stirred at room temperature for 16 hours and purified by flash chromatography (silica, 40 g, 0 to 40% EtOAc in hexane) to give the desired product.

### Step J2-2: N-[(1S,5R)-5-{[(S)-tert-butylsulfinyl]amino}-6,6,6-trifluoro-1-(hydroxymethyl)hexyl]-4-(hydroxymethyl)-N-(3-methylbutyl)benzenesulfonamide

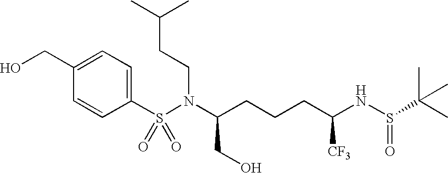

To a solution of 4-({[tert-butyl(diphenyl)silyl]oxy}methyl)-N-((1S,5E)-1-({[tert-butyl(diphenyl)silyl]oxy}methyl)-5-[(S)-tert-butyl sulfinyl]imino}pentyl)-N-(3-methylbutyl)benzenesulfonamide (60 mg, 0.063 mmol) in THF (1.2 mL) was added trifluoromethyltrimethylsilane (30 pt, 0.198 mmol) and tetramethylammonium fluoride (35 mg, 0.378 mmol). The reaction mixture was stirred at room temperature for 3 days and purified by preparative HPLC to give the desired product. MS: M+1=545.

### Step J2-3: N-[(1S,5R)-5-amino-6,6,6-trifluoro-1-(hydroxymethyl)hexyl]-4-(hydroxymethyl)-N-(3-methylbutyl)benzenesulfonamide hydrochloride

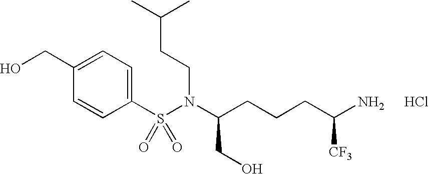

To a solution of N-[(1S,5R)-5-{[(S)-tert-butylsulfinyl]amino}-6,6,6-trifluoro-1-(hydroxymethyl)hexyl]-4-(hydroxymethyl)-N-(3-methylbutyl)benzenesulfonamide (12 mg, 0.022 mmol) in MeOH (440 μL) was added 4N HCL (4 mL) in dioxane. The reaction mixture was stirred at room temperature for 90 minutes and concentrated in vacuo to give the desired product. MS: M+1=441.

### Step J2-4: N-[(1R,5S)-6-hydroxy-5-[{[4-(hydroxymethyl)phenyl]sulfonyl}(3-methylbutyl)amino]-1-(trifluoromethyl)hexyl]-Nα-(methoxycarbonyl)-β-phenyl-L-phenylalaninamide

To a solution of N-[(1S,5R)-5-amino-6,6,6-trifluoro-1-(hydroxymethyl)hexyl]-4-(hydroxymethyl)-N-(3-methylbutyl)benzenesulfonamide hydrochloride (10 mg, 0.023 mmol) in DMF (450 μL) was added N-(methoxycarbonyl)-β-phenyl-L-phenylalanine (6.8 mg, 0.023 mmol), Hunig's base (8 μL, 0.045 mmol) and PyBrOP (12.7 mg, 0.027 mmol). The reaction mixture was stirred at room temperature overnight and purified by preparative HPLC to give the desired product N-[(1R,5S)-6-hydroxy-5-[{[4-(hydroxymethyl)phenyl]sulfonyl}(3-methylbutyl)amino]-1-(trifluoromethyl)hexyl]-Nα-(methoxycarbonyl)-β-phenyl-L-phenylalaninamide after EtOAc extraction of NaHCO₃ basified fractions. MS: M+1=722. ¹H NMR (400 MHz, d₄-MeOH) δ 7.81 (d, J=8.4 Hz, 2H), 7.53 (d, J=8.4 Hz, 2H), 7.38-7.34 (m, 4H), 7.30-7.10 (m, 6H), 5.06 (d, J=11.8 Hz, 1H), 4.70 (s, 2H), 4.33 (d, J=11.8 Hz, 1H), 4.10-4.00 (m, 1H), 3.65-3.56 (m, 2H), 3.52 (s, 3H), 3.43-3.36 (m, 2H), 3.25-3.15 (m, 1H), 3.15-3.03 (m, 1H), 1.60-1.05 (m, 8H), 0.92 (d, J=6.2 Hz, 6H), 0.60-0.45 (m, 2H).

### Example J3

### N-{(1R,5S)-1-cyclopropyl-6-hydroxy-5-[({4-[(1S)-1-hydroxyethyl]phenyl}sulfonyl)(3-methylbutyl)amino]hexyl}-4-fluoro-β-(4-fluorophenyl)-Nα-(methoxycarbonyl)-L-phenylalaninamide

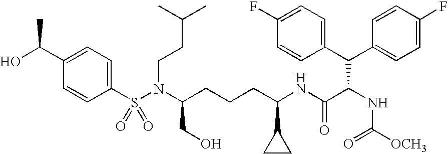

### Step J3-1: methyl N-[(4-acetylphenyl)sulfonyl]-6-(benzyloxy)-N-(3-methylbutyl)-L-norleucinate

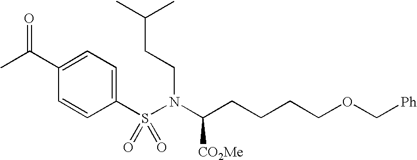

Methyl N-[(4-acetylphenyl)sulfonyl]-6-(benzyloxy)-N-(3-methylbutyl)-L-norleucinate was prepared from methyl 6-(benzyloxy)-L-norleucine and 4-acetylbenzenesulfonyl chloride using a procedure similar to that described in the preparation of Example J1. MS: M+Na=526.

### Step J3-2: methyl 6-(benzyloxy)-N-({4-[(1S)-1-hydroxyethyl]phenyl}sulfonyl)-N-(3-methylbutyl)-L-norleucinate

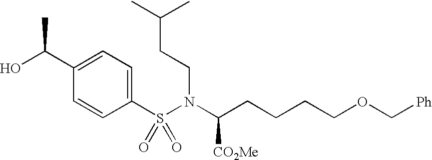

To a solution of methyl N-[(4-acetylphenyl)sulfonyl]-6-(benzyloxy)-N-(3-methylbutyl)-L-norleucinate (2.23 g, 4.43 mmol) and (R)-2-methyl-CBS-oxazaborolidine (9.74 mL, 9.74 mmol, 1M toluene) in THF (44 mL) cooled to 0° C. was added borane THF complex (3.54 mL, 3.54 mmol, 1M THF) dropwise. After 2 hours stirring at 0° C., additional borane THF complex (3.5 mL, 3.5 mmol, 1M THF) was added. After another 45 minutes stirring at 0° C. the reaction mixture was quenched with MeOH and acetone, concentrated in vacuo and purified by flash chromatography (120 g silica, 35 to 75% EtOAC in hexane) to provide the desired alcohol as a clear oil. MS M+1=506. Subsequent Mosher ester analysis indicated a 85:15 diastereomeric mixture.

### Step J3-3: N-[(1S)-5-(benzyloxy)-1-(hydroxymethyl)pentyl]-4-[(1S)-1-hydroxyethyl]-N-(3-methylbutyl)benzenesulfonamide

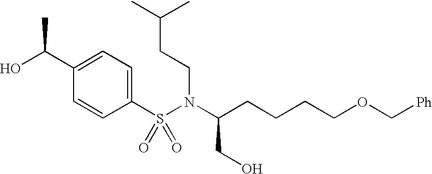

To a solution of methyl 6-(benzyloxy)-N-({4-[(1S)-1-hydroxyethyl]phenyl}-sulfonyl)-N-(3-methylbutyl)-L-norleucinate (2.15 g, 4.25 mmol) in THF (14 mL) was slowly added lithium borohydride (10.6 mL, 21.3 mmol, 2M THF). After 4 hours stirring at room temperature, the reaction mixture was cooled to 0° C., quenched with MeOH and EtOAc, warmed to room temperature, diluted with EtOAc and 50 mL 1N NaOH. After vigorous stirring for 10 minutes, the organic layer was separated, washed with brine, dried over sodium sulfate and concentrated in vacuo to afford the desired product as an oil. MS M+Na=500.

### Steps J3-4 to J3-10: N-{(1R,5S)-1-cyclopropyl-6-hydroxy-5-[({4-[(1S)-1-hydroxyethyl]phenyl}sulfonyl)(3-methylbutyl)amino]hexyl}-4-fluoro-β-(4-fluorophenyl)-Nα-(methoxycarbonyl)-L-phenylalaninamide

N-{(1R,5S)-1-cyclopropyl-6-hydroxy-5-[({4-[(1S)-1-hydroxyethyl]phenyl}sulfonyl)(3-methylbutyl)amino]hexyl}-4-fluoro-β-(4-fluorophenyl)-Nα-(methoxycarbonyl)-L-phenylalaninamide was prepared from N-[(1S)-5-(benzyloxy)-1-(hydroxymethyl)pentyl]-4-[(1S)-1-hydroxyethyl]-N-(3-methylbutyl)benzenesulfonamide, TBDPS-Cl, (R)-(+)-tert-butanesulfinamide, cyclopropyl magnesium bromide and 4-fluoro-β-(4-fluorophenyl)-N-(methoxycarbonyl)-L-phenylalanine using a procedure similar to that described in Example J1, steps J1-5 to J1-11. MS M+1=744. ¹H NMR (d₄ MeOH): δ 7.81 (d, J=8.2 Hz, 2H), 7.55 (d, J=8.2 Hz, 2H), 7.40-7.28 (m, 4H), 7.05-6.95 (m, 4H), 4.95-4.80 (m, 2H), 4.31 (d, J=11.9 Hz, 1H), 3.72-3.62 (m, 1H), 3.54 (s, 3H), 3.48-3.36 (m, 2H), 3.24-3.03 (m, 2H), 2.94-2.84 (m, 1H), 1.60-0.65 (m, 10H), 1.45 (d, J=6.5 Hz, 3H), 0.90 (d, J=6.2 Hz, 6H), 0.45-0.30 (m, 2H), 0.21-0.04 (m, 2H).

The following examples (Table J) were prepared using procedures similar to those described in the preparation of Examples J1 to J3, using the appropriate building blocks (MeO₂C-Ph-SO₂Cl or MeCO-Ph-SO₂Cl, R⁵MgX or CF₃TMS, R¹OH, HO₂C—CHR⁶—NHR⁷ or corresponding activated amino acid such as hydroxysuccinate ester). In some cases NHR⁷ was protected as Boc which necessitated an acidic Boc removal in the last step.

TABLE J

| Example | | |
|---|---|---|
| No. | Structure | M + 1 |
| J4 | N-{(1S,5S)-6-hydroxy-5-[{[4- | 668 |
| | (hydroxymethyl)phenyl]sulfonyl}(3- | |
| | methylbutyl)amino]-1-methylhexyl}-Nα- | |
| | (methoxycarbonyl)-β-phenyl-L-phenylalaninamide | |
| | 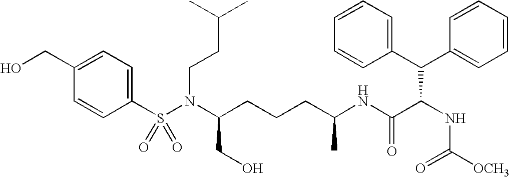 | |
| J5¹ | N-{(1R,5S)-6-hydroxy-5-[{[4- | 668 |
| | (hydroxymethyl)phenyl]sulfonyl}(3- | |
| | methylbutyl)amino]-1-methylhexyl}-Nα- | |
| | (methoxycarbonyl)-β-phenyl-L-phenylalaninamide | |
| | 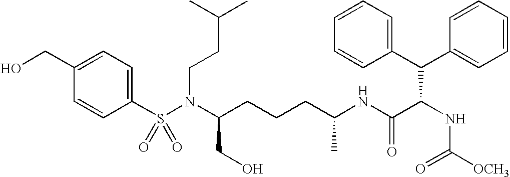 | |
| J6 | N-{(1R,5S)-1-tert-butyl-6-hydroxy-5-[{[4- | 710 |
| | (hydroxymethyl)phenyl]sulfonyl}(3- | |
| | methylbutyl)amino]hexyl}-Nα-(methoxycarbonyl)-β- | |
| | pbenyl-L-phenylalaninamide | |
| | 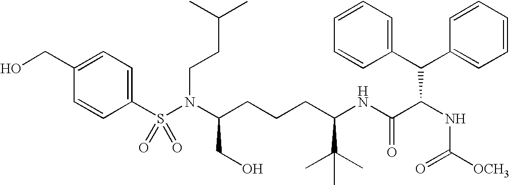 | |
| J7² | N-[(1S,5S)-6-hydroxy-5-[{[4- | 722 |
| | (hydroxymethyl)phenyl]sulfonyl}(3- | |
| | methylbutyl)amino]-1-(trifluoromethyl)hexyl]-Nα- | |
| | (methoxycarbonyl)-β-phenyl-L-phenylalaninamide | |
| | 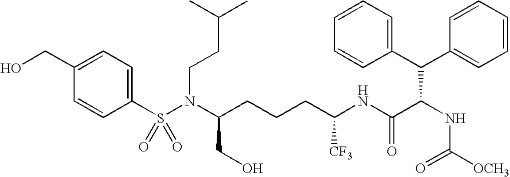 | |
| J8 | N-{(1R,5S)-1-cyclopropyl-6-hydroxy-5-[{[4- | 694 |
| | (hydroxymethyl)phenyl]sulfonyl}(3- | |
| | methylbutyl)amino]hexyl}-Nα-(methoxycarbonyl)-β- | |
| | phenyl-L-phenylalaninamide | |
| | 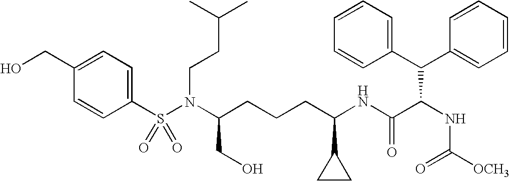 | |
| J9 | N-{(1R,5S)-6-hydroxy-5-[{[4- | 696 |
| | (hydroxymethyl)phenyl]sulfonyl}(3- | |
| | methylbutyl)amino]-1-isopropylhexyl}-Nα- | |
| | (methoxycarbonyl)-β-phenyl-L-phenylalaninamide | |
| | 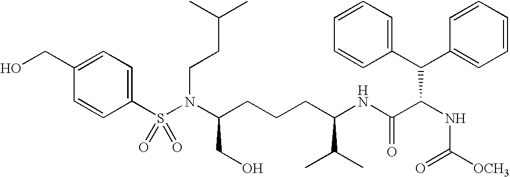 | |
| J10 | N-{(1R,5S)-6-hydroxy-5-[{[4- | 680 |
| | (hydroxymethyl)phenyl]sulfonyl}(3- | |
| | methylbutyl)amino]-1-vinylhexyl}-Nα- | |
| | (methoxycarbonyl)-β-phenyl-L-phenylalaninamide | |
| | 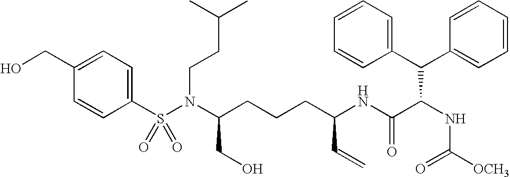 | |
| J11¹ | N-{(1S,5S)-6-hydroxy-5-[{[4- | 680 |
| | (hydroxymethyl)phenyl]sulfonyl}(3- | |
| | metbylbutyl)amino]-1-vinylhexyl}-Nα- | |
| | (methoxycarbonyl)-β-phenyl-L-phenylalaninamide | |
| | 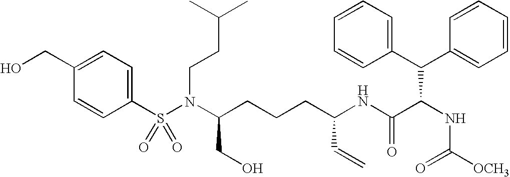 | |
| J12³ | N-[(1R,5S)-6-hydroxy-5-[{[4- | 772 |
| | (hydroxymethyl)phenyl]sulfonyl}(3- | |
| | methylbutyl)amino]-1-(pentafluoroethyl)hexyl]-Nα- | |
| | (methoxycarbonyl)-β-phenyl-L-phenylalaninamide | |
| | 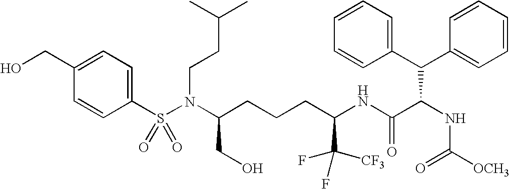 | |
| J13⁴ | N-{(1R,5S)-1-ethynyl-6-hydroxy-5-[{[4- | 678 |
| | (hydroxymethyl)phenyl]sulfonyl}(3- | |
| | methylbutyl)amino]hexyl}-Nα-(methoxycarbonyl)-β- | |
| | phenyl-L-phenylalaninamide | |
| | 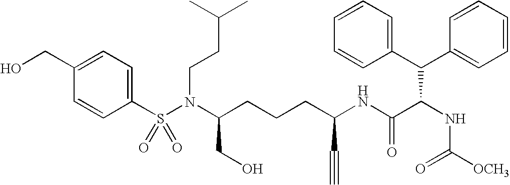 | |
| J14 | 2-chloro-N-{(1R,5S)-1-cyclopropyl-6-hydroxy-5- | 652 |
| | [{[4-(hydroxymethyl)phenyl]sulfonyl}(3- | |
| | methylbutyl)amino]hexyl}-Nα-(methoxycarbonyl)- | |
| | L-phenylalaninamide | |
| | 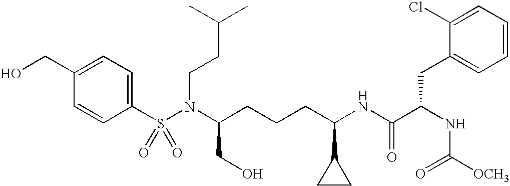 | |
| J15⁵ | 2-chloro-N-{(1R,5S)-1-cyclopropyl-6-hydroxy-5- | 666 |
| | [({4-[(1S)-1-hydroxyethyl]phenyl}sulfonyl)(3- | |
| | methylbutyl)amino]hexyl}-Nα-(methoxycarbonyl)- | |
| | L-pbenylalaninamide | |
| | 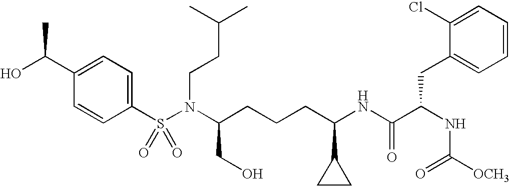 | |
| J16 | N-{(1R,55)-1-cyclopropyl-6-hydroxy-5-[{[4- | 730 |
| | (hydroxymethyl)phenyl]sulfonyl}(3- | |
| | methylbutyl)amino]hexyl}-4-fluoro-β-(4- | |
| | fluorophenyl)-Nα-(methoxycarbonyl)-L- | |
| | phenylalaninamide | |
| | 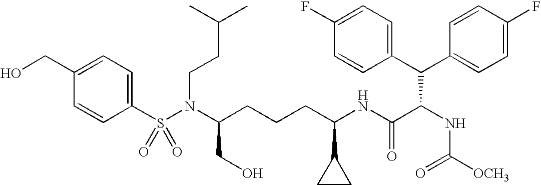 | |
| J17⁶ | N-{(1R,5S)-1-cyclopropyl-6-hydroxy-5-[{[4- | 672 |
| | (hydroxymethyl)phenyl]sulfonyl}(3- | |
| | methylbutyl)amino]hexyl}-4-fluoro-β-(4- | |
| | fluorophenyl)-L-phenylalaninamide hydrochloride | |
| | 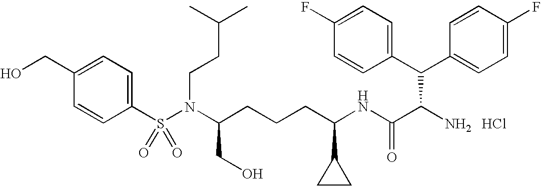 | |
| J18⁷ | N-{(1R,5S)-1-cyclopropyl-6-hydroxy-5-[({4-[(1S)-1- | 686 |
| | hydroxyethyl]phenyl}sulfonyl)(3- | |
| | methylbutyl)amino]hexyl}-4-fluoro-β-(4- | |
| | fluorophenyl)-L-phenylalaninamide hydrochloride | |
| | 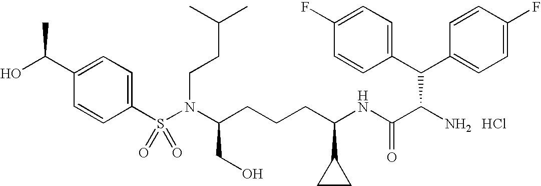 | |
| J19 | N-{(1S,5S)-1-ethyl-6-hydroxy-5-[{[4- | 668 |
| | (hydroxymethyl)phenyl]sulfonyl}(isobutyl)amino]hexyl]- | |
| | Nα-(methoxycarbonyl)-β-phenyl-L- | |
| | phenylalaninamide | |
| | 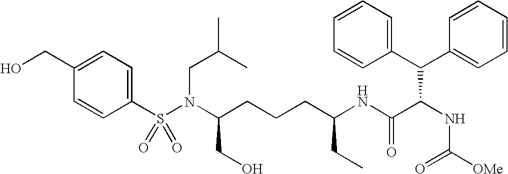 | |
| J20 | N-{(1S,5S)-1-ethyl-6-hydroxy-5-[{[4- | 704 |
| | (hydroxymethyl)phenyl]sulfonyl}(isobutyl)amino]hexyl}- | |
| | 4-fluoro-β-(4-fluorophenyl)-Nα- | |
| | (methoxycarbonyl)-L-phenylalaninamide | |
| | 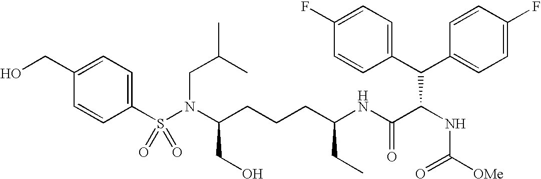 | |
| J21 | 2-chloro-N-{(1S,55)-1-ethyl-6-hydroxy-5-[{[4- | 626 |
| | (hydroxymethyl)phenyl]sulfonyl}(isobutyl)amino]hexyl}- | |
| | Nα-(methoxycarbonyl)-L-phenylalaninamide | |
| | 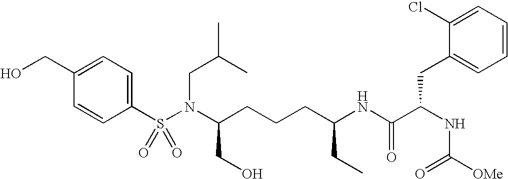 | |
| J22 | 2-chloro-N-{(1S,5S)-1-ethyl-6-hydroxy-5-[{[4- | 680 |
| | (hydroxymethyl)phenyl]sulfonyl}(isobutyl)amino]hexyl}- | |
| | Nα-(methoxycarbonyl)-L-phenylalaninamide | |
| | 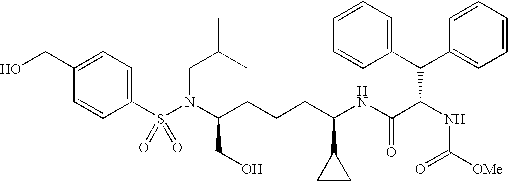 | |
| J2³ | N-{(1R,5S)-1-cyclopropyl-6-hydroxy-5-[{[4- | 716 |
| | (hydroxymethyl)phenyl]sulfonyl}(isobutyl)amino]hexyl}- | |
| | 4-fluoro-β-(4-fluorophenyl)-Nα- | |
| | (methoxycarbonyl)-L-phenylalaninamide | |
| | 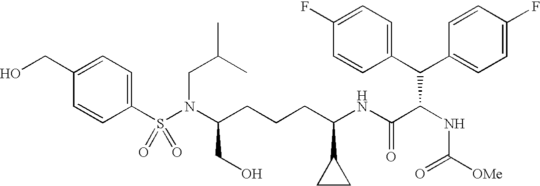 | |
| J24⁶ | N-{(1S,5S)-1-ethyl-6-hydroxy-5-[{[4- | 624 |
| | (hydroxymethyl)phenyl]sulfonyl}(isobutyl)amino]hexyl}- | |
| | Nα-methyl-β-phenyl-L-phenylalaninamide | |
| | hydrochloride | |
| | 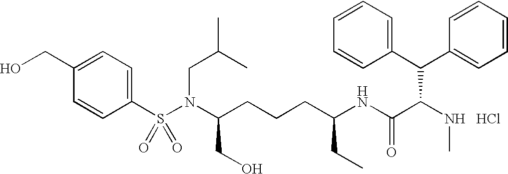 | |
| J25⁶ | N-{(1R,55)-1-cyclopropyl-6-hydroxy-5-[{[4- | 636 |
| | (hydroxymethyl)phenyl]sulfonyl}(isobutyl)amino]hexyl}- | |
| | Nα-methyl-β-phenyl-L-phenylalaninamide | |
| | 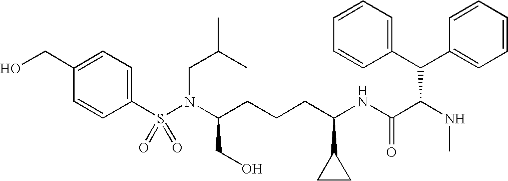 | |
| J26 | 2-chloro-N-{(1R,5S)-6-hydroxy-5-[{[4- | |
| | (hydroxymethyl)phenyl]sulfonyl}(3- | |
| | methylbutyl)amino]-1-isopropylhexyl}-Nα- | |
| | (methoxycarbonyl)-L-phenylalaninamide | |
| | 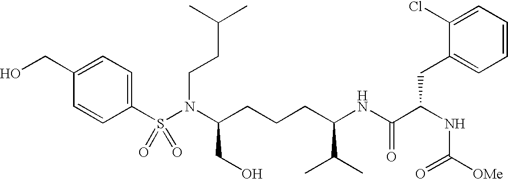 | |
| J27⁶ | N-{(1S,5S)-1-ethyl-6-hydroxy-5-[{[4- | 638 |
| | (hydroxymethyl)phenyl]sulfonyl}(3- | |
| | methylbutyl)amino]hexyl}-Nα-methyl-β-phenyl-L- | |
| | phenylalaninamide | |
| | 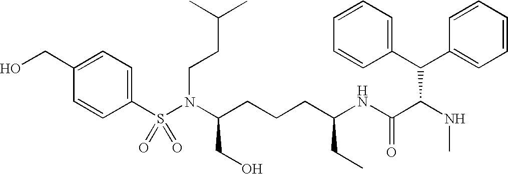 | |
| J28⁶ | N-{(1R,5S)-1-cyclopropyl-6-hydroxy-5-[{[4- | 650 |
| | (hydroxymethyl)phenyl]sulfonyl}(3- | |
| | methylbutyl)amino]hexyl}-Nα-methyl-β-phenyl-L- | |
| | phenylalaninamide | |
| | 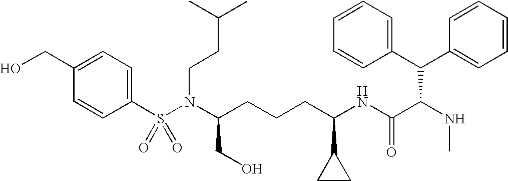 | |
| J29⁶ | N-{(1R,5S)-6-hydroxy-5-[{[4- | 652 |
| | (hydroxymethyl)phenyl]sulfonyl}(3- | |
| | methylbutyl)amino]-1-isopropylhexyl}-Nα-methyl- | |
| | β-phenyl-L-phenylalaninamide | |
| | 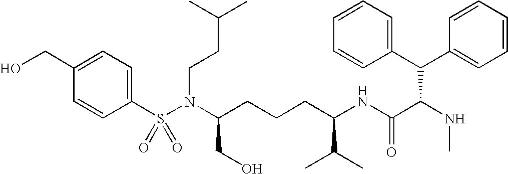 | |
| J30⁸ | N-[(1R,5S)-6-hydroxy-5-[{[4- | 708 |
| | (hydroxymethyl)phenyl]sulfonyl}(isobutyl)amino]- | |
| | 1-(trifluoromethyl)hexyl]-Nα-(methoxycarbonyl)-β- | |
| | phenyl-L-phenylalaninamide | |
| | 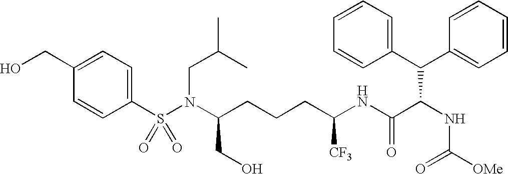 | |
| J31⁶ | N-{(1S,5S)-1-ethyl-6-hydroxy-5-[{[4- | 624 |
| | (hydroxymethyl)phenyl]sulfonyl}(3- | |
| | methylbutyl)amino]hexyl}-β-phenyl-L- | |
| | phenylalaninamide hydrochloride | |
| | 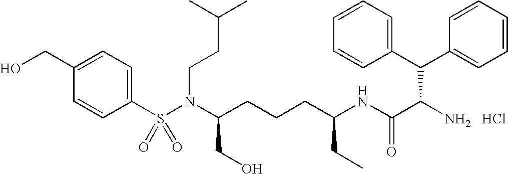 | |
| J32⁶ | N-{(1S,5S)-1-ethyl-6-hydroxy-5-[{[4- | 610 |
| | (hydroxymethyl)phenyl]sulfonyl}(isobutyl)amino]hexyl}- | |
| | β-phenyl-L-phenylalaninamide hydrochloride | |
| | 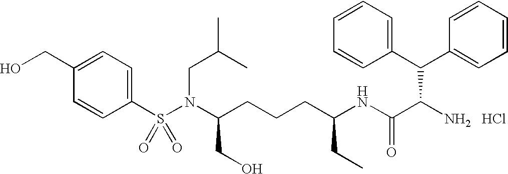 | |
| J33 | N-{(5S)-6-hydroxy-5-[{[4- | 640 |
| | (hydroxymethyl)phenyl]sulfonyl}(propyl)amino]-1- | |
| | methylhexyl}-Nα-(methoxycarbonyl)-β-phenyl-L- | |
| | phenylalaninamide | |
| | 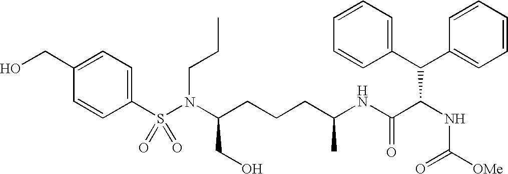 | |
| J34 | N-{(1S,5S)-1-ethyl-6-hydroxy-5-[{[4- | 654 |
| | (hydroxymethyl)phenyl]sulfonyl}(propyl)amino]hexyl}- | |
| | Nα-(methoxycarbonyl)-β-phenyl-L- | |
| | phenylalaninamide | |
| | 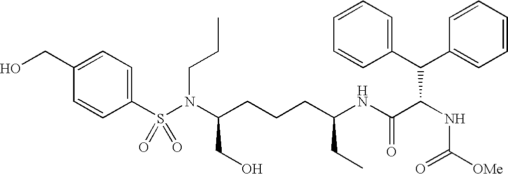 | |
| J35⁶ | N-{(1S,5S)-1-ethyl-6-hydroxy-5-[{[4- | 610 |
| | (hydroxymethyl)phenyl]sulfonyl}(isopropyl)amino] | |
| | hexyl}-Nα-methyl-β-phenyl-L-phenylalaninamide | |
| | hydrochloride | |
| | 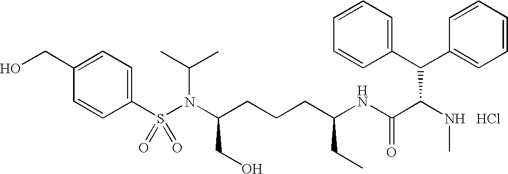 | |
| J36⁶ | N-{(1S,5S)-1-ethyl-6-hydroxy-5-[{[4- | 596 |
| | (hydroxymethyl)phenyl]sulfonyl}(isopropyl)amino] | |
| | hexyl}-β-phenyl-L-phenylalaninamide | |
| | 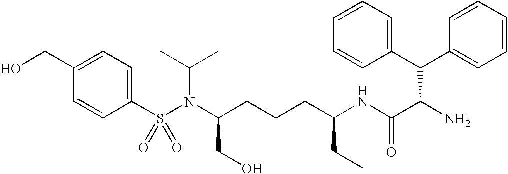 | |
| J3⁷ | 2-bromo-N-{(1R,5S)-1-cyclopropyl-6-hydroxy-5- | 668 |
| | [{[4- | |
| | (hydroxymethyl)phenyl]sulfonyl}(isopropyl)amino] | |
| | hexyl}-Nβ-(methoxycarbonyl)-L-phenylalaninamide | |
| | 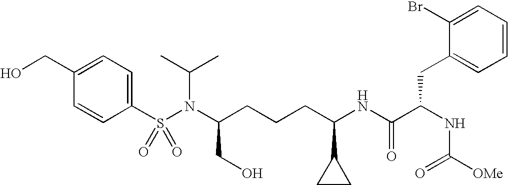 | |
| J38⁶ | N-{(1S,5S)-1-ethyl-6-hydroxy-5-[{[4- | 610 |
| | (hydroxymethyl)phenyl]sulfonyl}(propyl)amino]hexyl}- | |
| | Nα-methyl-β-phenyl-L-phenylalaninamide | |
| | 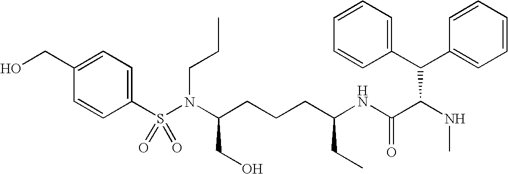 | |
| J39⁶ | N-{(1S,5S)-1-ethyl-6-hydroxy-5-[{[4- | 596 |
| | (hydroxymethyl)phenyl]sulfonyl}(propyl)amino]hexyl}- | |
| | β-phenyl-L-phenylalaninamide | |
| | 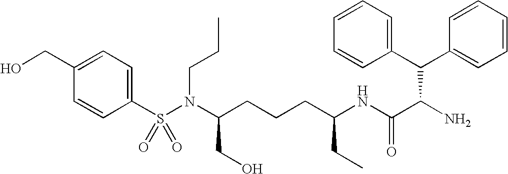 | |
| J40⁸ | N-[(1R,5S)-6-hydroxy-5-[{[4- | 694 |
| | (hydroxymethyl)phenyl]sulfonyl}(isopropyl)amino]- | |
| | 1-(trifluoromethyl)hexyl]-Nα-(methoxycarbonyl)-β- | |
| | phenyl-L-phenylalaninamide | |
| | 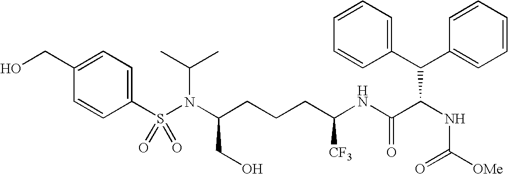 | |
| J41 | methyl [(1S)-2-({(1S,5S)-1-ethyl-6-hydroxy-5-[{[4- | 628 |
| | (hydroxymethyl)phenyl]sulfonyl}(propyl)amino]hexyl} | |
| | amino)-1-(1-naphthylmethyl)-2- | |
| | oxoethyl]carbamate | |
| | 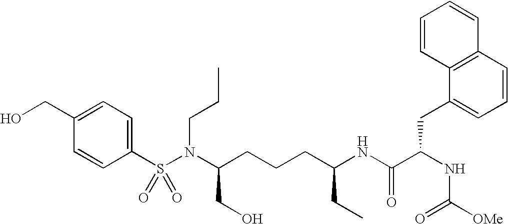 | |
| J42⁸ | N-[(1R,5S)-6-hydroxy-5-[{[4- | 694 |
| | (hydroxymethyl)phenyl]sulfonyl}(propyl)amino]-1- | |
| | (trifluoromethyl)hexyl]-Nα-(methoxycarbonyl)-α- | |
| | phenyl-L-phenylalaninamide | |
| | 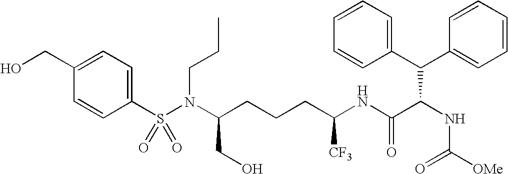 | |
| J43⁸ | 2-chloro-N-[(1R,5S)-6-hydroxy-5-[{[4- | 652 |
| | (hydroxymethyl)phenyl]sulfonyl}(propyl)amino]-1- | |
| | (trifluoromethyl)hexyl]-Nα-(methoxycarbonyl)-L- | |
| | phenylalaninamide | |
| | 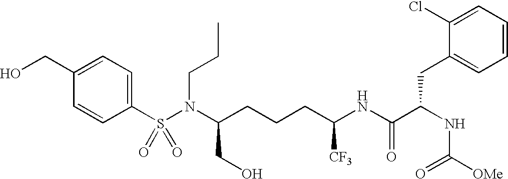 | |
| J44 | 4-chloro-β-(4-chlorophenyl)-N-{(1S,5S)-1-ethyl-6- | 750 |
| | hydroxy-5-[{[4-(hydroxymethyl)phenyl]sulfonyl}(3- | |
| | methylbutyl)amino]hexyl}-Nα-(metboxycarbonyl)- | |
| | L-phenylalaninamide | |
| | 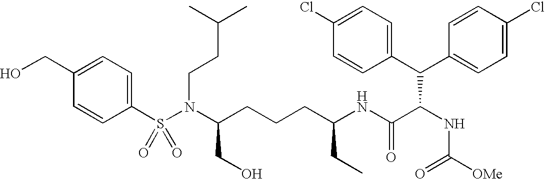 | |
| J45⁹ | 2,3-dichloro-N-{(1S,5S)-6-hydroxy-5-[{[4- | 632 |
| | (hydroxymethyl)phenyl]sulfonyl}(isopropyl)amino]- | |
| | 1-methylhexyl}-Nα- | |
| | (methoxycarbonyl)phenylalaninamide | |
| | 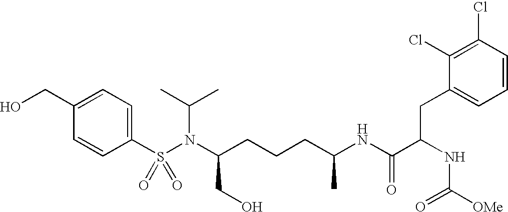 | |
| J46⁹ | 3-fluoro-β-(3-fluorophenyl)-N-{(1S,5S)-6-hydroxy- | 676 |
| | 5-[{[4- | |
| | (hydroxymethyl)phenyl]sulfonyl}(isopropyl)amino]- | |
| | 1-methylhexyl}-Nα- | |
| | (methoxycarbonyl)phenylalaninamide | |
| | 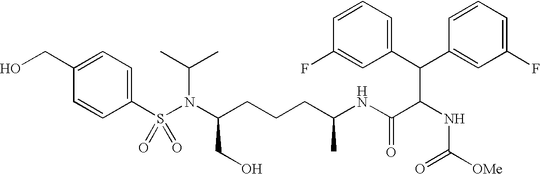 | |

¹Derived from S-Ellman sulfimine
²Derived from R-Ellman sulfimine
³Derived from CF₃CF₂-TMS and S-Ellman sulfimine, as exemplifed in Example J2
⁴Prepared using TMS-acetylene and EtMgBr to generate R⁵-MgBr
⁵Prepared as described in Example J3
⁶Prepared as described in Example J3 with the addition of Boc removal as the last step
⁷Prepared as described in Example J3 with the addition of Boc removal as the last step
⁸Prepared as described in Example J2
⁹Prepared as RS at R⁶ bearing center

### Example K1

### N-{(1S,5S)-6-hydroxy-5-[{[4-(hydroxymethyl)phenyl]sulfonyl}(isopropyl)amino]-1-methylhexyl}-Nα-(methoxycarbonyl)-β-phenyl-L-phenylalaninamide

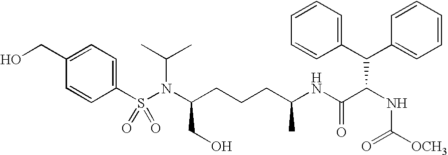

### Step K1-1 Ethyl-2-aminopent-4-enoate hydrochloride

The synthesis of this intermediate was prepared in a manner similar to that described in Example F1-1 with the modification of using methanol as the solvent.

### Step K1-2 Methyl 4-(N-(1-methoxy-1-oxopent-4-en-2-yl)sulfamoyl)benzoate

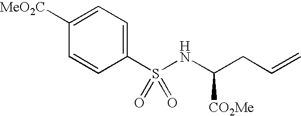

To a solution of ethyl (2S)-2-amino-4-pentenoate hydrochloride (12.01 g, 66.9 mmol) in 223 mL CH₂Cl₂ was added triethylamine (20.0 mL, 140 mmol) and 4-carbomethoxyphenylsulfonyl chloride (14.67 g, 66.2 mmol). The reaction was allowed to proceed at room temperature for 16 hours, then diluted with EA and washed with 0.5M HCl (2×), saturated aqueous NaHCO₃ and brine, dried over Na₂SO₄, filtered and concentrated to obtain the desired product that was used without further purification.

### Step K1-3 Methyl 4-({isopropyl[(1S-1-(methoxycarbonyl)-3-buten-1-yl]amino)sulfonyl)benzoate

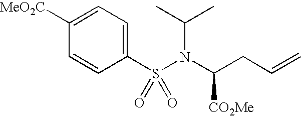

To a solution of methyl 4-(N-(1-methoxy-1-oxopent-4-en-2-yl)sulfamoyl)benzoate (1.97 g, 5.77 mmol) in 30 mL THF was added Ph₃P (3.03 g, 11.54 mmol), i-PrOH (1.73 g, 28.9 mmol) and DIAD (2.24 mL, 11.5 mmol). After overnight stirring, the reaction mixture was concentrated and purified residue by silica gel chromatography (gradient: 20 to 100% EA/hexanes) to obtain the desired product. MS (M+H=383).

### Step K1-4: 4-(hydroxymethyl)-N-[(1S-1-(hydroxymethyl)-3-buten-1-yl]-N-isopropylbenzenesulfonamide

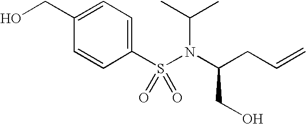

A stirring solution containing methyl 4-({isopropyl[(1S-1-(methoxycarbonyl)-3-buten-1-yl]amino)sulfonyl)benzoate (5.64 g, 15.27 mmol) and 51 mL anhydrous THF was chilled to 0° C. and maintained under an inert atmosphere (nitrogen). To the chilled solution was added 30.5 mL LiAlH₄ (1 M in THF, 30.5 mmol) via syringe. The resulting mixture was allowed to stir for 30 minutes at 0° C. To the reaction mixture was added 15 mL 4N HCl and the resulting mixture was stirred until it was homogeneous. EtOAc was added to the acidified reaction mixture, and the organic layers were separated from the aqueous layer. The organics were washed with brine, and dried over Na₂SO₄ to afford the diol, which was used without further purification in the next step. MS (M+1=314).

### Step K1-5

### 4-([tert-butyl(dimethyl)silyl]oxy)methyl)-N-[(1S-1-1({[tert-butyl(dimethyl)silyl]oxy)methyl)-3-buten-1-yl]-N-isopropylbenzenesulfonamide

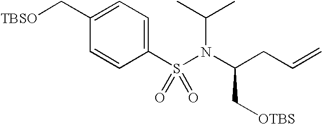

To a solution containing 4-(hydroxymethyl)-N-[(1S-1-(hydroxymethyl)-3-buten-1-yl]-N-isopropylbenzenesulfonamide (4.5 g, 14.36 mmol) and 15 mL anhydrous DCM was added sequentially tert-butyldimethyl chloride (6.49 g, 43.1 mmol), imidazole (2.93 g, 43.1 mmol), and DMAP (3.51 g, 28.7 mmol). The resulting solution was stirred for 12 hours at room temperature. The reaction mixture was concentrated under vacuum and chromatographed (gradient: 20%-50% ethyl acetate/hexanes) to afford benzyl silyl ether. MS (M+1=542).

### Step K1-6

### 4-({[tert-butyl(dimethyl)silyl]oxy}methyl)-N-[(1S,3S)-1-1({[tert-butyl(dimethyl)silyl]oxy)methyl)-5-oxo-3-penten-1-yl]-N-isopropylbenzenesulfonamide

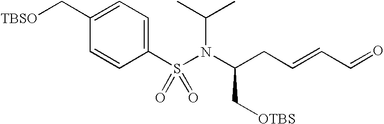

To a stirring solution 4-({[tert-butyl(dimethyl)silyl]oxy}methyl)-N-[(1S-1-1({[tert-butyl(dimethyl)silyl]oxy)methyl)-3-buten-1-yl]-N-isopropylbenzenesulfonamide (4.30 g, 5.44 mmol), crotonaldehyde (4 mL, 3.38 g, 48.3 mmol), in 75 mL DCM was added Grubbs' 2^{nd} Generation catalyst (0.231 g, 0.272 mmol). A reflux condenser was attached to the reaction vessel that also has a N₂ inlet. The reaction mixture was heated to reflux in a silicone oil bath under nitrogen for 30 minutes then allowed to cool to room temperature. The reaction mixture was then concentrated under vacuum and chromatographed (gradient: 20%-100% ethyl acetate/hexanes) to afford enal.

### Step K1-7 4-([tert-butyl(dimethyl)silyl]oxy}methyl)-N-[(1S,3S)-1-1({[tert-butyl(dimethyl)silyl]oxy)methyl)-5-oxo-3-pentyl]-N-isopropylbenzenesulfonamide

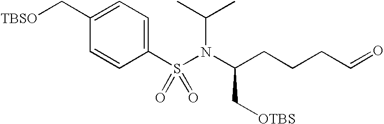

To a solution containing 4-({[tert-butyl(dimethyl)silyl]oxy}methyl)-N-[(1S,3S)-1-1({[tert-butyl(dimethyl)silyl]oxy)methyl)-5-oxo-3-penten-1-yl]-N-isopropylbenzenesulfonamide (3.00 g, 5.42 mmol) in 28.6 mL ethyl acetate was added 10% Pd/C (0.579 g, 0.544 mmol). The resulting mixture was hydrogenated under STP for 1.5 hours. The reaction mixture was then filtered through celite and concentrated under vacuum to afford the aldehyde.

### Step K1-8 4-({[tert-butyl(dimethyl)silyl]oxy)methyl)-N-[(1S,5E)-1-1({[tert-butyl(dimethyl)silyl]oxy)methyl)-5-{[(S)-tert-butylsulfinyl]imino)pentyl)-N-isopropylbenzenesilfonamide

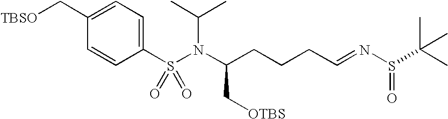

To a solution containing 4-({[tert-butyl(dimethyl)silyl]oxy}methyl)-N-[(1S,3S)-1-1({[tert-butyl(dimethyl)silyl]oxy)methyl)-5-oxo-3-pentyl]-N-isopropylbenzenesulfonamide (3.01 g, 5.42 mmol) in 13.5 mL anhydrous DCM was added, sequentially, MgSO₄ (3.26 g, 27.1 mmol), (S)-Ellman Sulfinamine (0.985 g, 8.13 mmol), and pyridinum p-toluene sulfonate (0.136 g, 0.542 mmol). The resulting mixture was stirred for 18 hours at room temperature. The reaction mixture was then concentrated under vacuum and chromatographed (gradient: 10% to 80% EtOAc/hexanes) to yield sulfinimine. MS (M+1=676).

### Step K1-9 4-({[tert-butyl(dimethyl)silyl]oxy}methyl)-N-[(1S,5E)-1-1({[tert-butyl(dimethyl)silyl]oxy)methyl)-5-{[(S)-tert-butylsulfinyl]amino)hexyl)-N-isopropylbenzenesilfonamide

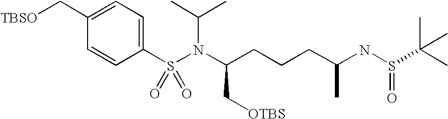

A solution of 4-({[tert-butyl(dimethyl)silyl]oxy)methyl)-N-[(1S,5E)-1-1({[tert-butyl(dimethyl)silyl]oxy)methyl)-5-{[(S)-tert-butylsulfinyl]imino)pentyl)-N-isopropylbenzenesilfonamide (2.40 g, 3.55 mmol) in 40 mL DCM was chilled to 0° C. and maintained under a nitrogen atmosphere. To this chilled solution was added methyl magnesium bromide (2.37 mL, 7.11 mmol, 3.0 M in diethyl ether) dropwise via syringe. The reaction mixture was allowed to stir for 18 hours, at which point the reaction was complete as determined by TLC. The reaction mixture was diluted with saturated NH₄Cl solution and extracted with DCM (3×10 mL). The combined organics were dried over Na₂SO₄ to afford sulfinamine which was used directly in the next step.

### Step K1-10 N-[(1S,5S)-5-amino-1-(hydroxymethyl)hexyl]-4-(hydroxylmethyl)-N-isopropylbenzenesulfonamide

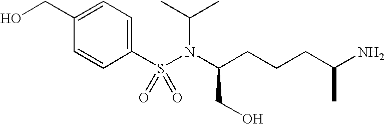

To a solution of 4-({[tert-butyl(dimethyl)silyl]oxy}methyl)-N-[(1S,5E)-1-1({[tert-butyl(dimethyl)silyl]oxy)methyl)-5-{[(S)-tert-butylsulfinyl]amino)hexyl)-N-isopropylbenzenesilfonamide (2.40 g, 3.47 mmol) in 6.0 mL methanol was added 2M HCl in dioxane (10.42 mL, 20.83 mmol) and the mixture was allowed to stir for 18 hours at room temperature. The reaction mixture was then concentrated under vacuum and chromatographed by Strong Cation Exchange chromatography (SCX) to afford the amine-diol. MS (M+1=359).

Step K1-11 N-{(1S,5S)-6-hydroxy-5-[{[4-(hydroxymethyl)phenyl]sulfonyl}(isopropyl)amino]-1-methylhexyl}-N-α-(methoxycarbonyl)-β-phenyl-L-phenylalaninamide

To a solution containing N-[(1S,5S)-5-amino-1-(hydroxymethyl)hexyl]-4-(hydroxylmethyl)-N-isopropylbenzenesulfonamide (1.80 g, 4.56 mmol), in 4 mL THF, and 4 mL saturated NaHCO₃ solution was added methyl[(1S)-2-[(2,5-dioxo-1-pyrrolidinyl)oxy]-1-(diphenylmethyl)-2-oxoethyl]carbamate (1.81 g, 4.56 mmol). The resulting mixture was allowed stir for 18 hours at room temperature. After 18 hours, the reaction mixture was diluted with water and ethyl acetate, and the organic and aqueous layers were separated. The organics were collected and dried over Na₂SO₄, then filtered, concentrated under vacuum, and purified by reverse phase chromatography to afford the desired product. The purification revealed 10:1 mixture of diastereomers, favoring the desired diastereomer. MS (M+1=640). ¹H NMR (400 MHz, CDCl₃) δ 7.82 (d, J=8.4 Hz, 2H), 7.57 (d, J=8.4 Hz, 2H), 7.35-7.18 (m, 10H), 6.07 (br s, 1H), 5.18 (d, J=9.2 Hz, 1H), 4.92-4.71 (m, 3H), 4.53 (br s, 1H), 4.38 (d, J=7 Hz, 1H) 3.59 (s, 3H), 3.53-3.49 (m, 4H), 3.29 (br s, 1H), 2.83 (br s, 1H), 2.89-2.60 (br s, 1H), 1.63-1.51 (m, 3H) 1.40-1.33 (m, 3H), 0.89 (m, 2H), 0.74 (d, J=6.8 Hz, 2H).

### Example K2

### N-{(1S,5S)-1-ethyl-6-hydroxy-5-[({4-[(1S)-1-hydroxyethyl]phenyl}sulfonyl)(isopropyl)-amino]hexyl}-Nα-(methoxycarbonyl)-β-phenyl-L-phenylalaninamide

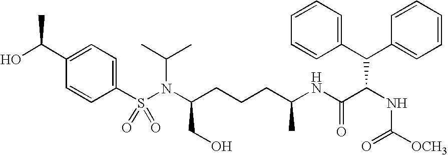

### Step K2-1 Ethyl (2S)-2-amino-4-pentenoate hydrochloride

The compound was prepared as described in Step F1-1 of Example F1.

### Step K2-2 ethyl (2S)-2-{[(4-acetylphenyl)sulfonyl]amino}-4-pentenoate

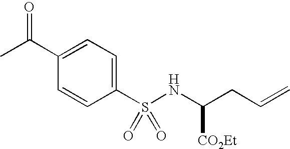

To a solution of ethyl (2S)-2-amino-4-pentenoate hydrochloride K2-1 (2 g, 11.13 mmol) and 111 mL DCM was added 4-acetylbenzenesulfonyl chloride (2.19 g, 10.02 mmol) and triethylamine (1.54 mL, 11.13 mmol). The solution was allowed to stir at room temperature for 18 hours. The reaction mixture was then washed three times each with saturated NaHCO₃ solution and brine. The organics were dried over Na₂SO₄ and chromatographed (gradient: 20%-50% EtOAc/hexanes) to afford ketone K2-2. LC/MS (M+1=326).

### Step K2-3 Ethyl-(2S)-2-[{4-[(15))-1-hydroxyethyl]phenyl}sulfonyl)amino]-4-pentenoate

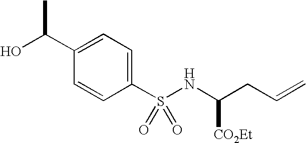

A solution of K2-2 sulfonamide (1.88 g, 5.80 mmol) in 58 mL anhydrous THF was chilled to 0° C. and kept under nitrogen atmosphere. To this solution was added (R)-(+)-2-methyl-CBS-oxazaborolidine (12.75 mL, 12.75 mmol, 1 M in toluene) via syringe. The resulting solution was allowed to stir for 30 minutes at 0° C., after which borane-THF complex (4.64 mL, 4.64 mmol, 1.0 M in THF) was added dropwise via syringe. The resulting solution was allowed to stir for 2 hours at 0° C. until the reaction was complete as determined by TLC. The reaction mixture was quenched by the addition of acetone and methanol. The reaction mixture was concentrated under vacuum and chromatographed (gradient: 20%-100% ethyl acetate/hexanes) to afford desired compound K2-3. LC/MS (M+23=350). The diastereomeric purity was established by Mosher ester analysis, according to the procedure set forth in Step K2-4 below.

### Step K2-4 Ethyl-(2S)-2-({[4-((1R)-1-{[tert-butyl(dimethyl)silyl]oxy}ethyl)phenyl]sulfonyl}amino)-4-pentenoate

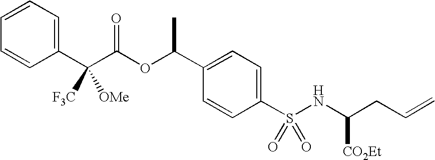

To a solution containing 100 mg benzyl alcohol K2-3 and 3.05 mL anhydrous DCM was added 59.7 mg 4-dimethylaminopyridine and 108 mg R-Mosher acid chloride (i.e., α-methoxytrifluorophenylacetyl chloride). The solution was allowed to stir at room temperature for 15 hours. The crude reaction mixture was analyzed by LC/MS (M+23=546). A diastereomeric ratio of >10:1 was observed using ¹H NMR.

### Step K2-5 ethyl (2S)-2-({[4-((1R)-1-{[tert- -butyl(dimethyl)silyl]oxy}ethyl)phenyl]sulfonyl}amino)-4-pentenoate

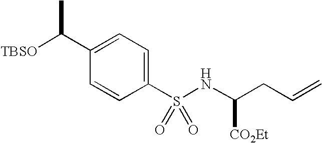

To a solution of benzyl alcohol K2-3 (1.92 g, 5.86 mmol) in 58.3 mL anhydrous DCM was added sequentially tert-butyldimethylsilyl chloride (1.32 g, 8.78 mmol), imidazole (797 mg, 11.71 mmol), and 4-dimethylaminopyridine (1.43 g, 11.71 mmol). The resulting solution was stirred for 12 hours at room temperature. The reaction mixture was then concentrated under vacuum and chromatographed (gradient: 20%-50% ethyl acetate/hexanes) to afford benzyl silyl ether K2-5. LC/MS (M+23=464).

### Step K2-6 ethyl (2S)-2-({[4-((1R)-1-{[tert- -butyl(dimethyl)silyl]oxy}ethyl)-N-isopropylphenyl]sulfonyl}amino)-4-pentenoate

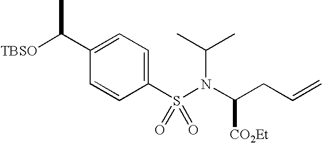

To a solution of benzylsilyl ether K2-5 (1.92 g, 4.35 mmol) in 43.5 mL anhydrous THF was added sequentially anhydrous 2-propanol (2.01 mL, 26.1 mmol), triphenylphosphine (2.85 g, 10.87 mmol) and diisopropylazodicarboxylate (2.198 g, 10.87 mmol). The resulting mixture was stirred for 12 hours at room temperature. The reaction mixture was then concentrated under pressure and chromatographed (gradient: 0%-65% ethyl acetate/hexanes) to afford benzylsilyl ether K2-6. LC/MS (M+1=484).

### Step K2-7 4-((S)-1-hydroxyethyl)-N—((S)-1-hydroxypent-4-en-2-yl)-N-isopropylbenzenesulfonamide

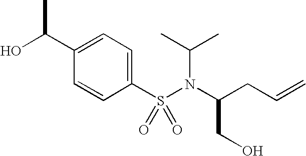

A solution of K2-6 (2.11 g, 4.36 mmol) in 43.6 mL anhydrous THF was chilled to 0° C. under an inert atmosphere (nitrogen), after which LiAlH₄ (1 M in THF, 8.72 mL, 8.72 mmol) was added via syringe. The resulting mixture was allowed to stir for 30 minutes at 0° C. To the reaction mixture was added 5 mL 1N HCl until the mixture solidified and 5 mL concentrated HCl until the reaction mixture was homogeneous. To the acidified reaction mixture was added ethyl acetate. The organic layers were separated from the aqueous layer. The organics were washed with brine, and dried over Na₂SO₄ to afford diol K2-7. This material was used without further purification in Step K2-8.

### Step K2-8 4-((S)-1-(tert-butyldimethyl silyloxy)ethyl)-N—((S)-1-(tert-butyldimethyl silyloxy)pent-4-en-2-yl)-N-isopropylbenzenesulfonamide

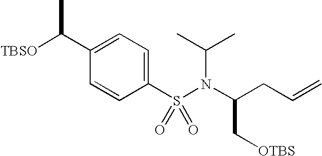

To a solution of benzyl alcohol K2-7 (1.93 g, 5.86 mmol) in 58.9 mL anhydrous DCM was added sequentially TBS chloride (2.22 g, 14.71 mmol), imidazole (0.801 g, 11.77 mmol), and DMAP (1.438 g, 11.77 mmol). The resulting solution was stirred for 12 hours at room temperature. The reaction mixture was concentrated under vacuum and chromatographed (gradient: 20%-50% EtOAc/hexanes) to afford benzyl silyl ether K2-8. LC/MS (M+23=464).

### Step K2-9 N—((S,E)-1-(tert-butyldimethylsilyloxy)-6-oxohex-4-en-2-yl)-4-((S)-1-(tert-butyldimethylsilyloxy)ethyl)-N-isopropylbenzenesulfonamide

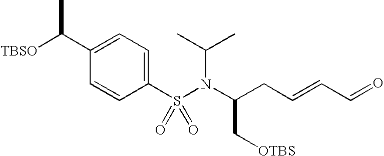

To a solution of silyl ether K2-8 (1.82 g, 3.27 mmol), crotonaldehyde (2.29 g, 32.7 mmol) in 25 mL DCM was added 0.277 g Grubbs' 2^{nd} Generation catalyst. A reflux condenser was attached to the reaction vessel that also has a N2 inlet. The reaction mixture was heated to reflux in a silicone oil bath under nitrogen for 30 minutes then allowed to cool to room temperature. The reaction mixture was concentrated under vacuum and chromatographed (gradient: 20%-100% ethyl acetate/hexanes) to afford enal K2-9.

### Step K2-10 N—((S,E)-1-(tert-butyldimethylsilyloxy)-6-oxohexan-2-yl)-4-((S)-1-(tert-butyldimethylsilyloxy)ethyl)-N-isopropylbenzenesulfonamide

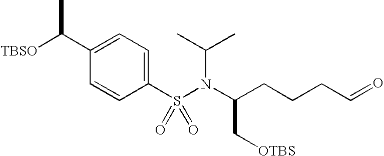

To a solution of enal K2-9 (1.66 g, 2.86 mmol) in 28.6 mL ethyl acetate was added 10% Pd/C (340 mg, 0.286 mmol). The resulting mixture was hydrogenated under STP for 1 hour. The reaction mixture was filtered through celite and concentrated under vacuum to afford aldehyde K2-10.

### Step K2-11 4-((1)-1-{[tert-butyl(dimethyl)silyl]oxy}ethyl)-N-((1S,5E)-1-({[tert butyl(dimethyl)silyl]oxy}methyl)-5-{[(R)-tert-butylsulfinyl]imino}pentyl)-N-isopropylbenzenesulfonamide

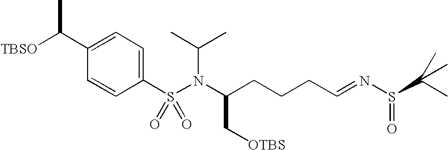

To a solution of K2-10 (1.588 g) in 13.5 mL anhydrous DCM was added, sequentially, MgSO₄ (1.63 g, 13.55 mmol), (R)-Ellman Sulfinamine (493 mg, 4.06 mmol), and PPTS (68 mg, 0.271 mmol). The resulting mixture was stirred for 18 hours at room temperature. The reaction mixture was concentrated under vacuum and chromatographed (10%-80% EtOAc/hexanes) to yield sulfinimine K2-11. LC/MS (M+1=690).

### Step K2-12 4-((1S)-1-{[tert-butyl(dimethyl)silyl]oxy}ethyl)-N-((1S,5S)-1-({[tert-butyl(dimethyl)silyl]oxy}methyl)-5-{[(R)-tert-butylsulfinyl]amino}heptyl)-N-isopropylbenzenesulfonamide

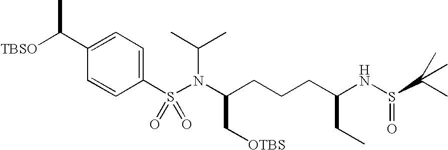

A solution of sulfinimine K2-11 (485 mg, 0.704 mmol) in 7 mL DCM was chilled to 0° C. and maintained under a nitrogen atmosphere. To this chilled solution ethylmagnesium bromide (0.469 mL, 1.407 mmol, 3.0 M in diethyl ether) was added dropwise via syringe. The stirring reaction mixture was allowed to warm to room temperature over 18 hours, at which point the reaction was complete as determined by TLC. The reaction mixture was diluted with saturated NH₄Cl solution and extracted with DCM (3×10 mL). The combined organics were dried over Na₂SO₄ to afford sulfinamine K2-12.

### Step K2-13 N-(1S,5S)-5-amino-1-(hydroxymethyl)heptyl]-4-[(1S))-1-hydroxyethyl]-N-isopropylbenzenesulfonamide

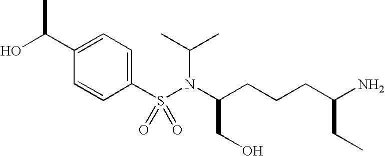

To a solution of sulfinamine K2-12 (433 mg, 0.602 mmol) in 6.02 mL methanol was added HCl (3.01 mL, 12.04 mmol, 4.0 M in dioxane) and let stir for 18 hours at room temperature. The reaction mixture was concentrated under vacuum and chromatographed by SCX to afford K2-13 amine. LC/MS (M+1=387).

### Step K2-14 N-{(1S,5S)-1-ethyl-6-hydroxy-5-[({4-[(1S)-1-hydroxyethyl]phenyl}sulfonyl)(isopropyl)amino]hexyl}-Nα-(methoxycarbonyl)-β-phenyl-L-phenylalaninamide

To a solution of K2-14 (100 mg, 0.236 mmol) in 1.18 mL THF and 1.18 mL saturated NaHCO₃ solution was added methyl[(1S)-2-[(2,5-dioxo-1-pyrrolidinyl)oxy]-1-(diphenylmethyl)-2-oxoethyl]carbamate (141 mg, 0.355 mmol). The resulting mixture was allowed to stir for 18 hours at room temperature. After 18 hours, the reaction mixture was diluted with water and ethyl acetate. The organic and aqueous were separated and then were collected and dried over Na₂SO₄, filtered, concentrated under vacuum, and purified by reverse phase chromatography to afford K2-14. The purification revealed 8:1 mixture of diastereomers, favoring the above compound. LC/MS (M+1=668). ¹H NMR CDCl₃: δ 7.78 (d, J=8.4 Hz, 2H), 7.50 (d, J=7.99 Hz, 2H), 7.32-7.15 (m, 10H), 6.07 (d, J=7.19 Hz, 1H), 5.15 (d, J=9.59 Hz, 2H), 4.96 (q, J=18.4 Hz, 1H), 4.86 (t, J=10.8 Hz, 1H), 4.59 (s, 1H), 4.37 (d, J=Hz, 1H), 3.58 (S, 6H), 3.35 (d, J=24.8 Hz, 2H), 2.81 (s, 1H), 1.66 (d, J=6.39 Hz, 3H), 0.959-0.924 (m, 4H),

### 0.695 (t, J=10.8 Hz, 4H).

### Example K3

### N-{(1S,5S)-5-[[(4-acetylphenyl)sulfonyl](isopropyl)amino]-1-ethyl-6-hydroxyhexyl}-Nα-(methoxycarbonyl)-β-phenyl-L-phenylalaninamide

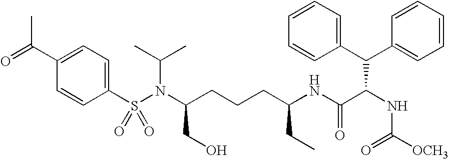

To a solution of N-{(1S,5S)-1-ethyl-6-hydroxy-5-[({4-[(1S)-1-hydroxyethyl]phenyl}sulfonyl)(isopropyl)amino]hexyl}-Nα-(methoxycarbonyl)-β-phenyl-L-phenylalaninamide (20 mg, 0.030 mmol, Example K₂) in 0.299 mL acetone was added MnO₂ (13 mg, 0.150 mmol). The resulting mixture was stirred for 18 hours at room temperature. The reaction mixture was filtered through celite and purified by reverse phase chromatography to afford ketone K3. LC/MS (M+1=666). ¹H NMR CDCl₃: δ 8.04 (d, J=7.58 Hz, 2H), 7.94 (d, J=7.98 Hz, 2H), 7.30-7.16 (m, 10H), 5.52 (d, J=9.18 Hz, 1H), 5.08 (d, J=8.78 Hz, 1H), 4.81 (t, J=9.58 Hz, 1H), 4.5 (d, J=9.98 Hz, 2H), 3.81 (t, J=Hz, 1H) 3.58 (s, 6H), 3.27 (s, 1H), 2.64 (s, 3H), 1.64 (s, 3H), 1.31 (d, J=6.39 Hz, 4H), 1.23 (d, J=7.98 Hz, 6H) 0.73 (t, J=7.58 Hz, 4H).

The following examples (Table K) were prepared using similar procedures as described in the preparation of Examples K1 to K3, using the appropriate building blocks (MeO₂C-Ph-SO₂C1 or MeCO-Ph-SO₂Cl, R⁵MgX or CF₃TMS, R¹OH, HO₂C—CHR⁶—NHR⁷ or corresponding activated amino acid such as hydroxysuccinate ester). In some cases NHR⁷ is originally protected as Boc which necessitates an acidic Boc removal in the last step.

TABLE K

| Example | | |
|---|---|---|
| No. | Structure | M + 1 |
| K4¹ | N-{(1S,5S)-6-hydroxy-5-[({4-[(1S)-1- | 654 |
| | hydroxyethyl]phenyl}sulfonyl)(isopropyl)amino]-1- | |
| | methylhexyl}-Nα-(methoxycarbonyl)-β-phenyl-L- | |
| | phenylalaninamide | |
| | 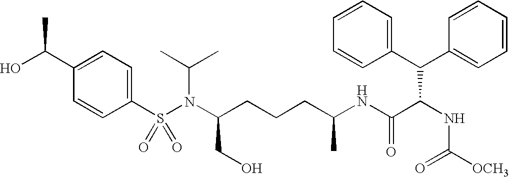 | |
| K5² | N-{(1S,5S)-5-[[(4- | 652 |
| | acetylphenyl)sulfonyl](isopropyl)amino]-6-bydroxy- | |
| | 1-methylhexyl}-Nα-(methoxycarbonyl)-β-phenyl-L- | |
| | phenylalaninamide | |
| | 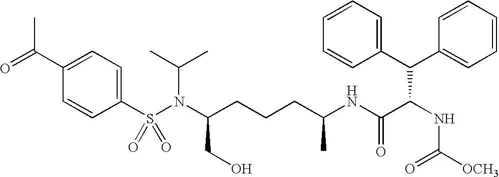 | |

¹The compound was prepared using a procedure analogous to that set forth in Example K2.
²The compound was prepared using a procedure analogous to that set forth in Example K3.

### Example L1

N-(1-{(4S)-4-[[(4-aminophenyl)sulfonyl](3-methylbutyl)amino]-5-hydroxypentyl}cyclopentyl)-Nα-(methoxycarbonyl)-β-phenyl-L-phenylalaninamide

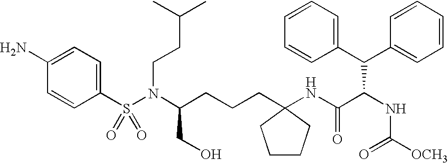

### Step L1-1: methyl (2E)-2-{[(benzyloxy)carbonyl]amino}-5-(1-nitrocyclopentyl)pent-2-enoate

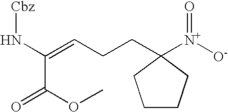

To a solution containing 4.73 mL (44.6 mmol) of nitrocyclopentane and 0.124 mL (0.892 mmol) of triethylamine was slowly added 0.596 mL (8.92 mmol) of acrolein, after which the reaction mixture was stirred for 16 hours. The reaction mixture was quenched with 1M HCl and diluted with DCM. The layers were separated and the organic layer was washed with saturated NaHCO₃ and brine. The organic extract was dried with MgSO₄ and concentrated. DBU (3.45 mL, 22.86 mmol) was added to a solution of (+/−)-Benzyloxycarbonyl-alpha-phosphonoglycine trimethyl ester in DCM (4 mL) at −20° C. The mixture was stirred for 5 minutes, and then a solution of the crude carbinol in 4 mL of DCM was added slowly to maintain the −20° C. temperature during addition. The mixture was allowed to warm to 0° C. and stir for 5 hours followed by 16 hours at room temperature. The reaction mixture was concentrated, then redissolved in EtOAc, and then washed with 1M HCl, saturated NaHCO₃, water and brine. The organic phase was dried over MgSO₄ and concentrated to an oil. The material was used in the next reaction without further purification. LCMS (M+1)=376.9

### Step L1-2 methyl 5-(1-aminocyclopentyl)-N-[(4-nitrophenyl)sulfonyl]norvalinate

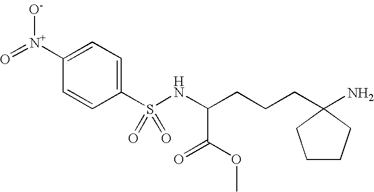

Compound L1-1 (2.95 g, 7.84 mmol) was dissolved in 40 mL of MeOH and treated with 550 mg of 20% Pd(OH)₂. The resulting mixture was hydrogenated at STP for 16 hours, filtered through a pad of celite and evaporated to afford the desired diamine. The diamine was dissolved in 40 mL of DCE and treated sequentially with 2.82 mL of TEA (20.24 mmol) and 1.79 g of 4-nitrobenzenesulfonyl chloride (8.10 mmol). After stirring for 16 hours, the reaction mixture was diluted with DCM and washed with water and brine. The organic phase was dried with MgSO₄, filtered and concentrated. The crude material was employed in Step L1-3 without further purification. LCMS (M+1)=399.8

### Step L1-3 methyl 5-(1-{[N-(methoxycarbonyl)-β-phenyl-L-phenylalanyl]amino}cyclopentyl)-N-[(4-nitrophenyl)sulfonyl]norvalinate

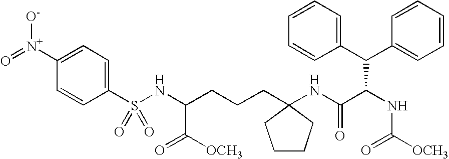

To a solution of the amine from Step L1-2 (1 g, 2.5 mmol) and 2,5-dioxopyrrolidin-1-yl N-(methoxycarbonyl)-β-phenyl-L-phenylalaninate (992 mg, 2.5 mmol) in 1:1 acetone/THF (20 mL) was added 15 mL of saturated NaHCO₃. After stirring for 2 hours at room temperature, the reaction mixture was diluted with DCM and washed with H₂O. The aqueous layer was extracted once with DCM, the organic phases were combined, dried with MgSO₄, filtered and evaporated. Column chromatography (gradient: 50% to 100% EtOAc/hexanes) afforded the desired product. LCMS (M+1)=680.9

### Step L1-4 methyl 5-(1-{[N-(methoxycarbonyl)-β-phenyl-L-phenylalanyl]amino}cyclopentyl)-N-(3-methylbutyl)-N-[(4-nitrophenyl)sulfonyl]norvalinate

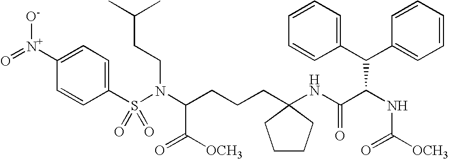

Sulfonamide L1-3 (610 mg, 0.896 mmol) was dissolved in 4.5 mL of THF and treated sequentially with triphenylphosphine (282 mg, 1.08 mmol), isoamyl alcohol (0.117 mL, 1.08 mmol), and DIAD (0.209 mL, 1.08 mmol), and the resulting solution was allowed to stir for 16 hours at room temperature. The reaction mixture was diluted with EtOAc and washed with water. The organic phase with dried with MgSO₄, filtered, concentrated and chromatographed (gradient: 50% to 100% EtOAc/hexanes) to afford the desired product. LCMS (M+1)=751.0

### Step L1-5 methyl N-[(4-aminophenyl)sulfonyl]-5-(1-{[N-(methoxycarbonyl)-β-phenyl-L-phenylalanyl]amino}cyclopentyl)-N-(3-methylbutyl)norvalinate

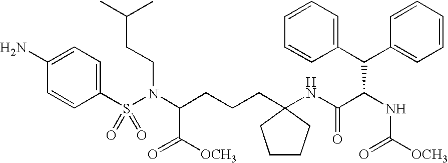

Compound L1-4 (544 mg, 0.724 mmol) was dissolved in 3.6 mL of MeOH and treated with 51 mg of 20% Pd(OH)₂. The resulting mixture was hydrogenated at STP for 16 hours, filtered through a pad of celite and evaporated to afford the desired aniline. LCMS (M+1)=721.1

### Step L1-6 N-(1-{(45)-4-[[(4-aminophenyl)sulfonyl](3-methylbutyl)amino]-5-hydroxypentyl}cyclopentyl)-Nα-(methoxycarbonyl)-β-phenyl-L-phenylalaninamide

To a solution containing 495 mg (0.687 mmol) of L1-5 ester in 3 mL of EtOH was added 0.34 mL of 2M LiBH₄. The reaction mixture was stirred at room temperature for 16 hours, after which 1 mL of water was added and the reaction mixture was stirred at room temperature for 1 hour. The solution was then extracted with EtOAc twice, and the organic phase was washed with water and brine, dried with MgSO₄ and concentrated. The crude material was subjected to reverse phase chromatography and the pure fractions were diluted with EtOAc and rendered basic by the addition of saturated NaHCO₃. The organic phase was separated, dried and evaporated to afford a diasteromeric mixture. Chiral chromatography afforded the desired diastereomer.

¹H NMR (CD3OD): δ 7.48 (d, J=8.6 Hz, 2H), 7.37-7.35 (m, 4H), 7.28-7.24 (m, 4H), 7.19-7.14 (m, 2H), 6.70 (d, J=8.5 Hz, 2H), 4.94 (d, J=11.7 Hz, 1H), 4.25 (d, J=11.4 Hz, 1H), 3.60-3.55 (m, 4H), 3.50-3.39 (m, 2H), 3.16-2.96 (m, 2H), 1.75-1.70 (m, 1H), 1.63-1.11 (m, 16H), 0.88 (d, J=6.14 Hz, 6H). LCMS (M+1)=693.3

### Example L2

### N-{(5S)-5-[[(4-aminophenyl)sulfonyl](isopropyl)amino]-6-hydroxy-1,1-dimethylhexyl}-β-phenyl-L-phenylalaninamide

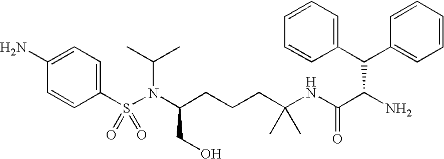

### Step L2-1 methyl (2E)-2-{[(benzyloxy)carbonyl]amino}-6-methyl-6-nitrohept-2-enoate

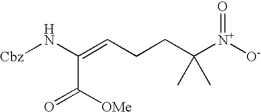

DBU (18.77 mL, 125 mmol) was added to a solution of (+/−)-Benzyloxycarbonyl-alpha-phosphonoglycine trimethyl ester in DCM (200 mL) at −20° C. The mixture was stirred for 5 minutes then a solution of 4-methyl-4-nitrovaleraldehyde in 26 mL of DCM was added slowly to maintain the −20° C. temperature during addition. The mixture was allowed to warm to 0° C. and stir for 5 hours followed by 16 hours at room temperature. The reaction mixture was concentrated, redissolved in EtOAc, and then washed with 1M HCl, saturated NaHCO₃, water and brine. The organic phase was dried over MgSO₄ and concentrated. Column chromatography (gradient: 20% to 100% EtOAc/hexanes) afforded the desired product. LCMS (M+1)=351.0

### Step L2-2 methyl (2S)-2-{[(benzyloxy)carbonyl]amino}-6-methyl-6-nitroheptanoate

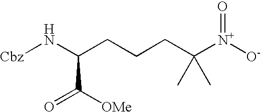

The olefin substrate from L2-1 (12.44 g, 35.5 mmol) and 1,2-Bis[(2S,5S)-2,5-dimethylphospholano]benzene(cyclooctadiene)rhodium(I)tetrafluoroborate (300 mg) were charged in a 50 mL MultiMax™ hydrogenation reaction vessel (Mettler Toledo), followed by 80 mL of MeOH. The mixture was hydrogenated at 50 psi for 24 hours at room temperature. The reaction mixture was concentrated and chromatographed (gradient: 40% to 100% EtOAc/hexanes) to afford the product with a 96% ee. LCMS (M+1)=353.1

### Step L2-3 methyl (2S)-6-amino-2-{[(benzyloxy)carbonyl]amino}-6-methylheptanoate

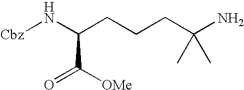

The nitro ester from L2-2 (11.66 g, 33.1 mmol) was dissolved in MeOH at 0° C., after which acetyl chloride (23.53 mL, 331 mmol) was added dropwise to the solution over 10 minutes to maintain a temperature between 0-12° C. Zinc dust (28.1 g, 430 mmol) was then added portionwise to maintain a temperature of approximately 0° C. After the addition was complete the reaction mixture was warmed to 55° C. for 2 hours. The slurry was cooled, filtered, concentrated and chromatographed to afford the desired product. LCMS (M+1)=323.1

### Step L2-4 methyl (2S)-2-{[(benzyloxy)carbonyl]amino}-6-[(tert-butoxycarbonyl)amino]-6-methylheptanoate

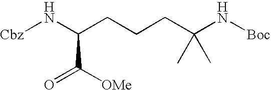

To a solution of amine L2-3 (8 g, 24.8 mmol) in 125 mL of DCM was added 5.19 mL (37.2 mmol) of TEA followed by Boc₂O (5.42 g, 24.8 mmol) and stirred at room temperature for 16 hours. The volume of DCM was reduced and the reaction mixture was chromatographed (gradient: 20% to 100% EtOAc/hexanes) to afford the protected amine. LCMS (M+1)=423.2

### Step L2-5 methyl (2S)-2-amino-6-[(tert-butoxycarbonyl)amino]-6-methylheptanoate

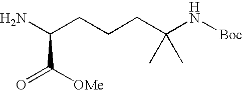

Compound L2-4 (5.48 g, 12.97 mmol) was dissolved in 65 mL of MeOH and treated with 911 mg of 20% Pd(OH)₂. The resulting mixture was hydrogenated at STP for 16 hours, filtered through a pad of celite and evaporated to afford the desired amine. LCMS (M+1) 289.1

### Step L2-6 methyl (2S)-6-[(tert-butoxycarbonyl)amino]-6-methyl-2-{[(4-nitrophenyl)sulfonyl]amino}heptanoate

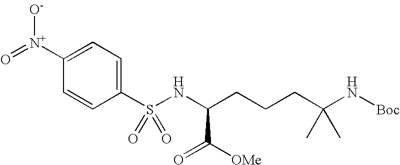

The amine L2-5 (3.43 g, 11.89 mmol) was dissolved in 60 mL of DCM and treated sequentially with 2.49 mL of TEA (17.84 mmol) and 2.64 g of 4-nitrobenzenesulfonyl chloride (11.89 mmol). After stirring for 16 hours, the reaction mixture was diluted with DCM and washed with water and brine. The organic phase was dried with MgSO₄, filtered and concentrated. Column chromatography (gradient: 20% to 100% EtOAc/hexanes) afforded the desired product. LCMS (M+1)=475.1

### Step L2-7 methyl (2S)-6-[(tert-butoxycarbonyl)amino]-2-{isopropyl[(4-nitrophenyl)sulfonyl]amino}-6-methylheptanoate

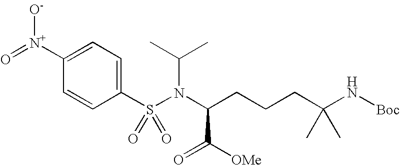

Sulfonamide L2-6 (3.4 g, 7.18 mmol) was dissolved in 36 mL of THF and treated sequentially with triphenylphosphine (2.26 g, 8.62 mmol), 2-propanol (0.66 mL, 8.62 mmol), and DIAD (1.68 mL, 8.62 mmol), and the resulting solution was allowed to stir for 16 hours at room temperature. The reaction mixture was diluted with EtOAc and washed with water. The organic phase with dried with MgSO₄, filtered, concentrated and chromatographed (gradient: 10% to 80% EtOAc/hexanes) to afford the desired product. LCMS (M+1)=516.2

### Step L2-8 methyl (2S)-2-[[(4-aminophenyl)sulfonyl](isopropyl)amino]-6-[(tert-butoxycarbonyl)amino]-6-methylheptanoate

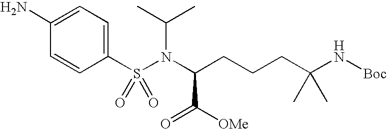

Compound L2-7 (2.31 g, 4.48 mmol) was dissolved in 22 mL of MeOH and treated with 315 mg of 20% Pd(OH)₂. The resulting mixture was hydrogenated at STP for 16 hours, filtered through a pad of celite and evaporated to afford the desired aniline. LCMS (M+1) 486.2

### Step L2-9 tert-butyl {(5S)-5-[[(4-aminophenyl)sulfonyl](isopropyl)amino]-6-hydroxy-1,1-dimethylhexyl}carbamate

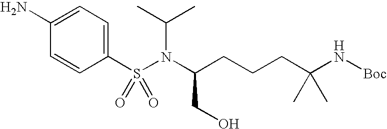

To a solution containing 2.16 g (4.45 mmol) of L2-8 ester in 22 mL of EtOH was added 8.91 mL of 2M LiBH₄. After the reaction mixture was stirred for 2 hours, 5 mL of water was added and the mixture stirred for 30 minutes. The solution was extracted with EtOAc twice, and the organic phase was washed with water and brine, dried with MgSO₄ and then concentrated. Column chromatography (gradient: 50% to 100% EtOAc/hexanes) afforded the desired alcohol. LCMS (M+1)=458.3

### Step L2-10 4-amino-N-((1S)-5-amino-1-(hydroxymethyl)-5-methylhexyl]-N-isopropylbenzenesulfonamide

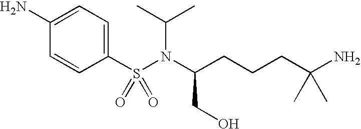

Compound L2-9 (1.62 g, 3.54 mmol) was dissolved in 20 mL of MeOH at 0° C. and then a stream of HCl gas was passed through the solution for 2 minutes. After stirring the reaction mixture an additional 2 hours, the solvent was removed to afford the desired amino alcohol HCl salt which was used in Step L2-11 without further purification. LCMS (M+1)=358.1

### Step L2-11 N-{(5S)-5-[[(4-aminophenyl)sulfonyl](isopropyl)amino]-6-hydroxy-1,1-dimethylhexyl}-Nα-(tert-butoxycarbonyl)-β-phenyl-L-phenylalaninamide

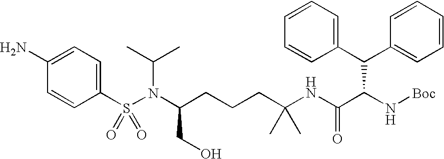

To a solution of the amine HCl salt from step L2-10 (50 mg, 0.127 mmol) and N-Boc-(S)-diphenylalanine (43 mg, 0.127 mmol) in 1 mL of DMF was added diisopropylethylamine (0.07 mL, 0.381 mmol) and BOP-reagent (56 mg, 0.127 mmol). After 2 hours, the reaction mixture was subjected to reverse phase chromatography. The pure fractions were diluted with EtOAc and rendered basic by the addition of saturated NaHCO₃. The organic phase was separated, dried and evaporated to afford the desired product. LCMS (M+1)=681.3

### Step L2-12 N-{(5S)-5-[[(4-aminophenyl)sulfonyl](isopropyl)amino]-6-hydroxy-1,1-dimethylhexyl}-β-phenyl-L-phenylalaninamide

Compound L2-11 (50 mg, 0.073 mmol) was dissolved in 1.5 mL of MeOH at 0° C. after which a stream of HCl gas was passed through the solution for 2 minutes. After stirring the reaction mixture an additional 2 hours, the solvent was removed to afford the desired product HCl salt as a white solid. ¹H NMR (CD3OD): δ 7.81 (d, J=8.5 Hz, 2H), 7.50-7.48 (m, 2H), 7.41-7.37 (m, 41-1), 7.34-7.24 (m, 4H), 7.13 (d, J=8.5 Hz, 2H), 4.68 (d, J=11.3 Hz, 1H), 4.28 (d, J=11.4 Hz, 1H), 3.76-3.70 (m, 1H), 3.60-3.59 (m, 2H), 3.43 (m, 1H), 1.56 (m, 2H), 1.40-1.04 (m, 10H), 1.01 (s, 3H), 0.92 (s, 3H). LCMS (M+1)=581.3

The following examples (Table L) were prepared using similar procedures as described in the preparation of Examples L1 to L2, using the appropriate building blocks (R¹OH, HO₂C—CHR⁶—NHR⁷ or corresponding activated amino acid such as hydroxysuccinate ester). In some cases NHR⁷ was originally protected as Boc which necessitated acidic Boc removal in the last step.

TABLE L

| Example | | |
|---|---|---|
| No. | Structure | M + 1 |
| L3¹ | N-(1-{4-[[(4-aminophenyl)sulfonyl](3- | 679 |
| | methylbutyl)amino]-5-hydroxypentyl}cyclobutyl)- | |
| | Nα-(methoxycarbonyl)-β-phenyl-L- | |
| | phenylalaninamide | |
| | 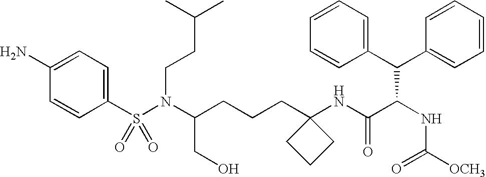 | |
| L4² | N-{(5S)-5-[[(4-aminophenyl)sulfonyl](3- | 667 |
| | methylbutyl)amino]-6-hydroxy-1,1-dimethylhexyl}- | |
| | Nα-(methoxycarbonyl)-β-phenyl-L- | |
| | phenylalaninamide | |
| | 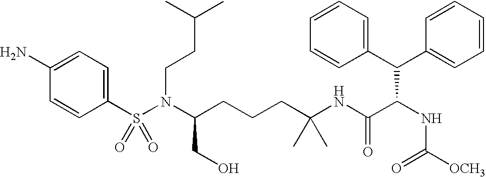 | |
| L5² | N-{(5S)-5-[[(4- | 653 |
| | aminophenyl)sulfonyl](isobutyl)amino]-6-hydroxy- | |
| | 1,1-dimethylhexyl}-Nα-(methoxycarbonyl)-β- | |
| | phenyl-L-phenylalaninamide | |
| | 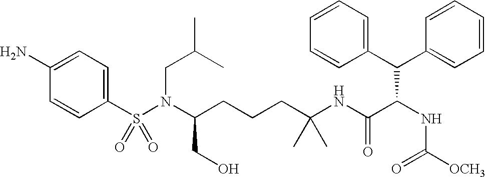 | |
| L6² | N-{(5S)-5-[[(4- | 639 |
| | aminophenyl)sulfonyl](propyl)amino]-6-hydroxy- | |
| | 1,1-dimethylhexyl}-Nα-(methoxycarbonyl)-β- | |
| | phenyl-L-phenylalaninamide | |
| | 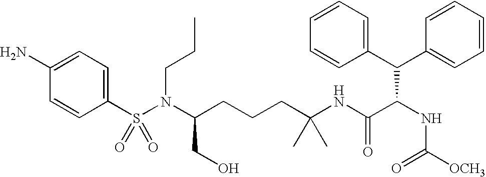 | |
| L7² | N-{(5S)-5-[[(4-aminophenyl)sulfonyl](3- | 657 |
| | fluoropropyl)amino]-6-hydroxy-1,1-dimethylhexyl}- | |
| | Nα-(methoxycarbonyl)-β-phenyl-L- | |
| | phenylalaninamide | |
| | 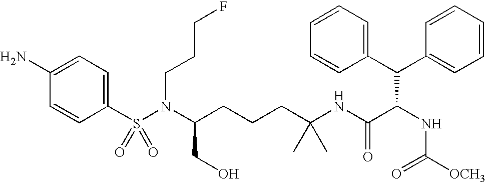 | |
| L8² | N-{(5S)-5-[[(4- | 639 |
| | aminophenyl)sulfonyl](isopropyl)amino9 -6-hydroxy- | |
| | 1,1-dimethylhexyl}-Nα-(methoxycarbonyl)-β- | |
| | phenyl-L-phenylalaninamide | |
| | 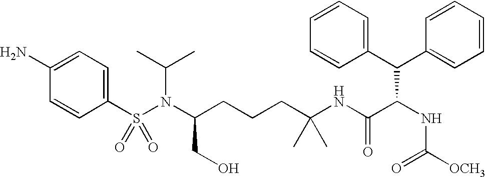 | |
| L9³ | N-{(5S)-5-[[(4- | 595 |
| | aminophenyl)sulfonyl](isopropyl)amino]-6-hydroxy- | |
| | 1,1-dimethylhexyl}-Nα-methyl-β-phenyl-L- | |
| | phenylalaninamide hydrochloride | |
| | 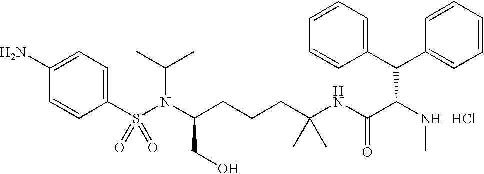 | |
| L10³ | N-{(5S)-5-[[(4- | 539 |
| | aminophenyl)sulfonyl](isopropyl)amino]-6-hydroxy- | |
| | 1,1 -dimethylhexyl}-2-chloro-L-phenylalaninamide | |
| | hydrochloride | |
| | 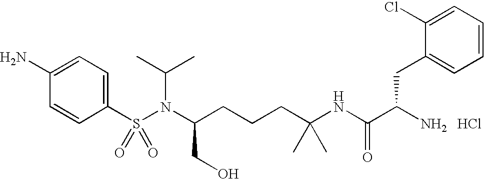 | |

¹The compound was prepared using a procedure analogous to that set forth in Example L1, with RS at hydroxymethyl center.
²The compound was prepared using a procedure analogous to that set forth in Example L2, except there was no Boc removal.
³The compound was prepared using a procedure analogous to that set forth in Example L2.

### Example M1

### N-[5-[[(4-aminophenyl)sulfonyl](3-methylbutyl)amino]-6-hydroxy-1-(hydroxymethyl)hexyl]-Nα-(methoxycarbonyl)-β-phenyl-L-phenylalaninamide

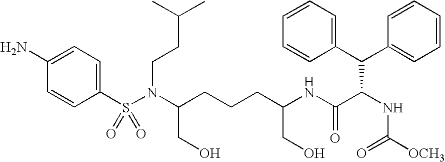

### Step M1-1: 1,7-Dimethoxy-1,7-dioxoheptane-2,6-diaminium dichloride

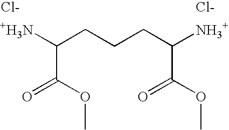

To a 0° C. solution containing 25.0 g (131 mmol) of 2,6-diaminoheptanedioic acid in 100 mL of methanol was added 500 mL 1.25M HCl in MeOH. The solution was warmed to room temperature and stirred for 16 hours. The reaction mixture was cooled to 0° C. and HCl gas was bubbled through for 10 minutes. The resulting mixture was warmed to room temperature and stirred for 16 hours and then concentrated to give the desired product. LCMS [M+H]⁺=219.

### Step M1-2: Dimethyl 2-amino-6-{[(benzyloxy)carboynyl]amino}heptanedioate

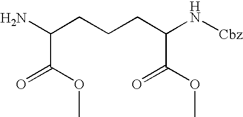

To a 0° C. solution containing 30.6 g (105 mmol) of the diester from step M1-1 in 200 mL of dichloromethane was added 30.8 mL (221 mmol) triethylamine, then 1.5 mL (10.51 mmol) benzylchloroformate dropwise. The solution was warmed to room temperature and stirred for 16 hours and then concentrated. The residue was partitioned between ether and 1N HCl. The aqueous layer was rendered basic with the addition of solid sodium bicarbonate then extracted with ether (4×). The organic extract was washed with brine, dried over Na₂SO₄, concentrated, and chromatographed (0% to 10% MeOH/EtOAc) to afford the desired product. LCMS [M+H]⁺=353.

### Step M1-3: Dimethyl 2-{[(benzyloxy)carbonyl]amino}-6-{[(4-nitrophenyl)sulfonyl]amino}heptanedioate

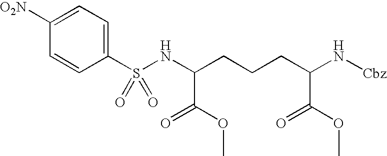

To a solution containing 1.15 g (3.26 mmol) of the amine from step M1-2 in 100 mL DCM was added 0.723 g (3.26 mmol) of p-nitrobenzenesulfonyl chloride followed by 0.46 mL (3.26 mmol) of triethylamine. The resulting mixture was allowed to stir at room temperature for 16 hours. The solution was washed with 1 N HCl, saturated NaHCO₃, water, and brine. The organic phase was dried over Na₂SO₄, concentrated and chromatographed to afford the desired product. LCMS [M+H]⁺=538.

### Step M1-4: Dimethyl 2-{[(benzyloxy)carbonyl]amino}-6-{(3-methylbutyl) [(4-nitrophenyl)sulfonyl]amino}heptanedioate

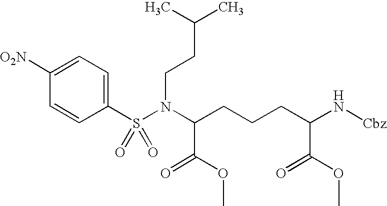

To a solution of 1.52 g (2.83 mmol) the product from step M1-3 in 8 mL THF was added 0.816 g (3.11 mmol) triphenylphosphine then 0.401 mL (3.68 mmol) isoamyl alcohol. The solution was degassed and 0.605 mL (3.11 mmol) DIAD was added. The solution was allowed to stir for 16 hours at room temperature, concentrated, and chromatographed to afford the desired product. LCMS [M+H]⁺=608.

### Step M1-5: Dimethyl 2-amino-6-[[(4-aminophenyl)sulfonyl](3-methylbutyl)amino]heptanedioate

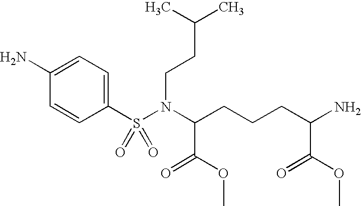

A degassed solution containing 2.0 g (3.29 mmol) of the product of step M1-4 dissolved in 6 mL of MeOH was treated with 2.31 g of 10% Pd(OH)₂ and hydrogenated at STP for 2 hours. The reaction mixture was filtered through celite and concentrated to afford the desired product. LCMS [M+H]⁺=444.

### Step M1-6: Dimethyl 2-[[(4-aminophenyl)sulfonyl](3-methylbutyl)amino]-6-{[N-(methoxycarbonyl)-β-phenyl-L-phenylalanyl]amino}heptanedioate

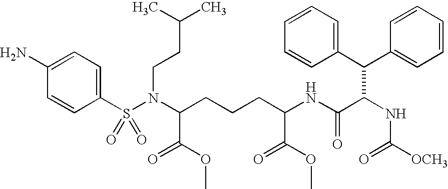

To a solution of 1.66 g (3.74 mmol) of the product of step M1-5 in 8 mL 1:1 THF:acetone was added 1.48 g (3.74 mmol) 2,5-dioxopyrrolidin-1-yl N-(methoxycarbonyl)-β-phenyl-L-phenylalaninate, then 4 mL saturated NaHCO₃. The suspension was allowed to stir 16 hours at room temperature, and then concentrated. The residue was dissolved in DCM, washed with water and brine. The organic phase was dried over Na₂SO₄, concentrated, and chromatographed to afford the desired product. LCMS [M+1]+=725.

### Step M1-7: N45-[[(4-aminophenyl)sulfonyl](3-methylbutyl)amino]-6-hydroxy-1-(hydroxymethyl)hexyl]-Nα-(methoxycarbonyl)-β-phenyl-L-phenylalaninamide

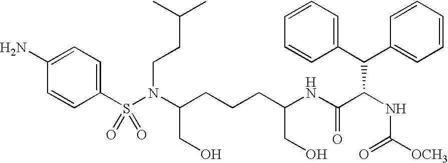

To a solution containing 1.44 g (1.987 mmol) of the product of step M1-6 in 6 mL THF was added 3.97 mL of 2M LiBH₄. The reaction mixture was allowed to stir for 30 minutes before 0.5 mL of MeOH was added. After an additional 1 hour of stirring, the reaction mixture was quenched with MeOH and concentrated. The residue was dissolved in DCM and washed with water and brine. The organic phase was dried over Na₂SO₄, concentrated, and subjected to reverse phase chromatography. Pure fractions were diluted with EtOAc and rendered basic by the addition of saturated NaHCO₃. The organic phase was separated, dried with Na₂SO₄ and evaporated to afford the desired product as a mixture of four diastereomers. The mixture of diastereomers was subjected to chiral chromatography on a chiralpak AD-H (Amylose tris(3,5-dimethylphenylcarbamate) column, 3 cm i.d.×25 cm, 5 μm, 40% IPA in CO₂) to give two pairs of the four possible diastereomers. Each of the pairs were concentrated and subjected to a second chiral chromatography with a different method (Kromasil® chiral (Akzo Nobel) TBB (O,O′-bis(4-tert-butylbenzoyl)-N,N′-diallyl-L-tartar diamide), 3 cm i.d.×25 cm, 5 μm, 25% IPA in CO₂). From each pair were isolated one active and one inactive isomer each. The active diastereoisomers are:

### Example M1-7A

### N-[(1R or 1S,5R or 5S)-5-[[(4-aminophenyl)sulfonyl](3-methylbutyl)amino]-6-hydroxy-1-(hydroxymethyl)hexyl]-Nα-(methoxycarbonyl)-β-phenyl-L-phenylalaninamide (second eluting isomer)

¹H NMR (CDCl₃): δ 7.62-7.61 (m, 2H), 7.33-7.23 (m, 10H), 6.75-6.60 (m, 2H), 5.84 (br s, 1H), 5.25-5.20 (m, 1H), 4.86-4.82 (t, J=9 Hz, 1H), 4.40-4.37 (m, 1H), 3.65-3.50 (m, 7H), 3.20-3.04 (m, 4H), 2.35 (br s, 4H), 1.56-1.21 (m, 8H), 1.00-0.89 (m, 7H). LCMS [M+H]⁺=669.

### Example M1-7B

### N-[(1R or 1S, 5R or 5S)-5-[[(4-aminophenyl)sulfonyl](3-methylbutyl)amino]-6-hydroxy-1-(hydroxymethyl)hexyl]-Nα-(methoxycarbonyl)-β-phenyl-L-phenylalaninamide (fourth eluting isomer)

¹H NMR (CDCl₃): δ 7.62-7.59 (m, 2H), 7.33-7.26 (m, 10H), 6.72-6.70 (m, 2H), 5.69 (br s, 1H), 5.13-5.11 (m, 1H), 4.77-4.3 (t, J=9 Hz, 1H), 4.44-4.42 (d, J=11 Hz, 1H), 4.30 (br s, 2H), 3.61-3.42 (m, 8H), 3.30-3.19 (m, 2H), 3.16-3.01 (m, 1H), 2.62 (br s, 1H), 2.30 (br s, 1H), 1.57 (br s, 3H), 1.26-0.90 (m, 10H), 0.57 (br s, 2H). LCMS [M+H]⁺=669.

### Assay Example 1

### Assay for Inhibition of Microbial Expressed HIV Protease

Inhibition studies of the reaction of the protease (which was expressed in Eschericia coli) with a peptide substrate [Val-Ser-Gln-Asn-(betanapthyl)Ala-Pro-Ile-Val]. The inhibitor is first preincubated with the enzyme in assay buffer (50 mM sodium acetate, pH 5.5, 100 mM NaCl, and 0.1% BSA) for 30 minutes at room temperature. Substrate is added to 440 micromolar in a total volume of 80 microliters containing 5 picomolar HIV-1 protease, and the reaction is incubated for 1 hour at 30° C. The reaction is quenched by addition of 120 microliters of 10% phosphoric acid, and product formation is determined after separation of product and substrate on a Vydac C18 column connected to an Alliance high performance liquid chromatography system (Waters Corporation). The extent of inhibition of the reaction is determined from the peak area of the products. HPLC of the products, independently synthesized, proved quantitation standards and confirmation of the product composition. Representative compounds of the present invention exhibit inhibition of HIV-1 protease in this assay. For example, as shown by their IC₅₀ values in Table 1 below, the compounds set forth in the foregoing Examples exhibit inhibition against the wild-type HIV-1 protease enzyme.

### Assay Example 2

Assay for Inhibition of HIV Replication

Assays for the inhibition of acute HIV infection of T-lymphoid cells were conducted in accordance with Vacca, J. P. et al., Proc. Natl. Acad. Sci. USA 1994, 91: 4096. Representative compounds of the present invention exhibit inhibition of HIV replication in this assay (also referred to herein as the “spread assay”). For example, as shown by their IC₉₅ values in Table 1 below, the compounds set forth in the foregoing Examples were tested in this assay and found to exhibit inhibition of HIV-1 replication.

### Assay Example 3

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention do not exhibit cytotoxicity. For example, all of the exemplified compounds were tested in this assay and none was found to exhibit cytotoxicity.

TABLE 1

| | Enzyme Inhibition - | Spread² - |
|---|---|---|
| Example No.¹ | IC₅₀ (nM) | IC₉₅ (nM) |
| A1 | 0.43 | 140 |
| A2 | 5 | 252 |
| B1 | 0.51 | 500 |
| B2 | 0.52 | 368 |
| C1 | 0.2 | 193 |
| D1(S) | 0.008 | 7 |
| D1(R) | 0.386 | 98 |
| D2 | 0.016 | 10 |
| D3 | 0.054 | 12 |
| D4³ | >3 | 270 |
| D5 | 0.042 | 9 |
| D6 | 0.106 | 56 |
| D7 | 0.064 | 19 |
| D8 | 0.327 | 46 |
| D9 | 0.035 | 8 |
| D10 | 0.099 | 37 |
| D11 | 0.109 | 18 |
| D12 | 0.012 | 6 |
| D13 | 0.867 | 213 |
| D14 | 0.519 | 95 |
| D15 | 0.268 | 39 |
| D16 | 0.329 | 152 |
| D17 | 0.380 | 154 |
| E1 | 0.030 | 11 |
| E2 | 0.017 | 39 |
| E3 | 0.057 | 11 |
| E4 | 0.201 | 22 |
| E5 | 0.053 | 47 |
| E6 | 0.352 | 27 |
| E7 | 0.066 | 39 |
| E8 | 1.397 | 195 |
| E9 | 0.354 | 117 |
| F1 | 0.132 | 32 |
| F2 | 0.028 | 23 |
| F3 | 0.196 | 25 |
| F4 | 0.055 | 65 |
| F5 | 0.163 | 54 |
| F6 | 0.010 | 10 |
| F7 | 0.862 | 251 |
| F8 | 1.446 | 249 |
| F9 | 0.064 | 76 |
| F10 | 0.010 | 17 |
| F11 | 0.021 | 38 |
| F12 | 2.106 | 325 |
| F13 | 0.010 | 15 |
| F14 | 1.761 | 148 |
| F15 | 0.010 | 16 |
| F16 | 0.128 | 58 |
| F17 | 0.010 | 18 |
| G1 | 0.050 | 37 |
| G2 | 0.082 | 14 |
| G3 | 0.158 | 44 |
| G4 | 0.405 | 41 |
| G5 | 0.378 | 64 |
| G6 | 0.376 | 161 |
| G7 | 0.024 | 15 |
| G8 | 3.2 | 191 |
| G9 | 0.629 | 102 |
| G10 | 0.389 | 49 |
| G11 | 1.298 | 93 |
| G12 | 0.055 | 15 |
| G13 | 0.291 | 71 |
| G14 | 0.010 | 16 |
| G15 | 0.584 | 77 |
| G16 | 0.010 | 8 |
| G17 | 0.077 | 137 |
| G18 | 0.755 | 199 |
| G19 | 0.076 | 43 |
| G20 | 2.581 | 309 |
| H1 | 0.022 | 14 |
| H2 | 0.010 | 4 |
| H3 | 0.076 | 18 |
| H4 | 0.173 | 29 |
| H5 | 0.020 | 7 |
| H6 | 1.012 | 138 |
| H7 | 0.131 | 31 |
| I1 | 0.634 | 155 |
| I2 | 2.252 | 422 |
| I3 | 0.134 | 50 |
| I4 | 0.058 | 29 |
| I5 | 0.011 | 85 |
| I6 | 1.029 | 187 |
| I7 | 0.206 | 43 |
| I8 | 0.346 | 43 |
| I9 | 3.200 | 323 |
| I10 | 0.733 | 105 |
| J1 | 0.015 | 9 |
| J2 | 0.012 | 8 |
| J3 | 0.011 | 25 |
| J4 | 0.007 | 6 |
| J5 | 0.291 | 192 |
| J6 | 0.976 | 327 |
| J7 | 0.623 | 359 |
| J8 | 0.015 | 10 |
| J9 | 0.069 | 23 |
| J10 | 0.005 | 4 |
| J11 | 0.185 | 107 |
| J12 | 0.036 | 43 |
| J13 | 0.010 | 6 |
| J14 | 0.183 | 63 |
| J15 | 0.372 | 42 |
| J16 | 0.010 | 25 |
| J17 | 0.010 | 6 |
| J18 | 0.010 | 8 |
| J19 | 0.034 | 11 |
| J20 | 0.020 | 50 |
| J21 | 0.419 | 49 |
| J22 | 0.032 | 15 |
| J23 | 0.025 | 60 |
| J24 | 0.055 | 16 |
| J25 | 0.080 | 17 |
| J26 | 1.330 | 110 |
| J27 | 0.025 | 6 |
| J28 | 0.010 | 8 |
| J29 | 0.039 | 16 |
| J30 | 0.010 | 21 |
| J31 | 0.010 | 23 |
| J32 | 0.010 | 25 |
| J33 | 0.014 | 26 |
| J34 | 0.041 | 20 |
| J35 | 0.569 | 25 |
| J36 | 0.098 | 27 |
| J37 | 0.595 | 37 |
| J38 | 0.098 | 34 |
| J39 | 0.016 | 39 |
| J40 | 0.015 | 34 |
| J41 | 0.564 | 54 |
| J42 | 0.010 | 15 |
| J43 | 0.126 | 116 |
| J44 | 0.081 | 126 |
| J45 | 0.041 | 80 |
| J46 | 0.010 | 18 |
| K1 | 0.018 | 13 |
| K2 | 0.133 | 41 |
| K3 | 0.929 | 112 |
| K4 | 0.015 | 19 |
| K5 | 0.284 | 41 |
| L1 | 0.370 | 274 |
| L2 | 0.018 | 23 |
| L3 | 0.234 | 471 |
| L4 | 0.010 | 41 |
| L5 | 0.154 | 185 |
| L6 | 0.095 | 196 |
| L7 | 0.062 | 178 |
| L8 | 0.267 | 264 |
| L9 | 0.100 | 26 |
| L10 | 3.200 | 241 |
| M1-7A | 0.349 | 302 |
| M1-7B | 0.013 | 230 |

¹No cytotoxicity was observed for any of these compounds in the cytotoxicity assay set forth in Assay Example 3 up to a concentration of 10 μM.
²Conducted using 10% FBS.
³It is believed that the relative lack of activity of D4 in the enzyme inhibition assay can be attributed to the acidic pH at which the assay is run.

Certain compounds of the present invention including certain of the exemplified compounds (e.g., certain compounds encompassed by Formula III) having substitution at the epsilon position (i.e., one or both of R⁵ and R^{5A} in Compound I are other than H) have exhibited better potency in the foregoing assays and/or a better PK profile in animal models than structurally similar compounds that have no branching in the beta, gamma, delta, and epsilon positions (i.e., R³=R⁴=R⁵=R^{5A}=H). Of particular interest in this regard are certain of the compounds encompassed by Formula V.

While the foregoing specification teaches the principles of the present invention, with examples provided for the purpose of illustration, the practice of the invention encompasses all of the usual variations, adaptations and/or modifications that come within the scope of the following claims. All publications, patents and patent applications cited herein are incorporated by reference in their entirety into the disclosure.

1. A compound of Formula I: 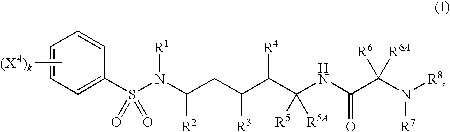
or a pharmaceutically acceptable salt thereof, wherein:
R¹ is C₁₋₆ alkyl, C₁₋₆ fluoroalkyl, C₃₋₆ cycloalkyl, or C₁₋₆ alkyl substituted with C₃₋₆ cycloalkyl;
R² is CH(R^{J})—Z, and Z is OH, NH₂, or OR^{P};
R^{J} is H, C₁₋₆ alkyl, C₁₋₆ fluoroalkyl, or C₁₋₆ alkyl substituted with C₃₋₅ cycloalkyl;
R^{P} is P(O)(OH)₂, P(O)(OM)₂, or C(O)R^{Q};
M is an alkali metal or an alkaline earth metal;
R^{Q} is:
(1) C₁₋₆ alkyl,
(2) C₃₋₆ cycloalkyl,
(3) C₁₋₆ alkyl substituted with C₃₋₆ cycloalkyl,
(4) O—C₁₋₆ alkyl,
(5) O—C₁₋₆ alkyl substituted with O—C₁₋₆ alkyl,
(6) O—C₁₋₆ fluoroalkyl,
(7) C(O)O—C₁₋₆ alkyl,
(8) C(O)—C₁₋₆ alkylene-N(H)—C₁₋₆ alkyl,
(9) C(O)—C₁₋₆ alkylene-N(—C₁₋₆ alkyl)₂,
(10) C₁₋₆ alkyl substituted with C(O)O—C₁₋₆ alkyl,
(11) C₁₋₆ alkyl substituted with C(O)OH,
(12) C₁₋₆ alkyl substituted with C(O)—C₁₋₆ alkyl,
(13) N(H)—C₁₋₆ alkyl,
(14) N(—C₁₋₆ alkyl)₂,
(15) C₁₋₆ alkyl substituted with NH₂, N(H)—C₁₋₆ alkyl, or N(—C₁₋₆ alkyl)₂,
(16) AryA,
(17) C₁₋₆ alkyl substituted with AryA,
(18) O—C₁₋₆ alkyl substituted with AryA,
(19) HetA,
(20) C₁₋₆ alkyl substituted with HetA,
(21) O—C₁₋₆ alkyl substituted with HetA,
(22) HetB, or
(23) O-HetB;
R³ is H, C₁₋₆ alkyl, C₁₋₆ fluoroalkyl, or C₁₋₆ alkyl substituted with C₃₋₆ cycloalkyl;
R⁴ is H, C₁₋₆ alkyl, C₁₋₆ fluoroalkyl, or C₁₋₆ alkyl substituted with C₃₋₆ cycloalkyl;
R⁵ is H, C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl;
R^{5A} is H or C₁₋₆ alkyl;
alternatively, R⁵ and R^{5A} together with the carbon atom to which they are both attached form C₃₋₆ cycloalkyl;
and provided that:
(A) at least one of R³, R⁴, R⁵ and R^{5A} is other than H;
(B) when either or both R⁵ and R^{5A} are other than H, then at least one of R³ and R⁴ is H; and
(C) when R³ and R⁴ are both other than H, then R⁵ and R^{5A} are both H;
each X^{A} is independently:
(1) C₁₋₆ alkyl,
(2) C₃₋₆ cycloalkyl,
(3) C₁₋₆ haloalkyl,
(4) OH
(5) O—C₁₋₆ alkyl,
(6) O—C₁₋₆ haloalkyl,
(7) O—C₃₋₆ cycloalkyl,
(8) SH,
(9) S—C₁₋₆ alkyl,
(10) S—C₁₋₆ haloalkyl,
(11) S—C₃₋₆ cycloalkyl,
(12) halo,
(13) CN,
(14) NO₂,
(15) NH₂,
(16) N(H)—C₁₋₆ alkyl,
(17) N(—C₁₋₆ alkyl)₂,
(18) N(H)C(O)—C₁₋₆ alkyl,
(19) N(H)CH(O),
(20) CH(O),
(21) C(O)—C₁₋₆ alkyl,
(22) C(O)OH,
(23) C(O)O—C₁₋₆ alkyl,
(24) SO₂H,
(25) SO₂—C₁₋₆ alkyl, or
(26) C₁₋₆ alkyl substituted with:
(a) C₃₋₆ cycloalkyl,
(b) C₁₋₆ haloalkyl,
(c) OH
(d) O—C₁₋₆ alkyl,
(e) O—C₁₋₆ haloalkyl,
(f) O—C₃₋₆ cycloalkyl,
(g) SH,
(h) S—C₁₋₆ alkyl,
(i) S—C₁₋₆ haloalkyl,
(j) S—C₃₋₆ cycloalkyl,
(k) halo,
(l) CN,
(m) NO₂,
(n) NH₂,
(o) N(H)—C₁₋₆ alkyl,
(p) N(—C₁₋₆ alkyl)₂,
(q) N(H)C(O)—C₁₋₆ alkyl,
(r) N(H)CH(O),
(s) CH(O),
(t) C(O)—C₁₋₆ alkyl,
(u) C(O)OH,
(v) C(O)O—C₁₋₆ alkyl,
(w) SO₂H, or
(x) SO₂—C₁₋₆ alkyl;
or, alternatively, when two or more X^{A} substituents are present on the phenyl ring and two of the X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
k is an integer equal to 0, 1, 2, or 3;
R⁶ is: 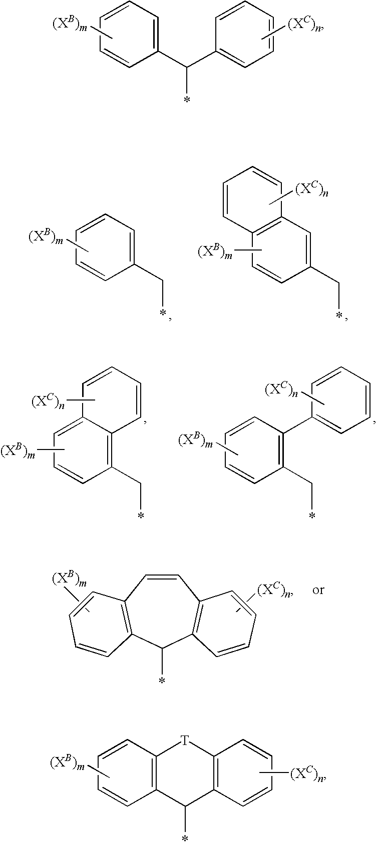
wherein the asterisk (*) denotes the point of attachment to the rest of the compound;
R^{6A} is H or C₁₋₆ alkyl;
alternatively, R⁶ and R^{6A} together with the carbon to which they are attached form a C₃₋₆ cycloalkyl which is optionally substituted with phenyl, wherein the phenyl is optionally substituted with from 1 to 3 X^{B}.
each X^{B} and each X^{C} are independently selected from the group consisting of:
(1) C₁₋₆ alkyl,
(2) C₃₋₆ cycloalkyl,
(3) C₁₋₆ haloalkyl,
(4) OH,
(5) O—C₁₋₆ alkyl,
(6) O—C₁₋₆ haloalkyl,
(7) O—C₃₋₆ cycloalkyl,
(8) SH,
(9) S—C₁₋₆ alkyl,
(10) S—C₁₋₆ haloalkyl,
(11) S—C₃₋₆ cycloalkyl,
(12) halo,
(13) CN,
(14) NO₂,
(15) NH₂,
(16) N(H)—C₁₋₆ alkyl,
(17) N(—C₁₋₆ alkyl)₂,
(18) N(H)C(O)—C₁₋₆ alkyl,
(19) N(H)CH(O),
(20) CH(O),
(21) C(O)—C₁₋₆ alkyl,
(22) C(O)OH,
(23) C(O)O—C₁₋₆ alkyl,
(24) SO₂H,
(25) SO₂—C₁₋₆ alkyl; and
(26) C₁₋₆ alkyl substituted with:
(a) C₁₋₆ haloalkyl,
(b) OH
(c) O—C₁₋₆ alkyl,
(d) O—C₁₋₆ halo alkyl,
(e) O—C₃₋₆ cycloalkyl,
(f) SH,
(g) S—C₁₋₆ alkyl,
(h) halo,
(i) CN,
(j) NO₂,
(k) NH₂,
(l) N(H)—C₁₋₆ alkyl,
(m) N(—C₁₋₆ alkyl)₂,
(n) C(O)—C₁₋₆ alkyl,
(o) C(O)OH,
(p) C(O)O—C₁₋₆ alkyl, or
(q) SO₂—C₁₋₆ alkyl;
T is O, S, S(O), or SO₂;
m is an integer equal to 0, 1, 2, or 3;
n is an integer equal to 0, 1, 2, or 3;
R⁷ is H, C₁₋₆ alkyl, C₃₋₆ cycloalkyl, C₁₋₆ alkyl substituted with C₃₋₆ cycloalkyl, or C(O)—R^{K};
R⁸ is H or C₁₋₆ alkyl;
R^{K} is:
(1) C₁₋₆ alkyl,
(2) C₃₋₆ cycloalkyl,
(3) C₁₋₆ alkyl substituted with C₃₋₆ cycloalkyl,
(4) O—C₁₋₆ alkyl,
(5) O—C₁₋₆ alkyl substituted with O—C₁₋₆ alkyl,
(6) O—C₁₋₆ fluoro alkyl,
(7) C(O)O—C₁₋₆ alkyl,
(8) C₁₋₆ alkyl substituted with C(O)O—C₁₋₆ alkyl,
(9) C₁₋₆ alkyl substituted with C(O)OH,
(10) C₁₋₆ alkyl substituted with C(O)—C₁₋₆ alkyl,
(11) N(H)—C₁₋₆ alkyl,
(12) N(—C₁₋₆ alkyl)₂,
(13) C₁₋₆ alkyl substituted with NH₂, N(H)—C₁₋₆ alkyl, or N(—C₁₋₆ alkyl)₂,
(14) AryA,
(15) C₁₋₆ alkyl substituted with AryA,
(16) O—C₁₋₆ alkyl substituted with AryA,
(17) HetA,
(18) C₁₋₆ alkyl substituted with HetA,
(19) O—C₁₋₆ alkyl substituted with HetA,
(20) HetB,
(21) O-HetB, or
(22) O—C₁₋₆ alkyl substituted with HetB;
each AryA is an aryl which is independently phenyl or naphthyl, wherein the phenyl or naphthyl is optionally substituted with from 1 to 4 Y^{B} wherein each Y^{B} independently has the same definition as X^{B};
each HetA is a heteroaryl which is independently (i) a 5- or 6-membered heteroaromatic ring containing from 1 to 3 heteroatoms independently selected from N, O and S, or (ii) is a heterobicyclic ring selected from quinolinyl, isoquinolinyl, and quinoxalinyl; wherein the heteroaromatic ring (i) or the bicyclic ring (ii) is optionally substituted with from 1 to 4 Y^{C} wherein each Y^{C} independently has the same definition as X^{B}; and
each HetB is independently a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, and wherein the saturated or unsaturated heterocyclic ring is optionally substituted with from 1 to 4 substituents each of which is independently halogen, CN, C₁₋₆ alkyl, OH, oxo, O—C₁₋₆ alkyl, C₁₋₆ haloalkyl, O—C₁₋₆ haloalkyl, C(O)NH₂, C(O)N(H)—C₁₋₆ alkyl, C(O)N(—C₁₋₆ alkyl)₂, C(O)H, C(O)—C₁₋₆ alkyl, CO₂H, CO₂—C₁₋₆ alkyl, SO₂H, or SO₂—C₁₋₆ alkyl.

2. The compound according to claim 1, which is a compound of Formula I-A: 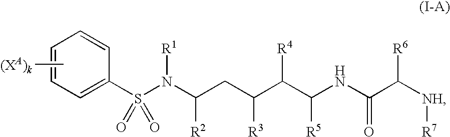
or a pharmaceutically acceptable salt thereof, wherein:
R¹ is C₁₋₆ alkyl or C₁₋₆ alkyl substituted with C₃₋₆ cycloalkyl;
R³ is H, C₁₋₆ alkyl, C₁₋₆ fluoroalkyl, or C₁₋₆ alkyl substituted with C₃₋₅ cycloalkyl;
R⁴ is H, C₁₋₆ alkyl, C₁₋₆ fluoroalkyl, or C₁₋₆ alkyl substituted with C₃₋₅ cycloalkyl;
R⁵ is H, C₁₋₆ alkyl, C₁₋₆ fluoroalkyl, or C₁₋₆ alkyl substituted with C₃₋₅ cycloalkyl;
provided that:
(A) at least one of R³, R⁴, and R⁵ is C₁₋₆ alkyl, C₁₋₆ fluoroalkyl, or C₁₋₆ alkyl substituted with C₃₋₅ cycloalkyl; and
(B) at least one of R³, R⁴, and R⁵ is H; and
each X^{A} is independently as defined in claim 1;
or, alternatively, when two or more X^{A} substituents are present on the phenyl ring and two of the X^{A} are attached to adjacent carbon atoms of the phenyl ring, the two X^{A} are optionally taken together to form —OCH₂O— or —OCH₂CH₂O—;
R⁶ is: 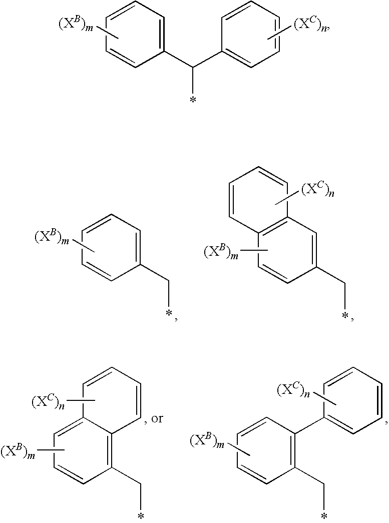
wherein the asterisk (*) denotes the point of attachment to the rest of the compound; and
R^{K} is:
(1) C₁₋₆ alkyl,
(2) C₃₋₆ cycloalkyl,
(3) C₁₋₆ alkyl substituted with C₃₋₆ cycloalkyl,
(4) O—C₁₋₆ alkyl,
(5) O—C₁₋₆ alkyl substituted with O—C₁₋₆ alkyl,
(6) O—C₁₋₆ fluoroalkyl,
(7) C(O)O—C₁₋₆ alkyl,
(8) C₁₋₆ alkyl substituted with C(O)O—C₁₋₆ alkyl,
(9) C₁₋₆ alkyl substituted with C(O)OH,
(10) C₁₋₆ alkyl substituted with C(O)—C₁₋₆ alkyl,
(11) N(H)—C₁₋₆ alkyl,
(12) N(—C₁₋₆ alkyl)₂,
(13) C₁₋₆ alkyl substituted with NH₂, N(H)—C₁₋₆ alkyl, or N(—C₁₋₆ alkyl)₂,
(14) AryA,
(15) C₁₋₆ alkyl substituted with AryA,
(16) O—C₁₋₆ alkyl substituted with AryA,
(17) HetA,
(18) C₁₋₆ alkyl substituted with HetA,
(19) O—C₁₋₆ alkyl substituted with HetA,
(20) HetB, or
(21) O-HetB.

3. The compound according to claim 1, or a pharmaceutically acceptable salt thereof, wherein
R¹ is C₁₋₆ alkyl, C₁₋₆ fluoroalkyl, C₃₋₅ cycloalkyl, or CH₂—C₃₋₅ cycloalkyl;
R² is CH₂—Z, CH(CH₃)—Z, CH(CF₃)—Z; wherein Z is OH, NH₂, or OR^{P}; and wherein R^{P} is P(O)(OH)₂, P(O)(ONa)₂, P(O)(OK)₂, C(O)—C₁₋₆ alkyl, C(O)O—C₁₋₆ alkyl, C(O)N(—C₁₋₆ alkyl)₂, C(O)-pyridyl, or C(O)—C₁₋₆ alkylene-NH₂;
R³ is H, C₁₋₄ alkyl, C₁₋₄ fluoroalkyl, or CH₂—C₃₋₅ cycloalkyl;
R⁴ is H, C₁₋₄ alkyl, C₁₋₄ fluoroalkyl, or CH₂—C₃₋₅ cycloalkyl;
R⁵ is H, C₁₋₄ alkyl, C₁₋₄ fluoroalkyl, C₁₋₄ alkyl substituted with OH, C₂₋₄ alkenyl, C₂₋₄ alkynyl, C₃₋₅ cycloalkyl, or CH₂—C₃₋₅ cycloalkyl;
R^{5A} is H or C₁₋₄ alkyl;
alternatively, R⁵ and R^{5A} together with the carbon atom to which they are both attached form C₃₋₅ cycloalkyl;
and provided that:
(A) at least one of R³, R⁴, R⁵ and R^{5A} is other than H;
(B) when either or both R⁵ and R^{5A} are other than H, then at least one of R³ and R⁴ is H; and
(C) when R³ and R⁴ are both other than H, then R⁵ and R^{5A} are both H;
R⁶ is: 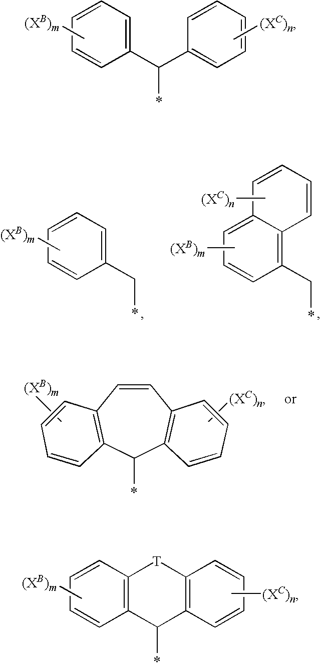
wherein the asterisk (*) denotes the point of attachment to the rest of the compound;
R^{6A} is H or C₁₋₄ alkyl;
alternatively, R⁶ and R^{6A} together with the carbon to which they are attached form a C₃₋₅ cycloalkyl which is optionally substituted with phenyl, wherein the phenyl is optionally substituted with from 1 to 2 X^{B};
each X^{B} and each X^{C} are independently selected from the group consisting of:
(1) C₁₋₃ alkyl,
(2) cyclopropyl,
(3) CF₃,
(4) OH,
(5) O—C₁₋₃ alkyl,
(6) OCF₃,
(7) Cl,
(8) Br,
(9) F,
(10) CN,
(11) NO₂,
(12) NH₂,
(13) N(H)—C₁₋₃ alkyl,
(14) N(—C₁₋₃ alkyl)₂,
(15) C(O)—C₁₋₃ alkyl,
(16) CO₂H,
(17) C(O)O—C₁₋₃ alkyl,
(18) CH₂OH, and
(19) CH₂O—C₁₋₃ alkyl;
m is an integer equal to 0, 1, or 2;
n is an integer equal to 0, 1, or 2;
each X^{A} is independently:
(1) C₁₋₃ alkyl,
(2) cyclopropyl,
(3) CF₃,
(4) OH,
(5) O—C₁₋₃ alkyl,
(6) OCF₃,
(7) Cl,
(8) Br,
(9) F,
(10) CN,
(11) NO₂,
(12) NH₂,
(13) N(H)—C₁₋₃ alkyl,
(14) N(—C₁₋₃ alkyl)₂,
(15) C(O)—C₁₋₃ alkyl,
(16) CO₂H,
(17) C(O)O—C₁₋₃ alkyl, or
(18) C₁₋₃ alkyl substituted with
(a) cyclopropyl,
(b) CF₃,
(c) OH,
(d) O—C₁₋₃ alkyl,
(e) OCF₃,
(f) Cl,
(g) Br,
(h) F,
(i) CN,
(j) NO₂,
(k) NH₂,
(l) N(H)—C₁₋₃ alkyl,
(m) N(—C₁₋₃ alkyl)₂,
(n) C(O)—C₁₋₃ alkyl,
(o)CO₂H, or
(p) C(O)O—C₁₋₃ alkyl;
k is an integer equal to 0, 1, or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
R⁷ is H, C₁₋₆ alkyl, C(O)—C₁₋₆ alkyl, C(O)O—C₁₋₆ alkyl, C(O)N(—C₁₋₆ alkyl)₂, C(O)—HetA, C(O)OCH₂-HetA, C(O)—HetB, or C(O)OCH₂-HetB;
R⁸ is H or C₁₋₄ alkyl;
HetA is a heteroaryl selected from the group consisting of pyrrolyl, imidazolyl, pyridyl, pyrazinyl, quinolyl, isoquinolyl, and quinoxalinyl, wherein the heteroaryl is optionally substituted with from 1 to 3 substituents each of which is independently CH₃, CF₃, OH, OCH₃, OCF₃, Cl, Br, F, CN, NH₂, N(H)CH₃, N(CH₃)₂, C(O)CH₃, CO₂CH₃, or SO₂CH₃; and
HetB is a saturated heterocyclic ring selected from the group consisting of tetrahydrofuranyl, pyrrolidinyl, piperidinyl, piperazinyl, morpholinyl, or thiomorpholinyl in which the S is optionally oxidized to S(O) or S(O)₂, and wherein the ring is optionally substituted with 1 or 2 substituents each of which is independently CH₃, CH₂CH₃, oxo, C(O)N(CH₃)₂, C(O)CH₃, CO₂CH₃, or S(O)₂CH₃.

4. The compound according to claim 3, which is a compound of Formula I-A: 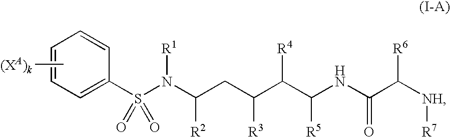
or a pharmaceutically acceptable salt thereof, wherein:
R¹ is C₁₋₆ alkyl;
R³ is H, CH₃, CF₃, CH₂-cyclopropyl, or CH₂-cyclobutyl;
R⁴ is H, CH₃, CF₃, CH₂-cyclopropyl, or CH₂-cyclobutyl;
R⁵ is H, CH₃, CF₃, CH₂-cyclopropyl, or CH₂-cyclobutyl;
provided that:
(A) at least one of R³, R⁴, and R⁵ is CH₃, CF₃, CH₂-cyclopropyl, or CH₂-cyclobutyl; and
(B) at least one of R³, R⁴, and R⁵ is H;
R⁶ is: 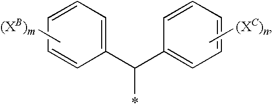
wherein the asterisk (*) denotes the point of attachment to the rest of the compound;
each X^{A} is independently:
(1) C₁₋₃ alkyl,
(2) cyclopropyl,
(3) CF₃,
(4) OH,
(5) O—C₁₋₃ alkyl,
(6) OCF₃,
(7) Cl,
(8) Br,
(9) F,
(10) CN,
(11) NO₂,
(12) NH₂,
(13) N(H)—C₁₋₃ alkyl,
(14) N(—C₁₋₃ alkyl)₂,
(15) C(O)—C₁₋₃ alkyl,
(16) CO₂H,
(17) C(O)O—C₁₋₃ alkyl, or
(18) C₁₋₃ alkyl substituted with
(a) cyclopropyl,
(b) CF₃,
(c) OH,
(d) O—C₁₋₃ alkyl,
(e) OCF₃,
(f) Cl,
(g) Br,
(h) F,
(i) CN,
(j) NO₂,
(k) NH₂,
(l) N(H)—C₁₋₃ alkyl,
(m) N(—C₁₋₃ alkyl)₂,
(n) C(O)—C₁₋₃ alkyl,
(o) CO₂H, or
(p) C(O)O—C₁₋₃ alkyl;
k is an integer equal to 0, 1, or 2; and
R⁷ is H, C(O)—C₁₋₆ alkyl, C(O)O—C₁₋₆ alkyl, C(O)N(—C₁₋₆ alkyl)₂, C(O)—HetA, or C(O)—HetB.

5. The compound according to claim 3, or a pharmaceutically acceptable salt thereof, wherein:
R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclopropyl, cyclobutyl, CH₂-cyclopropyl, or CH₂-cyclobutyl;
R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃;
R³ is H or CH₃;
R⁴ is H or CH₃;
R⁵ is H, CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl;
R^{5A} is H or CH₃;
alternatively, R⁵ and R^{5A} together with the carbon atom to which they are both attached form C₃₋₅ cycloalkyl;
and provided that:
(A) at least one of R³, R⁴, R⁵ and R^{5A} is other than H;
(B) when either or both R⁵ and R^{5A} are other than H, then at least one of R³ and R⁴ is H; and
(C) when R³ and R⁴ are both other than H, then R⁵ and R^{5A} are both H;
R⁶ is: 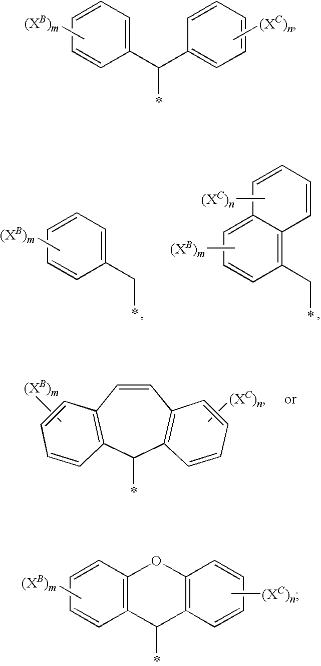
R^{6A} is H;
alternatively, R⁶ and R^{6A} together with the carbon to which they are attached form cyclopropyl which is substituted with phenyl, wherein the phenyl is optionally substituted with from 1 to 2 X^{B};
each X^{B} and each X^{C} are independently selected from the group consisting of:
(1) CH₃,
(2) CH₂CH₃,
(3) CF₃,
(4) OH,
(5) OCH₃,
(6) OCF₃,
(7) Cl,
(8) Br,
(9) F,
(10) CN,
(11) NH₂,
(12) N(H)CH₃,
(13) N(CH₃)₂,
(14) C(O)CH₃,
(15) C(O)OCH₃,
(16) CH₂OH, and
(17) CH₂OCH₃;
each X^{A} is independently:
(1) CH₃,
(2) CH₂CH₃,
(3) CF₃,
(4) OH,
(5) OCH₃,
(6) OCF₃,
(7) Cl,
(8) Br,
(9) F,
(10) CN,
(11) NH₂,
(12) N(H)CH₃,
(13) N(CH₃)₂,
(14) C(O)CH₃,
(15) C(O)OCH₃,
(16) CH₂OH,
(17) CH₂OCH₃,
(18) CH₂NH₂,
(19) CH₂N(H)CH₃,
(20) CH₂N(CH₃)₂,
(21) CH(CH₃)OH,
(22) CH(CH₃)OCH₃,
(23) CH(CH₃)NH₂,
(24) CH(CH₃)N(H)CH₃, or
(25) CH(CH₃)N(CH₃)₂;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
R⁷ is H, CH₃, C(O)CH₃, C(O)OCH₃, C(O)OC(CH₃)₃, C(O)N(CH₃)₂, C(O)-morpholinyl, C(O)-pyridyl, or C(O)O—CH₂-pyridyl; and
R⁸ is H or CH₃.

6. The compound according to claim 5, or a pharmaceutically acceptable salt thereof, wherein:
R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl;
R² is CH₂OH, CH(CH₃)OH, or CH₂NH₂;
R³ is H or CH₃;
R⁴ is H or CH₃;
R⁵ is H, CH₃, CH₂CH₃, CH(CH₃)₂, CH₂CH₂CH₃, C(CH₃)₃, CF₃, CF₂CF₃, CH₂OH, ethenyl, ethynyl, or cyclopropyl;
R^{5A} is H or CH₃, with the proviso that when R^{5A} is CH₃, then R⁵ is CH₃;
alternatively, R⁵ and R^{5A} together with the carbon atom to which they are both attached form cyclobutyl or cyclopentyl;
and provided that:
(A) at least one of R³, R⁴, R⁵ and R^{5A} is other than H;
(B) when either or both R⁵ and R^{5A} are other than H, then at least one of R³ and R⁴ is H; and
(C) when R³ and R⁴ are both other than H, then R⁵ and R^{5A} are both H;
R⁶ is: 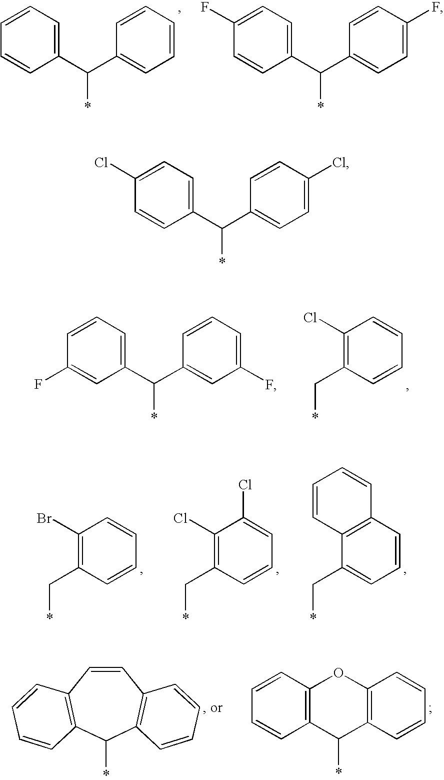
R^{6A} is H;
alternatively, R⁶ and R^{6A} together with the carbon to which they are attached form cyclopropyl substituted with phenyl;
there are 1 or 2×A groups on the phenylsulfonyl moiety wherein one X^{A} is in the para position on the phenyl ring and is CH₃, Cl, Br, F, NH₂, C(O)CH₃, CH₂OH, or CH(CH₃)OH; and the other, optional X^{A} is in the meta position on the phenyl ring and is Cl, Br, or F;
or, alternatively, when two X^{A} substituents are present on the phenyl ring and the two X^{A} are attached to adjacent carbon atoms, the two X^{A} are optionally taken together with the carbon atoms to which they are attached to form a thiazole that is fused to the phenyl ring to provide 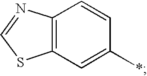
R⁷ is H, CH₃, C(O)OCH₃, C(O)OC(CH₃)₃, or C(O)O—CH₂-pyridyl; and
R⁸ is H or CH₃.

7. The compound according to claim 6, or a pharmaceutically acceptable salt thereof, wherein:
R² is CH₂OH;
R³ is H;
R⁴ is H;
and provided that either or both R⁵ and R^{5A} are other than H;
R^{6A} is H;
R⁷ is C(O)OCH₃ and
R⁸ is H.

8. The compound according to claim 1, which is a compound of Formula II: 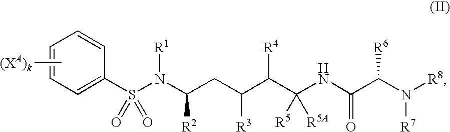
or a pharmaceutically acceptable salt thereof.

9. The compound according to claim 1, which is a compound of Formula III: 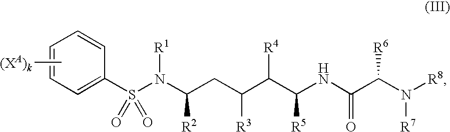
or a pharmaceutically acceptable salt thereof; wherein:
R⁵ is C₁₋₆ alkyl, C₁₋₆ fluoroalkyl, C₁₋₆ alkyl substituted with OH, C₂₋₆ alkenyl, C₂₋₆ alkynyl, C₃₋₆ cycloalkyl, or C₁₋₆ alkyl substituted with C₃₋₆ cycloalkyl.

10. The compound according to claim 9, or a pharmaceutically acceptable salt thereof, wherein:
R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclopropyl, cyclobutyl, CH₂-cyclopropyl, or CH₂-cyclobutyl;
R² is CH₂OH, CH(CH₃)OH, CH₂NH₂, CH(CH₃)NH₂, CH₂OR^{P}, or CH(CH₃)—OR^{P}; wherein R^{P} is P(O)(OH)₂, P(O)(ONa)₂, or C(O)CH₃;
R³ is H or CH₃;
R⁴ is H or CH₃;
R⁵ is CH₃, CH₂CH₃, CH(CH₃)₂, CH₂CH₂CH₃, C(CH₃)₃, CF₃, CF₂CF₃, CH₂OH, ethenyl, ethynyl, cyclopropyl, cyclobutyl, CH₂-cyclopropyl, or CH₂-cyclobutyl;
and provided that at least one of R³ and R⁴ is H;
R⁶ is: 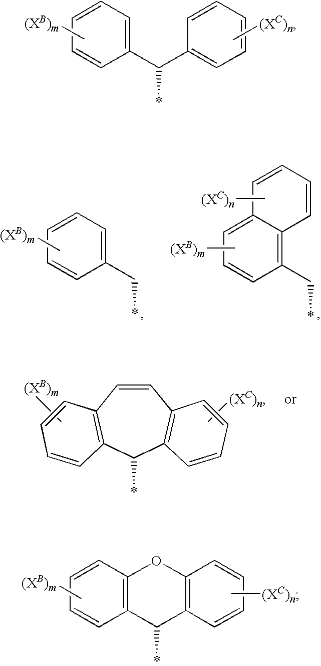
each X^{B} and each X^{C} are independently selected from the group consisting of:
(1) CH₃,
(2) CH₂CH₃,
(3) CF₃,
(4) OH,
(5) OCH₃,
(6) OCF₃,
(7) Cl,
(8) Br,
(9) F,
(10) CN,
(11) NH₂,
(12) N(H)CH₃,
(13) N(CH₃)₂,
(14) C(O)CH₃,
(15) C(O)OCH₃,
(16) CH₂OH, and
(17) CH₂OCH₃;
m is 0, 1 or 2;
n is 0, 1, or 2;
each X^{A} is independently:
(1) CH₃,
(2) CH₂CH₃,
(3) CF₃,
(4) OH,
(5) OCH₃,
(6) OCF₃,
(7) Cl,
(8) Br,
(9) F,
(10) CN,
(11) NH₂,
(12) N(H)CH₃,
(13) N(CH₃)₂,
(14) C(O)CH₃,
(15) C(O)OCH₃,
(16) CH₂OH,
(17) CH₂OCH₃,
(18) CH₂NH₂,
(19) CH₂N(H)CH₃,
(20) CH₂N(CH₃)₂,
(21) CH(CH₃)OH,
(22) CH(CH₃)OCH₃,
(23) CH(CH₃)NH₂,
(24) CH(CH₃)N(H)CH₃, or
(25) CH(CH₃)N(CH₃)₂;
k is 0, 1, or 2;
or, alternatively, when two X^{A} substituents are present on the phenyl ring and the two X^{A} are attached to adjacent carbon atoms of the phenyl ring, the two X^{A} are optionally taken together with the carbon atoms to which they are attached to form a 5- or 6-membered, saturated or unsaturated heterocycle fused to the phenyl ring, wherein the heterocycle contains from 1 to 2 heteroatoms independently selected from N, O and S;
R⁷ is H, CH₃, C(O)CH₃, C(O)OCH₃, C(O)OC(CH₃)₃, C(O)N(CH₃)₂, C(O)-morpholinyl, C(O)-pyridyl, or C(O)O—CH₂-pyridyl; and
R⁸ is H or CH₃.

11. The compound according to claim 10, or a pharmaceutically acceptable salt thereof, wherein:
R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl;
R² is CH₂OH, CH(CH₃)OH, or CH₂NH₂;
R³ is H or CH₃;
R⁴ is H or CH₃;
R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl;
and provided that at least one of R³ and R⁴ is H;
R⁶ is: 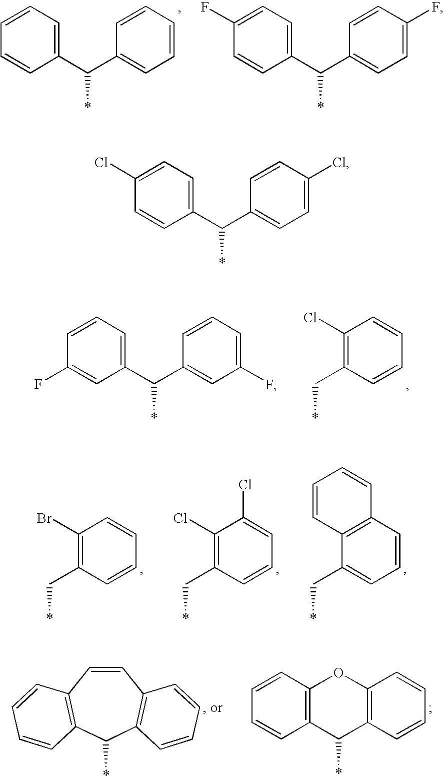
there are 1 or 2×A groups on the phenylsulfonyl moiety wherein one X^{A} is in the para position on the phenyl ring and is CH₃, Cl, Br, F, NH₂, C(O)CH₃, CH₂OH, or CH(CH₃)OH; and the other, optional X^{A} is in the meta position on the phenyl ring and is Cl, Br, or F;
or, alternatively, when two X^{A} substituents are present on the phenyl ring and the two X^{A} are attached to adjacent carbon atoms, the two X^{A} are optionally taken together with the carbon atoms to which they are attached to form a thiazole that is fused to the phenyl ring to provide 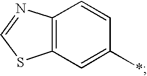
R⁷ is H, CH₃, C(O)OCH₃, C(O)OC(CH₃)₃, or C(O)O—CH₂-pyridyl; and
R⁸ is H or CH₃.

12. The compound according to claim 11, or a pharmaceutically acceptable salt thereof, wherein:
R² is CH₂OH;
R³ is H;
R⁴ is H;
R⁷ is C(O)OCH₃ and
R⁸ is H.

13. The compound according to claim 9, which is a compound of Formula IV: 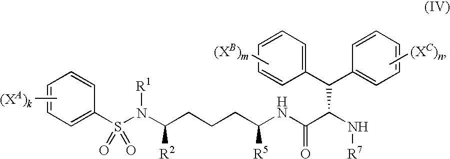
or a pharmaceutically acceptable salt thereof.

14. The compound according to claim 13, which is a compound of Formula V: 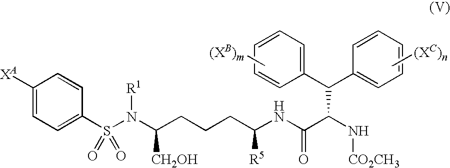
or a pharmaceutically acceptable salt thereof, wherein:
R¹ is CH₃, CH₂CH₃, CH(CH₃)₂, CH₂CH₂CH₃, CH₂CH(CH₃)₂, CH₂CH₂CH(CH₃)₂, CH₂CH₂CH₂F, cyclobutyl, or CH₂-cyclopropyl;
R⁵ is CH₃, CH₂CH₃, CH(CH₃)₂, CH₂CH₂CH₃, C(CH₃)₃, CF₃, CF₂CF₃, CH₂OH, ethenyl, ethynyl, or cyclopropyl;
X^{A} is NH₂, C(O)CH₃, CH₂OH, or CH(CH₃)OH;
each X^{B} and each X^{C} are independently selected from the group consisting of:
(1) CH₃,
(2) CH₂CH₃,
(3) CF₃,
(4) OH,
(5) OCH₃,
(6) OCF₃,
(7) Cl,
(8) Br,
(9) F,
(10) CN,
(11) NH₂,
(12) N(H)CH₃,
(13) N(CH₃)₂,
(14) C(O)CH₃,
(15) C(O)OCH₃,
(16) CH₂OH, and
(17) CH₂OCH₃;
m is an integer equal to 0, 1, or 2; and
n is an integer equal to 0, 1, or 2.

15. The compound according to claim 14, or a pharmaceutically acceptable salt thereof, wherein
R¹ is CH(CH₃)₂, CH₂CH(CH₃)₂, or CH₂CH₂CH(CH₃)₂.

16. The compound according to claim 15, or a pharmaceutically acceptable salt thereof, wherein m and n are either both 0 or both 1; and X^{B} and X^{C} are (i) both F and both para substituents, (ii) both F and both meta substituents, or (iii) both Cl and both para substituents.

17. A compound selected from the group consisting of:
(2S)-2-amino-N-((5S)-6-hydroxy-3-methyl-5-{(3-methylbutyl)[(4-methylphenyl)sulfonyl]-amino}hexyl)-3,3-diphenylpropanamide;
methyl {(1S)-1-(diphenylmethyl)-2-[((5S)-6-hydroxy-3-methyl-5-{(3-methylbutyl)[(4-methylphenyl)-sulfonyl]amino}hexylamino]-2-oxo ethyl}carbamate;
methyl {(1S)-1-(diphenylmethyl)-2-[((5S)-6-hydroxy-2-methyl-5-{(3-methylbutyl)[(4-methylphenyl)sulfonyl]amino}hexylamino]-2-oxo ethyl}carbamate;
methyl [(1S)-2-({(5S)-5-[[4-aminophenyl)sulfonyl]-((3S)-3-methylbutyl)amino]-6-hydroxy-1-methylhexyl)amino)-1-(diphenylmethyl)-2-oxoethyl]carbamate;
methyl [(1S)-2-({(5S)-5-[[4-aminophenyl)sulfonyl]-((3R)-3-methylbutyl)amino]-6-hydroxy-1-methylhexyl)amino)-1-(diphenylmethyl)-2-oxoethyl]carbamate;
methyl [(1S)-2-({(5S)-5-[[4-aminophenyl)sulfonyl]-((3S)-3-ethylbutyl)amino]-6-hydroxy-1-methylhexyl)amino)-1-(diphenylmethyl)-2-oxoethyl]carbamate;
N-{(1S,5S)-5-[[(4-amino-3-fluorophenyl)sulfonyl](3-methylbutyl)amino]-1-ethyl-6-hydroxyhexyl}-Nα-(methoxycarbonyl)-b-phenyl-L-phenylalaninamide;
N-{(1S,5S)-6-amino-5-[[(4-aminophenyl)sulfonyl](3-methylbutyl)amino]-1-methylhexyl}-2-chloro-Nα-(methoxycarbonyl)-L-phenylalaninamide;
N-{(1S,5S)-5-[(1,3-benzothiazol-6-ylsulfonyl)(3-methylbutyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](3-methylbutyl)amino]-6-hydroxy-1-propylhexyl}-Nα-(methoxycarbonyl)-β-phenyl-L-phenylalaninamide;
N-{(1S,5S)-5-[[(4-aminophenyl)sulfonyl](3-methylbutyl)amino]-6-hydroxy-1-methylhexyl}-2-chloro-Nα-(methoxycarbonyl)-L-phenylalaninamide;
N-{(1S,5S)-5-[[(4-aminophenyl)sulfonyl](isobutyl)amino]-6-hydroxy-1-methylhexyl}-2-chloro-Nα-(methoxycarbonyl)-L-phenylalaninamide;
N-{(1S,5S)-5-[[(4-aminophenyl)sulfonyl](isobutyl)amino]-6-hydroxy-1-methylhexyl}-Nα-(methoxycarbonyl)-β-phenyl-L-phenylalaninamide;
N-{(1R,5S)-5-[[(4-aminophenyl)sulfonyl](3-methylbutyl)amino]-6-hydroxy-1-isopropylhexyl}-Nα-(methoxycarbonyl)-β-phenyl-L-phenylalaninamide;
N-{(1S,5S)-5-[[(4-aminophenyl)sulfonyl](isobutyl)amino]-1-ethyl-6-hydroxyhexyl}-Nα-(methoxycarbonyl)-β-phenyl-L-phenylalaninamide;
N-{(1S,5S)-5-[[(4-amino-3-fluorophenyl)sulfonyl](3-methylbutyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](3-fluoropropyl)amino]-6-hydroxy-1-propylhexyl}-Nα-(methoxycarbonyl)-β-phenyl-L-phenylalaninamide;
N-{(1R,5S)-5-[[(4-aminophenyl)sulfonyl](isobutyl)amino]-6-hydroxy-1-isopropylhexyl}-Nα-(methoxycarbonyl)-β-phenyl-L-phenylalaninamide;
N-{(1S,5S)-5-[[(4-aminophenyl)sulfonyl](3-fluoropropyl)amino]-1-ethyl-6-hydroxyhexyl}-Nα-(methoxycarbonyl)-β-phenyl-L-phenylalaninamide;
N-{(1R,5S)-5-[[(4-amino-3-fluorophenyl)sulfonyl](3-methylbutyl)amino]-6-hydroxy-1-isopropylhexyl}-Nα-(methoxycarbonyl)-β-phenyl-L-phenylalaninamide;
N-{(1S,5S)-5-[[(4-amino-3-bromophenyl)sulfonyl](3-methylbutyl)amino]-1-ethyl-6-hydroxyhexyl}-Nα-(methoxycarbonyl)-β-phenyl-L-phenylalaninamide;
methyl [(1S)-2-({(5S)-5-[[3-fluoro-4-aminophenyl)sulfonyl]-((3S)-3-cyclopropylbutyl)amino]-6-hydroxy-1-methylhexyl)amino)-1-(diphenylmethyl)-2-oxo ethyl]carbamate;
N-{(1R,5S)-1-cyclopropyl-5-[{[3-fluoro-4-(hydroxymethyl)phenyl]sulfonyl}(3-methylbutyl)amino]-6-hydroxyhexyl}-4-fluoro-β-(4-fluorophenyl)-Nα-(methoxycarbonyl)-L-phenylalaninamide;
N-{(1R,5S)-5-[[(4-amino-3-fluorophenyl)sulfonyl](3-methylbutyl)amino]-1-cyclopropyl-6-hydroxyhexyl}-Nα-(methoxycarbonyl)-β-phenyl-L-phenylalaninamide;
N-{(1R,5S)-5-[[(4-chlorophenyl)sulfonyl](3-methylbutyl)amino]-1-cyclopropyl-6-hydroxyhexyl}-Nα-(methoxycarbonyl)-β-phenyl-L-phenylalaninamide;
N-{(1R,5S)-5-[[(4-acetylphenyl)sulfonyl](3-methylbutyl)amino]-1-cyclopropyl-6-hydroxyhexyl}-4-fluoro-β-(4-fluorophenyl)-Nα-(methoxycarbonyl)-L-phenylalaninamide;
N-{(1R,5S)-5-[(1,3-benzothiazol-6-ylsulfonyl)(isobutyl)amino]-1-cyclopropyl-6-hydroxyhexyl}-Nα-(methoxycarbonyl)-β-phenyl-L-phenylalaninamide;
N-{(1R,5S)-5-[[(4-aminophenyl)sulfonyl](3-methylbutyl)amino]-1-cyclopropyl-6-hydroxyhexyl}-4-fluoro-β-(4-fluorophenyl)-Nα-(methoxycarbonyl)-L-phenylalaninamide;
N-{(1R,5S)-5-[(1,3-benzothiazol-6-ylsulfonyl)(3-methylbutyl)amino]-1-cyclopropyl-6-hydroxyhexyl}-2-chloro-Nα-(methoxycarbonyl)-L-phenylalaninamide;
methyl [(1S)-2-({(1R,5S)-5-[[(4-aminophenyl)sulfonyl](3-methylbutyl)amino]-1-cyclopropyl-6-hydroxyhexyl}amino)-1-(1-naphthylmethyl)-2-oxo ethyl]carbamate;
N-{(1S,5S)-5-[[(4-aminophenyl)sulfonyl](isopropyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](propyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](3-methylbutyl)amino]-1-ethyl-6-hydroxyhexyl}-2-bromo-Nα-(methoxycarbonyl)-L-phenylalaninamide;
N-{(1S,5S)-5-[[(4-aminophenyl)sulfonyl](3-fluoropropyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](propyl)amino]-1-ethyl-6-hydroxyhexyl}-Nα-(methoxycarbonyl)-β-phenyl-L-phenylalaninamide;
methyl [2-({(1S,5S)-5-[[(4-aminophenyl)sulfonyl](3-methylbutyl)amino]-1-ethyl-6-hydroxyhexyl}amino)-1-(5H-dibenzo[a,d][7]annulen-5-yl)-2-oxo ethyl]carbamate;
N-{(1S,5S)-5-[[(4-aminophenyl)sulfonyl](propyl)amino]-1-ethyl-6-hydroxyhexyl}-2-bromo-Nα-(methoxycarbonyl)-L-phenylalaninamide;
N-{(1S,5S)-5-[[(4-aminophenyl)sulfonyl](propyl)amino]-1-ethyl-6-hydroxyhexyl}-2-chloro-Nα-(methoxycarbonyl)-L-phenylalaninamide;
tert-butyl {(1R,2R)-1-[({(1S,5S)-5-[[(4-aminophenyl)sulfonyl](3-methylbutyl)amino]-1-ethyl-6-hydroxyhexyl}amino)carbonyl]-2-phenylcyclopropyl}carbamate;
N-{(1S,5S)-5-[[(4-aminophenyl)sulfonyl](3-methylbutyl)amino]-1-ethyl-6-hydroxyhexyl}-β-phenyl-Nα-[(pyridin-4-ylmethoxy)carbonyl]-L-phenylalaninamide;
methyl [2-({(1S,5S)-5-[[(4-aminophenyl)sulfonyl](3-methylbutyl)amino]-1-ethyl-6-hydroxyhexyl}amino)-2-oxo-1-(9H-xanthen-9-yl)ethyl]carbamate;
N-{(5S)-5-[[(4-aminophenyl)sulfonyl](3-methylbutyl)amino]-1-ethyl-6-hydroxyhexyl}-Nα-(methoxycarbonyl)-Nα-methyl-β-phenyl-L-phenylalaninamide;
N-{(1S,5S)-5-[[(4-aminophenyl)sulfonyl](3-methylbutyl)amino]-1-ethyl-6-hydroxyhexyl}-β-phenyl-L-phenylalaninamide;
N-{(1R,5S)-5-[[(4-aminophenyl)sulfonyl](isopropyl)amino]-6-hydroxy-1-isopropylhexyl}-Nα-(methoxycarbonyl)-β-phenyl-L-phenylalaninamide;
N-{(1S,5S)-5-[[(4-aminophenyl)sulfonyl](isopropyl)amino]-6-hydroxy-1-methylhexyl}-3-fluoro-β-(3-fluorophenyl)-Nα-(methoxycarbonyl)-L-phenylalaninamide;
N-{(1S,5S)-5-[[(4-aminophenyl)sulfonyl](isopropyl)amino]-6-hydroxy-1-methylhexyl}-2,3-dichloro-Nα-(methoxycarbonyl)-L-phenylalaninamide;
N-{(1S,5S)-5-[[(4-aminophenyl)sulfonyl](propyl)amino]-6-hydroxy-1-methylhexyl}-3-fluoro-β-(3-fluorophenyl)-Nα-(methoxycarbonyl)-L-phenylalaninamide;
N-[(1S,5S)-5-(ethyl{[4-(hydroxymethyl)phenyl]sulfonyl}amino)-6-hydroxy-1-methylhexyl]-Nα-(methoxycarbonyl)-β-phenyl-L-phenylalaninamide;
2-chloro-N-{(1S,5S)-6-hydroxy-5-[{[4-(hydroxymethyl)phenyl]sulfonyl}(isobutyl)amino]-1-methylhexyl}-Nα-(methoxycarbonyl)-L-phenylalaninamide;
N-{(1R,5S)-5-[(1,3-benzothiazol-6-ylsulfonyl)(3-methylbutyl)amino]-1-cyclopropyl-6-hydroxyhexyl}-Nα-(methoxycarbonyl)-β-phenyl-L-phenylalaninamide;
N-{(1S,5S)-5-[[(4-aminophenyl)sulfonyl](methyl)amino]-6-hydroxy-1-methylhexyl}-Nα-(methoxycarbonyl)-β-phenyl-L-phenylalaninamide;
N-{(1S,5S)-5-[[(4-aminophenyl)sulfonyl](ethyl)amino]-6-hydroxy-1-methylhexyl}-Nα-(methoxycarbonyl)-β-phenyl-L-phenylalaninamide;
N-{(1S,5S)-6-hydroxy-5-[{[4-(hydroxymethyl)phenyl]sulfonyl}(methyl)amino]-1-methylhexyl}-Nα-(methoxycarbonyl)-β-phenyl-L-phenylalaninamide;
N-{(1S,5S)-5-[[(4-aminophenyl)sulfonyl](cyclopropylmethyl)amino]-6-hydroxy-1-methylhexyl}-Nα-(methoxycarbonyl)-β-phenyl-L-phenylalaninamide;
2-chloro-N-{(1S,5S)-6-hydroxy-5-[{[4-(hydroxymethyl)phenyl]sulfonyl}(methyl)amino]-1-methylhexyl}-Nα-(methoxycarbonyl)-L-phenylalaninamide;
2-chloro-N-[(1S,5S)-5-(ethyl{[4-(hydroxymethyl)phenyl]sulfonyl}amino)-6-hydroxy-1-methylhexyl]-Nα-(methoxycarbonyl)-L-phenylalaninamide;
2-chloro-N-{(1S,5S)-6-hydroxy-5-[{[4-(hydroxymethyl)phenyl]sulfonyl}(isopropyl)amino]-1-methylhexyl}-Nα-(methoxycarbonyl)-L-phenylalaninamide;
N-{(1S,5S)-5-[[(4-aminophenyl)sulfonyl](isopropyl)amino]-6-hydroxy-1-methylhexyl}-2-chloro-Nα-(methoxycarbonyl)-L-phenylalaninamide;
2-bromo-N-{(1S,5S)-6-hydroxy-5-[{[4-(hydroxymethyl)phenyl]sulfonyl}(isobutyl)amino]-1-methylhexyl}-Nα-(methoxycarbonyl)-L-phenylalaninamide;
2-bromo-N-{(1S,5S)-6-hydroxy-5-[{[4-(hydroxymethyl)phenyl]sulfonyl}(isopropyl)amino]-1-methylhexyl}-Nα-(methoxycarbonyl)-L-phenylalaninamide;
N-[(1S,5S)-((3-fluoropropyl){[4-(hydroxymethyl)phenyl]sulfonyl}amino)-6-hydroxy-1-methylhexyl]-Nα-(methoxycarbonyl)-β-phenyl-L-phenylalaninamide;
N-{(1S,5S)-5-[[(4-aminophenyl)sulfonyl](isopropyl)amino]-6-hydroxy-1-methylhexyl}-2-bromo-Nα-(methoxycarbonyl)-L-phenylalaninamide;
N-{(1S,5S)-6-hydroxy-5-[{[4-(hydroxymethyl)phenyl]sulfonyl}(isobutyl)amino]-1-methylhexyl}-Nα-(methoxycarbonyl)-β-phenyl-L-phenylalaninamide;
N-[(1S,5S)-5-(cyclobutyl{[4-(hydroxymethyl)phenyl]sulfonyl}amino)-6-hydroxy-1-methylhexyl]-Nα-(methoxycarbonyl)-β-phenyl-L-phenylalaninamide;
N-{(1S,5S)-5-[[(4-aminophenyl)sulfonyl](isopropyl)amino]-1-ethyl-6-hydroxyhexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(isopropyl)amino]hexyl}-Nα-(methoxycarbonyl)-β-phenyl-L-phenylalaninamide;
N-{(1S,5S)-6-hydroxy-5-[{[4-(hydroxymethyl)phenyl]sulfonyl}(propyl)amino]-1-methylhexyl}-Nα-(methoxycarbonyl)-β-phenyl-L-phenylalaninamide;
N-[(1R,5S)-5-[[(4-aminophenyl)sulfonyl](propyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](3-methylbutyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](isopropyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](ethyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](isobutyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](methyl)amino]-6-hydroxy-1-(trifluoromethyl)hexyl]-Nα-(methoxycarbonyl)-β-phenyl-L-phenylalaninamide;
N-{(1S,5S)-5-[[(4-aminophenyl)sulfonyl](isopropyl)amino]-6-hydroxy-1-methylhexyl}-Nα-methyl-β-phenyl-L-phenylalaninamide;
2-chloro-N-{(1R,5S)-1-cyclopropyl-6-hydroxy-5-[{[4-(hydroxymethyl)phenyl]sulfonyl}-(isobutyl)amino]hexyl}-Nα-(methoxycarbonyl)-L-phenylalaninamide;
2-chloro-N-{(1R,5S)-1-cyclopropyl-6-hydroxy-5-[{[4-(hydroxymethyl)phenyl]sulfonyl}(isopropyl)amino]hexyl}-Nα-(methoxycarbonyl)-L-phenylalaninamide;
N-{(1R,5S)-1-cyclopropyl-6-hydroxy-5-[{[4-(hydroxymethyl)phenyl]sulfonyl}(isopropyl)amino]hexyl}-Nα-(methoxycarbonyl)-β-phenyl-L-phenylalaninamide;
N-{(1R,5S)-1-cyclopropyl-6-hydroxy-5-[{[4-(hydroxymethyl)phenyl]sulfonyl}(propyl)amino]hexyl}-Nα-(methoxycarbonyl)-β-phenyl-L-phenylalaninamide;
N-[(1R,5S)-1-cyclopropyl-5-(ethyl{[4-(hydroxymethyl)phenyl]sulfonyl}amino)-6-hydroxyhexyl]-Nα-(methoxycarbonyl)-β-phenyl-L-phenylalaninamide;
N-{(1R,5S)-1-cyclopropyl-6-hydroxy-5-[{[4-(hydroxymethyl)phenyl]sulfonyl}(methyl)amino]hexyl}-Nα-(methoxycarbonyl)-β-phenyl-L-phenylalaninamide;
N-{(1R,5S)-5-[[(4-aminophenyl)sulfonyl](propyl)amino]-1-cyclopropyl-6-hydroxyhexyl}-Nα-(methoxycarbonyl)-β-phenyl-L-phenylalaninamide;
N-{(1R,5S)-5-[[(4-aminophenyl)sulfonyl](isopropyl)amino]-1-cyclopropyl-6-hydroxyhexyl}-Nα-(methoxycarbonyl)-β-phenyl-L-phenylalaninamide;
N-{(1R,5S)-5-[[(4-aminophenyl)sulfonyl](methyl)amino]-1-cyclopropyl-6-hydroxyhexyl}-Nα-(methoxycarbonyl)-β-phenyl-L-phenylalaninamide;
N-{(1R,5S)-5-[[(4-aminophenyl)sulfonyl](ethyl)amino]-1-cyclopropyl-6-hydroxyhexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide;
N-[(1R,5S)-6-hydroxy-5-[{[4-(hydroxymethyl)phenyl]sulfonyl}(3-methylbutyl)amino]-1-(trifluoromethyl)hexyl]-Nα-(methoxycarbonyl)-β-phenyl-L-phenylalaninamide;
N-{(1R,5S)-1-cyclopropyl-6-hydroxy-5-[({4-[(1S)-1-hydroxyethyl]phenyl}sulfonyl)(3-methylbutyl)amino]hexyl}-4-fluoro-β-(4-fluorophenyl)-Nα-(methoxycarbonyl)-L-phenylalaninamide;
N-{(1S,5S)-6-hydroxy-5-[{[4-(hydroxymethyl)phenyl]sulfonyl}(3-methylbutyl)amino]-1-methylhexyl}-Nα-(methoxycarbonyl)-β-phenyl-L-phenylalaninamide;
N-{(1R,5S)-6-hydroxy-5-[{[4-(hydroxymethyl)phenyl]sulfonyl}(3-methylbutyl)amino]-1-methylhexyl}-Nα-(methoxycarbonyl)-β-phenyl-L-phenylalaninamide;
N-{(1R,5S)-1-tert-butyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide;
N-[(1S,5S)-6-hydroxy-5-[{[4-(hydroxymethyl)phenyl]sulfonyl}(3-methylbutyl)amino]-1-(trifluoromethyl)hexyl]-Nα-(methoxycarbonyl)-β-phenyl-L-phenylalaninamide;
N-{(1R,5S)-1-cyclopropyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide;
N-{(1R,5S)-6-hydroxy-5-[{[4-(hydroxymethyl)phenyl]sulfonyl}(3-methylbutyl)amino]-1-isopropylhexyl}-Nα-(methoxycarbonyl)-β-phenyl-L-phenylalaninamide;
N-{(1R,5S)-6-hydroxy-5-[{[4-(hydroxymethyl)phenyl]sulfonyl}(3-methylbutyl)amino]-1-vinylhexyl}-Nα-(methoxycarbonyl)-β-phenyl-L-phenylalaninamide;
N-{(1S,5S)-6-hydroxy-5-[{[4-(hydroxymethyl)phenyl]sulfonyl}(3-methylbutyl)amino]-1-vinylhexyl}-Nα-(methoxycarbonyl)-β-phenyl-L-phenylalaninamide;
N-[(1R,5S)-6-hydroxy-5-{[4-(hydroxymethyl)phenyl]sulfonyl}(3-methylbutyl)amino]-1-(pentafluoroethyl)hexyl]-Nα-(methoxycarbonyl)-β-phenyl-L-phenylalaninamide;
N-{(1R,5S)-1-ethynyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide;
2-chloro-N-{(1R,5S)-1-cyclopropyl-6-hydroxy-5-[{[4-(hydroxymethyl)phenyl]sulfonyl}(3-methylbutyl)amino]hexyl}-Nα-(methoxycarbonyl)-L-phenylalaninamide;
2-chloro-N-{(1R,5S)-1-cyclopropyl-6-hydroxy-5-[({4-[(1S)-1-hydroxyethyl]phenyl}sulfonyl)(3-methylbutyl)amino]hexyl}-Nα-(methoxycarbonyl)-L-phenylalaninamide;
N-{(1R,5S)-1-cyclopropyl-6-hydroxy-5-[{[4-(hydroxymethyl)phenyl]sulfonyl}(3-methylbutyl)amino]hexyl}-4-fluoro-β-(4-fluorophenyl)-Nα-(methoxycarbonyl)-L-phenylalaninamide;
N-{(1R,5S)-1-cyclopropyl-6-hydroxy-5-[{[4-(hydroxymethyl)phenyl]sulfonyl}(3-methylbutyl)amino]hexyl}-4-fluoro-β-(4-fluorophenyl)-L-phenylalaninamide;
N-{(1R,5S)-1-cyclopropyl-6-hydroxy-5-[({4-[(1S)-1-hydroxyethyl]phenyl}sulfonyl)(3-methylbutyl)amino]hexyl}-4-fluoro-β-(4-fluorophenyl)-L-phenylalaninamide;
N-{(1S,5S)-1-ethyl-6-hydroxy-5-[{[4-(hydroxymethyl)phenyl]sulfonyl}(isobutyl)amino]hexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(isobutyl)amino]hexyl}-4-fluoro-β-(4-fluorophenyl)-Nα-(methoxycarbonyl)-L-phenylalaninamide;
2-chloro-N-{(1S,5S)-1-ethyl-6-hydroxy-5-[{[4-(hydroxymethyl)phenyl]sulfonyl}(isobutyl)amino]hexyl}-Nα-(methoxycarbonyl)-L-phenylalaninamide;
2-chloro-N-{(1S,5S)-1-ethyl-6-hydroxy-5-[{[4-(hydroxymethyl)phenyl]sulfonyl}(isobutyl)amino]hexyl}-Nα-(methoxycarbonyl)-L-phenylalaninamide;
N-{(1R,5S)-1-cyclopropyl-6-hydroxy-5-[{[4-(hydroxymethyl)phenyl]sulfonyl}(isobutyl)amino]hexyl}-4-fluoro-β-(4-fluorophenyl)-Nα-(methoxycarbonyl)-L-phenylalaninamide;
N-{(1S,5S)-1-ethyl-6-hydroxy-5-[{[4-(hydroxymethyl)phenyl]sulfonyl}(isobutyl)amino]hexyl}-Nα-methyl-β-phenyl-L-phenylalaninamide;
N-{(1R,5S)-1-cyclopropyl-6-hydroxy-5-[{[4-(hydroxymethyl)phenyl]sulfonyl}(isobutyl)amino]hexyl}-Nα-methyl-β-phenyl-L-phenylalaninamide;
2-chloro-N-{(1R,5S)-6-hydroxy-5-[{[4-(hydroxymethyl)phenyl]sulfonyl}(3-methylbutyl)amino]-1-isopropylhexyl}-Nα-(methoxycarbonyl)-L-phenylalaninamide;
N-{(1S,5S)-1-ethyl-6-hydroxy-5-[{[4-(hydroxymethyl)phenyl]sulfonyl}(3-methylbutyl)amino]hexyl}-Nα-methyl-β-phenyl-L-phenylalaninamide;
N-{(1R,5S)-1-cyclopropyl-6-hydroxy-5-[{[4-(hydroxymethyl)phenyl]sulfonyl}(3-methylbutyl)amino]hexyl}-Nα-methyl-β-phenyl-L-phenylalaninamide;
N-{(1R,5S)-6-hydroxy-5-[{[4-(hydroxymethyl)phenyl]sulfonyl}(3-methylbutyl)amino]-1-isopropylhexyl}-Nα-methyl-β-phenyl-L-phenylalaninamide;
N-[(1R,5S)-6-hydroxy-5-[{[4-(hydroxymethyl)phenyl]sulfonyl}(isobutyl)amino]-1-(trifluoromethyl)hexyl]-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(3-methylbutyl)amino]hexyl}-β-phenyl-L-phenylalaninamide;
N-{(1S,5S)-1-ethyl-6-hydroxy-5-[{[4-(hydroxymethyl)phenyl]sulfonyl}(isobutyl)amino]hexyl}-β-phenyl-L-phenylalaninamide;
N-{(5S)-6-hydroxy-5-[{[4-(hydroxymethyl)phenyl]sulfonyl}(propyl)amino]-1-methylhexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(propyl)amino]hexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(isopropyl)amino]hexyl}-Nα-methyl-β-phenyl-L-phenylalaninamide;
N-{(1S,5S)-1-ethyl-6-hydroxy-5-[{[4-(hydroxymethyl)phenyl]sulfonyl}(isopropyl)amino]hexyl}-β-phenyl-L-phenylalaninamide;
2-bromo-N-{(1R,5S)-1-cyclopropyl-6-hydroxy-5-[{[4-(hydroxymethyl)phenyl]sulfonyl}(isopropyl)amino]hexyl}-Nα-(methoxycarbonyl)-L-phenylalaninamide;
N-{(1S,5S)-1-ethyl-6-hydroxy-5-[{[4-(hydroxymethyl)phenyl]sulfonyl}(propyl)amino]hexyl}-Nα-methyl-β-phenyl-L-phenylalaninamide;
N-{(1S,5S)-1-ethyl-6-hydroxy-5-[{[4-(hydroxymethyl)phenyl]sulfonyl}(propyl)amino]hexyl}-β-phenyl-L-phenylalaninamide;
N-[(1R,5S)-6-hydroxy-5-[{[4-(hydroxymethyl)phenyl]sulfonyl}(isopropyl)amino]-1-(trifluoromethyl)hexyl]-Nα-(methoxycarbonyl)-β-phenyl-L-phenylalaninamide;
methyl [(1S)-2-({(1S,5S)-1-ethyl-6-hydroxy-5-[{[4-(hydroxymethyl)phenyl]sulfonyl}(propyl)amino]hexyl}amino)-1-(1-naphthylmethyl)-2-oxoethyl]carbamate;
N-[(1R,5S)-6-hydroxy-5-{[4-(hydroxymethyl)phenyl]sulfonyl}(propyl)amino]-1-(trifluoromethyl)hexyl]-Nα-(methoxycarbonyl)-β-phenyl-L-phenylalaninamide;
2-chloro-N-[(1R,5S)-6-hydroxy-5-[{[4-(hydroxymethyl)phenyl]sulfonyl}(propyl)amino]-1-(trifluoromethyl)hexyl]-Nα-(methoxycarbonyl)-L-phenylalaninamide;
4-chloro-β-(4-chlorophenyl)-N-{(1S,5S)-1-ethyl-6-hydroxy-5-[{[4-(hydroxymethyl)phenyl]sulfonyl}(3-methylbutyl)amino]hexyl}-Nα-(methoxycarbonyl)-L-phenylalaninamide;
2,3-dichloro-N-{(1S,5S)-6-hydroxy-5-[{[4-(hydroxymethyl)phenyl]sulfonyl}(isopropyl)amino]-1-methylhexyl}-Nα-(methoxycarbonyl)phenylalaninamide;
3-fluoro-β-(3-fluorophenyl)-N-{(1S,5S)-6-hydroxy-5-[{[4-(hydroxymethyl)phenyl]sulfonyl}(isopropyl)amino]-1-methylhexyl}-Nα-(methoxycarbonyl)phenylalaninamide;
N-{(1S,5S)-6-hydroxy-5-[{[4-(hydroxymethyl)phenyl]sulfonyl}(isopropyl)amino]-1-methylhexyl}-Nα-(methoxycarbonyl)-β-phenyl-L-phenylalaninamide;
N-{(1S,5S)-1-ethyl-6-hydroxy-5-[({4-[(1S)-1-hydroxyethyl]phenyl}sulfonyl)(isopropyl)-amino]hexyl}-Nα-(methoxycarbonyl)-β-phenyl-L-phenylalaninamide;
N-{(1S,5S)-5-[[(4-acetylphenyl)sulfonyl](isopropyl)amino]-1-ethyl-6-hydroxyhexyl}-Nα-(methoxycarbonyl)-β-phenyl-L-phenylalaninamide;
N-{(1S,5S)-6-hydroxy-5-[({-4-[(1S)-1-hydroxyethyl]phenyl}sulfonyl)(isopropyl)amino]-1-methylhexyl}-Nα-(methoxycarbonyl)-β-phenyl-L-phenylalaninamide;
N-{(1S,5S)-5-[[(4-acetylphenyl)sulfonyl](isopropyl)amino]-6-hydroxy-1-methylhexyl}-Nα-(methoxycarbonyl)-β-phenyl-L-phenylalaninamide;
N-(1-{(4S)-4-[[(4-aminophenyl)sulfonyl](3-methylbutyl)amino]-5-hydroxypentyl}cyclopentyl)-Nα-(methoxycarbonyl)-β-phenyl-L-phenylalaninamide;
N-{(5S)-5-[[(4-aminophenyl)sulfonyl](isopropyl)amino]-6-hydroxy-1,1-dimethylhexyl}-β-phenyl-L-phenylalaninamide;
N-(1-{4-[[(4-aminophenyl)sulfonyl](3-methylbutyl)amino]-5-hydroxypentyl}cyclobutyl)-Nα-(methoxycarbonyl)-β-phenyl-L-phenylalaninamide;
N-{(5S)-5-[[(4-aminophenyl)sulfonyl](3-methylbutyl)amino]-6-hydroxy-1,1-dimethylhexyl}-Nα-(methoxycarbonyl)-β-phenyl-L-phenylalaninamide;
N-{(5S)-5-[[(4-aminophenyl)sulfonyl](isobutyl)amino]-6-hydroxy-1,1-dimethylhexyl}-Nα-(methoxycarbonyl)-β-phenyl-L-phenylalaninamide;
N-{(5S)-5-[[(4-aminophenyl)sulfonyl](propyl)amino]-6-hydroxy-1,1-dimethylhexyl}-Nα-(methoxycarbonyl)-β-phenyl-L-phenylalaninamide;
N-{(5S)-5-[[(4-aminophenyl)sulfonyl](3-fluoropropyl)amino]-6-hydroxy-1,1-dimethylhexyl}-Nα-(methoxycarbonyl)-β-phenyl-L-phenylalaninamide;
N-{(5S)-5-[[(4-aminophenyl)sulfonyl](isopropyl)amino]-6-hydroxy-1,1-dimethylhexyl}-Nα-(methoxycarbonyl)-β-phenyl-L-phenylalaninamide;
N-{(5S)-5-[[(4-aminophenyl)sulfonyl](isopropyl)amino]-6-hydroxy-1,1-dimethylhexyl}-Nα-methyl-β-phenyl-L-phenylalaninamide;
N-{(5S)-5-[[(4-aminophenyl)sulfonyl](isopropyl)amino]-6-hydroxy-1,1-dimethylhexyl}-2-chloro-L-phenylalaninamide;
N-[5-[[(4-aminophenyl)sulfonyl](3-methylbutyl)amino]-6-hydroxy-1-(hydroxymethyl)hexyl]-Nα-(methoxycarbonyl)-β-phenyl-L-phenylalaninamide;
and pharmaceutically acceptable salts thereof.

18. The compound according to claim 17, which is selected from the group consisting of:
N-{(1S,5S)-5-[[(4-aminophenyl)sulfonyl](isopropyl)amino]-6-hydroxy-1-methylhexyl}-Nα-(methoxycarbonyl)-β-phenyl-L-phenylalaninamide;
methyl [(1S)-2-({(5S)-5-[[4-aminophenyl)sulfonyl]-((3S)-3-ethylbutyl)amino]-6-hydroxy-1-methylhexyl)amino)-1-(diphenylmethyl)-2-oxo ethyl]carbamate;
N-{(1S,5S)-5-[[(4-aminophenyl)sulfonyl](propyl)amino]-6-hydroxy-1-methylhexyl}-Nα-(methoxycarbonyl)-β-phenyl-L-phenylalaninamide;
N-{(5S)-5-[[(4-aminophenyl)sulfonyl](isopropyl)amino]-6-hydroxy-1,1-dimethylhexyl}-β-phenyl-L-phenylalaninamide;
methyl [(1S)-2-({(5S)-5-[[3-fluoro-4-aminophenyl)sulfonyl]-((3S)-3-cyclopropylbutyl)amino]-6-hydroxy-1-methylhexyl)amino)-1-(diphenylmethyl)-2-oxoethyl]carbamate;
N-[(1R,5S)-5-[[(4-aminophenyl)sulfonyl](propyl)amino]-6-hydroxy-1-(trifluoromethyl)hexyl]-Nα-(methoxycarbonyl)-β-phenyl-L-phenylalaninamide;
N-[(1R,5S)-5-[[(4-aminophenyl)sulfonyl](isopropyl)amino]-6-hydroxy-1-(trifluoromethyl)hexyl]-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(3-methylbutyl)amino]hexyl}-Nα-(methoxycarbonyl)-β-phenyl-L-phenylalaninamide;
N-{(1S,5S)-1-ethyl-6-hydroxy-5-[{[4-(hydroxymethyl)phenyl]sulfonyl}(3-methylbutyl)amino]hexyl}-Nα-methyl-β-phenyl-L-phenylalaninamide;
N-{(1S,5S)-6-hydroxy-5-[{[4-(hydroxymethyl)phenyl]sulfonyl}(isopropyl)amino]-1-methylhexyl}-Nα-(methoxycarbonyl)-β-phenyl-L-phenylalaninamide;
N-{(1S,5S)-6-hydroxy-5-[({-4-(1S)-1-hydroxyethyl]phenyl}sulfonyl)(isopropyl)amino]-1-methylhexyl}-Nα-(methoxycarbonyl)-β-phenyl-L-phenylalaninamide;
and pharmaceutically acceptable salts thereof.

19. A pharmaceutical composition comprising an effective amount of a compound according to claim 1, or a pharmaceutically acceptable salt thereof, and a pharmaceutically acceptable carrier.

20. A method for the treatment or prophylaxis of infection by HIV or for the treatment, prophylaxis, or delay in the onset of AIDS in a subject in need thereof, which comprises administering to the subject an effective amount of the compound according to claim 1 or a pharmaceutically acceptable salt thereof.

21. (canceled)
